(19) United States
(12) Patent Application Publication
(10) Pub. No.: US 2009/0105259 A1
(43) Pub. Date: Apr. 23, 2009

(54) Acyclic 1,4-Diamines and Uses Thereof

(76) Inventors: Jae U. Jeong, Collegeville, PA (US); Robert W. Marquis, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/065,772

(22) PCT Filed: Sep. 8, 2006
(86) PCT No.: PCT/US06/35156

§ 371 Date: Mar. 5, 2008

Related U.S. Application Data
(60) Provisional application No. 60/715,085, filed on Sep. 8, 2005.

(51) Int. Cl. A61K 31/381; C07D 471/04; C07D 487/04; C07D 333/52; A61K 31/5375; A61P 19/02; C07D 265/30; A61K 31/4355; A61K 31/5025

(52) U.S. Cl. 514/238.2; 546/115; 544/236; 549/57; 544/159; 514/302; 514/248; 514/443

(57) ABSTRACT

This invention relates to novel compounds useful in the treatment of diseases associated with TRPV4 channel receptor. More specifically, this invention relates to certain acyclic diamines, which are agonists of TRPV4 channel receptors.

### FIELD OF THE INVENTION

This invention relates to novel compounds useful in the treatment of diseases associated with TRPV4 channel receptor. More specifically, this invention relates to certain acyclic diamines, which are agonists of TRPV4 channel receptors.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain). TRPV4 channel receptor is one of six known members of the vanilloid family of transient receptor potential channels and shares 51% identity at the nucleotide level with TRPV1, the capsaicin receptor. Examples of polypeptides and polynucleotides encoding forms of human vanilloid receptors, including TRPV4 channel receptor from human can be found in EP 1170365 as well as WO 00/32766. Like the other family members TRPV4 channel receptor is a Ca2+ permeable, non-selective, ligand-gated cation channel, which responds to diverse stimuli such as reduced osmolality, elevated temperature, and small molecule ligands, See, for instance, Voets, et al., J. Biol. Chem. (2002) 277 33704-47051; Watanabe, et al., J. Biol. Chem. (2002) 277:47044-47051; Watanabe, et al., J. Bio. Chem. (2002) 277: 13569-47051; Xu, et al., J. Biol. Chem. (2003) 278:11520-11527.

From a screen of body tissues, the human TRPV4 channel receptor is most prominently expressed in cartilage. A screen of primary and clonal cell cultures shows significant expression only in chondrocytes.

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes results in the degradation of the cartilage matrix. Aggrecanases (ADAMTSs), in conjunction with MMPs, degrade aggrecan, an aggregating proteoglycan present in articular cartilage. In osteoarthritic (OA) articular cartilage, a loss of proteoglycan staining is observed in the superficial zone in early OA and adjacent to areas of cartilage erosion in moderate to severe OA. The reduction in proteoglycan content is associated with an increase in degradation of type II collagen by specialized MMPs, termed collagenases (e.g. MMP-13). Collagenases are believed to make the initial cleavage within the triple-helix of intact collagen. It is hypothesized that the initial cleavage of collagen by collagenases facilitates the further degradation of the collagen fibrils by other proteases; accordingly, preventing or reducing the increased production of matrix degrading enzymes and/or attenuating the inhibition of matrix production may also promote functional recovery. Modulation of TRPV4 channel receptor has been shown to play a role in attenuating cartilage breakdown and matrix degrading enzymes. See PCT Publication No. WO2006/029,209.

Excessive degradation of extra cellular matrix is implicated in the pathogenesis of many diseases, including chronic, neuropathic, and postoperative pain; rheumatoid arthritis; osteoarthritis; neuralgia; neuropathies; algesia; nerve injury; ischaemia; neurodegeneration; cartilage degeneration; stroke; incontinence; inflammatory disorders; irritable bowel syndrome; obesity; periodontal disease; aberrant angiogenesis; tumor invasion and metastasis; corneal ulceration; and complications of diabetes.

Thus, there is a need to discover new compounds useful in modulating TRPV4 channel receptors.

### SUMMARY OF THE INVENTION

This invention comprises a class of acyclic 1,4-diamines that are useful in the treatment of diseases associated with TRPV4 channel receptors. This invention is also a pharmaceutical composition comprising acyclic 1,4-diamines according to formula (I) and a pharmaceutically acceptable carrier. This invention is also a method of treating diseases associated with TRPV4 channel receptor in mammals, particularly in humans.

Specifically, the invention is directed to compounds according to Formula I:

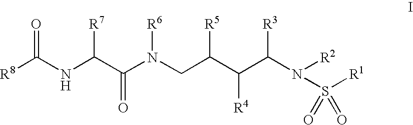

or pharmaceutically acceptable salts, hydrates, or solvates thereof, wherein:
R¹ is aryl optionally substituted with CN, NO₂, halogen, CH₃, CF₃ or H;
R² is H, C₁-C₆ alkyl, C₃-C₇ cycloalkyl, or C₃-C₇ heterocycloalkyl;
R³ is H, OH, C₁-C₆ OH, O—C₁-C₆ alkyl, CO₂CH₃, CONHCH₃, SH, S—C₁-C₆ alkyl, or F;
R⁴ is H, OH, C₁-C₆ OH, O—C₁-C₆ alkyl, SH, S—C₁-C₆ alkyl, or F;
R⁵ is H, OH, C₁-C₆ OH, O—C₁-C₆ alkyl, SH, S—C₁-C₆ alkyl, or F;
R⁶ is H or C₁-C₆ alkyl;
R⁷ is optionally substituted C₁-C₆ alkyl, O—C₁-C₆ alkyl, C—S—C₁-C₆ alkyl cyclohexylmethyl, amide, urea, or cyclopentylmethyl; and
R⁸ is optionally substituted C₃₋₇cycloalkyl, optionally substituted C₃₋₇cycloalkenyl, optionally substituted Het-C₃₋₇alkyl, optionally substituted Het-C₃₋₇alkenyl, optionally substituted aryl, optionally substituted aryloxy; optionally substituted arylamino; optionally substituted heterocycloalkyl, optionally substituted heteroaryl, optionally substituted cylcoalkyl, or optionally substituted indenyl.

In addition, the invention is directed to compounds according to Formula II

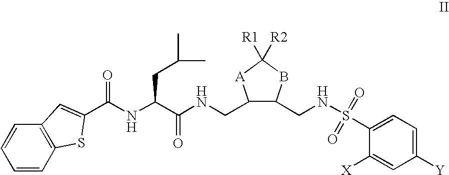

and pharmaceutically acceptable salts, hydrates, or solvates thereof, wherein:

### R¹ is H or CH₃;

### R² is H or CH₃;

### A is C or O;

### B is C or O;

### X is H, Cl or F; and

### Y is H, Cl or F.

The invention is also directed to compounds according Formula III

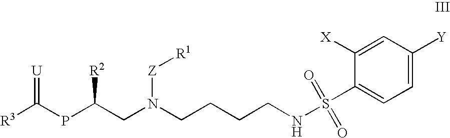

and pharmaceutically acceptable salts, hydrates, or solvates thereof, wherein:

### X is H, C₁, CF₃, NO₂, or CN;

### Y is H, Cl, or F;

### Z is C═S, C═O or O═S═O;

### U is O or S;

R¹ is optionally substituted cyloalkyl, C₁-C₁₂ alkyl, C₁-C₁₂ alkoxy, C₁-C₁₂ alkylamino, optionally substituted aryl, optionally substituted arylamino, optionally substituted heteroaryl; or optionally substituted heterocycloalkyl;
R² is H, C₁-C₁₂ alkyl, C₁-C₁₂ alkoxy, optionally substituted heterocycloalkylamino, optionally substituted heteroaryl, or optionally substituted aryl;

### P is NH or O; and

R³ is C₁-C₁₂ alkylamino, cycloalkylamino, optionally substituted aryl amino, optionally substituted heteroarylamino, heterocyclicalkyl, or optionally substituted aryloxy;
wherein when P is NH, R² may form a five or six member heterocyclic ring with P forming a piperidinyl or pyrrolidinyl group.

### DETAILED DESCRIPTION OF THE INVENTION

Abbreviations and symbols utilized herein are in accordance with the common usage of such abbreviations and symbols by those skilled in the chemical arts. For example, “EDC” rmeans N-ethyl-N′(dimethylaminopropyl)-carbodiimide, “HOOBt” refers to hydroxy-3,4-dihydroxy-4-oxo-1,2,3-benzotriazine, “DMF” means dimethyl formamide, “DMSO” means dimethyl sulfoxide, “TEA” means triethylamine, “NMM” means N-methylmorpholine, “HOBT” means 1-hydroxybenzotriazole and “THF” means tetrahydrofuran.

### TERMS AND DEFINITIONS

As used herein, “acyclic 1,4-diamines” refer to compounds having two nitrogen atoms separated by four optionally substituted atoms, more commonly, four carbon atoms. By way of example, the following fragments constitute acyclic 1,4-diamines:

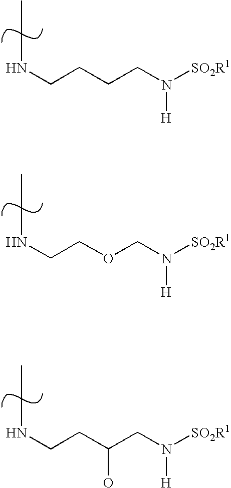

The term “C₁-C₆ alkyl” is used herein to refer to a substituted or unsubstituted, straight or branched chain radical of 1 to 6 carbon atoms, including, methyl, ethyl, n-propyl, isopropyl, n-butyl, isobutyl, t-butyl, pentyl, n-pentyl, isopentyl, neopentyl, and n-hexyl and isomers thereof; (similarly, C₁-C₄ alkyl means a radical of 1 to 4 carbon atoms). Similarly, the term “C₃-C₇ cycloalkyl” is used herein to a substituted or unsubstituted saturated monovalent cyclic ring of 3 to 7 carbon atoms, including cyclopropyl, cyclobutyl, cyclopentyl, cyclohexyl, and cycloheptyl.

“Aryl” or “Ar”, unless otherwise defined, means phenyl or naphthyl. Aryl groups may be optionally substituted with up to five groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl; (C₁₋₄)alkylsulfonyl; (C₂₋₄)alkenylsulfonyl; or aminosulfonyl wherein the amino group is optionally substituted by (C₁₋₄)alkyl or (C₂₋₄)alkenyl; phenyl, phenyl(C₁₋₄)alkyl or phenyl(C₁₋₄)alkoxy. C₃-C₇ cycloalkyl includes cyclopropyl, cyclobutyl, cyclopentyl, and cyclohexyl.

Unless otherwise defined, suitable substituents for any C₁₋₆ alkyl, and C₃₋₇ cycloalkyl groups includes up to three substituents selected from the group consisting of hydroxy, halo, nitro, cyano, carboxy, amino (wherein amino may be substituted as described hereinabove), amidino, sulfonamido, (C₁₋₆)alkoxy, trifluoromethyl, acyloxy, quanidino, (C₃₋₇)cycloalkyl, aryl, and C₃-C₇ heterocycloalkyl.

The term “C₁-C₆ alkyl” as used herein at all occurrences means a substituted and unsubstituted, straight or branched chain radical of 1 to 6 carbon atoms, unless the chain length is limited thereto (e.g., C₁-C₄ means a radical of 1 to 4 carbon atoms), including, but not limited to methyl, ethyl, n-propyl, isopropyl, n-butyl, isobutyl and t-butyl, pentyl, n-pentyl, isopentyl, neopentyl and hexyl and isomers thereof.

The term “alkoxy” is used herein at all occurrences to mean a straight or branched chain radical of 1 to 6 carbon atoms, unless the chain length is limited thereto, bonded to an oxygen atom, including, but not limited to, methoxy, ethoxy, n-propoxy, isopropoxy, and the like.

The term “C₁-C₆ alkoxy” is used herein at all occurrences to mean a straight or branched chain radical of 1 to 6 carbon atoms, unless the chain length is limited thereto (e.g. C₁-C₄ means a radical of 1 to 4 carbon atoms), bonded to an oxygen atom, including, but not limited to, methoxy, ethoxy, n-propoxy, isopropoxy, and the like.

In the substituents defined herein, the terms “alkyl” and “alkoxy” are also meant to include both monovalent and divalent straight or branched carbon chain radicals. For example, the term “C₁-C₆ hydroxyalkyl” is meant to include a substituent having the bonding arrangement “HO—CH₂-” or “HO—CH₂(CH₃)CHCH₂—” and the term “Ph-C₁-C₆ alkoxy” is meant to include a substituent having the bonding arrangement: “Ph-CH₂—O—” or “Ph-(CH₃)CH—O—”. In contrast, the term “C₀” denotes the absence of an alkyl radical; for instance, in the moiety Ph-C₀-C₈ alkoxy, when C is 0, the substituent can be phenoxy; in the moiety Ph-C₀-C₆ alkyl, when C is 0, the substituent can be phenyl.

The alkyl and alkoxy substituents/moieties as defined herein may be optionally unsubstituted or substituted. If substituents for an alkyl or alkoxy substituent/moiety are not specified, the alkyl or alkoxy substituent/moiety is intended to be unsubstituted.

“Acyl” includes formyl and (C₁₋₆) alkylcarbonyl group.

“Alkyl” refers to a saturated hydrocarbon chain having from 1 to 12 member atoms. Alkyl groups may be optionally substituted with one or more substituents as defined herein. Use of the prefix “C₁₋ₓ” or “C₁-Cₓ” with alkyl refers to an alkyl group having from 1 to x member atoms. For example, C₁₋₆ alkyl refers to an alkyl group having from 1 to 6 member atoms. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl. Unless otherwise defined, the term C₁₋₆alkyl (or alternatively as (C₁₋₆)alkyl) when used alone or when forming part of other groups (such as the ‘alkoxy’ group) includes substituted or unsubstituted, straight or branched chain alkyl groups containing 1 to 6 carbon atoms.

“Alkenyl” refers to an unsaturated hydrocarbon chain having from 2 to 12 member atoms and having one or more carbon-carbon double bond within the chain. In certain embodiments alkenyl groups have one carbon-carbon double bond within the chain. In other embodiments, alkenyl groups have more than one carbon-carbon double bond within the chain. Alkenyl groups may be optionally substituted with one or more substituents as defined herein. Use of the prefix “C₂₋ₓ” or “C₂-Cₓ” with alkenyl refers to an alkenyl group having from 2 to x member atoms. For example, C₂-C₆alkenyl (or (C₂₋₆)alkenyl) refers to an alkenyl group having from 2 to 6 member atoms. Alkenyl groups may be straight or branched. Representative branched alkenyl groups have one, two, or three branches. Alkenyl includes, but is not limited to, ethylenyl, propenyl, butenyl, pentenyl, and hexenyl.

“Alkynyl” refers to an unsaturated hydrocarbon chain having from 2 to 12 member atoms and having one or more carbon-carbon triple bond within the chain. In certain embodiments alkynyl groups have one carbon-carbon triple bond within the chain. In other embodiments, alkynyl groups have more than one carbon-carbon triple bond within the chain. For the sake of clarity, unsaturated hydrocarbon chains having one or more carbon-carbon triple bond within the chain and one or more carbon-carbon double bond within the chain are alkynyl groups. Alkynyl groups may be optionally substituted with one or more substituents as defined herein. Use of the prefix “C₂₋ₓ” or “C₂-Cₓ” with alkynyl refers to an alkynyl group having from 2 to x member atoms. For example, C₂-C₆alkynyl (or (C₂₋₆)alkynyl) refers to an alkynyl group having from 2 to 6 member atoms. Alkynyl groups may be straight or branched. Representative branched alkynyl groups have one, two, or three branches. Alkynyl includes, but is not limited to, ethynyl, propynyl, butynyl, pentynyl, and hexynyl.

“Amino acid” refers to the D- or L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine.

“Aryl” or “Ar” means optionally substituted phenyl or naphthyl. “Cycloalkyl” refers to a saturated hydrocarbon ring having from 3 to 7 member atoms. Cycloalkyl groups are monocyclic ring systems. Cycloalkyl groups may be optionally substituted with one or more substituents as defined herein. Use of the prefix “C₃₋ₓ” or “C₃-Cₓ” with cycloalkyl refers to a cycloalkyl group having from 3 to x member atoms. For example, C₃-C₆cycloalkyl refers to a cycloalkyl group having from 3 to 6 member atoms. Cycloalkyl includes, but is not limited to, cyclopropyl, cyclobutyl, cyclopentyl, and cyclohexyl.

“Cycloalkenyl” refers to an unsaturated hydrocarbon ring having from 3 to 7 member atoms and having a carbon-carbon double bond within the ring. In certain embodiments cycloalkenyl groups have one carbon-carbon double bond within the ring. In other embodiments, cycloalkenyl groups have more than one carbon-carbon double bond within the ring. However, cycloalkenyl rings are not aromatic. Cycloalkenyl groups are monocyclic ring systems. Cycloalkenyl groups may be optionally substituted with one or more substituents as defined herein. Use of the prefix “C₃₋ₓ” or “C₃-Cₓ” with cycloalkenyl refers to a cycloalkenyl group having from 3 to x member atoms. For example, C₃-C₆cycloalkenyl refers to a cycloalkenyl group having from 3 to 6 member atoms. Cycloalkenyl includes, but is not limited to, cyclopropenyl, cyclobutenyl, cyclopentenyl, and cyclohexenyl.

Unless otherwise defined, suitable substituents for any (C₁₋₆)alkyl, (C₂₋₆)alkenyl, and (C₃₋₇)cycloalkyl groups includes up to three substituents selected from the group consisting of hydroxy, halogen, nitro, cyano, carboxy, amino, amidino, sulphonamido, (C₁₋₆)alkoxy, trifluoromethyl, acyloxy, quanidino, (C₃₋₇)cycloalkyl, aryl, and heterocyclic.

“Enantiomeric excess” or “ee” is the excess of one enantiomer over the other expressed as a percentage. As a result, since both enantiomers are present in equal amounts in a racemic mixture, the enantiomeric excess is zero (0% ee). Accordingly, if one enantiomer were enriched so as to constitute 95% of the product, then the ee would be 90% (the amount of the enriched enantiomer, 95%, minus the amount of the other enantiomer, 5%).

“Enantiomerically enriched” refers to products having enantiomeric excess (ee) of greater than zero. For example, enantiomerically enriched refers to products whose ee is greater than about 50%, greater than about 75%, and greater than about 90%.

“Enantiomerically pure” refers to products whose enantiomeric excess is 100%.

The term “Heterocycloalkyl” is used herein to refer to a stable monovalent saturated heterocyclic ring and consist of carbon atoms and from one to three heteroatoms selected from the group consisting of N, O and S, wherein N may optionally be oxidized or quaternized. Heterocycloalkyl may be optionally unsubstituted or substituted as defined herein. Compounds within the invention containing a heterocycloalkyl group may occur in two or more tautometric forms depending on the nature of the heterocycloalkyl group; all such tautomeric forms are included within the scope of the invention. Representative examples include pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, and oxabicylo[2.2.1]heptyl.

“Heteroaryl” refers to an unsaturated planar ring containing from 1 to 4 heteroatoms (as used herein to mean, S, O, or N) in the ring and 4n+2 π electrons, where n is 1, 2, or 3. Heteroaryl groups may be optionally substituted with one or more substituents as defined hereinabove for aryl. Representative heteroaryl groups include pyridinyl, pyrimidinyl, pyradizinyl, thiophenyl, furanyl, 1H-pyrazolyl, benzo[b]furanyl, benzimidazolyl, indolyl, indazolyl, pyrrolyl, oxazolyl, isoxazolyl, oxadiazolyl, thiazolyl, isothiazolyl, thiadiazolyl, imidazolyl, and benzo[b]thiophenyl.

“Halo” or “halogen” refers to fluoro, chloro, bromo, or iodo.

“Haloalkyl moieties” include 1-3 halogen atoms.

The term “Het” as used herein at all occurrences, unless otherwise provided, means a stable heterocyclic ring, which may be either saturated or unsaturated, and consist of carbon atoms and from one to three heteroatoms selected from the group consisting of N, O and S, and wherein the nitrogen may optionally be oxidized or quaternized. Het may be optionally unsubstituted or substituted as defined herein. Suitable “Het” include heterocycloalkyl groups, which are non-aromatic, monovalent monocyclic radicals, which are saturated or partially unsaturated, containing 5 to 6 ring atoms and 1 to 3 heteroatoms selected from nitrogen, oxygen and sulfur, including, but not limited to, pyrrolidyl, imidazolinyl, oxazolinyl, piperidyl, piperazinyl, morpholinyl, tetrahydro-2H-1,4-thiazinyl, tetrahydrofuryl, dihydrofuryl, tetrahydropyranyl, dihydropyranyl, 1,3-dioxolanyl, 1,3-dioxanyl, 1,4-dioxanyl, 1,3-oxathiolanyl, 1,3-oxathianyl. Suitable “Het” also include the heteroaryl groups defined below. In this invention, suitable “Het” may be monocyclic, heteroaryl groups, such as thienyl, furyl, imidazolyl, oxazolyl, thiazolyl, pyridyl, pyrazinyl or pyrimidinyl. The terms “hetero” or “heteroatom” as used herein interchangeably at all occurrences mean oxygen, nitrogen and sulfur.

“Heteroaryl” refers to an aromatic ring containing from 1 to 4 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems or are fused, spiro, or bridged bicyclic ring systems. Monocyclic heteroaryl rings have from 5 to 7 member atoms. Bicyclic heteroaryl rings have from 7 to 11 member atoms. Bicyclic heteroaryl rings include those rings wherein phenyl and a monocyclic heterocycloalkyl ring are attached forming a fused, spiro, or bridged bicyclic ring system, and those rings wherein a monocyclic heteroaryl ring and a monocyclic cycloalkyl, cycloalkenyl, heterocycloalkyl, or heteroaryl ring are attached forming a fused, Spiro, or bridged bicyclic ring system. Heteroaryl includes, but is not limited to, pyrrolyl, pyrazolyl, imidazolyl, oxazolyl, isoxazolyl, thiazolyl, isothiazolyl, furanyl, furazanyl, thienyl, triazolyl, tetrahydrofuranyl, pyridinyl, pyrimidinyl, pyridazinyl, pyrazinyl, triazinyl, tetrazinyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benopyranyl, benzoxazolyl, benzofuranyl, isobenzofuranyl, benzothiazolyl, benzothienyl (also named as benzo[b]thiophenyl or benzothiophenyl), furopyridinyl, and napthyridinyl. Substituents on the heteroaryl ring may be up to three substituents, and includes independently, for example, (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy(C₁₋₄)alkyl; mercapto(C₁₋₄)alkyl; (C₁₋₄)alkoxy; nitro; cyano; carboxy; amino or aminocarbonyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; phenyl, phenyl(C₁₋₄)alkyl or phenyl(C₁₋₄)alkoxy. Substitutents include cyano and (C₁₋₄)alkyl.

Unless otherwise defined, the term “heterocyclic” as used herein includes optionally substituted aromatic and non-aromatic, single and fused, rings suitably containing up to four hetero-atoms in each ring selected from oxygen, nitrogen and sulphur, which rings may be unsubstituted or C-substituted by, for example, up to three groups selected from (C₁₋₄)alkylthio; halo; carboxy(C₁₋₄)alkyl; halo(C₁₋₄)alkoxy; halo(C₁₋₄)alkyl; (C₁₋₄)alkyl; (C₂₋₄)alkenyl; (C₁₋₄)alkoxycarbonyl; formyl; (C₁₋₄)alkylcarbonyl; (C₂₋₄)alkenyloxycarbonyl; (C₂₋₄)alkenylcarbonyl; (C₁₋₄)alkylcarbonyloxy; (C₁₋₄)alkoxycarbonyl(C₁₋₄)alkyl; hydroxy; hydroxy; (C₁₋₄)alkyl; mercapto(C₁₋₄)alkyl; (C₁₋₄)alkoxy; nitro; cyano, carboxy; amino or aminocarbonyl; (C₁₋₄)alkylsulphonyl; (C₂₋₄)alkenylsulphonyl; or aminosulphonyl wherein the amino group is optionally substituted by (C₁₋₄)alkyl or (C₂₋₄)alkenyl; optionally substituted aryl, aryl(C₁₋₄)alkyl or aryl(C₁₋₄)alkoxy and oxo groups.

Each heterocyclic ring suitably has from 4 to 7, or 5 or 6, ring atoms. A fused heterocyclic ring system may include carbocyclic rings and need include only one heterocyclic ring.

“Heteroatom” refers to a nitrogen, sulphur, or oxygen atom.

“Heterocycloalkyl” refers to a saturated or unsaturated ring containing from 1 to 4 heteroatoms as member atoms in the ring. However, heterocycloalkyl rings are not aromatic. Heterocycloalkyl groups containing more than one heteroatom may contain different heteroatoms. Heterocycloalkyl groups may be optionally substituted with one or more substituents as defined herein. Heterocycloalkyl groups are monocyclic ring systems or are fused, spiro, or bridged bicyclic ring systems. Monocyclic heterocycloalkyl rings have from 5 to 7 member atoms. Bicyclic heterocycloalkyl rings have from 7 to 11 member atoms. In certain embodiments, heterocycloalkyl is saturated. In other embodiments, heterocycloalkyl is unsaturated but not aromatic. Heterocycloalkyl includes, but is not limited to, pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, piperazinyl, morpholinyl, thiamorpholinyl, azepinyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, and oxabicylo[2.2.1]heptyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be substituted with one to three substituents as defined herein. “Optionally substituted” in reference to a group includes the unsubstituted group (e.g. “optionally substituted C₁-C₄alkyl” includes unsubstituted C₁-C₄alkyl). It should be understood that the term “substituted” includes the implicit provision that such substitution be in accordance with the permitted valence of the substituted atom and the substituent and that the substitution results in a stable compound (i.e. one that does not spontaneously undergo transformation such as by rearrangement, or cyclization). A single atom may be substituted with more than one substituent as long as such substitution is in accordance with the permitted valence of the atom. Suitable substituents include —OR, —C(O)R, —C(O)OR, —CH(R)OR, —SR, —S(O)R, —S(O)₂R, —N(R)(R), —N(R)C(O)OR, —N(R)C(O)R, —OC(O)N(R)(R), —N(H)C(═NR)N(R)(R)—C(O)N(R)(R), C(R)═NR, aryl, cyano, cycloalkyl, cycloalkenyl, halo, heterocycloalkyl, heteroaryl, nitro, and oxo; wherein each R is independently selected from H, alkyl, alkenyl, alkynyl, cycloalkyl, cycloalkenyl, aryl, heterocycloalkyl, and heteroaryl.

“Oxo” refers to the substituent group ═O.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Compounds within the invention may occur in two or more tautometric forms; all such tautomeric forms are included within the scope of the invention.

Where an amino group forms part of a single or fused non-aromatic heterocyclic ring as defined above suitable optional substituents in such substituted amino groups include H; trifluoromethyl; (C₁₋₄)alkyl optionally substituted by hydroxy, (C₁₋₆)alkoxy, (C₁₋₆)alkylthio, halo or trifluoromethyl; (C₂₋₄)alkenyl; aryl; aryl (C₁₋₄)alkyl; (C₁₋₄)alkoxycarbonyl; (C₁₋₄)alkylcarbonyl; formyl; (C₁₋₆)alkylsulphonyl; or aminocarbonyl wherein the amino group is optionally substituted by (C₁₋₄)alkoxycarbonyl, (C₁₋₄)alkylcarbonyl, (C₂₋₄)alkenyloxycarbonyl, (C₂₋₄)alkenylcarbonyl, (C₁₋₄)alkyl or (C₂₋₄)alkenyl and optionally further substituted by (C₁₋₄)alkyl or (C₂₋₄)alkenyl.

The term “Ph” represents a phenyl ring.

As used herein, “agonist” to a TRPV4 channel receptor includes any compound capable of activating or enhancing the biological activities of a TRPV4 channel receptor. As used herein, “activating” the TRPV4 channel receptor may include, but is not limited to, such outcomes as increasing the amount of Ca²⁺ influx into a cell comprising a TRPV4 channel receptor, reducing the amount of ADAMTSs produced and/or released by the cell, reducing the amount of MMPs produced and/or released by the cell, inhibiting the basal or growth factor-stimulated proliferation of the cell, reducing the amount of nitric oxide (NO) produced by a cell, and attenuating the inhibition of matrix synthesis.

As used herein, “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals, the inflammatory response is capable of mediating considerable tissue damage. Examples of inflammatory mediators include, but are not limited to prostaglandins (e.g., PGE2), leukotrienes (e.g., LTB4), inflammatory cytokines, such as tumor necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type IX, and type XI collagens, along with accessory proteins such as cartilage oligeromeric matrix protein (COMP), link protein, and fibronectin. Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes capable of cleaving extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation. Thus, an inhibition of activity or a reduction in expression of these enzymes may have utility in OA therapy.

As used herein, “reduce” or “reducing” the production of matrix degrading enzymes refers to a decrease in the amount of matrix degrading enzyme(s) produced and/or released by a cell, which has exhibited an increase in matrix degrading enzyme production or release in response to a catabolic stimulus, which may include, but is not limited to, physical injury, mechanical and/or osmotic stress, or exposure to an inflammatory mediator.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

The term “EC₅₀” is used herein to refer to the molar concentration of an agonist that produces 50% of the maximum possible response for that agonist.

Some of the compounds of this invention may be crystallized or recrystallized from solvents such as aqueous and organic solvents. In such cases solvates may be formed. This invention includes within its scope stoichiometric solvates including hydrates as well as compounds containing variable amounts of water that may be produced by processes such as lyophilisation.

Since the compounds of formula (I) are intended for use in pharmaceutical compositions it will readily be understood that they are each provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the formula (I) or pharmaceutically acceptable derivative thereof.

Pharmaceutically acceptable derivatives of the above-mentioned compounds of formula (I) include the free base form or their acid addition or quaternary ammonium salts, for example their salts with mineral acids such as hydrochloric, hydrobromic, sulfuric nitric or phosphoric acids, or organic acids, such as acetic, fumaric, succinic, maleic, citric, benzoic, p-toluenesulfonic, methanesulfonic, naphthalenesulfonic acid or tartaric acids. Compounds of formula (I) may also be prepared as the N-oxide. Compounds of formula (I) having a free carboxy group may also be prepared as an in vivo hydrolyzable ester. The invention extends to all such derivatives.

Examples of suitable pharmaceutically acceptable in vivo hydrolyzable ester-forming groups include those forming esters which break down readily in the human body to leave the parent acid or its salt. Examples of suitable in vivo hydrolyzable ester groups include, for example, acyloxy C₁₋₆ alkyl groups such as acetoxymethyl, pivaloyloxymethyl, α-acetoxyethyl, α-pivaloyloxyethyl, 1-(cyclohexylcarbonyloxy)prop-1-yl, and (1-aminoethyl)carbonyloxymethyl; C₁₋₆ alkoxycarbonyloxyC₁₋₆ alkyl groups, such as ethoxycarbonyloxymethyl, α-ethoxycarbonyloxyethyl and propoxycarbonyloxyethyl; di C₁₋₆ alkylamino C₁₋₆ alkyl, including dimethylaminomethyl, dimethylaminoethyl, diethylaminomethyl or diethylaminoethyl; 2-((C₁₋₆)alkoxycarbonyl)-2-(C₂₋₆)alkenyl groups such as 2-(isobutoxycarbonyl)pent-2-enyl and 2-(ethoxycarbonyl)but-2-enyl; lactone groups such as phthalidyl and dimethoxyphthalidyl.

A further suitable pharmaceutically acceptable in vivo hydrolyzable ester-forming group is that of the formula:

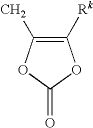

wherein R^{k} is hydrogen, C₁₋₆ alkyl or phenyl.

Certain of the above-mentioned compounds of formula (I) may exist in the form of optical isomers including diastereoisomers, and mixtures of isomers in all ratios including racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

The composition may be formulated for administration by any route, such as oral, topical or parenteral. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration, and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents such as syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulfate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases such as cocoa-butter or other glyceride.

For parenteral administration, fluid unit dosage forms are prepared utilizing the compound and a sterile vehicle, water being preferred. The compound, depending on the vehicle and concentration used, can be either suspended or dissolved in the vehicle. In preparing solutions the compound can be dissolved in water for injection and filter sterilized before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilized powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilization cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compounds according to Formula I may contain one or more asymmetric center and may, therefore, exist as individual enantiomers, diasteriomers, or other stereoisomeric forms, or as mixtures thereof. For example, when R⁴ is other than H, the carbon to which R⁴ is attached is asymmetric. The same logic holds for when R⁶ is other than H. In addition, asymmetric carbon atoms may also be present in a substituent such as an alkyl group. Where the stereochemistry of chiral carbons present in Formula I, or in any chemical structure illustrated herein, is not specified, the chemical structure is intended to encompass compounds containing any stereoisomer and all mixtures thereof of each chiral center present in the compound. Thus, compounds according to Formula I containing one or more chiral center may be used as racemic mixtures, enantiomerically enriched mixtures, or as enantiomerically pure individual stereoisomers.

Individual stereoisomers of a compound according to Formula I which contain one or more asymmetric center may be resolved by methods known to those skilled in the art. For example, such resolution may be carried out by formation of diastereoisomeric salts or complexes which may be separated, for example, by crystallization; by formation of diastereoisomeric derivatives which may be separated, for example, by crystallization, gas-liquid or liquid chromatography; by selective reaction of one enantiomer with an enantiomer-specific reagent, for example by enzamatic oxidation or reduction, followed by separation of the modified and unmodified enantiomers; or gas-liquid or liquid chromatography in a chiral environment, for example, on a chiral support such as silica with a bound chiral ligand or in the presence of a chiral solvent. The skilled artisan will appreciate that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer to the other by asymmetric transformation.

The compounds according to Formula I may also contain double bonds or other centers of geometric asymmetry. Formula I includes both trans (E) and cis (Z) geometric isomers. Likewise, all tautomeric forms are also included in Formula I whether such tautomers exist in equilibrium or predominately in one form.

The skilled artisan will appreciate that pharmaceutically-acceptable salts of the compounds according to Formula I can be prepared. Indeed, in certain embodiments of the invention, pharmaceutically-acceptable salts of the compounds according to Formula I may be preferred over the respective free base or free acid because such salts impart greater stability or solubility to the molecule thereby facilitating formulation into a dosage form. Accordingly, the invention is further directed to pharmaceutically-acceptable salts of the compounds according to Formula I.

As used herein, the term “pharmaceutically-acceptable salts” refers to salts that retain the desired biological activity of the subject compound and exhibit minimal undesired toxicological effects. The term “pharmaceutically-acceptable salts” includes both pharmaceutically-acceptable acid addition salts and pharmaceutically-acceptable base addition salts. These pharmaceutically-acceptable salts may be prepared in situ during the final isolation and purification of the compound, or by separately reacting the purified compound in its free acid or free base form with a suitable base or acid, respectively.

In certain embodiments, compounds according to Formula I may contain an acidic functional group and are therefore capable of forming pharmaceutically-acceptable base addition salts by treatment with a suitable base. Suitable bases include ammonia and hydroxides, carbonates and bicarbonates of a pharmaceutically-acceptable metal cation, such as alkali metal and alkaline earth metal cations. Suitable metal cations include sodium, potassium, lithium, calcium, magnesium, aluminum, and zinc. Suitable bases further include pharmaceutically-acceptable organic primary, secondary, and tertiary amines including aliphatic amines, aromatic amines, aliphatic diamines, and hydroxy alkylamines. Suitable pharmaceutically-acceptable organic bases include methylamine, ethylamine, diethylamine, ethylenediamine, ethanolamine, diethanolamine, and cyclohexylamine.

In certain embodiments, compounds according to Formula I may contain a basic functional group and are therefore capable of forming pharmaceutically-acceptable acid addition salts by treatment with a suitable acid. Suitable acids include pharmaceutically-acceptable inorganic acids, pharmaceutically-acceptable organic acids, and pharmaceutically-acceptable organic sulfonic acids. Suitable inorganic acids include hydrochloric acid, hydrobromic acid, nitric acid, sulfuric acid, sulfamic acid, and phosphoric acid. Suitable organic acids include, acetic acid, hydroxyacetic acid, propionic acid, butyric acid, isobutyric acid, maleic acid, hydroxymaleic acid, acrylic acid, fumaric acid, malic acid, tartaric acid, citric acid, salicylic acid, paminosalicyclic acid, glycollic acid, lactic acid, heptanoic acid, phthalic acid, oxalic acid, succinic acid, benzoic acid, oacetoxybenzoic acid, chlorobenzoic acid, methylbenzoic acid, dinitrobenzoic acid, hydroxybenzoic acid, methoxybenzoic acid, phenylacetic acid, mandelic acid, formic acid, stearic acid, ascorbic acid, palmitic acid, oleic acid, pyruvic acid, pamoic acid, malonic acid, lauric acid, glutaric acid, and glutamic acid. Suitable organic sulfonic acids include, methanesulfonic acid, ethanesulfonic acid, 2-hydroxyethanesulfonic acid, benzenesulfonic acid, paminobenzenesulfonic (i.e. sulfanilic acid), ptoluenesulfonic acid, and napthalene-2-sulfonic acid.

When in the solid state, the compounds of the invention may exist as either amorphous material or in crystalline form, or as a mixture thereof. The skilled artisan will appreciate that pharmaceutically-acceptable solvates of the compounds of the invention may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” The invention includes all such solvates.

The skilled artisan will further appreciate that certain compounds of the invention that exist in crystalline form, including the various solvates thereof, may exhibit polymorphism (i.e., the capacity to occur in different crystalline structures). These different crystalline forms are typically known as “polymorphs.” The invention includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, such as solvents, used in making the compound. In addition, one polymorph may spontaneously convert to another polymorph under certain conditions.

### General Reaction Scheme for Preparing the Compounds of the Invention:

Specifically, the invention is directed to compounds according to Formula I:

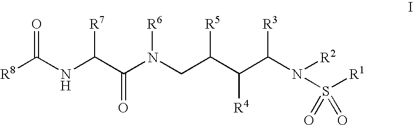

or pharmaceutically acceptable salts, hydrates, or solvates thereof, wherein:
R¹ is aryl optionally substituted with CN, NO₂, halogen, CH₃, CF₃ or H;
R² is H, C₁-C₆ alkyl, C₃-C₇ cycloalkyl, or C₃-C₇ heterocycloalkyl;
R³ is H, OH, C₁-C₆ OH, O—C₁-C₆ alkyl, CO₂CH₃, CONHCH₃, SH, S—C₁-C₆ alkyl, or F;
R⁴ is H, OH, C₁-C₆ OH, O—C₁-C₆ alkyl, SH, S—C₁-C₆ alkyl, or F;
R⁵ is H, OH, C₁-C₆ OH, O—C₁-C₆ alkyl, SH, S—C₁-C₆ alkyl, or F;
R⁶ is H or C₁-C₆ alkyl;
R⁷ is optionally substituted C₁-C₆ alkyl, O—C₁-C₆ alkyl, C—S—C₁-C₆ alkyl cyclohexylmethyl, amide, urea, or cyclopentylmethyl; and
R⁸ is optionally substituted C₃₋₇cycloalkyl, optionally substituted C₃₋₇cycloalkenyl, optionally substituted Het-C₃₋₇alkyl, optionally substituted Het-C₃₋₇alkenyl, optionally substituted aryl, optionally substituted aryloxy; optionally substituted arylamino; optionally substituted heterocycloalkyl, optionally substituted heteroaryl, optionally substituted cylcoalkyl, or optionally substituted indenyl

In one aspect of the invention compounds of Formula I R¹ is aryl substituted with one or more substituents selected from the group consisting of halo, cyano, methyl and CF₃;

### R² is H;

### R³ is H, C₂OH, CO₂CH₃ or CONHCH₃;

### R⁴ is H or OH;

### R⁵ is H or OH,

### R⁶ is H;

R⁷ is isobutyl, butenyl, thiazol, C—O—C₁-C₆ alkyl, hydroxydimethylbutyl, dichloropropyl, trifluoropropyl, phenylethyl, or phenylpropyl; and
R⁸ is phenyl, optionally substituted phenyl, benzothienyl, C₁₋₁₂alkyl substituted benzothienyl, benzothiazolyl; alkyl substituted benzothiazolyl; furanyl, halogen substituted furanyl, aryl substituted furanyl; tetrahydrofuran-2-yl; benzofuranyl, alkoxy substituted benzofuranyl, halogen substituted benzofuranyl, alkyl substituted benzofuranyl; benzo[b]thiophenyl, alkoxy substituted benzo[b]thiophenyl; optionally substituted isoquinolinyl, quinolinyl; indolyl, alkyl substituted indolyl; alkyl substituted indolyl further substituted with dimethylethyl carboxylate; indolyl further substituted with one to three carboxy groups, methylphenyl propenoyl, pyridinyl, alkyl substituted pyridinyl, thiopyranyl, pyridazinyl; thienopyridinyl, quinolizinyl, optionally substituted imidazolyl, imidazothiazolyl, pyrrolyl, cylcopenta[b]thiophenyl, cyclopentyl substituted with one to three alkoxy groups, cyclohexyl substituted with one to three alkoxy groups, cylcopentylpropanoyl, cyclohexylpropanoyl, cylcopentylmethyloxy, cyclohexylmethyloxy, cyclohexyldimethylpropanoyl, cyclopentylamino, cyclohexylamino, cyclopentylmethylamino, cyclohexylmethylamino, indenyl, cyclohexene, piperidinyl, propylpiperidinyl further substituted with methylbutylcarbozylate, thiophenyl, thiophenyl further substituted with phenyl, alkyl substituted thiophenyl, halogen substituted thiophenyl, halogen substituted benzothiophenyl, thieno[3,2-b]thiophenyl, isoxazolyl, alkyl substituted isoxazolyl, and oxazolyl; and pharmaceutically acceptable salts, hydrates, or solvates thereof.

In another aspect R² through R⁶ can have either orientation. In another aspect of Formula I, R⁸ is phenyl substituted with one to three substituents selected from the group consisting of: C₂O, NO₂, dimethylpropanoyl, methylpiperazinyl, phenyl, piperazine further substituted with dimethylethylcarbonyl, amino, halogen, CH₃, C₁-C₁₂alkyl, C₁-C₁₂alkoxy, amino sulfonyl, and alkylsulfonyl groups and pharmaceutically acceptable salts, hydrates, or solvates thereof. In another aspect of Formula I, R⁸ is isoquinoline further substituted with one to three substituents selected from the group consisting of dimethylethylcarbonyl and phenylcarbonyl and pharmaceutically acceptable salts, hydrates, or solvates thereof. In another aspect a composition is provided comprising Formula I and a pharmaceutically acceptable carrier, diluents or excipient.

In addition, the invention is directed to compounds according to Formula II

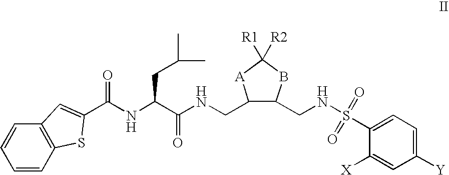

and pharmaceutically acceptable salts, hydrates, or solvates thereof, wherein:

### R¹ is H or CH₃;

### R² is H or CH₃;

### A is C or O;

### B is C or O;

### X is H, Cl or F; and

### Y is H, Cl or F.

In one aspect five-membered rings comprising A and B of Formula II include but are not limited to acetonides, dioxolanes, tetrahydrofurans, and cyclopentanes.

The invention is also directed to compounds according Formula III

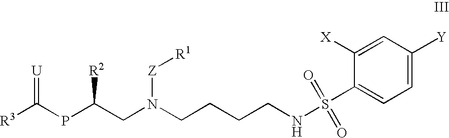

and pharmaceutically acceptable salts, hydrates, or solvates thereof, wherein:

### X is H, C₁, CF₃, NO₂, or CN;

### Y is H, Cl, or F;

### Z is C═S, C═O or O═S═O;

### U is O or S;

R¹ is optionally substituted cyloalkyl, C₁-C₁₂ alkyl, C₁-C₁₂ alkoxy, C₁-C₁₂ alkylamino, optionally substituted aryl, optionally substituted arylamino, optionally substituted heteroaryl; or optionally substituted heterocycloalkyl;
R² is H, C₁-C₁₂ alkyl, C₁-C₁₂ alkoxy, optionally substituted heterocycloalkylamino, optionally substituted heteroaryl, or optionally substituted aryl;

### P is NH or O; and

R³ is C₁-C₁₂ alkylamino, cycloalkylamino, optionally substituted aryl amino, optionally substituted heteroarylamino, heterocyclicalkyl, or optionally substituted aryloxy;
wherein when P is NH, R² may form a five or six member heterocyclic ring with P forming a piperidinyl or pyrrolidinyl group.

In another aspect Formula III provides compounds wherein

### X is H, C₁, CF₃, NO₂, or CN;

### Y is H, Cl, or F;

### Z is C═S, C═O or O═S═O;

### U is O or S;

R¹ is cyclohexylamino, methyl, carbonyl, ethylamino, methylethylamino, phenylamino, cylcopropylamino, dimethylethylamino, substituted phenyl, furanylmethyl amino, thienyl amino, substituted piperidinyl, azinyl, or fluoroethyl amino;
R² is H, isobutyl, methyloxypropyl, phenylmethyloxypropyl, phenylmethyloxymethyl, morpholinylpropyl, or morpholinylmethyl;

### P is NH or O; and

R³ is phenylamino, methylethylamino, cyclohexylamino, ethylamino, substituted phenylamino, pyridinylamino, thienylamino, trifluoroacetylpiperidinyl, cyclopentylamino, methyloxypropyl, phenylmethyloxy, morpholinyl, or methylamino;
wherein when P is NH, R² may form a five or six member heterocyclic ring with P forming a piperidinyl or pyrrolidinyl group.

Exemplary compounds of the present invention include:

| Example No. | Compound Name |
|---|---|
| 1. | N-((1S)-1-{[(4-{[(2,4- |
| | Dichlorophenyl)sulfonyl]amino}butyl)amino]carbonyl}-3-methylbutyl)-1- |
| | benzothiophene-2-carboxamide |
| 2. | N-((1S)-1-{[(4-{[(2-Bromo-4- |
| | fluorophenyl)sulfonyl]amino}butyl)amino]carbonyl}-3-methylbutyl)-1- |
| | benzothiophene-2-carboxamide |
| 3. | N-((1S)-1-{[(4-{[(4-Bromo-2- |
| | chlorophenyl)sulfonyl]amino}butyl)amino]carbonyl}-3-methylbutyl)-1- |
| | benzothiophene-2-carboxamide |
| 4. | N-[(1S)-1-({[4-({[4-Fluoro-2- |
| | (trifluoromethyl)phenyl]sulfonyl}amino)butyl]amino} carbonyl)-3- |
| | methylbutyl]-1-benzothiophene-2-carboxamide |
| 5. | N-((1S)-1-{[(4-{[(2-Chloro-4- |
| | fluorophenyl)sulfonyl]amino}butyl)amino]carbonyl}-3-methylbutyl)-1- |
| | benzothiophene-2-carboxamide |
| 6. | N-((1S)-1-{[(4-{[(2,4- |
| | Dichlorophenyl)sulfonyl]amino}butyl)amino]carbonyl}-3-methylbutyl)-3- |
| | methylfuro[3,2-b]pyridine-2-carboxamide |
| 7. | N-((1S)-1-{[(4-{[(2,4- |
| | dichlorophenyl)sulfonyl]amino}butyl)amino]carbonyl}-3-methylbutyl)-1- |
| | methyl-1H-indole-2-carboxamide |
| 8. | N-((1S)-1-{[(4-{[(2,4- |
| | dichlorophenyl)sulfonyl]amino}butyl)amino]carbonyl}-3-methylbutyl)-1- |
| | benzofuran-2-carboxamide |
| 9. | N-((1S)-1-{[(4-{[(2,4- |
| | Dichlorophenyl)sulfonyl]amino}butyl)amino]carbonyl}-3-methylbutyl)-3- |
| | methylfuro[3,2-b]pyridine-2-carboxamide |
| 10. | N-((1S)-1-{[(4-{[(2,4- |
| | Dichlorophenyl)sulfonyl]amino}butyl)amino]carbonyl}-3- |
| | methylbutyl)imidazo[1,2-b]pyridazine-2-carboxamide |
| 11. | N-((1S)-1-{[(4-{[(2,4- |
| | Dichlorophenyl)sulfonyl]amino}butyl)amino]carbonyl}-3- |
| | methylbutyl)thieno[3,2-b]pyridine-2-carboxamide |
| 12. | (2S)—N-((1S)-1-{[(4-{[(2,4- |
| | Dichlorophenyl)sulfonyl]amino}butyl)amino]carbonyl}-3- |
| | methylbutyl)octahydro-2H-quinolizine-2-carboxamide |
| 13. | N-((1S)-1-{[(4-{[(2,4- |
| | Dichlorophenyl)sulfonyl]amino}butyl)amino]carbonyl}-3- |
| | methylbutyl)imidazo[1,2-a]pyridine-2-carboxamide |
| 14. | N-((1S)-1-{[(4-{[(2,4- |
| | Dichlorophenyl)sulfonyl]amino}butyl)amino]carbonyl}-3- |
| | methylbutyl)imidazo[2,1-b][1,3]thiazole-6-carboxamide |
| 15. | N-((1S)-1-{[(4-{[(2,4 |
| | Dichlorophenyl)sulfonyl]amino}butyl)amino]carbonyl}-3-methylbutyl)- |
| | 4H-thieno[3,2-b]pyrrole-5-carboxamide |
| 16. | N-[(1S)-1-({[4-({[4-Fluoro-2- |
| | (trifluoromethyl)phenyl]sulfonyl}amino)butyl]amino}carbonyl)-3- |
| | methylbutyl]-4H-thieno[3,2-b]pyrrole-5-carboxamide |
| 17. | N-((1S)-1-{[(4-{[(2-Chloro-4- |
| | fluorophenyl)sulfonyl]amino}butyl)amino]carbonyl}-3-methylbutyl)-4H- |
| | thieno[3,2-b]pyrrole-5-carboxamide |
| 18. | N¹-(4-{[(2,4-Dichlorophenyl)sulfonyl]amino}butyl)-N²-({4-[(4-methyl-1- |
| | piperazinyl)methyl]phenyl}carbonyl)-L-leucinamide |
| 19. | 1,1-Dimethylethyl 4-(4{[((1S)-1-{[(4-{[(2,4- |
| | dichlorophenyl)sulfonyl]amino}butyl)amino] carbonyl}-3- |
| | methylbutyl)amino]carbonyl}phenyl)-1-piperazinecarboxylate |
| 20. | N-[(1S)-1-({[4-({[4-Fluoro-2- |
| | (trifluoromethyl)phenyl]sulfonyl}amino)butyl]amino} carbonyl)-3- |
| | methylbutyl]-5,6-dihydro-4H-cyclopenta[b]thiophene-2-carboxamide |
| 21. | N²-(3-Cyclopentylpropanoyl)-N¹-[4-({[4-fluoro-2- |
| | (trifluoromethyl)phenyl]sulfonyl}amino)butyl]-L-leucinamide |
| 22. | N²-(3-Cyclohexylpropanoyl)-N¹-[4-({[4-fluoro-2- |
| | (trifluoromethyl)phenyl]sulfonyl}amino)butyl]-L-leucinamide |
| 23. | N²-(3-Cyclohexylpropanoyl)-N¹-(4-{[2,4- |
| | dichlorophenyl)sulfonyl]amino}butyl)-L-leucinamide |
| 24. | N²-(3-Cyclopentylpropanoyl)-N¹-(4-{[(2,4- |
| | dichlorophenyl)sulfonyl]amino}butyl)-L-leucinamide |
| 25. | N¹-(4-{[2-Chloro-4-fluorophenyl)sulfonyl]amino}butyl)-N²-(3- |
| | cyclohexylpropanoyl)-L-leucinamide |
| 26. | N¹-(4-{[(2-Chloro-4-fluorophenyl)sulfonyl]amino}butyl)-N²-(3- |
| | cyclopentylpropanoyl)-L-leucinamide |
| 27. | N¹-(4-{[(2-Chloro-4-fluorophenyl)sulfonyl]amino}butyl)-N²- |
| | (cyclohexylacetyl)-L-leucinamide |
| 28. | N¹-(4-{[(2-Chloro-4-fluorophenyl)sulfonyl]amino}butyl)-N²-[(4-phenyl-2- |
| | thienyl)carbonyl]-L-leucinamide |
| 29. | N¹-(4-{[(2-Chloro-4-fluorophenyl)sulfonyl]amino}butyl)-N²-[(2E)-3-(4- |
| | methylphenyl)-2-propenoyl]-L-leucinamide |
| 30. | 1,1-Dimethylethyl 5-{[((1S)-1-{[(4-{[(2-chloro-4- |
| | fluorophenyl)sulfonyl]amino}butyl)amino]carbonyl}-3- |
| | methylbutyl)amino]carbonyl}-3,4-dihydro-2(1H)- |
| | isoquinolinecarboxylate |
| 31. | N¹-(4-{[(2-Chloro-4-fluorophenyl)sulfonyl]amino}butyl)-N²-[(5-phenyl-2- |
| | thienyl)carbonyl]-L-leucinamide |
| 32. | N¹-(4-{[(2-Chloro-4-fluorophenyl)sulfonyl]amino}butyl)-N²-(2,3-dihydro- |
| | 1H-inden-2-ylacetyl)-L-leucinamide |
| 33. | N¹-(4-{[(2-Chloro-4-fluorophenyl)sulfonyl]amino}butyl)-N²-(1- |
| | cyclohexen-1-ylcarbonyl)-L-leucinamide |
| 34. | 1,1-Dimethylethyl 3-{3-[((1S)-1-{[(4-{[(2-chloro-4- |
| | fluorophenyl)sulfonyl]amino}butyl)amino]carbonyl}-3- |
| | methylbutyl)amino]-3-oxopropyl}-1-piperidinecarboxylate |
| 35. | N-((1S)-1-{[(4-{[(2-chloro-4- |
| | fluorophenyl)sulfonyl]amino}butyl)amino]carbonyl}-3-methylbutyl)-2,3- |
| | dihydro-1H-indene-2-carboxamide |
| 36. | N¹-(4-{[(2-chloro-4-fluorophenyl)sulfonyl]amino}butyl)-N²-[3-(2- |
| | chlorophenyl)propanoyl]-L-leucinamide |
| 37. | N¹-(4-{[(2-chloro-4-fluorophenyl)sulfonyl]amino}butyl)-N²-[3-(3- |
| | chlorophenyl)propanoyl]-L-leucinamide |
| 38. | 1,1-Dimethylethyl 2-{3-[((1S)-1-{[(4-{[(2-chloro-4- |
| | fluorophenyl)sulfonyl]amino}butyl)amino]carbonyl}-3- |
| | methylbutyl)amino]-3-oxopropyl}-1-piperidinecarboxylate |
| 39. | N¹-(4-{[(2-Chloro-4-fluorophenyl)sulfonyl]amino}butyl)-N²-[3-(4- |
| | chlorophenyl)propanoyl]-L-leucinamide |
| 40. | N¹-(4-{[(2-Chloro-4-fluorophenyl)sulfonyl]amino}butyl)-N²- |
| | (cyclopentylacetyl)-L-leucinamide |
| 41. | 1,1-Dimethylethyl 2-{[((1S)-1-{[(4-{[(2-chloro-4- |
| | fluorophenyl)sulfonyl]amino}butyl)amino]carbonyl}-3- |
| | methylbutyl)amino]carbonyl}octahydro-1H-indole-1-carboxylate |
| 42. | N¹-(4-{[(2-chloro-4-fluorophenyl)sulfonyl]amino}butyl)-N²-[3-4- |
| | methylphenyl)propanoyl]-L-leucinamide |
| 43. | N-((1S)-1-{[(4-{[(2-chloro-4- |
| | fluorophenyl)sulfonyl]amino}butyl)amino]carbonyl}-3- |
| | methylbutyl)furo[3,2-b]pyridine-2-carboxamide |
| 44. | N¹-(4-{[(2-chloro-4-fluorophenyl)sulfonyl]amino}butyl)-N²-(tetrahydro- |
| | 2H-thiopyran-4-ylacetyl)-L-leucinamide |
| 45. | N-((1S)-1-{[(4-{[(2-chloro-4- |
| | fluorophenyl)sulfonyl]amino}butyl)amino]carbonyl}-3-methylbutyl)-2,3- |
| | dihydro-1H-indole-2-carboxamide |
| 46. | 1-Acetyl-N-((1S)-1-{[(4-{[(2-chloro-4- |
| | fluorophenyl)sulfonyl]amino}butyl)amino]carbonyl}-3-methylbutyl)-2,3- |
| | dihydro-1H-indole-2-carboxamide |
| 47. | N¹-(4-{[(2-Chloro-4-fluorophenyl)sulfonyl]amino}butyl)-N²-(2,2- |
| | dimethyl-3-phenylpropanoyl)-L-leucinamide |
| 48. | N²-[(4-Acetylphenyl)carbonyl]-N¹-(4-{[(2-chloro-4- |
| | fluorophenyl)sulfonyl]amino}butyl)-L-leucinamide |
| 49. | N¹-(4-{[(2-chloro-4-fluorophenyl)sulfonyl]amino}butyl)-N²-[3-(3- |
| | nitrophenyl)propanoyl]-L-leucinamide |
| 50. | N-((1S)-1-{[(4-{[(2-chloro-4- |
| | fluorophenyl)sulfonyl]amino}butyl)amino]carbonyl}-3-methylbutyl)-1,3- |
| | benzothiazole-2-carboxamide |
| 51. | N¹-(4-{[(2-chloro-4-fluorophenyl)sulfonyl]amino}butyl)-N²-(3-cyclohexyl- |
| | 2,2-dimethylpropanoyl)-L-leucinamide |
| 52. | N¹-(4-{[(2-chloro-4-fluorophenyl)sulfonyl]amino}butyl)-N²- |
| | (cyclopentylcarbonyl)-L-leucinamide |
| 53. | N-((1S)-1-{[(4-{[(2-chloro-4- |
| | fluorophenyl)sulfonyl]amino}butyl)amino]carbonyl}-3-methylbutyl)-2- |
| | (phenylmethyl)-1,2,3,4-tetrahydro-5-isoquinolinecarboxamide |
| 54. | N-((1S)-1-{[(4-{[(2-chloro-4- |
| | fluorophenyl)sulfonyl]amino}butyl)amino]carbonyl}-3-methylbutyl)-2- |
| | (phenylcarbonyl)-1,2,3,4-tetrahydro-5-isoquinolinecarboxamide |
| 55. | N-((1S)-1-{[(4-{[(2-chloro-4- |
| | fluorophenyl)sulfonyl]amino}butyl)amino]carbonyl}-3-methylbutyl)-1- |
| | benzofuran-2-carboxamide |
| 56. | N¹-(4-{[(2,4-dichlorophenyl)sulfonyl]amino}butyl)-N²- |
| | [(phenyloxy)acetyl]-L-leucinamide |
| 57. | N¹-(4-{[(2,4-dichlorophenyl)sulfonyl]amino}butyl)-N²-({[2- |
| | (methyloxy)phenyl]oxy}acetyl)-L-leucinamide |
| 58. | N²-{[(4-chloro-2-methylphenyl)oxy]acetyl}-N¹-(4-{[(2,4- |
| | dichlorophenyl)sulfonyl]amino}butyl)-L-leucinamide |
| 59. | N¹-(4-{[(2,4-dichlorophenyl)sulfonyl]amino}butyl)-N²-{[(2- |
| | methylphenyl)oxy]acetyl}-L-leucinamide |
| 60. | N²-{[(2-chlorophenyl)oxy]acetyl}-N¹-(4-{[(2,4- |
| | dichlorophenyl)sulfonyl]amino}butyl)-L-leucinamide |
| 61. | N¹-(4-{[(2,4-dichlorophenyl)sulfonyl]amino}butyl)-N²-[(1-methyl-1H- |
| | imidazol-4-yl)sulfonyl]-L-leucinamide |
| 62. | N²-[(cyclohexylamino)carbonyl]-N¹-(4-{[(2,4- |
| | dichlorophenyl)sulfonyl]amino}butyl)-L-leucinamide |
| 63. | N¹-(4-{[(2-chloro-4-fluorophenyl)sulfonyl]amino}butyl)-N²- |
| | [(cyclohexylamino)carbonyl]-L-leucinamide |
| 64. | N-((1S)-1-{[(4-{[(2-chloro-4- |
| | fluorophenyl)sulfonyl]amino}butyl)amino]carbonyl}-3- |
| | methylbutyl)octahydro-2(1H)-isoquinolinecarboxamide |
| 65. | N-((1S)-1-{[(4-{[(2-chloro-4- |
| | fluorophenyl)sulfonyl]amino}butyl)amino]carbonyl}-3-methylbutyl)-3,4- |
| | dihydro-2(1H)-isoquinolinecarboxamide |
| 66. | N²-[(cyclohexylamino)carbonyl]-N¹-[4-({[4-fluoro-2- |
| | (trifluoromethyl)phenyl]sulfonyl}amino)butyl]-L-leucinamide |
| 67. | N¹-[4-({[4-fluoro-2-(trifluoromethyl)phenyl]sulfonyl}amino)butyl]-N²- |
| | [(phenylamino)carbonyl]-L-leucinamide |
| 68. | N²-[(cyclopentylamino)carbonyl]-N¹-[4-({[4-fluoro-2- |
| | (trifluoromethyl)phenyl]sulfonyl}amino)butyl]-L-leucinamide |
| 69. | N²-{[(cyclohexylmethyl)amino]carbonyl}-N¹-[4-({[4-fluoro-2- |
| | (trifluoromethyl)phenyl]sulfonyl}amino)butyl]-L-leucinamide |
| 70. | N²-{[cyclohexyl(methyl)amino]carbonyl}-N¹-[4-({[4-fluoro-2- |
| | (trifluoromethyl)phenyl]sulfonyl}amino)butyl]-L-leucinamide |
| 71. | N²-{[cyclopentyl(methyl)amino]carbonyl}-N¹-[4-({[4-fluoro-2- |
| | (trifluoromethyl)phenyl]sulfonyl}amino)butyl]-L-leucinamide |
| 72. | N¹-[4-({[4-fluoro-2-(trifluoromethyl)phenyl]sulfonyl}amino)butyl]-N²- |
| | {[(phenylmethyl)amino]carbonyl}-L-leucinamide |
| 73. | N²-{[(cyclopentylmethyl)oxy]carbonyl}-N¹-[4-({[4-fluoro-2- |
| | (trifluoromethyl)phenyl]sulfonyl}amino)butyl]-L-leucinamide |
| 74. | N²-{[(cyclohexylmethyl)oxy]carbonyl}-N¹-[4-({[4-fluoro-2- |
| | (trifluoromethyl)phenyl]sulfonyl}amino)butyl]-L-leucinamide |
| 75. | N¹-(4-{[(2-chloro-4-fluorophenyl)sulfonyl]amino}butyl)-N²- |
| | {[(cyclopentylmethyl)oxy]carbonyl}-L-leucinamide |
| 76. | N¹-(4-{[(2-chloro-4-fluorophenyl)sulfonyl]amino}butyl)-N²- |
| | {[(cyclohexylmethyl)oxy]carbonyl}-L-leucinamide |
| 77. | N-((1S)-1-{[(4-{[(2,4- |
| | dichlorophenyl)sulfonyl]amino}butyl)amino]carbonyl}-3-methyl-3-buten- |
| | 1-yl)-1-benzothiophene-2-carboxamide |
| 78. | N-[(1S)-2-[(4-{[(2,4-dichlorophenyl)sulfonyl]amino}butyl)amino]-2-oxo- |
| | 1-(1,3-thiazol-4-ylmethyl)ethyl]-1-benzothiophene-2-carboxamide |
| 79. | N-((1S)-2-[(4-{[(2-chloro-4-fluorophenyl)sulfonyl]amino}butyl)amino]-1- |
| | {[(1,1-dimethylethyl)oxy]methyl}-2-oxoethyl)-1-benzothiophene-2- |
| | carboxamide |
| 80. | N-{(1R)-2-[(4-{[(2-chloro-4-fluorophenyl)sulfonyl]amino}butyl)amino]-1- |
| | [(methylthio)methyl]-2-oxoethyl}-1-benzothiophene-2-carboxamide |
| 81. | N-((1S,2R)-1-{[(4-{[(2-chloro-4- |
| | fluorophenyl)sulfonyl]amino}butyl)amino]carbonyl}-2-hydroxy-3- |
| | methylbutyl)-1-benzothiophene-2-carboxamide |
| 82. | N-((1S,2S)-1-{[(4-{[(2-chloro-4- |
| | fluorophenyl)sulfonyl]amino}butyl)amino]carbonyl}-2-hydroxy-3,3- |
| | dimethylbutyl)-1-benzothiophene-2-carboxamide |
| 83. | N-((1S)-3,3-dichloro-1-{[(4-{[(2,4- |
| | dichlorophenyl)sulfonyl]amino}butyl)amino]carbonyl}propyl)-1- |
| | benzothiophene-2-carboxamide |
| 84. | N-((1S)-3,3-dichloro-1-{[(4-{[(2-chloro-4- |
| | fluorophenyl)sulfonyl]amino}butyl)amino]carbonyl}propyl)-1- |
| | benzothiophene-2-carboxamide |
| 85. | (2S)-4,4-dichloro-N-(4-{[(2-chloro-4- |
| | fluorophenyl)sulfonyl]amino}butyl)-2- |
| | {[(cyclohexylamino)carbonyl]amino}butanamide |
| 86. | N-(1-{[(4-{[(2,4-dichlorophenyl)sulfonyl]amino}butyl)amino]carbonyl}- |
| | 3,3,3-trifluoropropyl)-1-benzothiophene-2-carboxamide |
| 87. | N-((1S)-1-{[(4-{[(2,4- |
| | dichlorophenyl)sulfonyl]amino}butyl)amino]carbonyl}-3,3- |
| | difluoropropyl)-1-benzothiophene-2-carboxamide |
| 88. | N-((1R)-1-{[(4-{[(2,4- |
| | dichlorophenyl)sulfonyl]amino}butyl)amino]carbonyl}-3-methylbutyl)-1- |
| | benzothiophene-2-carboxamide |
| 89. | N-{(1S)-2-[(4-{[(2-chloro-4-fluorophenyl)sulfonyl]amino}butyl)amino]-2- |
| | oxo-1-phenylethyl}-1-benzothiophene-2-carboxamide |
| 90. | N-(1-benzothien-2-ylcarbonyl)-N-(4-{[(2-chloro-4- |
| | fluorophenyl)sulfonyl]amino}butyl)-L-phenylalaninamide |
| 91. | N-((1S)-1-{[(4-{[(2,4- |
| | difluorophenyl)sulfonyl]amino}butyl)amino]carbonyl}-3-methylbutyl)-1- |
| | benzothiophene-2-carboxamide |
| 92. | N-((1S)-1-{[(4-{[(4-fluoro-2- |
| | methylphenyl)sulfonyl]amino}butyl)amino]carbonyl}-3-methylbutyl)-1- |
| | benzothiophene-2-carboxamide |
| 93. | N-{(1S)-1-[({4-[{[4-fluoro-2- |
| | (trifluoromethyl)phenyl]sulfonyl}(methyl)amino]butyl}amino)carbonyl]-3- |
| | methylbutyl}-1-benzothiophene-2-carboxamide |
| 94. | N-{(1S)-1-[({4-[[(2,4- |
| | dichlorophenyl)sulfonyl](methyl)amino]butyl}amino)carbonyl]-3- |
| | methylbutyl}-1-benzothiophene-2-carboxamide |
| 95. | N-{(1S)-1-[({4-[[(2-chloro-4- |
| | fluorophenyl)sulfonyl](methyl)amino]butyl}amino)carbonyl]-3- |
| | methylbutyl}-1-benzothiophene-2-carboxamide |
| 96. | N-{(1S)-1-[({4-[[(2- |
| | cyanophenyl)sulfonyl](methyl)amino]butyl}amino)carbonyl]-3- |
| | methylbutyl}-1-benzothiophene-2-carboxamide |
| 97. | 2-(3-biphenylyl)-N-(4-{[(2,4-dichlorophenyl)sulfonyl]amino}butyl)-4- |
| | methylpentanamide |
| 98. | N-((1S)-1-{[((2S)-4-{[(2-chloro-4-fluorophenyl)sulfonyl]amino}-2- |
| | hydroxybutyl)amino]carbonyl}-3-methylbutyl)-1-benzothiophene-2- |
| | carboxamide |
| 99. | N-((1S)-1-{[((2R)-4-{[(2-chloro-4-fluorophenyl)sulfonyl]amino}-2- |
| | hydroxybutyl)amino]carbonyl}-3-methylbutyl)-1-benzothiophene-2- |
| | carboxamide |
| 100. | N-((1S)-3,3-dichloro-1-{[((2S)-4-{[(2,4-dichlorophenyl)sulfonyl]amino}- |
| | 2-hydroxybutyl)amino]carbonyl}propyl)-1-benzothiophene-2- |
| | carboxamide |
| 101. | N²-[(cyclohexylamino)carbonyl]-N¹-((2S)-4-{[(2,4- |
| | dichlorophenyl)sulfonyl]amino}-2-hydroxybutyl)-L-leucinamide |
| 102. | N-((1S)-1-{[((2S)-4-{[(2-chloro-4-fluorophenyl)sulfonyl]amino}-2- |
| | hydroxybutyl)amino]carbonyl}-3-methylbutyl)-1-benzofuran-2- |
| | carboxamide |
| 103. | N-((1S)-1-{[((3S)-4-{[(2-chloro-4-fluorophenyl)sulfonyl]amino}-3- |
| | hydroxybutyl)amino]carbonyl}-3-methylbutyl)-1-benzothiophene-2- |
| | carboxamide |
| 104. | N-((1S)-1-{[((3R)-4-{[(2-chloro-4-fluorophenyl)sulfonyl]amino}-3- |
| | hydroxybutyl)amino]carbonyl}-3-methylbutyl)-1-benzothiophene-2- |
| | carboxamide |
| 105. | N-((1S)-1-{[((4R)-4-{[(2-chloro-4-fluorophenyl)sulfonyl]amino}-5- |
| | hydroxypentyl)amino]carbonyl}-3-methylbutyl)-1-benzothiophene-2- |
| | carboxamide |
| 106. | N-((1S)-1-{[((4S)-4-{[(2-chloro-4-fluorophenyl)sulfonyl]amino}-5- |
| | hydroxypentyl)amino]carbonyl}-3-methylbutyl)-1-benzothiophene-2- |
| | carboxamide |
| 107. | Methyl N⁵-[N-(1-benzothien-2-ylcarbonyl)-L-leucyl]-N²-[(2-chloro-4- |
| | fluorophenyl)sulfonyl]-D-ornithinate |
| 108. | N⁵-[N-(1-benzothien-2-ylcarbonyl)-L-leucyl]-N²-[(2-chloro-4- |
| | fluorophenyl)sulfonyl]-N¹-methyl-D-ornithinamide |
| 109. | N-{(1S)-1-[({[(4R,5R)-5-({[(2,4-dichlorophenyl)sulfonyl]amino}methyl)- |
| | 2,2-dimethyl-1,3-dioxolan-4-yl]methyl}amino)carbonyl]-3-methylbutyl}- |
| | 1-benzothiophene-2-carboxamide |
| 110. | N-((1S)-1-{[((2R,3R)-4-{[(2,4-dichlorophenyl)sulfonyl]amino}-2,3- |
| | dihydroxybutyl)amino]carbonyl}-3-methylbutyl)-1-benzothiophene-2- |
| | carboxamide |
| 111. | N-{(1S)-1-[({[(4R,5R)-5-({[(2-chloro-4- |
| | fluorophenyl)sulfonyl]amino}methyl)-2,2-dimethyl-1,3-dioxolan-4- |
| | yl]methyl}amino)carbonyl]-3-methylbutyl}-1-benzothiophene-2- |
| | carboxamide |
| 112. | N-((1S)-1-{[((2R,3R)-4-{[(2-chloro-4-fluorophenyl)sulfonyl]amino}-2,3- |
| | dihydroxybutyl)amino]carbonyl}-3-methylbutyl)-1-benzothiophene-2- |
| | carboxamide |
| 113. | N-{(1S)-1-[({[(4R,5R)-5-({[(2,4-dichlorophenyl)sulfonyl]amino}methyl)- |
| | 1,3-dioxolan-4-yl]methyl}amino)carbonyl]-3-methylbutyl}-1- |
| | benzothiophene-2-carboxamide |
| 114. | N-{(1S)-1-[({[(4R,5R)-5-({[(2-chloro-4- |
| | fluorophenyl)sulfonyl]amino}methyl)-1,3-dioxolan-4- |
| | yl]methyl}amino)carbonyl]-3-methylbutyl}-1-benzothiophene-2- |
| | carboxamide |
| 115. | N-{(1S)-1-[({[(2S,3R)-2-({[(2-chloro-4- |
| | fluorophenyl)sulfonyl]amino}methyl)tetrahydro-3- |
| | furanyl]methyl}amino)carbonyl]-3-methylbutyl}-1-benzothiophene-2- |
| | carboxamide |
| 116. | N-{(1S)-1-[({[(2S,3R)-3-({[(2-chloro-4- |
| | fluorophenyl)sulfonyl]amino}methyl)tetrahydro-2- |
| | furanyl]methyl}amino)carbonyl]-3-methylbutyl}-1-benzothiophene-2- |
| | carboxamide |
| 117. | N-{(1S)-1-[({[2-({[(2,4- |
| | dichlorophenyl)sulfonyl]amino}methyl)cyclopentyl]methyl}amino)carbonyl]- |
| | 3-methylbutyl}-1-benzothiophene-2-carboxamide |
| 118. | N-{(1S)-1-[({[2-({[(2-chloro-4- |
| | fluorophenyl)sulfonyl]amino}methyl)cyclopentyl]methyl}amino)carbonyl]- |
| | 3-methylbutyl}-1-benzothiophene-2-carboxamide |
| 119. | 2,4-dichloro-N-{4-[[(cyclohexylamino)carbonyl]((2S)-4-methyl-2- |
| | {[(phenylamino) |
| | carbonyl]amino}pentyl)amino]butyl}benzenesulfonamide |
| 120. | 2,4-dichloro-N-{4-[[(4-fluorophenyl)sulfonyl]((2S)-4-methyl-2- |
| | {[(phenylamino) |
| | carbonyl]amino}pentyl)amino]butyl}benzenesulfonamide |
| 121. | N-(4-{[(2,4-dichlorophenyl)sulfonyl]amino}butyl)-N-((2S)-4-methyl-2- |
| | {[(phenylamino) carbonyl]amino}pentyl)acetamide |
| 122. | methyl (4-{[(2,4-dichlorophenyl)sulfonyl]amino}butyl)((2S)-4-methyl-2- |
| | {[(phenylamino) carbonyl]amino}pentyl)carbamate |
| 123. | 2,4-dichloro-N-{4-[[(ethylamino)carbonyl]((2S)-4-methyl-2- |
| | {[(phenylamino) |
| | carbonyl]amino}pentyl)amino]butyl}benzenesulfonamide |
| 124. | 2,4-dichloro-N-{4-[{[(1-methylethyl)amino]carbonyl}((2S)-4-methyl-2- |
| | {[(phenylamino)carbonyl] |
| | amino}pentyl)amino]butyl}benzenesulfonamide |
| 125. | 2,4-dichloro-N-(4-{((2S)-4-methyl-2- |
| | {[(phenylamino)carbonyl]amino}pentyl)[(phenylamino) |
| | carbonyl]amino}butyl)benzenesulfonamide |
| 126. | N-(4-{[(2,4-dichlorophenyl)sulfonyl]amino}butyl)-N-((2S)-4-methyl-2- |
| | {[(phenylamino)carbonyl] amino}pentyl)cyclohexanecarboxamide |
| 127. | 2,4-dichloro-N-(4-{{[(1-methylethyl)amino]carbonyl}[(2S)-4-methyl-2- |
| | ({[(1-methylethyl)amino] |
| | carbonyl}amino)pentyl]amino}butyl)benzenesulfonamide |
| 128. | 2,4-dichloro-N-[4-(((2S)-2-{[(cyclohexylamino)carbonyl]amino}-4- |
| | methylpentyl){[(1- |
| | methylethyl)amino]carbonyl}amino)butyl]benzenesulfonamide |
| 129. | 2,4-dichloro-N-[4-(((2S)-2-{[(ethylamino)carbonyl]amino}-4- |
| | methylpentyl){[(1-methylethyl) |
| | amino]carbonyl}amino)butyl]benzenesulfonamide |
| 130. | 2,4-dichloro-N-[4-([(2S)-2-({[(4-fluorophenyl)amino]carbonyl}amino)-4- |
| | methylpentyl]{[(1- |
| | methylethyl)amino]carbonyl}amino)butyl]benzenesulfonamide |
| 131. | 2,4-dichloro-N-[4-({[(1-methylethyl)amino]carbonyl}{(2S)-4-methyl-2- |
| | [({[4-(1H-pyrrol-1- |
| | yl)phenyl]amino}carbonyl)amino]pentyl}amino)butyl]benzenesulfonamide |
| 132. | 2,4-dichloro-N-(4-{{[(1-methylethyl)amino]carbonyl}[(2S)-4-methyl-2- |
| | ({[(1,3,5-trimethyl-1H-pyrazol-4- |
| | yl)amino]carbonyl}amino)pentyl]amino}butyl)benzenesulfonamide |
| 133. | 2,4-dichloro-N-{4-[[(cyclopropylamino)carbonothioyl]((2S)-4-methyl-2- |
| | {[(phenylamino) |
| | carbonyl]amino}pentyl)amino]butyl}benzenesulfonamide |
| 134. | 2,4-dichloro-N-{4-[{[(1,1-dimethylethyl)amino]carbonyl}((2S)-4-methyl- |
| | 2-{[(phenylamino) |
| | carbonyl]amino}pentyl)amino]butyl}benzenesulfonamide |
| 135. | 2,4-dichloro-N-{4-[({[4-(dimethylamino)phenyl]amino}carbonyl)((2S)-4- |
| | methyl-2- |
| | {[(phenylamino)carbonyl]amino}pentyl)amino]butyl}benzenesulfonamide |
| 136. | 2,4-dichloro-N-{4-[{[(2-furanylmethyl)amino]carbonyl}((2S)-4-methyl-2- |
| | {[(penylamino)carbonyl]amino}pentyl)amino]butyl}benzenesulfonamide |
| 137. | 2,4-dichloro-N-(4-{((2S)-4-methyl-2- |
| | {[(penylamino)carbonyl]amino}pentyl)[(3-thienylamino) |
| | carbonyl]amino}butyl)benzenesulfonamide |
| 138. | 2,4-dichloro-N-{4-[((2S)-4-methyl-2- |
| | {[(phenylamino)carbonyl]amino}pentyl)({[1-(trifluoroacetyl)-4- |
| | piperidinyl]amino}carbonyl)amino]butyl}benzenesulfonamide |
| 139. | 2,4-dichloro-N-{4-[{[(1,1-dioxidotetrahydro-3- |
| | thienyl)amino]carbonyl}((2S)-4-methyl-2- |
| | {[(phenylamino)carbonyl]amino}pentyl)amino]butyl}benzenesulfonamide |
| 140. | 2,4-dichloro-N-{4-[[(methylamino)carbonothioyl]((2S)-4-methyl-2- |
| | {[(phenylamino) |
| | carbonyl]amino}pentyl)amino]butyl}benzenesulfonamide |
| 141. | 2,4-dichloro-N-{4-[{[(1-methylethyl)amino]carbonyl}((2S)-4-methyl-2- |
| | {[(2-pyridinylamino) |
| | carbonothioyl]amino}pentyl)amino]butyl}benzenesulfonamide |
| 142. | 2,4-dichloro-N-{4-[{[(1-methylethyl)amino]carbonyl}((2S)-4-methyl-2- |
| | {[(phenylamino) |
| | carbonothioyl]amino}pentyl)amino]butyl}benzenesulfonamide |
| 143. | 2,4-dichloro-N-{4-[{[(1-methylethyl)amino]carbonyl}((2S)-4-methyl-2- |
| | {[(3-thienylamino) |
| | carbonyl]amino}pentyl)amino]butyl}benzenesulfonamide |
| 144. | 2,4-dichloro-N-[4-({[(1-methylethyl)amino]carbonyl}{(2S)-4-methyl-2- |
| | [({[1-(trifluoroacetyl)-4- |
| | piperidinyl]amino}carbonyl)amino]pentyl}amino)butyl]benzenesulfonamide |
| 145. | 2,4-dichloro-N-{4-[{[(1-methylethyl)amino]carbonyl}((2S)-4-methyl-2- |
| | {[(2-pyridinylamino) |
| | carbonyl]amino}pentyl)amino]butyl}benzenesulfonamide |
| 146. | 2,4-dichloro-N-[4-(((2S)-2-{[(cyclopentylamino)carbonyl]amino}-4- |
| | methylpentyl){[(1-methyl |
| | ethyl)amino]carbonyl}amino)butyl]benzenesulfonamide |
| 147. | N-(4-{[(2,4-dichlorophenyl)sulfonyl]amino}butyl)-N-((2S)-4-methyl-2- |
| | {[(phenylamino)carbonyl]amino}pentyl)-2-phenylacetamide |
| 148. | 2,4-dichloro-N-[4-([(2R)-2-hydroxy-3-(methyloxy)propyl]{[(1- |
| | methylethyl)amino]carbonyl}amino)butyl]benzenesulfonamide |
| 149. | (1R)-2-((4-{[(2,4-dichlorophenyl)sulfonyl]amino}butyl){[(1- |
| | methylethyl)amino]carbonyl}amino)-1-[(methyloxy)methyl]ethyl |
| | phenylcarbamate |
| 150. | (1R)-2-{(4-{[(2,4- |
| | dichlorophenyl)sulfonyl]amino}butyl)[(ethylamino)carbonyl]amino}-1- |
| | [(methyloxy)methyl]ethyl phenylcarbamate |
| 151. | (1R)-2-{(4-{[(2,4- |
| | dichlorophenyl)sulfonyl]amino}butyl)[(phenylamino)carbonyl]amino}-1- |
| | [(methyloxy)methyl]ethyl phenylcarbamate |
| 152. | 2,4-dichloro-N-[4-({(2R)-2-hydroxy-3-[(phenylmethyl)oxy]propyl}{[(1- |
| | methylethyl)amino]carbonyl}amino)butyl]benzenesulfonamide |
| 153. | (1R)-2-((4-{[(2,4-dichlorophenyl)sulfonyl]amino}butyl){[(1- |
| | methylethyl)amino]carbonyl}amino)-1-{[(phenylmethyl)oxy]methyl}ethyl |
| | phenylcarbamate |
| 154. | 2,4-dichloro-N-[4-([(2S)-2-hydroxy-3-(4-morpholinyl)propyl]{[(1- |
| | methylethyl)amino]carbonyl}amino)butyl]benzenesulfonamide |
| 155. | (1S)-2-((4-{[(2,4-dichlorophenyl)sulfonyl]amino}butyl){[(1- |
| | methylethyl)amino]carbonyl}amino)-1-(4-morpholinylmethyl)ethyl |
| | phenylcarbamate |
| 156. | (1R)-2-((4-{[(2-chloro-4-fluorophenyl)sulfonyl]amino}butyl){[(1- |
| | methylethyl)amino]carbonyl}amino)-1-[(methyloxy)methyl]ethyl |
| | phenylcarbamate |
| 157. | (1R)-2-([4-({[4-fluoro-2- |
| | (trifluoromethyl)phenyl]sulfonyl}amino)butyl]{[(1- |
| | methylethyl)amino]carbonyl}amino)-1-[(methyloxy)methyl]ethyl |
| | phenylcarbamate |
| 158. | (1R)-2-[{[(1-methylethyl)amino]carbonyl}(4-{[(2- |
| | nitrophenyl)sulfonyl]amino}butyl)amino]-1-[(methyloxy)methyl]ethyl |
| | phenylcarbamate |
| 159. | (1R)-2-((4-{[(2,4-dichlorophenyl)sulfonyl]amino}butyl){[(1- |
| | methylethyl)amino]carbonyl}amino)-1-[(methyloxy)methyl]ethyl (4- |
| | fluorophenyl)carbamate |
| 160. | (1S)-2-((4-{[(2,4-dichlorophenyl)sulfonyl]amino}butyl){[(1- |
| | methylethyl)amino]carbonyl}amino)-1-[(methyloxy)methyl]ethyl |
| | phenylcarbamate |
| 161. | (1R)-2-{(4-{[(2-chloro-4- |
| | fluorophenyl)sulfonyl]amino}butyl)[(ethylamino)carbonyl]amino}-1- |
| | [(methyloxy)methyl]ethyl phenylcarbamate |
| 162. | (1R)-2-{[(ethylamino)carbonyl][4-({[4-fluoro-2- |
| | (trifluoromethyl)phenyl]sulfonyl}amino)butyl]amino}-1- |
| | [(methyloxy)methyl]ethyl phenylcarbamate |
| 163. | O-{(1R)-2-{(4-{[(2,4- |
| | dichlorophenyl)sulfonyl]amino}butyl)[(ethylamino)carbonyl]amino}-1- |
| | [(methyloxy)methyl]ethyl}phenylthiocarbamate |
| 164. | (1R)-2-[{[(1-methylethyl)amino]carbonyl}(4-{methyl[(2- |
| | nitrophenyl)sulfonyl]amino}butyl)amino]-1-[(methyloxy)methyl]ethyl |
| | phenylcarbamate |
| 165. | (1R)-2-({4-[[(2-cyanophenyl)sulfonyl](methyl)amino]butyl}{[(1- |
| | methylethyl)amino]carbonyl}amino)-1-[(methyloxy)methyl]ethyl |
| | phenylcarbamate |
| 166. | 2-[[(cyclohexylamino)carbonyl](4-{[(2,4- |
| | dichlorophenyl)sulfonyl]amino}butyl)amino]ethyl phenylcarbamate |
| 167. | 2-[[(cyclohexylamino)carbonyl](4-{[(2,4- |
| | dichlorophenyl)sulfonyl]amino}butyl)amino]ethyl cyclohexylcarbamate |
| 168. | 2-[[(cyclohexylamino)carbonyl](4-{[(2,4- |
| | dichlorophenyl)sulfonyl]amino}butyl)amino]ethyl methylcarbamate |
| 169. | 2-chlorophenyl [2-((4-{[(2,4-dichlorophenyl)sulfonyl]amino}butyl){[(1- |
| | methylethyl)amino]carbonyl}amino)ethyl]carbamate |
| 170. | 2,4-dichloro-N-{4-[{[(1-methylethyl)amino]carbonyl}((2R)-3 |
| | (methyloxy)-2- |
| | {[(phenylamino)carbonyl]amino}propyl)amino]butyl}benzenesulfonamide |
| 171. | 2-[((4-{[(2,4-dichlorophenyl)sulfonyl]amino}butyl){[(1- |
| | methylethyl)amino]carbonyl}amino)methyl]-N-phenyl-1- |
| | piperidinecarboxamide |
| 172. | (3R)-3-((4-{[(2,4-dichlorophenyl)sulfonyl]amino}butyl){[(1- |
| | methylethyl)amino]carbonyl}amino)-N-phenyl-1-piperidinecarboxamide |
| 173. | (3S)-3-((4-{[(2,4-dichlorophenyl)sulfonyl]amino}butyl){[(1- |
| | methylethyl)amino]carbonyl}amino)-N-phenyl-1-piperidinecarboxamide |
| 174. | 2-[((4-{[(2,4-dichlorophenyl)sulfonyl]amino}butyl){[(1- |
| | methylethyl)amino]carbonyl}amino)methyl]-N-phenyl-1- |
| | pyrrolidinecarboxamide |
| 175. | 2-({(4-{[(2,4- |
| | dichlorophenyl)sulfonyl]amino}butyl)[(ethylamino)carbonyl]amino}methyl)- |
| | N-phenyl-1-pyrrolidinecarboxamide |
| 176. | N-(4-{[(2,4-dichlorophenyl)sulfonyl]amino}butyl)-2,2-dimethyl-N-((2S)- |
| | 4-methyl-2- |
| | {[(phenylamino)carbonyl]amino}pentyl)hydrazinecarboxamide |
| 177. | 2,4-dichloro-N-{4-[{[(2-fluoroethyl)amino]carbonyl}((2S)-4-methyl-2- |
| | {[(phenylamino)carbonyl]amino}pentyl)amino]butyl}benzenesulfonamide |
| 178. | 2-chloro-4-fluoro-N-{4-[{[(1-methylethyl)amino]carbonyl}((2S)-4-methyl- |
| | 2- |
| | {[(phenylamino)carbonyl]amino}pentyl)amino]butyl}benzenesulfonamide |
| 179. | 4-fluoro-N-{4-[{[(1-methylethyl)amino]carbonyl}((2S)-4-methyl-2- |
| | {[(phenylamino)carbonyl] amino}pentyl)amino]butyl}-2- |
| | (trifluoromethyl)benzenesulfonamide |

The compounds contained within this application may be prepared by the general syntheses outlined below in Schemes 1-24. Coupling of N-hydroxysuccinamide (2) with benzothiophene 2-carboxylic acid (1) in the presence of a coupling agent such as 3 provides the activated benzothiophene analog 4. Treatment of 4 with an amine such as leucine (5) provides the intermediate 6. Coupling of 6 with an amine such as 7 in the presence of a coupling agent common to the art provides intermediate 8. Deprotection of the N-Boc protecting group gives the amine salt 9 which is converted to the sulfonamide 11 via methods common to art.

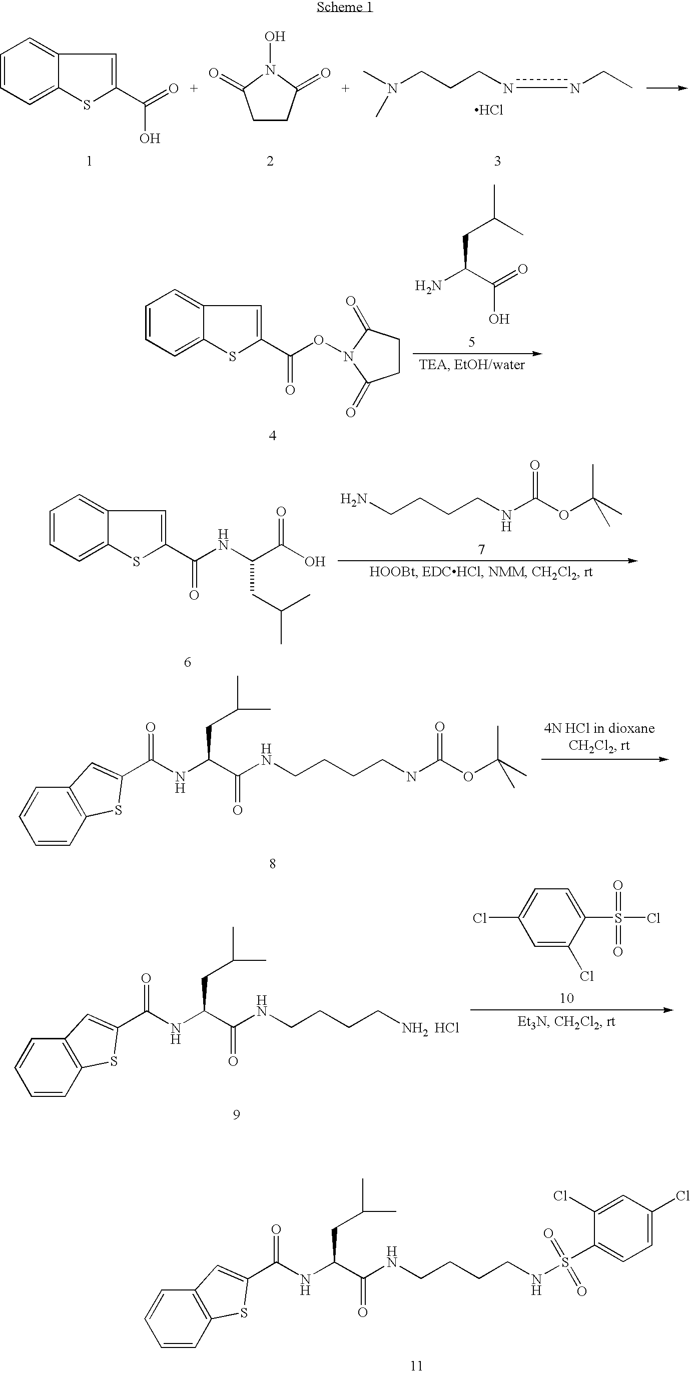

Alternatively analogs of this application may be prepared as outlined in Scheme 2. Sulfonylation of 1,4-butandiamine (12) with a sulfonylchloride such as 10 provides intermediate 13. Coupling of 13 with a carboxylic acid such as 14 in the presence of a coupling agent common to the art provides intermediate 15. Deprotection of 15 with acid followed by coupling of the resulting amine salt (not shown) with a carboxylic acid such as 16 in the presence of a coupling agent provides 17.

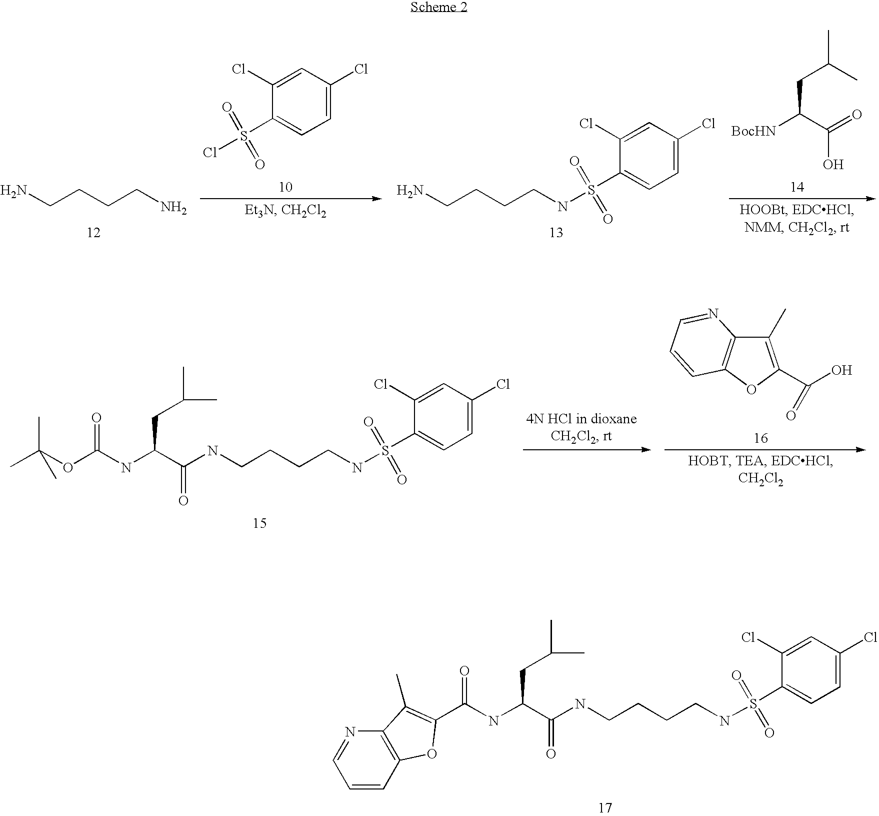

As shown in Scheme 3, treatment of a carboxylic acid such as benzithiophene-2-carboxylic acid with N-hydroxysuccinimide and EDC provides the activated ester 1-[(1-benzothien-2-ylcarbonyl)oxy]-2,5-pyrrolidinedione 4 as shown in Scheme 1. Subsequent coupling with (2S)-2-amino-4,4-dichloro-butanoic acid 18 prepared by the D. Winkler's procedure (Synthesis, 1996, 1419) provides the carboxylic acid 19. Treatment of the representative amine 13 (shown in Scheme 2) with the carboxylic acid 19 by the standard peptide coupling condition provides the final product 20.

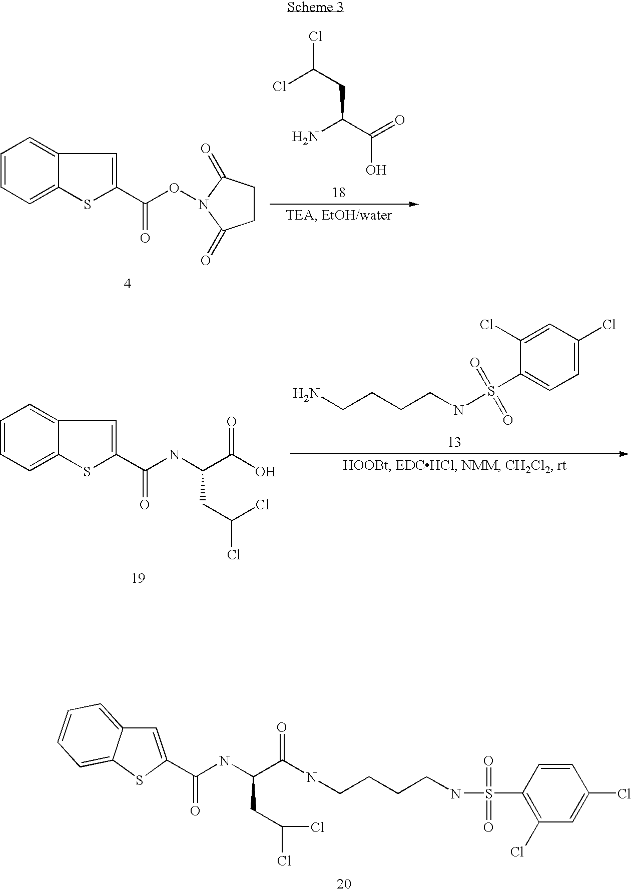

As shown in Scheme 4, treatment of 1,4-diaminobutane (12) with 2,4-disubstituted sulfonyl chloride such as 2-chloro-4-fluorosulfonyl chloride results in the formation of the representative mono-sulfonamide 21. A standard peptide coupling condition with Boc-phenylglycine using EDC, HOOBt, and NMM provides the representative amide 22. Removal of the tert-butyl carbonyl group under standard conditions such as HCl or TFA followed by the subsequent coupling of the resultant amine 22 with the activated ester 1-[(1-benzothien-2-ylcarbonyl)oxy]-2,5-pyrrolidinedione 4 provides the final product 24.

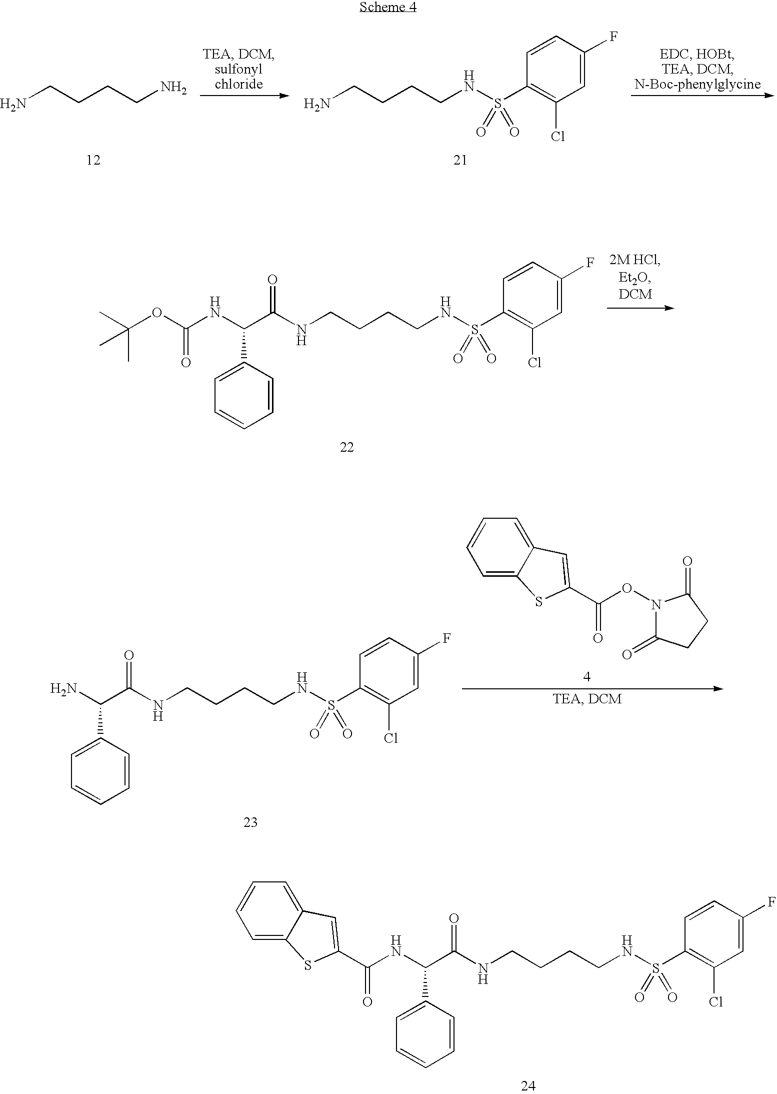

As shown in Scheme 5, treatment of 3-biphenylylacetic acid (25) with 3-bromo-2-methyl-1-propene in the presence of LDA results in the formation of the alkylated carboxylic acid 26 followed by hydrogenation (Pd/C, H₂) to provide the saturated carboxylic acid 27. Treatment of the representative amine 13 (shown in Scheme 2) with the carboxylic acid 27 by the standard peptide coupling condition provides the final product 28.

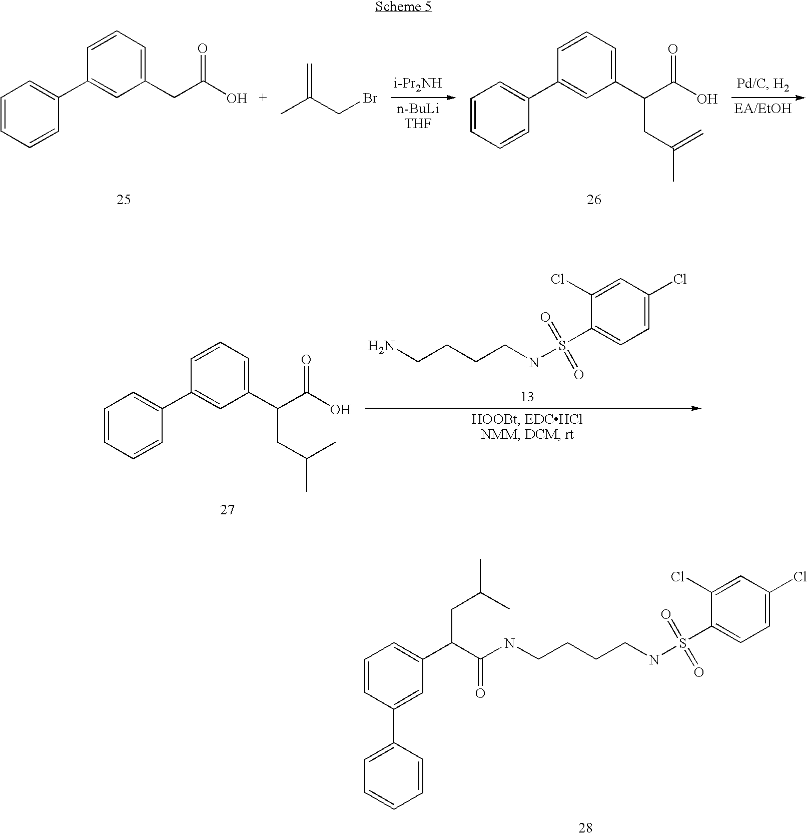

As shown in Scheme 6, treatment of (R)-(+)-glycidol (29) with phthalamide in the presence of triphenylphosphine and DEAD results in the formation of the epoxide 30 followed by heating with 2-hydroxy-2-methylpropane nitrile to provide the hydroxyl nitrile 31. Reduction of the nitrile 31 using PtO₂ and H₂ provides the amine 4, which is converted to the sulfonamide 33 from the coupling with 2,4-disubstituted sulfonyl chloride such as 2-chloro-4-fluorosulfonyl chloride. Treatment of the sulfonamide with hydrazine provides the free amine 34 followed by the standard peptide coupling reaction with the carboxylic acid 6 to give the final product 35.

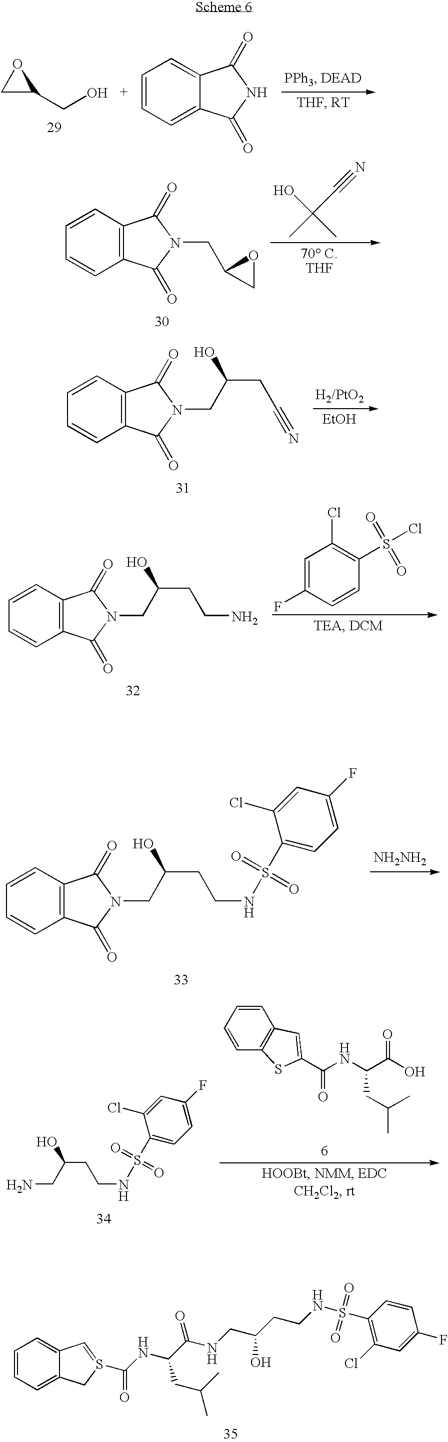

As shown in Scheme 7, treatment of the free amine 32 (shown in Scheme 6) with Boc-Leu by a standard peptide coupling condition using EDC, HOOBt, and NMM provides the amide 36. Treatment of the amide 36 with hydrazine provides the free amine 37 followed by the coupling with 2-chloro-4-fluorosulfonyl chloride to give the sulfonamide 38. Removal of the tert-butyl carbonyl group under standard conditions such as HCl or TFA followed by treatment of the resultant amine (not shown) with a carboxylic acid 1 by the standard peptide coupling condition provides the final product 39.

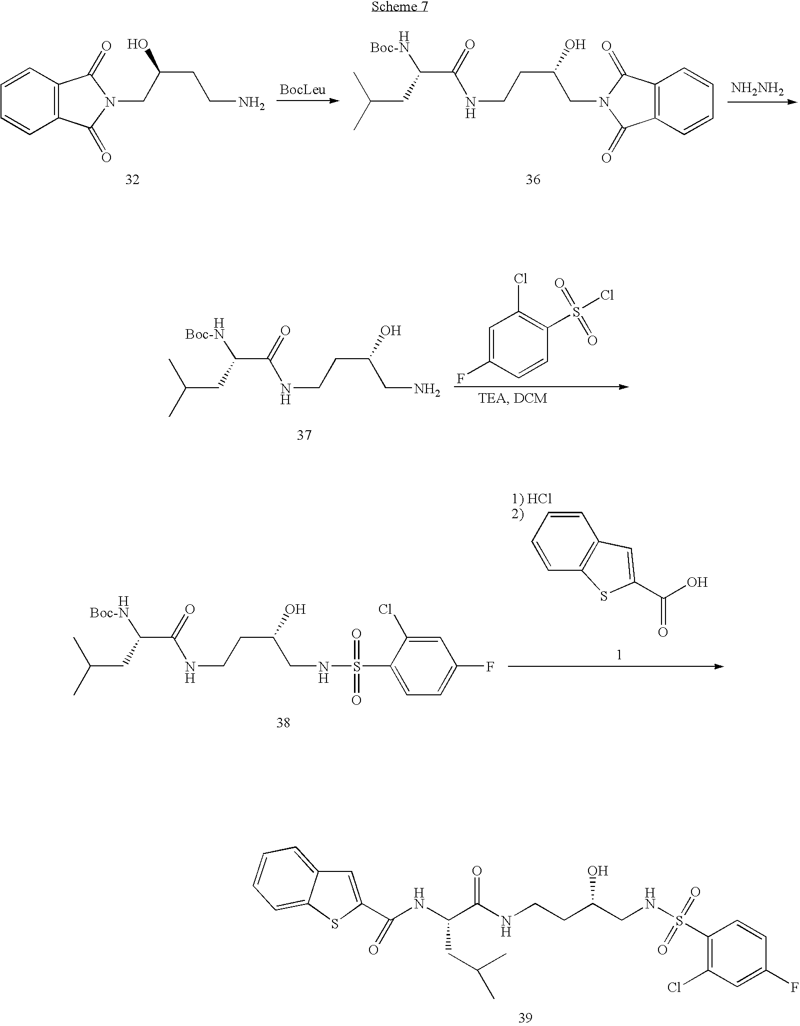

As shown in Scheme 8, treatment of the amino acid 40 with 2-chloro-4-fluorosulfonyl chloride provides the sulfonamide 41 followed by the reduction with BH₃ to give the primary alcohol 42. Removal of the tert-butyl carbonyl group under standard conditions such as HCl or TFA followed by treatment of the resultant amine 43 with the carboxylic acid 7 by the standard peptide coupling condition provides the final product 44.

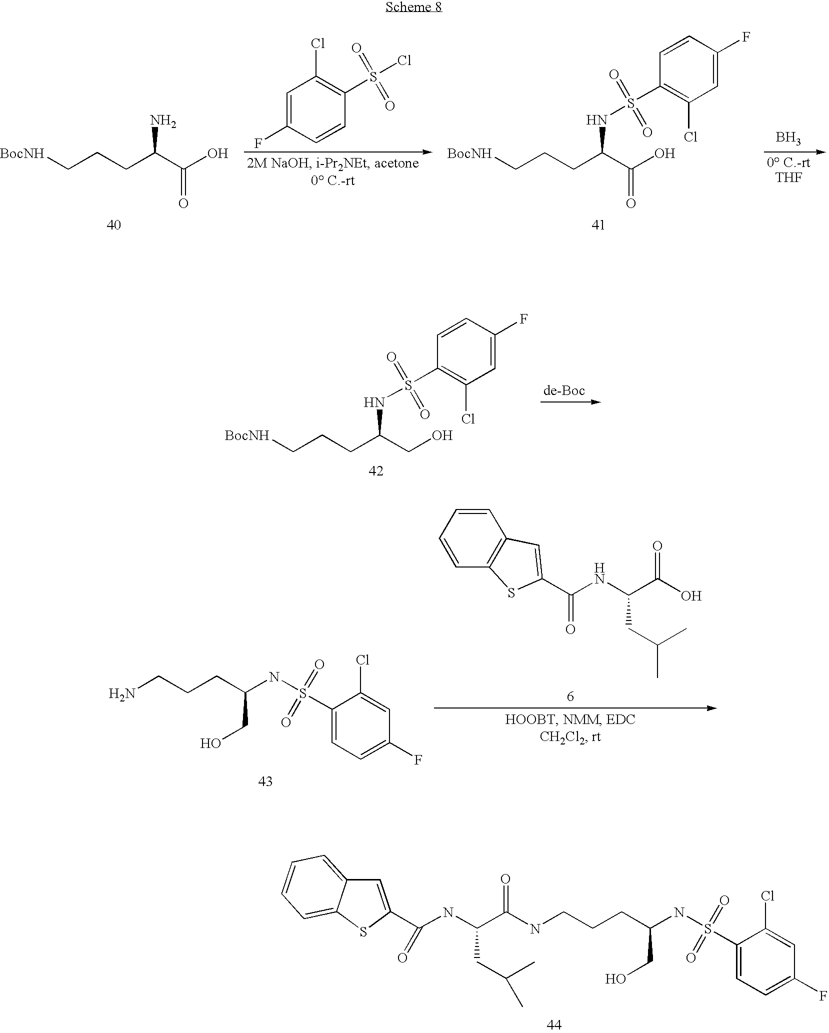

As shown in Scheme 9, treatment of the carboxylic acid 41 with TMSCHN₂ provides the methyl ester (not shown). Removal of the tert-butyl carbonyl group under standard conditions such as HCl or TFA followed by treatment of the resultant amine with the carboxylic acid 6 by the standard peptide coupling condition provides the amide 45. After hydrolysis of the ester 45 to the carboxylic acid 46 under a basic condition such as K₂CO₃, the carboxylic acid 46 is converted to the final amide 47 with a primary amine such as methylamine using the standard peptide coupling condition.

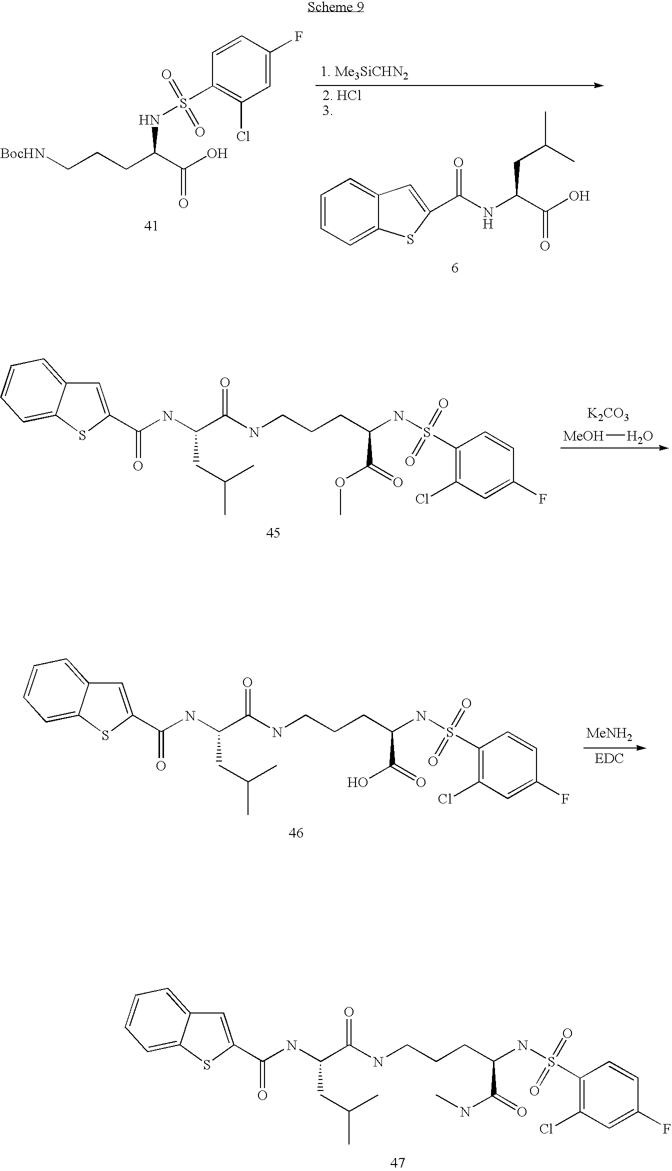

As shown in Scheme 10, treatment of the bis-tosylate 48 with sodium azide followed by displacement of the tosylate 49 with potassium phthalimide provides the azide 50. The reduction of the azide 50 with Pd/C and H2 in the presence of (Boc)₂O provides the Boc-protected amine 51. Treatment of the phthalimide 51 with hydrazine provides the free amine 52 followed by the coupling reaction with 2,4-dichlorosulfonyl chloride to give the sulfonamide 53. Removal of the tert-butyl carbonyl group under standard conditions such as HCl or TFA followed by treatment of the resultant amine (not shown) with a carboxylic acid 6 by the standard peptide coupling condition provides the acetonide 54. The acetonide 54 is converted to the final diol 55 in the presence of an acid such as p-TsOH.

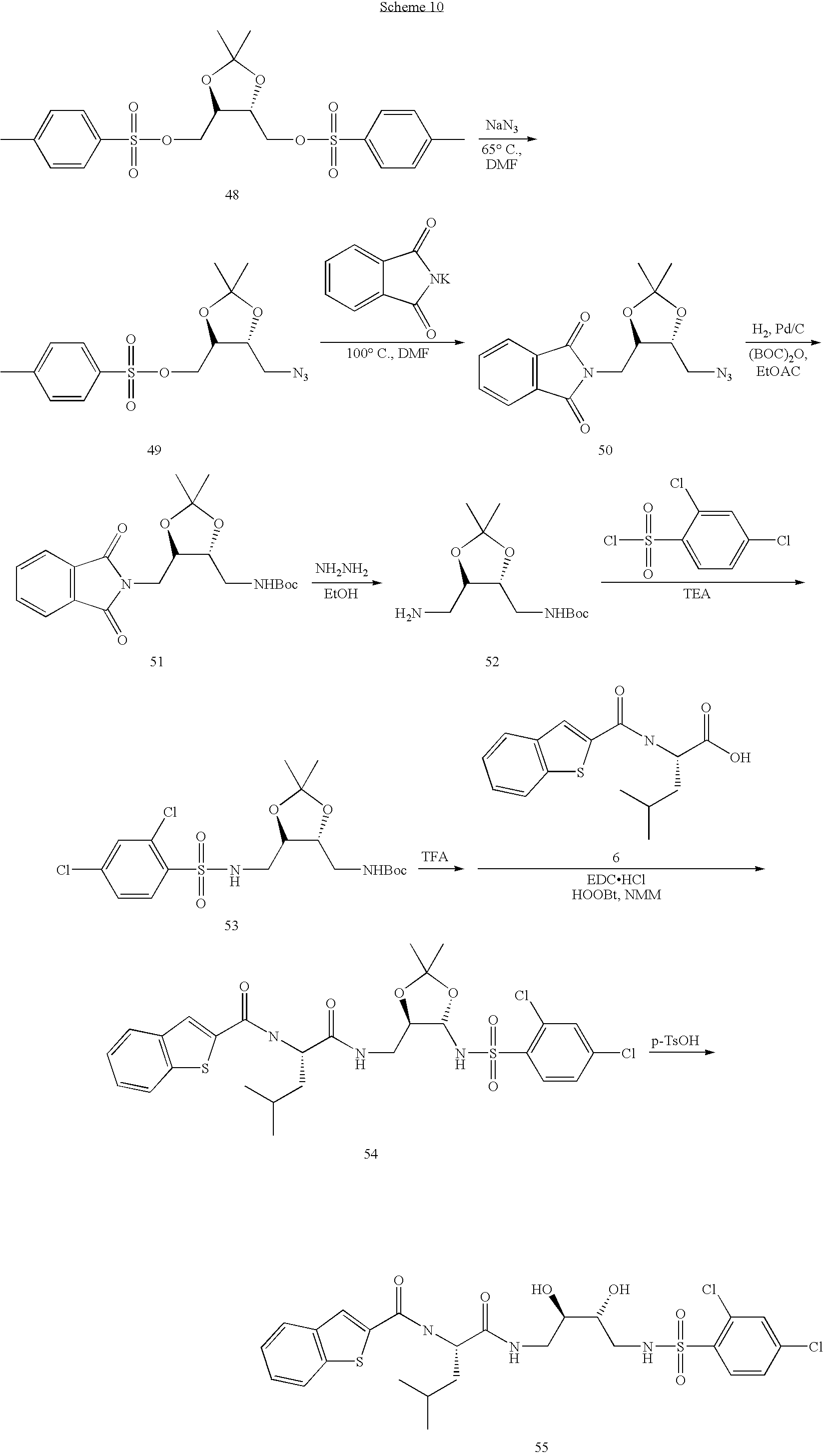

As shown in Scheme 11, treatment of bis-tosylate 48 with an acid such as p-TsOH followed by the cyclization of the resultant diol 56 with bis(methyloxy)methane in the presence of BF3.Et2O provides the dioxolane 57. After following the general procedure shown in Scheme 10, the final product 58 is obtained.

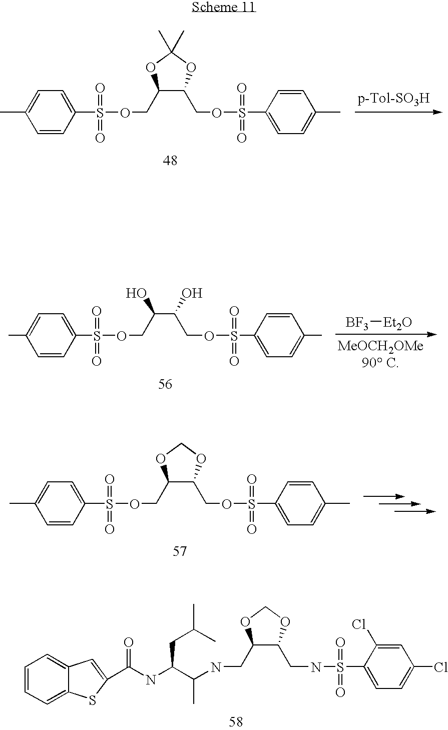

As shown in Scheme 12, asymmetric epoxidation of olefin 59 using standard Sharpless asymmetric epoxidation conditions provides the optically pure epoxide 60. The epoxide 60 is reacted with a nucleophile such as allyl magnesium bromide followed by the selective protection of the primary alcohol using benzoyl chloride to give the benzoate 61. After the olefin 61 is converted to the aldedyde, the 2-(methyloxy)tetrahydrofuran 62 is obtained under an acidic condition in methanol. The 2-(methyloxy)tetrahydrofuran 62 is reduced to tetrahydrofuran in the presence of Et₃SiH and BF₃OEt followed by hydrolysis of benzoate to give the primary alcohol 63. After the primary alcohol 63 is converted to the azide 64 via the mesylate (not shown), the azide 64 is reduced to the amine 65 in the presence of PPh₃ in wet THF. Treatment of the resultant amine 65 with a carboxylic acid 6 by the standard peptide coupling condition provides the benzyl ether 66. Removal of the benzyl group from the benzyl ether 66 with Pd/C and H₂ provides the primary alcohol 67 which is converted to the mesylate (not shown) followed by the conversion to the azide 68 using sodium azide. The azide 68 is reduced to the amine (not shown) in the presence of PPh₃ in wet THF. Treatment of the resultant amine 68 with 2-chloro-4-fluorosulfonyl chloride provides the final compound 69.

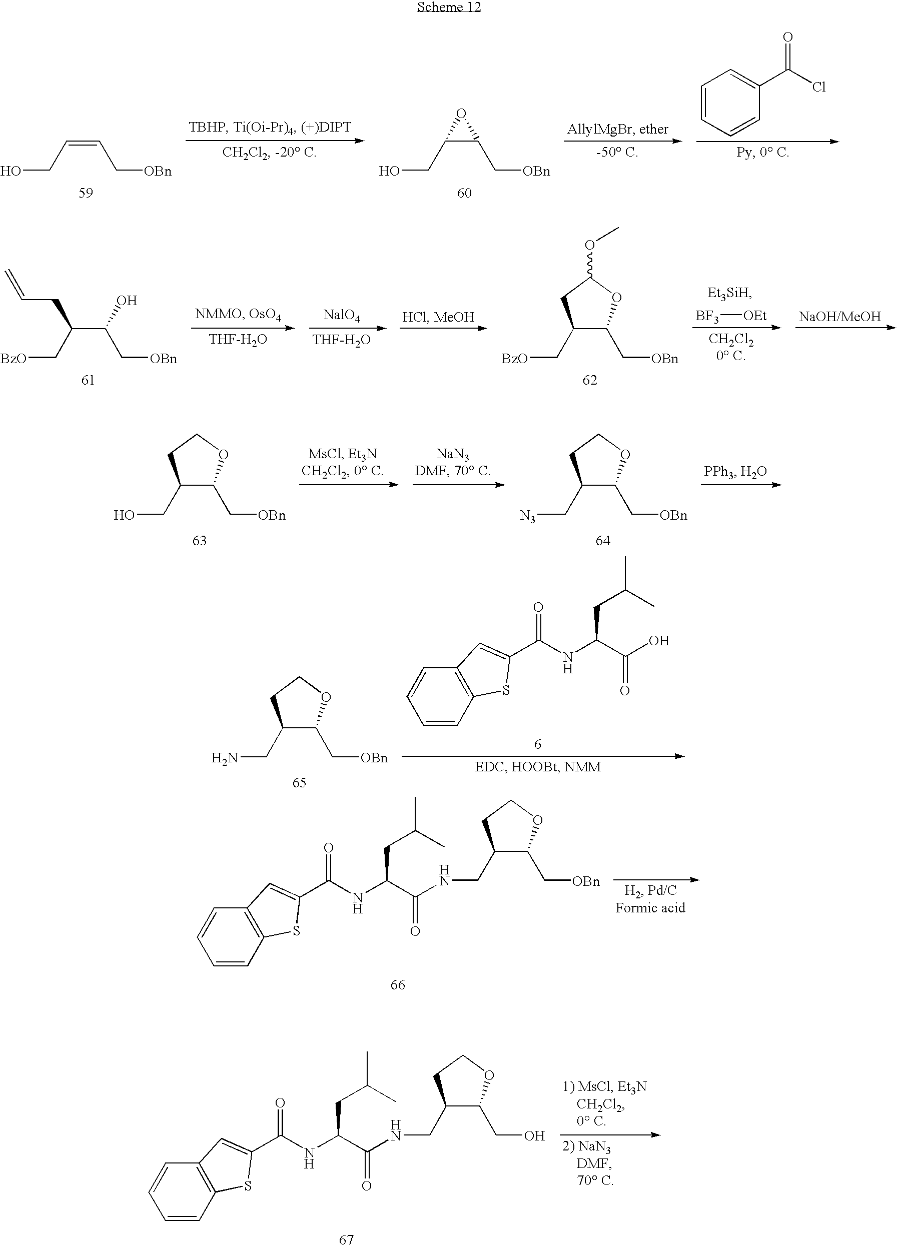

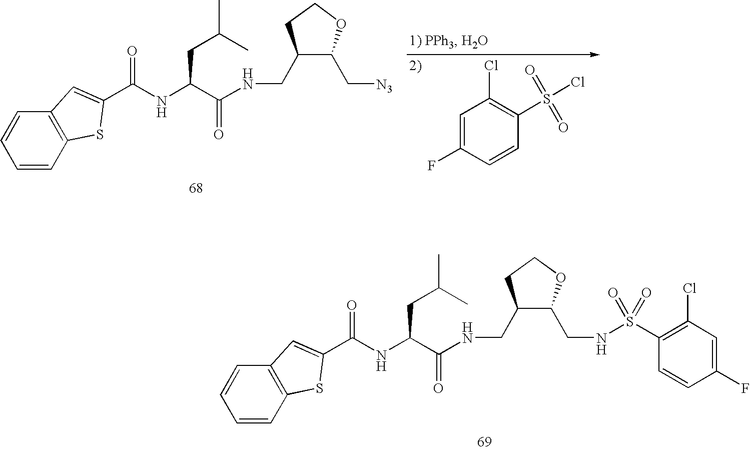

As shown in Scheme 13, treatment of the primary amine 65 (shown in Scheme 7) with (Boc)₂O provides the Boc-protected amine 70 followed by removal of the benzyl group using Pd/C and H₂ to give the primary alcohol 71. The primary alcohol 71 is converted to the azide 72 using the general procedure shown in Scheme 12. The azide 72 is reduced to the amine 73 in the presence of PPh₃ in wet THF. Treatment of the resultant amine 73 with a carboxylic acid 6 by the standard peptide coupling condition provides the amide 74. Removal of the tert-butyl carbonyl group under standard conditions such as HCl or TFA followed by treatment of the resultant amine (not shown) with 2-chloro-4-fluorosulfonyl chloride provides the final compound 75.

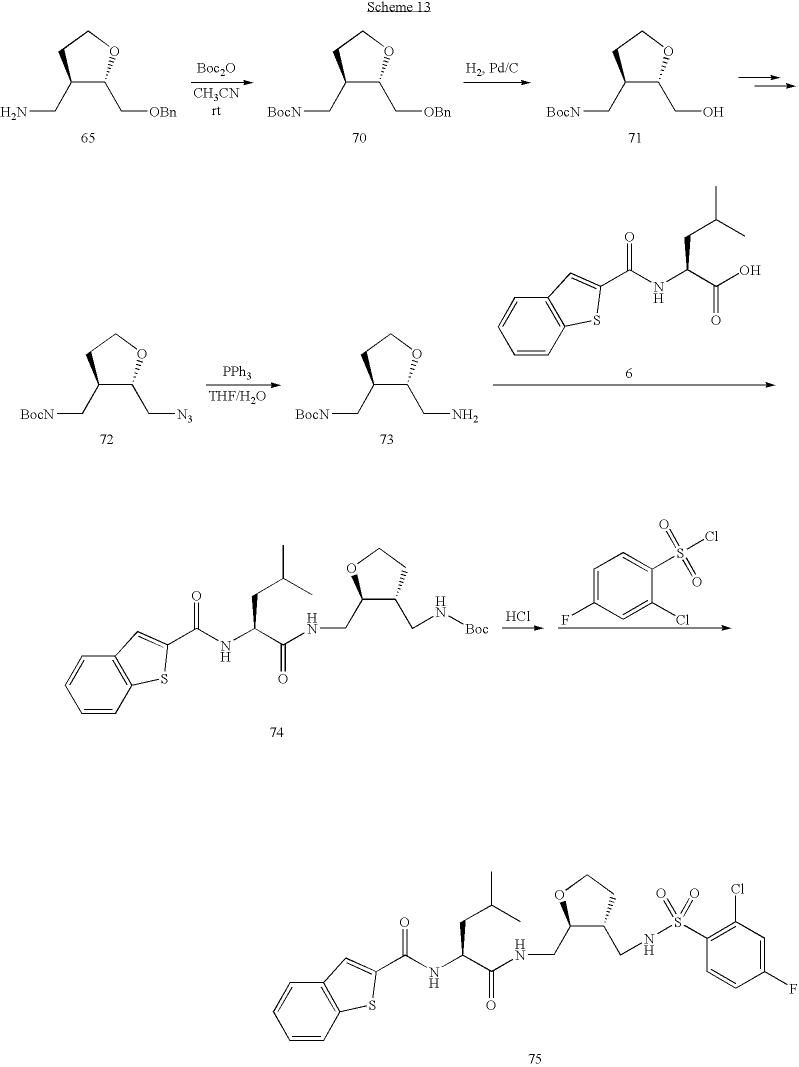

As shown in Scheme 14, the carboxylic acid 76 is reduced to the diol 77 using the reducing reagent such as BH₃. The diol 77 is converted to the mono-azide 78 using the general procedure as shown in Scheme 12 followed by displacement of the mesylate 78 with potassium phthalimide provides the azide 79. The final compound 80 is obtained using the general procedure shown in Scheme 10.

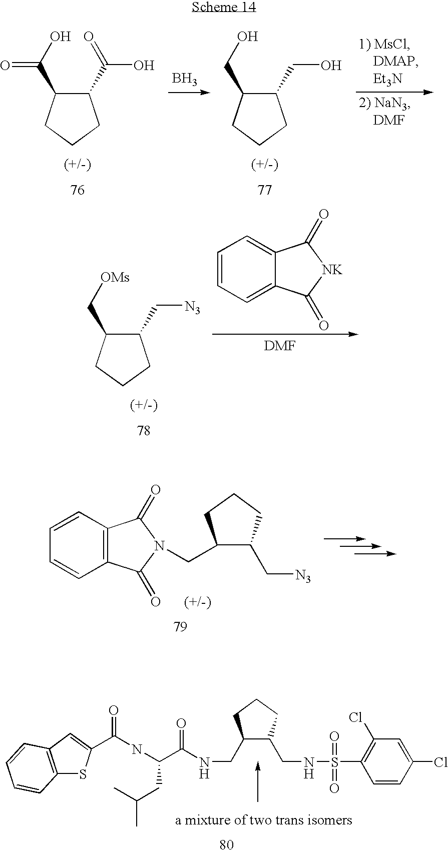

As shown in Scheme 15, the ester 81 is reduced to the aldehyde 82 using Dibal followed by reductive amination with the free amine 13 such as N-(4-aminobutyl)-2,4-dichlorobenzenesulfonamide in the presence of NaCNBH₃ and AcOH to give the secondary amine 83. The free amine 83 is coupled with FmocCl under the base such as Et₃N to provide the carbamate 84. Removal of the tert-butyl carbonyl group under conditions common to the art such as HCl or TFA provides the amine (not shown), which is coupling to an isocyanate such as phenylisocyanate under a basic condition such as Et₃N to provide the urea 85. Removal of the Fmoc group under conditions common to the art such as piperidine provides the amine 86 followed by coupling with an isocyanate such as cyclohexylisocyanate to provide the final product 87.

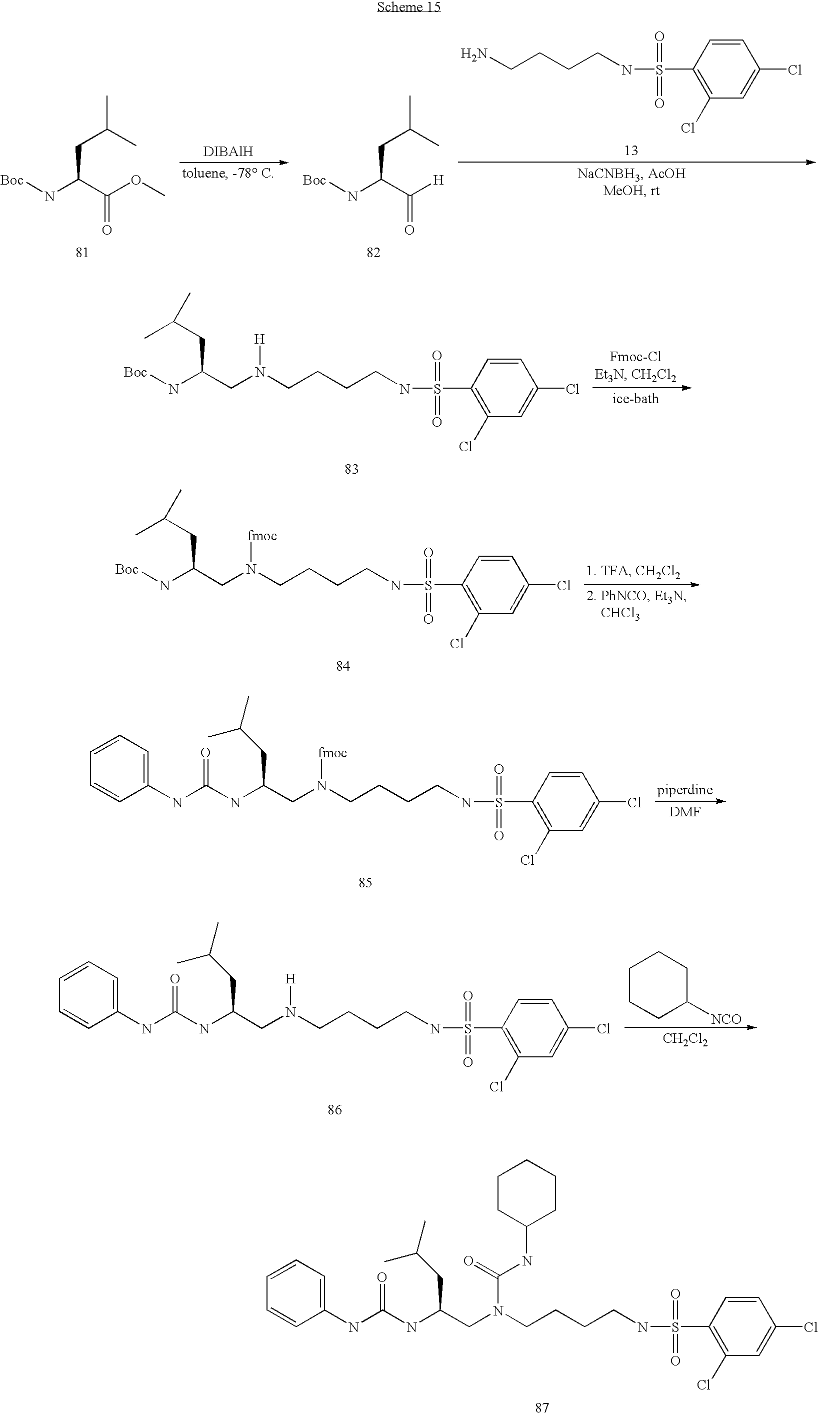

As shown in Scheme 16, an epoxide such as (R)-(−)-glycidyl methyl ether (88) is coupled with a free amine such as N-(4-aminobutyl)-2,4-dichlorobenzenesulfonamide (13) to provide the amine 89. The free amine 89 can be coupling to an isocyanate such as iso propylisocyanate and subsequent be coupling to an isocyanate such as phenylisocyanate under a basic condition such as NaH to give the final product 91.

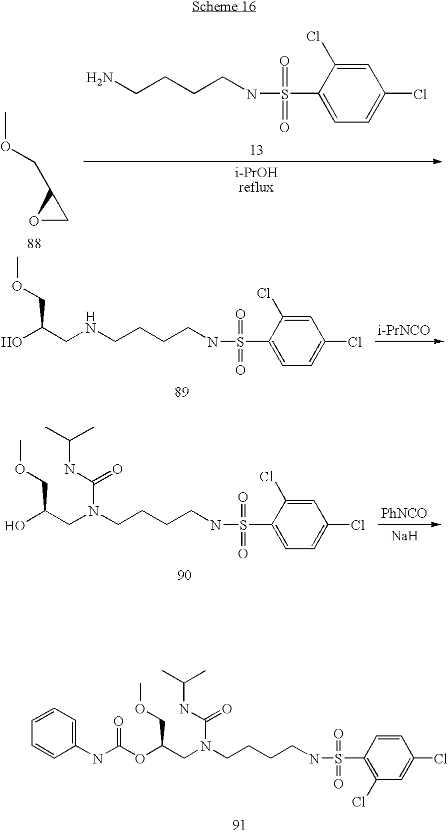

As shown in Scheme 17, an epoxide such as (2R)-2-oxiranylmethyl 3-nitrobenzenesulfonate (92) is coupled with a free amine such as morpholine to provide the epoxide 93. The epoxide 93 can be coupled with a free amine such as N-(4-aminobutyl)-2,4-dichlorobenzenesulfonamide (13) to provide the amine 94. The free amine 94 can be coupling to an isocyanate such as isopropylisocyanate and subsequent be coupling to an isocyanate such as phenylisocyanate under a basic condition such as NaH to give the final product 96.

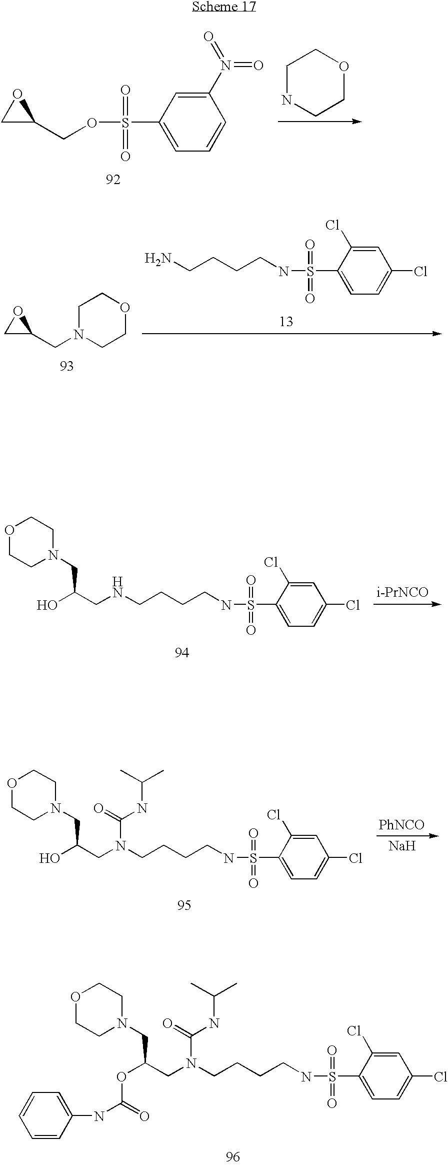

As shown in Scheme 18, some targets may be accessed by the following route. Treatment of an amine such as Boc-protected 1,4-diaminobutane with an epoxide such as (R)-(−)-glycidyl methyl ether (88) provides the free amine 97. The next two coupling procedures can be accomplished to provide the urea 98 and 99 as shown in Scheme 16 followed by removal of the tert-butyl carbonyl group under standard conditions such as HCl or TFA and subsequent treatment of the amine 100 with 2,4-disubstituted sulfonyl chloride such as 2-chloro-4-fluorosulfonyl chloride to provide the final compound 101.

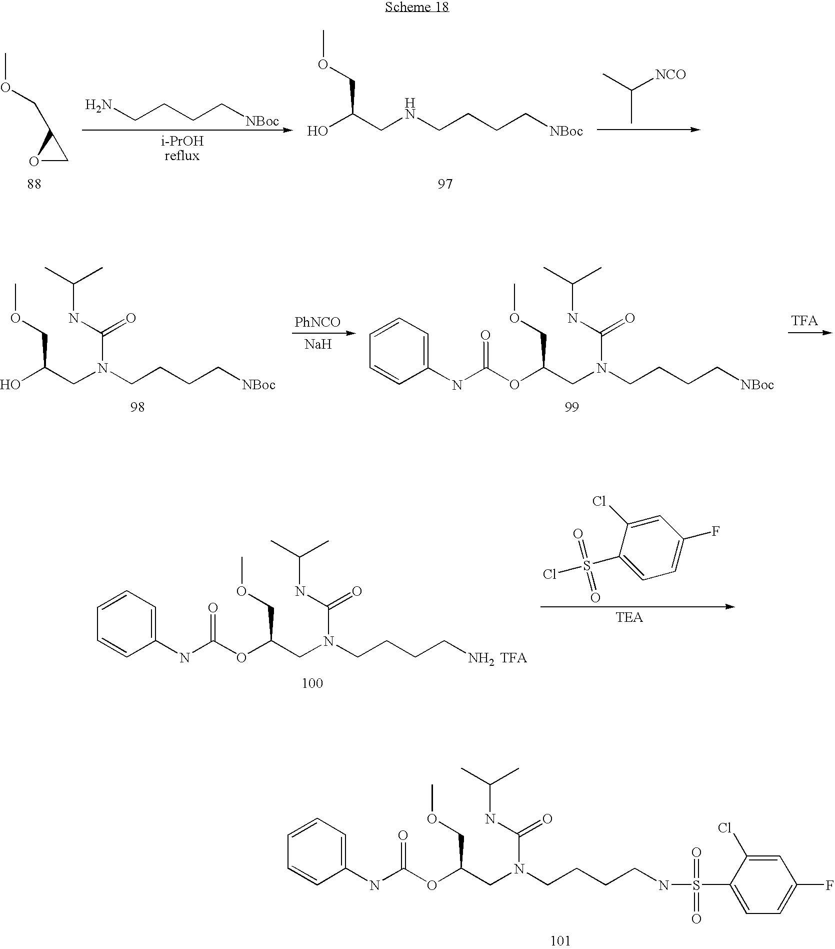

As shown in Scheme 19, various N-methyl sulfonamides may be accessed by the following route from the sulphonamide 102. Removal of the nosyl group can be accomplished by standard condition such as thiophenol and subsequent treatment of the free amine 103 with an electrophilic reagent such as 2-cyanobenzenesulfonyl chloride to provide the final compound 104.

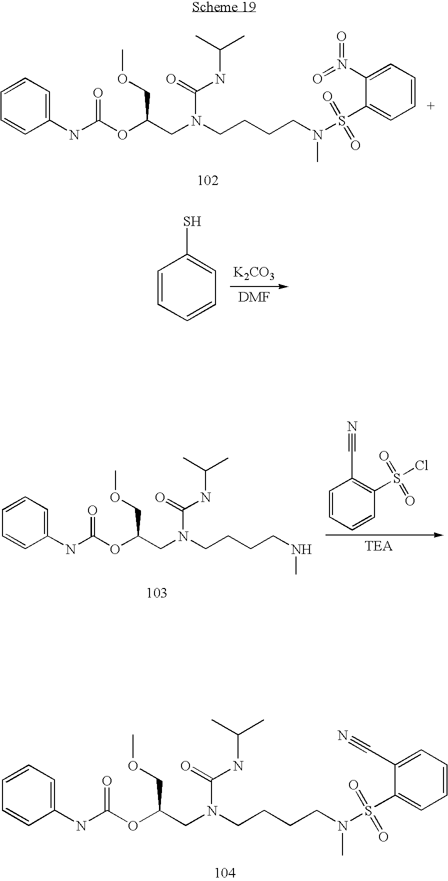

As shown in Scheme 20, reductive amination using the aldehyde 105 with the free amine 13 in the presence of NaCNBH₃ and AcOH to give the secondary amine 106. The free amine 106 is coupled with an isocyanate such as cyclohexylisocyanate to provide the urea 107. Removal of the benzyl group under conditions common to the art such as BBr₃ provides the alcohol 108, which is coupled with an isocyanate such as phenylisocyanate to give the final compound 109.

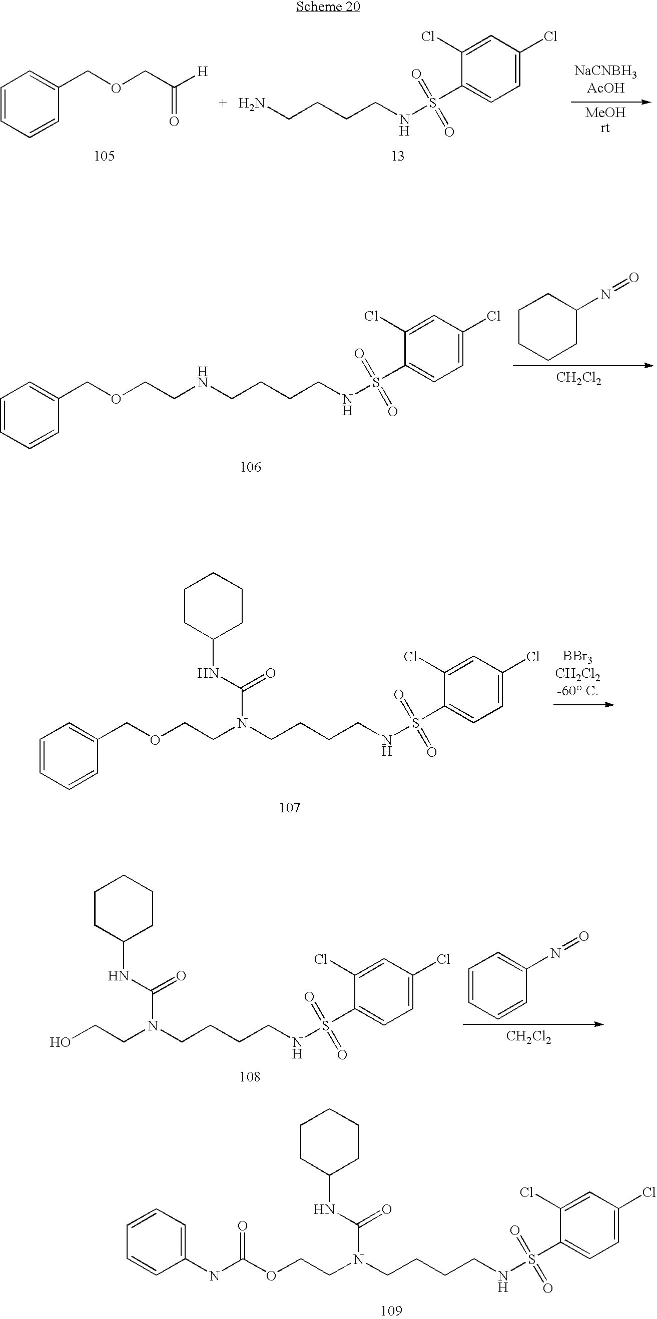

As shown in Scheme 21, some targets may be accessed by the following route. Reductive amination using the alternative aldehyde 110 with the free amine 13 in the presence of NaCNBH₃ and AcOH provides the secondary amine 111. The free amine 111 is coupled with an isocyanate such as iso-propylisocyanate to provide the urea 112. Removal of the tert-butyl carbonyl group under conditions common to the art such as HCl or TFA provides the amine (not shown), which is subsequently coupling to a chloroformate such as 2-chlorophenyl chloroformate under a basic condition such as Et3N to provide the final compound 113.

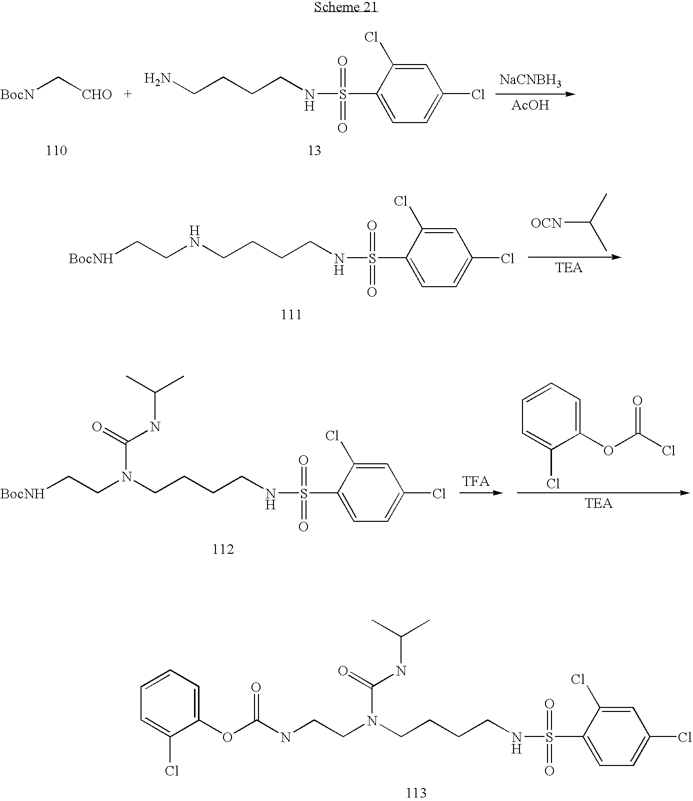

As shown in Scheme 22, some bis-ureas may be accessed by the following route. An epoxide such as (2S)-2-[(methyloxy)methyl]oxirane (114) is coupled with a free amine such as Boc-protected 1,4-diaminobutane to provide the amine 115. The free amine 115 is coupling to an isocyanate such as isopropylisocyanate followed by subsequent conversion of the alcohol 116 to the free amine (not shown) under standard conditions as shown in Scheme 12. The free amine (not shown) is coupling to an isocyanate such as phenylisocyanate to provide the bis-urea 117. Removal of the tert-butyl carbonyl group under standard conditions such as HCl or TFA followed by treatment of the resultant amine (not shown) with 2,4-dichlorobenzenesulfonyl chloride provides the final compound 118.

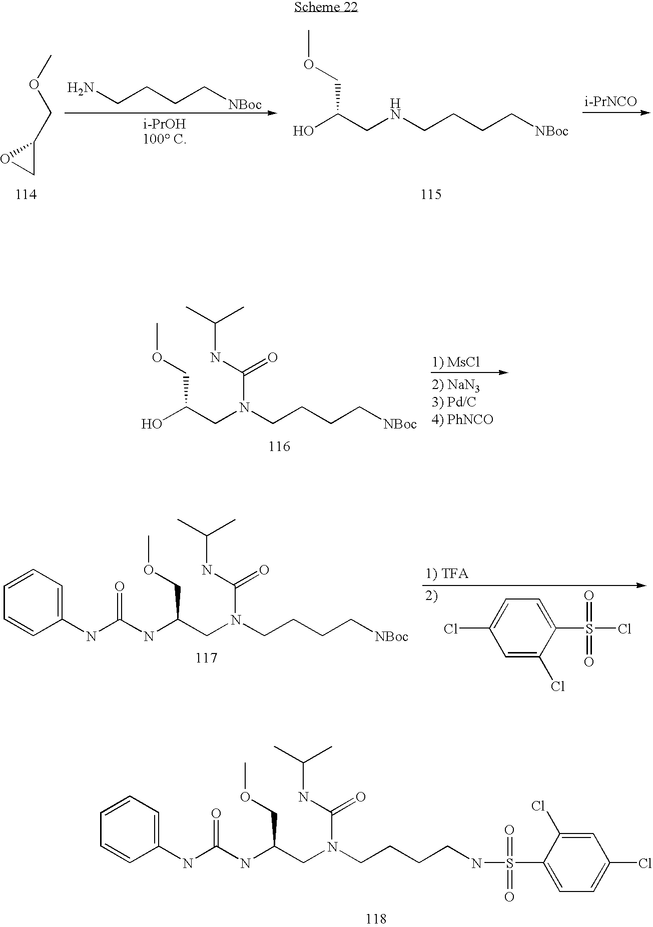

As shown in Scheme 23, some cyclic targets may be accessed by the following route. The aldehyde 120 can be prepared from the cyclic amino alcohol 119 by the protection of the free amine 119 with Boc under standard conditions and oxidation using an oxidizing reagent such as Dess-Martin Periodinane. Reductive amination using the aldehyde 120 with a free amine such as N-(4-aminobutyl)-2,4-dichlorobenzenesulfonamide in the presence of NaCNBH₃ and AcOH to give the secondary amine 121. The free amine 121 is coupled with an isocyanate such as iso-propylisocyanate to provide the urea 122. Removal of the tert-butyl carbonyl group under conditions common to the art such as HCl or TFA provides the amine (not shown), which is subsequently coupling to an isocyanate such as phenylisocyanate to provide the final compound 123.

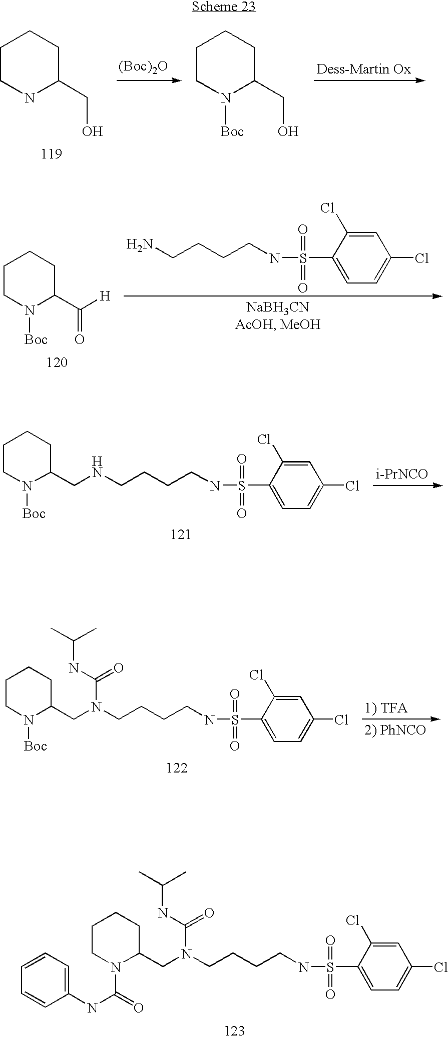

As shown in Scheme 24, additional cyclic targets may be accessed by the following route. The aldehyde 127 is prepared from the coupling reaction with [4,4-bis(ethyloxy)butyl]amine (124) under a base such as Et₃N and ethyl 1,3-dioxo-1,3-dihydro-2H-isoindole-2-carboxylate (125) followed by hydrolysis in an acidic condition. Reductive amination of the aldehyde 127 with the free amine (not shown) prepared from 1,1-dimethylethyl (3R)-3-piperidinylcarbamate (128) by the standard conditions described in Scheme 18 in the presence of NaCNBH₃ and AcOH to give the secondary amine 130. The free amine 130 is coupled with an isocyanate such as iso-propylisocyanate to provide the urea 131. Treatment of the urea 131 with hydrazine provides the free amine (not shown) followed by the coupling reaction with 2,4-dichlorosulfonyl chloride to provide the final compound 132.

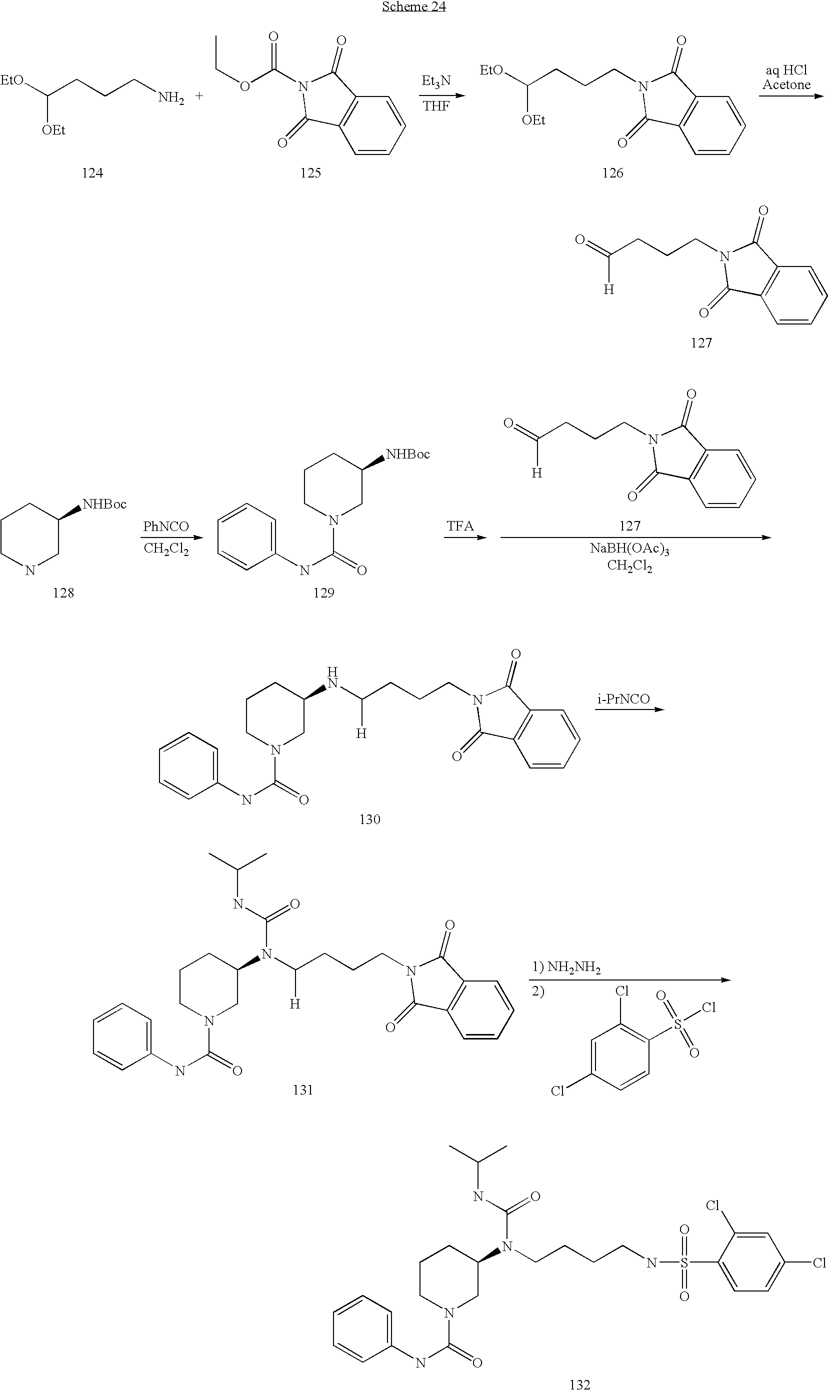

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and a pharmaceutically-acceptable excipient.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from about 0.1 mg to about 50 mg. The pharmaceutical compositions of the invention typically contain one compound of the invention. However, in certain embodiments, the pharmaceutical compositions of the invention contain more than one compound of the invention. For example, in certain embodiments the pharmaceutical compositions of the invention contain two compounds of the invention. In addition, the pharmaceutical compositions of the invention may optionally further comprise one or more additional pharmaceutically active compounds. Conversely, the pharmaceutical compositions of the invention typically contain more than one pharmaceutically-acceptable excipient. However, in certain embodiments, the pharmaceutical compositions of the invention contain one pharmaceutically-acceptable excipient.

As used herein, “pharmaceutically-acceptable excipient” means a pharmaceutically acceptable material, composition or vehicle involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically-acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically-acceptable excipients may be chosen for their ability to facilitate the carrying or transporting the compound or compounds of the invention once administered to the patient from one organ, or portion of the body, to another organ, or portion of the body. Certain pharmaceutically-acceptable excipients may be chosen for their ability to enhance patient compliance.

Suitable pharmaceutically-acceptable excipients include, but are not limited to, the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press). The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

### Biological Assays

The compounds of this invention may be tested in one of several biological assays.

Ca²⁺ influx mediated through TRPV4 channel receptors can be measured using articular chondrocytes from such species as, but not limited to, human, rat, canine, rabbit, monkey, and bovine, using standard techniques in the art such as, but not limited to, Fura-2 (Invitrogen/Molecular Probes, Eugene, Oreg.) fluorescence using a FlexStation (manufactured by Molecular Devices, Sunnyvale, Calif.). Table 1 lists biological data for several representative compounds obtained using this method in bovine articular chondrocytes.

TABLE 1
bovine articular chondrocytes

| Compound Example No. | EC50 values |
|---|---|
| Example 3 | +++ |
| Example 5 | ++ |

| | |
|---|---|
| Legend | |
| EC₅₀ values (in micromolar) | Symbol |
| 0.03-0.06 | +++ |
| 0.07-0.10 | ++ |

Table 2 lists biological data for several representative compounds obtained using this method in human articular chondrocytes.

TABLE 2
human articular chondrocytes

| Compound Example No. | EC50 values |
|---|---|
| Example 3 | +++ |
| Example 6 | ++ |
| Example 50 | + |

| | |
|---|---|
| Legend | |
| EC₅₀ values (in micromolar) | Symbol |
| 0.03-0.09 | +++ |
| 0.10-1.0 | ++ |
| 1.0-10 | + |

Other techniques used to measure TRPV4 channel receptor activation in chondrocytes include, but are not limited to: FLIPR assay, measuring a compound's capability to reduce the amount of ADAMTSs produced and/or released in response to a catabolic stimulus by a cell comprising a TRPV4 channel receptor; measuring a compound's capability to reduce the amount of MMPs produced and/or released in response to a catabolic stimulus by a cell comprising a TRPV4 channel receptor; measuring a compound's capability to effect the amount of nitric oxide (NO) produced in response to a catabolic stimulus by a cell comprising a TRPV4 channel receptor; and measuring a compound's capability to attenuate the inhibition of matrix synthesis in response to a catabolic stimulus by a cell comprising a TRPV4 channel receptor.

The compounds of this invention generally show TRPV4 channel receptor modulator activity having EC50 values in the range of 0.01 μM to 10 μM. The full structure/activity relationship has not yet been established for the compounds of this invention; nevertheless, one of ordinary skill in the art can readily determine which compounds of formula (I) are modulators of the TRPV4 channel receptor with an EC₅₀ value advantageously in the range of 0.01 μM to 10 μM using an assay described herein. All exemplary compounds of the present invention were assessed using at least one of the biological assays presented above. Compounds presented in the Examples had EC₅₀ values of about 0.01 μM to 10 μM as measured by Flex Station using bovine and/or human articular chondrocytes.

### Methods of Use

The compounds of the present invention are useful as agonists of TRPV4 channel receptors and are further useful in the treatment of disease associated with TRPV4 channel receptors. Thus, the present invention further relates to a method of treating a patient comprising administering to the patient an effective amount of a compound of formula I to activate a TRPV4 channel receptor. Also provided is a method for treating a patient comprising contacting at least one cell expressing a TRPV4 channel receptor of the patient with a therapeutically effective amount of an a compound of formula I.

The method of the present invention may be used to treat a patient suffering from any or all of the following: a disease affecting cartilage or matrix degradation; pain, including chronic pain, neuropathic pain, and postoperative pain; osteoarthritis; rheumatoid arthritis; neuralgia; neuropathies; algesia; nerve injury; ischaemia; neurodegeneration; cartilage degeneration; and inflammatory disorders. The method of treatment of the invention comprises administering a safe and effective amount of a compound according to Formula I or a pharmaceutically-acceptable salt thereof to the patient.

As used herein, “treatment” means: (1) the amelioration or prevention of the condition being treated or one or more of the biological manifestations of the condition being treated; (2) the interference with (a) one or more points in the biological cascade that leads to or is responsible for the condition being treated; or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” means an amount of the compound sufficient to significantly induce a positive modification in the condition to be treated but low enough to avoid serious side effects (at a reasonable benefit/risk ratio) within the scope of sound medical judgment. A safe and effective amount of a compound of the invention will vary with the particular compound chosen; the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

As used herein, “patient” refers to a human or other animal. The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, otic, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change. Typical daily dosages may vary depending upon the particular route of administration chosen. Typical daily dosages for oral administration range from about 0.4 to about 400 mg/kg. Typical daily dosages for parenteral administration range from about 0.01 to about 100 mg/kg; preferably between 0.1 and 20 mg/kg. The compounds of the invention may be administered alone or in combination with one or more additional active agents.

### EXAMPLES

The following examples illustrate the invention. These examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made without departing from the spirit and scope of the invention.

### Example 1

### Preparation of N-((1S)-1-{[(4-{[(2,4-dichlorophenyl)sulfonyl]amino}butyl)amino]carbonyl}-3-methylbutyl)-1-benzothiophene-2-carboxamide

### a. 1-[(1-Benzothien-2-ylcarbonyl)oxy]-2,5-pyrrolidinedione

To a solution of 1-benzothiophene-2-carboxylic acid (10 g, 56.18 mmol) in CH₂Cl₂ (281 mL) in a dried 1 L round bottom flask, N-hydroxysuccinimide (7.11 g, 61.8 mmol) and 1-[3-(dimethylamino)propyl]-3-ethylcarbodiimide hydrochloride (12.92 g, 67.40 mmol) were added. The reaction mixture was stirred under nitrogen at rt (room temperature) for 4 hr. After evaporating CH₂Cl₂ (up to ½) under reduced pressure, the residue was washed by brine twice. The organic solution was dried over MgSO₄. After filtration, concentration, and drying under the reduced pressure, the white solid (15.4 g) was carried out to the next step without further purification.

### b. N-(1-Benzothien-2-ylcarbonyl)-L-leucine

To a solution of 1-[(1-benzothien-2-ylcarbonyl)oxy]-2,5-pyrrolidinedione (15.4 g, 56.18 mmol) and L-leucine (7.66 g, 58.43 mmol) in EtOH (140 ml), CH₂Cl₂ (85 ml), and deionized water (55 ml), triethylamine (9.4 ml, 67.42 mmol) was slowly added at between 5° C. and 10° C. After 10 minutes, the mixture was warmed up to rt and stirred for 18 hr. The mixture was diluted with 50 ml of water, and then pH was adjusted to 1 with 6N HCl, followed by the extraction with methylene chloride (2×100 mL). The organic solution was dried over MgSO₄, filtered, and concentrated. After drying under the reduced pressure, the white solid product (16.4 g) was carried out to the next step without further purification.

### c. 1,1-Dimethylethyl (4-{[N-(1-benzothien-2-ylcarbonyl)-L-leucyl]amino}butyl)carbamate

To a solution of N-(1-benzothien-2-ylcarbonyl)-L-leucine (crude, 407 mg, 1.40 mmol) and N-(4-aminobutyl)carbamic acid tert-butyl ester (2.24 mL, 1.27 mmol) in CH₂Cl₂ (10 mL) were added HOOBt (10 mg, 0.06 mmol), EDC.HCl (280 mg, 1.46 mmol), and NMM (0.18 mL, 1.65 mmol) at rt. After stirring for 5 hr at rt, the reaction mixture was quenched with cold 1N HCl. After extraction with CH₂Cl₂ (20 mL×2), the organic solution was washed with sat'd NaHCO₃ and brine, followed by drying over MgSO₄, filtration, and concentration. The resultant residue was purified by flash column chromatograph (Biotage, 20% to 60% EtOAc/Hex) to provide 387 mg (66%) of the desired product.

### d. N-((1S)-1-{[(4-Aminobutyl)amino]carbonyl}-3-methylbutyl)-1-benzothiophene-2-carboxamide hydrochloride

To a solution of 1,1-dimethylethyl(4-{[N-(1-benzothien-2-ylcarbonyl)-L-leucyl]amino}butyl)carbamate (110 mg, 0.239 mmol) in CH₂Cl₂ (1.5 mL) was added 4N. HCl in dioxane (0.7 mL) at rt. After stirring for 30 min at rt, the reaction mixture was concentrated and dried under the reduced pressure. The resultant amorphous solid (93 mg) was carried out to the next reaction without further purification.

### e. N-((1S)-1-{[(4-{[(2,4-Dichlorophenyl)sulfonyl]amino}butyl)amino]carbonyl}-3-methylbutyl)-1-benzothiophene-2-carboxamide

To a solution of N-((1S)-1-{[(4-aminobutyl)amino]carbonyl}-3-methylbutyl)-1-benzothiophene-2-carboxamide hydrochloride (0.239 mmol) in CH₂Cl₂ were added 2,4-dichlorobenzenesulfonyl chloride (65 mg, 0.263 mmol) and Et₃N (0.13 mL, 0.96 mmol) at rt. After stirring for 30 minutes at rt, the reaction mixture was purified by flash column chromatography (Biotage, 20% EtOAC/Hex to 60% EtOAC/Hex) without aqueous work-up to provide 133 mg of a desired product (98% for two steps); LCMS: [MH]⁺=570.

### Example 2

### Preparation of N-((1S)-1-{[(4-{[(2-Bromo-4-fluorophenyl)sulfonyl]amino}butyl)amino]carbonyl}-3-methylbutyl)-1-benzothiophene-2-carboxamide

The title compound was prepared following the procedure of Example 1 except for the use of 2-bromo-4-chlorobenzenesulfonyl chloride in place of 2,4-dichlorobenzene sulfonyl chloride; LCMS: [MH]⁺=599.

### Example 3

### Preparation of N-((1S)-1-{[(4-{[(4-Bromo-2-chlorophenyl)sulfonyl]amino}butyl)amino]carbonyl}-3-methylbutyl)-1-benzothiophene-2-carboxamide

The title compound was prepared following the procedure of Example 1 except for the use of 2-chloro-4-bromobenzenesulfonyl chloride in place of 2,4-dichlorobenzene sulfonyl chloride; LCMS: [MH]⁺=614.

### Example 4

### Preparation of N-[(1S)-1-({[4-({[4-Fluoro-2-(trifluoromethyl)phenyl]sulfonyl}amino)butyl]amino}carbonyl)-3-methylbutyl]-1-benzothiophene-2-carboxamide

The title compound was prepared following the procedure of Example 1 except for the use of 2-trifluoromethyl-4-fluoro-benzenesulfonyl chloride in place of 2,4-dichlorobenzene sulfonyl chloride; LCMS: [MH]⁺=588.

### Example 5

### Preparation of N-((1S)-1-{[(4-{[(2-Chloro-4-fluorophenyl)sulfonyl]amino}butyl)amino]carbonyl}-3-methylbutyl)-1-benzothiophene-2-carboxamide

The title compound was prepared following the procedure of Example 1 except for the use of 2-chloro-4-fluoro-benzenesulfonyl chloride in place of 2,4-dichlorobenzene sulfonyl chloride; LCMS: [MH]⁺=554.

### Example 6

### Preparation of N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide

### a. N-(4-aminobutyl)-2,4-dichlorobenzenesulfonamide

To a solution of 1,4-diaminobutane (3.60 g, 40.84 mmol) in CH₂Cl₂ (100 mL) were added triethylamine (5.70 ml, 40.84 mmol) and 2,4-dichlorobenzene sulfonyl chloride (5.01 g, 20.4 mmol) at 0° C. After stirring for 2 hr at 0° C., H₂O was added to the reaction mixture. The reaction mixture was acidified to pH ˜2 with 1N aq. HCl. After extraction with CH₂Cl₂, the aqueous solution was basified to pH ˜10-11 with 1N. aq. NaOH, then extracted twice with dichloromethane and once with ethyl acetate. The combined organic solution was dried over MgSO₄, and then concentrated under the reduced pressure to yield the title compound (5.18 g, 85%, oil/solid) which was used without further purification; LCMS: [MH]⁺=297.

### b. N¹-(4-{[(2,4-dichlorophenyl)sulfonyl]amino}butyl)−/−{[(1,1-dimethylethyl)oxy]carbonyl}-L-leucinamide

To a solution of N-(4-aminobutyl)-2,4-dichlorobenzenesulfonamide (2.59 g, 8.72 mmol) in dichloromethane (15 mL) was added N-Boc-L-leucine (2.02 g, 8.72 mmol) and HOOBt (35.9 mg, 0.22 mmol) at rt. After cooling the reaction mixture in an ice-bath, NMM (1.92 ml, 17.44 mmol) and EDC.HCl (1.76 g, 9.16 mmol) were added. After stirring overnight at rt, the reaction mixture was washed with 10% (w/w) aqueous citric acid solution (10 mL) and brine. The organic solution was dried over MgSO₄, concentrated under the reduced pressure, and then purified by silica gel column chromatograpgy (15%-65% EtOAc/Hex) to give the title compound (3.82 g, 86%, white-solid); LCMS: [MH]⁺=511.

### c. N¹-(4-{[(2,4-dichlorophenyl)sulfonyl]amino}butyl)-L-leucinamide hydrochloride

To a solution of N¹-(4-{[(2,4-dichlorophenyl)sulfonyl]amino}butyl)-Af-{[(1,1-dimethylethyl)oxy]carbonyl}-L-leucinamide (3.82 g, 7.49 mmol) in methanol (15 mL) was added 4N HCl in 1,4-dioxane (18.7 ml) at rt. After stirring for 2 h at rt, the mixture was concentrated under the reduced pressure, and then azeotroped twice with toluene to give the title compound (3.43 g, quantitative) as a white solid; LCMS: [MH]⁺=411.

### d. N-((1S)-1-{[(4-{[(2,4-dichlorophenyl)sulfonyl]amino}butyl)amino]carbonyl}-3-methylbutyl)-3-methylfuro[3,2-b]pyridine-2-carboxamide

To a solution of N¹-(4-{[(2,4-dichlorophenyl)sulfonyl]amino}butyl)-L-leucinamide hydrochloride (85 mg, 0.190 mmol) in dichloromethane (2 mL) was added 3-methylfuro[3,2-b]pyridine-2-carboxylic acid (36.0 mg, 0.20 mmol), HOBT (27 mg, 0.20 mmol), EDC.HCl (38.4 mg, 0.20 mmol), and triethylamine (0.106 ml, 0.76 mmol) at rt. After stirring for overnight at rt, the reaction mixture was quenched with 10% (w/w) aqueous citric acid solution (5 mL). The reaction mixture was extracted with CH₂Cl₂. The organic solution was washed with brine and dried over MgSO₄. After filtration and concentration under the reduced pressure, the desired product was obtained by silica gel column chromatograpgy (40 mg, 37%); LCMS: [MH]⁺=569.

### Example 7

### Preparation of N-((1S)-1-1{-[(4-{[(2,4-dichlorophenyl)sulfonyl]amino}butyl)amino]carbonyl}-3-methylbutyl)-1-methyl-1H-indole-2-carboxamide

The title compound was prepared following the procedure of Example 1 except for the use of 1-methyl-1H-indole-2-carboxylic acid in place of 3-methylfuro[3,2-b]pyridine-2-carboxylic acid; LCMS: [MH]⁺=567.

### Example 8

### Preparation of N-((1S)-1-{[(4-{[(2,4-dichlorophenyl)sulfonyl]amino}butyl)amino]carbonyl}-3-methylbutyl)-1-benzofuran-2-carboxamide

The title compound was prepared following the procedure of Example 1 except for the use of benzofuran-2-carboxylic acid in place of the 3-methylfuro[3,2-b]pyridine-2-carboxylic acid; LCMS: [MH]⁺=554.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

### Example 9

### N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide

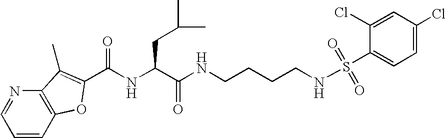

### a. N-(4-Aminobutyl)-2,4-dichlorobenzenesulfonamide

To a solution of 1,4-diaminobutane (3.60 g, 40.84 mmol) in CH₂Cl₂ (100 mL) were added triethylamine (5.70 ml, 40.84 mmol) and 2,4-dichlorobenzene sulfonyl chloride (5.01 g, 20.4 mmol) at 0° C. After stirring for 2 h at 0° C., H₂O was added to the reaction mixture. The reaction mixture was acidified to pH ˜2 with 1N aq. HCl. After extraction with CH₂Cl₂, the aqueous solution was basified to pH ˜10-11 with 1N aq. NaOH, and then extracted twice with dichloromethane and once with ethyl acetate. The combined organic solution was dried over MgSO₄, then concentrated under reduced pressure to yield the title compound (5.18 g, 85%), which was used without further purification; LCMS: [MH]⁺=297.

### b. N¹-(4-{[(2,4-Dichlorophenyl)sulfonyl]amino}butyl)-N²-{[(1,1-dimethylethyl)oxy]carbonyl}-L-leucinamide

To a solution of N-(4-aminobutyl)-2,4-dichlorobenzenesulfonamide (2.59 g, 8.72 mmol) in dichloromethane (15 mL) were added N-Boc-L-leucine (2.02 g, 8.72 mmol) and HOOBt (35.9 mg, 0.22 mmol) at rt. The reaction mixture was cooled in an ice bath, then NMM (1.92 mL, 17.44 mmol) and EDC-HCl (1.76 g, 9.16 mmol) was added. After stirring overnight at rt, the reaction mixture was washed with 10% (w/w) aqueous citric acid solution (10 mL) and brine. The organic layer was dried over MgSO₄, filtered, concentrated in vacuo, and then purified by silica gel column chromatograpgy (15%-65% EtOAc/Hex) to give the title compound (3.82 g, 86%, white solid); LCMS: [MH]⁺=511.

### c. N¹-(4-{[(2,4-Dichlorophenyl)sulfonyl]amino}butyl)-L-leucinamide hydrochloride

To a solution of N¹-(4-{[(2,4-dichlorophenyl)sulfonyl]amino}butyl)-N²-{[(1,1-dimethylethyl)oxy]carbonyl}-L-leucinamide (3.82 g, 7.49 mmol) in methanol (15 mL) was added 4 N. HCl in 1,4-dioxane (19 mL) at rt. After stirring for 2 h at rt, the mixture was concentrated under reduced pressure, and then azeotroped twice with toluene to give the title compound as a white solid (3.43 g, quantitative); LCMS: [MH]⁺=411.

### d. N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide

To a solution of N¹-(4-{[(2,4-dichlorophenyl)sulfonyl]amino}butyl)-L-leucinamide hydrochloride (85 mg, 0.19 mmol) in dichloromethane (2 mL) were added 3-methylfuro[3,2-b]pyridine-2-carboxylic acid (36 mg, 0.20 mmol), HOBT (27 mg, 0.20 mmol), EDC.HCl (38 mg, 0.20 mmol), and triethylamine (0.11 mL, 0.76 mmol) at rt. After stirring for overnight at rt, the reaction mixture was quenched with 10% (w/w) aqueous citric acid solution (5 mL). The reaction mixture was extracted with CH₂Cl₂. The organic layer was washed with brine and dried over MgSO₄. After filtration and concentration in vacuo, the desired product was obtained by silica gel column chromatograpgy (40 mg, 37%); LCMS: [MH]⁺=569.

### Example 10

### N-((1S)-1-{[(4-{[(2,4-Dichlorophenyl)sulfonyl]amino}butyl)amino]carbonyl}-3-methylbutyl)imidazo[1,2-b]pyridazine-2-carboxamide

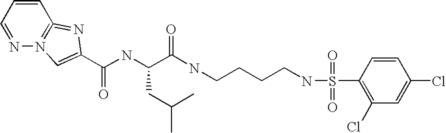

The title compound was prepared following the procedure of Example 9 except for the use of imidazo[1,2-b]pyridazine-2-carboxylic acid in place of 3-methylfuro[3,2-b]pyridine-2-carboxylic acid; LCMS: [MH]⁺=555.

### Example 11

### N-((1S)-1-{[(4-{[(2,4-Dichlorophenyl)sulfonyl]amino}butyl)amino]carbonyl}-3-methylbutyl)thieno[3,2-b]pyridine-2-carboxamide

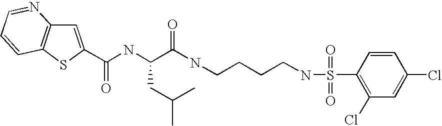

The title compound was prepared following the procedure of Example 9 except for the use of thieno[3,2-b]pyridine-2-carboxylic acid in place of 3-methylfuro[3,2-b]pyridine-2-carboxylic acid; LCMS: [MH]⁺=572.

### Example 12

### (2S)—N-((1S)-1-{[(4-{[(2,4-Dichlorophenyl)sulfonyl]amino}butyl)amino]carbonyl}-3-methylbutyl)octahydro-2H-quinolizine-2-carboxamide

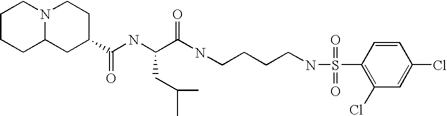

The title compound was prepared following the procedure of Example 9 except for the use of (2S)-octahydro-2H-quinolizine-2-carboxylic acid hydrochloride in place of 3-methylfuro[3,2-b]pyridine-2-carboxylic acid; LCMS: [MH]⁺=575.

### Example 13

### N-((1S)-1-{[(4-{[(2,4-Dichlorophenyl)sulfonyl]amino}butyl)amino]carbonyl}-3-methylbutyl)imidazo[1,2-a]pyridine-2-carboxamide

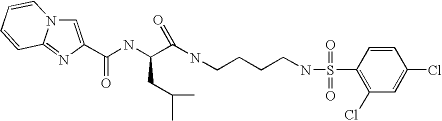

The title compound was prepared following the procedure of Example 9 except for the use of imidazo[1,2-a]pyridine-2-carboxylic acid in place of 3-methylfuro[3,2-b]pyridine-2-carboxylic acid; LCMS: [MH]⁺=554.

### Example 14

### N-((1S)-1-{[(4-{[(2,4-Dichlorophenyl)sulfonyl]amino}butyl)amino]carbonyl}-3-methylbutyl)imidazo[2,1-b][1,3]thiazole-6-carboxamide

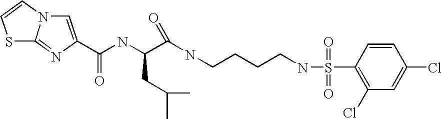

The title compound was prepared following the procedure of Example 9 except for the use of imidazo[2,1-b][1,3]thiazole-6-carboxylic acid in place of 3-methylfuro[3,2-b]pyridine-2-carboxylic acid; LCMS: [MH]⁺=560.

### Example 15

### N-((1S)-1-{[4-{[(2,4-Dichlorophenyl)sulfonyl]amino}butyl)amino]carbonyl}-3-methylbutyl)-4H-thieno[3,2-b]pyrrole-5-carboxamide

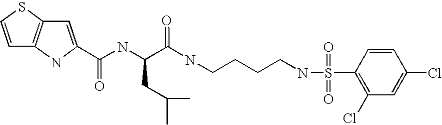

The title compound was prepared following the procedure of Example 9 except for the use of 4H-thieno[3,2-b]pyrrole-5-carboxylic acid in place of 3-methylfuro[3,2-b]pyridine-2-carboxylic acid; LCMS: [MH]⁺=559.

### Example 16

### N-[(1S)-1-({[4-({[4-Fluoro-2-(trifluoromethyl)phenyl]sulfonyl}amino)butyl]amino}carbonyl)-3-methylbutyl]-4H-thieno[3,2-b]pyrrole-5-carboxamide

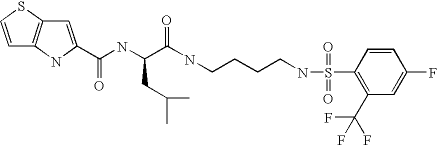

The title compound was prepared following the procedure of Example 9 with the following exceptions. In the first step, 4-fluoro-2-(trifluoromethyl)benzenesulfonyl chloride was used in place of 2,4-dichlorobenzenesulfonyl chloride. After deprotection of BOC, 4H-thieno[3,2-b]pyrrole-5-carboxylic acid was used in place of 3-methylfuro[3,2-b]pyridine-2-carboxylic acid; LCMS: [MH]⁺=577.

### Example 17

### N-((1S)-1-[(4-{[(2-Chloro-4-fluorophenyl)sulfonyl]amino}butyl)amino]carbonyl-3-methylbutyl)-4H-thieno[3,2-b]pyrrole-5-carboxamide

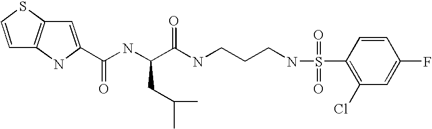

The title compound was prepared following the procedure of Example 9 with the following exceptions. In the first step 2-chloro-4-fluoro-benzenesulfonyl chloride was used in place of 2,4-dichlorobenzenesulfonyl chloride. After deprotection of BOC, 4H-thieno[3,2-b]pyrrole-5-carboxylic acid was used in place of 3-methylfuro[3,2-b]pyridine-2-carboxylic acid; LCMS: [MH]⁺=543.

### Example 18

### N¹-(4-{[(2,4-Dichlorophenyl)sulfonyl]amino}butyl)-N²-({4-[(4-methyl-1-piperazinyl)methyl]phenyl}carbonyl)-L-leucinamide( )

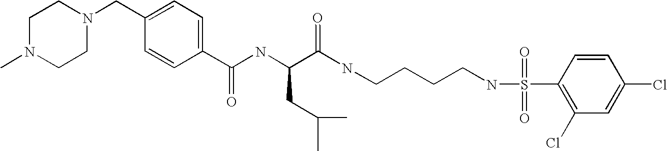

The title compound was prepared following the procedure of Example 9 except for the use of 4(N-methylpiperazinyl)-methyl benzoic acid in place of 3-methylfuro[3,2-b]pyridine-2-carboxylic acid; LCMS: [MH]⁺=626.

### Example 19

### 1,1-Dimethylethyl 4-(4-{[((1S)-1-{[(4-{[(2,4-dichlorophenyl)sulfonyl]amino}butyl)amino]-carbonyl}-3-methylbutyl)amino]carbonyl}phenyl)-1-piperazinecarboxylate

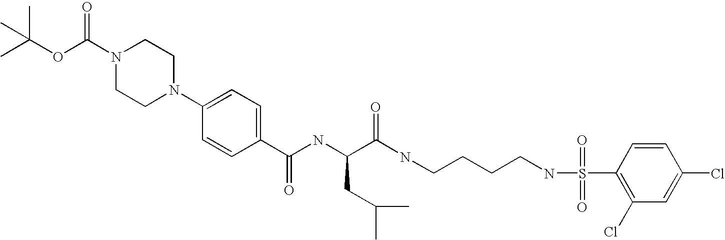

The title compound was prepared following the procedure of Example 9 except for the use of 4-(4-carboxyphenyl)piperazine-1-carboxylic acid-t-butyl ester in place of 3-methylfuro[3,2-b]pyridine-2-carboxylic acid; LCMS: [MH]⁺=698.

### Example 20

### N-[(1S)-1-({[4-({[4-Fluoro-2-(trifluoromethyl)phenyl]sulfonyl}amino)butyl]amino}carbonyl)-3-methylbutyl]-5,6-dihydro-4H-cyclopenta[b]thiophene-2-carboxamide

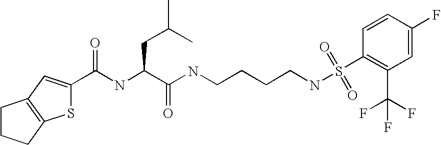

The title compound was prepared following the procedure of Example 9 with the following exceptions. In the first step, 4-fluoro-2-(trifluoromethyl)benzenesulfonyl chloride was used in place of 2,4-dichlorobenzenesulfonyl chloride. After deprotection of BOC, 5,6-dihydro-4H-cyclopenta[b]thiophene-2-carboxylic acid was used in place of 3-methylfuro[3,2-b]pyridine-2-carboxylic acid; LCMS: [MH]⁺: 578.

### Example 21

### N²-(3-Cyclopentylpropanoyl)-N¹-[4-({[4-fluoro-2-(trifluoromethyl)phenyl]sulfonyl}amino)butyl]-L-leucinamide

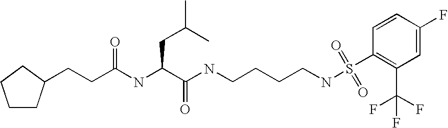

The title compound was prepared following the general procedure of Example 20 except substituting 5,6-dihydro-4H-cyclopenta[b]thiophene-2-carboxylic acid with 3-cyclopentylpropanoic acid; LCMS (m/z): 552 (M+H).

### Example 22

### N²-(3-Cyclohexylpropanoyl)-N¹-[4-({[4-fluoro-2-(trifluoromethyl)phenyl]sulfonyl}amino)butyl]-L-leucinamide

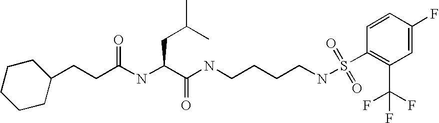

The title compound was prepared following the general procedure of Example 20 except substituting 3-cyclopentylpropanoic acid with 3-cyclohexylpropanoic acid; LCMS (m/z): 566 (M+H).

### Example 23

### N²-(3-Cyclohexylpropanoyl)-N¹-(4-{[(2,4-dichlorophenyl)sulfonyl]amino}butyl)-L-leucinamide

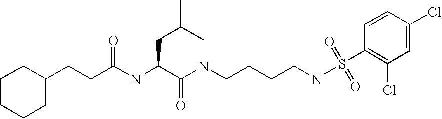

The title compound was prepared following the general procedure of Example 22 except substituting 4-fluoro-2-(trifluoromethyl)benzenesulfonyl chloride with 2,4-dichlorobenzenesulfonyl chloride; LCMS (m/z): 548 (M+H).

### Example 24

### N²-(3-Cyclopentylpropanoyl)-N¹-(4-{[(2,4-dichlorophenyl)sulfonyl]amino}butyl)-L-leucinamide

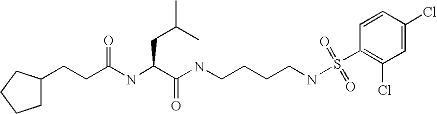

The title compound was prepared following the general procedure of Example 21 except substituting 4-fluoro-2-(trifluoromethyl)benzenesulfonyl chloride with 2,4-dichloro-benzenesulfonyl chloride; LCMS (m/z): 534 (M+H).

### Example 25

### N¹-(4-{[(2-Chloro-4-fluorophenyl)sulfonyl]amino}butyl)-N-(3-cyclohexylpropanoyl)-L-leucinamide

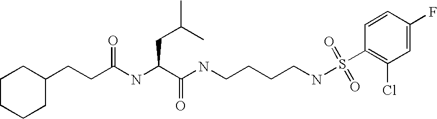

The title compound was prepared following the general procedure of Example 22 except substituting 4-fluoro-2-(trifluoromethyl)benzenesulfonyl chloride with 2-chloro-4-florobenzenesulfonyl chloride; LCMS (m/z): 532 (M+H).

### Example 26

### N¹-(4-{[(2-chloro-4-fluorophenyl)sulfonyl]amino}butyl)-N²⁻⁽3-cyclopentylpropanoyl)-L-leucinamide

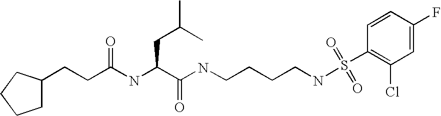

The title compound was prepared following the general procedure of Example 21 except substituting 4-fluoro-2-(trifluoromethyl)benzenesulfonyl chloride with 2-chloro-4-florobenzenesulfonyl chloride; LCMS (m/z): 518 (M+H).

### Example 27

### N¹-(4-{[(2-chloro-4-fluorophenyl)sulfonyl]amino}butyl)-N²-(cyclohexylacetyl)-L-leucinamide

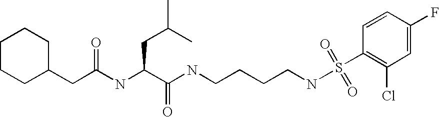

The title compound was prepared following the general procedure of Example 26 except substituting 3-cyclopentylpropanoic acid with cyclohexylacetic acid; LCMS (m/z): 518 (M+H).

### Example 28

### N¹-(4-{[(2-Chloro-4-fluorophenyl)sulfonyl]amino}butyl)-N²-[(4-phenyl-2-thienyl)carbonyl]-L-leucinamide

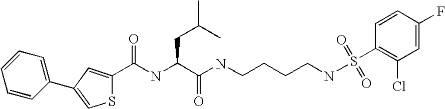

The title compound was prepared following the general procedure of Example 26 except substituting 3-cyclopentylpropanoic acid with 4-phenyl-2-thiophenecarboxylic acid; LCMS (m/z): 580 (M+H).

### Example 29

### N¹-(4-{[(2-Chloro-4-fluorophenyl)sulfonyl]amino}butyl)-N²-[(2E)-3-(4-methylphen propenoyl]-L-leucinamide

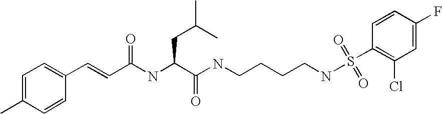

The title compound was prepared following the general procedure of Example 26 except substituting 3-cyclopentylpropanoic acid with (2E)-3-(4-methylphenyl)-2-propenoic acid; LCMS (m/z): 538 (M+H).

### Example 30

### 1,1-Dimethylethyl 5-{[((1S)-1-{[(4-{[(2-chloro-4-fluorophenyl)sulfonyl]amino}butyl)amino]carbonyl}-3-methylbutyl)amino]carbonyl}-3,4-dihydro-2(1H)-isoquinolinecarboxylate

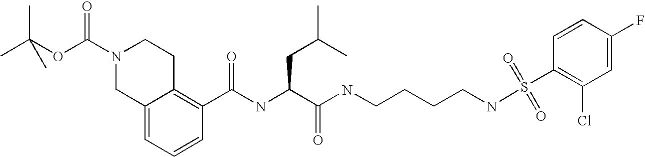

The title compound was prepared following the general procedure of Example 26 except substituting 3-cyclopentylpropanoic acid with 2-{[(1,1-dimethylethyl)oxy]carbonyl}-1,2,3,4-tetrahydro-5-isoquinolinecarboxylic acid; LCMS (m/z): 653(M+H).

### Example 31

### N¹-(4-{[(2-chloro-4-fluorophenyl)sulfonyl]amino}butyl)-N²-[(5-phenyl-2-thienyl)carbonyl]-L-leucinamide

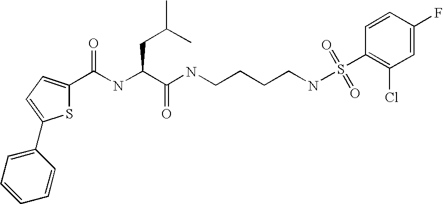

The title compound was prepared following the general procedure of Example 26 except substituting 3-cyclopentylpropanoic acid with 5-phenyl-2-thiophenecarboxylic acid; LCMS (m/z): 580 (M+H).

### Example 32

### N¹-(4-{[(2-chloro-4-fluorophenyl)sulfonyl]amino}butyl)-W-(2,3-dihydro-1H-inden-2-ylacetyl)-L-leucinamide

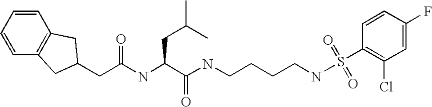

The title compound was prepared following the general procedure of Example 26 except substituting 3-cyclopentylpropanoic acid with 2,3-dihydro-1H-inden-2-ylacetic acid; LCMS (m/z): 552 (M+H).

### Example 33

### N¹-(4-{[(2-chloro-4-fluorophenyl)sulfonyl]amino}butyl)-N²-(1-cyclohexen-1-ylcarbonyl)-L-leucinamide

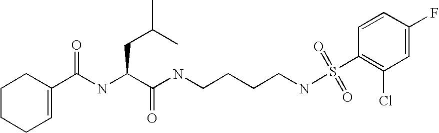

The title compound was prepared following the general procedure of Example 26 except substituting 3-cyclopentylpropanoic acid with 1-cyclohexene-1-carboxylic acid; LCMS (m/z): 502 (M+H).

### Example 34

### 1,1-Dimethylethyl 3-{3-[((1S)-1-{[(4-{[(2-chloro-4-fluorophenyl)sulfonyl]amino}butyl)amino]carbonyl}-3-methylbutyl)amino]-3-oxopropyl}-1-piperidinecarboxylate

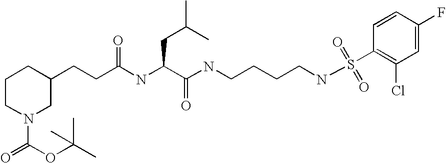

The title compound was prepared following the general procedure of Example 18 except substituting 3-cyclopentylpropanoic acid with 3-(1-{[(1,1-dimethylethyl)oxy]carbonyl}-3-piperidinyl)propanoic acid; LCMS (m/z): 633 (M+H).

### Example 35

### N-((1S)-1-{[(4-{[(2-chloro-4-fluorophenyl)sulfonyl]amino}butyl)amino]carbonyl}-3-methylbutyl)-2,3-dihydro-1H-indene-2-carboxamide

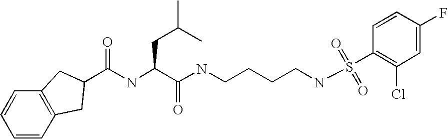

The title compound was prepared following the general procedure of Example 26 except substituting 3-cyclopentylpropanoic acid with 2,3-dihydro-1H-indene-2-carboxylic acid; LCMS (m/z): 538 (M+H).

### Example 36

### N¹-(4-{[(2-chloro-4-fluorophenyl)sulfonyl]amino}butyl)-N²-[3-(2-chlorophenyl)propanoyl]-L-leucinamide

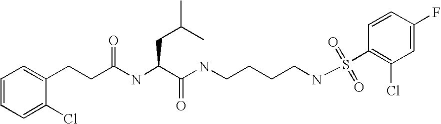

The title compound was prepared following the general procedure of Example 26 except substituting 3-cyclopentylpropanoic acid with 3-(2-chlorophenyl)propanoic acid; LCMS (m/z): 560 (M+H).

### Example 37

### N¹— (4-{[(2-chloro-4-fluorophenyl)sulfonyl]amino}butyl)-N²-[3-(3-chlorophenyl)propanoyl]-L-leucinamide

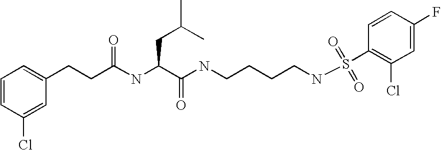

The title compound was prepared following the general procedure of Example 26 except substituting 3-cyclopentylpropanoic acid with 3-(3-chlorophenyl)propanoic acid; LCMS (m/z): 560 (M+H).

### Example 38

### 1,1-Dimethylethyl 2-{3-[((1S)-1-{[(4-{[(2-chloro-4-fluorophenyl)sulfonyl]amino}butyl)amino]carbonyl}-3-methylbutyl)amino]-3-oxopropyl}-1-piperidinecarboxylate

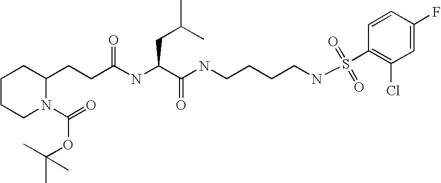

The title compound was prepared following the general procedure of Example 26 except substituting 3-cyclopentylpropanoic acid with 3-(1-{[(1,1-dimethylethyl)oxy]carbonyl}-2-piperidinyl)propanoic acid; LCMS (m/z): 633 (M+H)

### Example 39

### N¹-(4-{[(2-chloro-4-fluorophenyl)sulfonyl]amino}butyl)-N²-[3-(4-chlorophenyl)propanoyl]-L-leucinamide

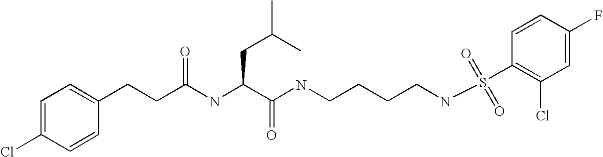

The title compound was prepared following the general procedure of Example 26 except substituting 3-cyclopentylpropanoic acid with 3-(4-chlorophenyl)propanoic acid; LCMS (m/z): 560 (M+H).

### Example 40

### N¹-(4-{[(2-Chloro-4-fluorophenyl)sulfonyl]amino}butyl)-N²-(cyclopentylacetyl)-L-leucinamide

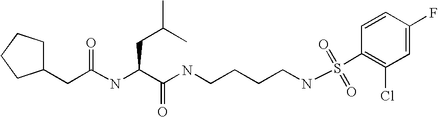

The title compound was prepared following the general procedure of Example 26 except substituting 3-cyclopentylpropanoic acid with cyclopentylacetic acid; LCMS (m/z): 504(M+H).

### Example 41

### 1,1-Dimethylethyl 2-{[((1S)-1-{[(4-{[(2-chloro-4-fluorophenyl)sulfonyl]amino}butyl)amino]carbonyl}-3-methylbutyl)amino]carbonyl}octahydro-1H-indole-1-carboxylate

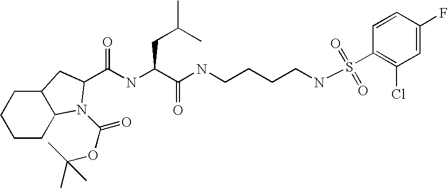

The title compound was prepared following the general procedure of Example 26 except substituting 3-cyclopentylpropanoic acid with 1-{[(1,1-dimethylethyl)oxy]carbonyl}octahydro-1H-indole-2-carboxylic acid; LCMS (m/z): 645 (M+H).

### Example 42

### N¹-(4-{[(2-Chloro-4-fluorophenyl)sulfonyl]amino}butyl)-N²-[3-(4-methylphenyl)propanoyl]-L-leucinamide

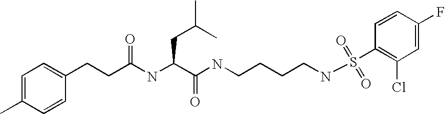

The title compound was prepared following the general procedure of Example 26 except substituting 3-cyclopentylpropanoic acid with 3-(4-methylphenyl)propanoic acid; LCMS (m/z): 540 (M+H).

### Example 43

### N-((1S)-1-{[(4-{[(2-chloro-4-fluorophenyl)sulfonyl]amino}butyl)amino]carbonyl}-3-methylbutyl)furo[3,2-b]pyridine-2-carboxamide

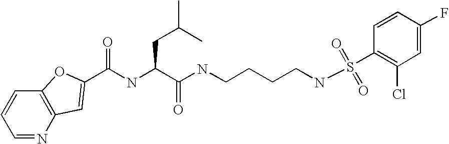

The title compound was prepared following the general procedure of Example 26 except substituting 3-cyclopentylpropanoic acid with furo[3,2-b]pyridine-2-carboxylic acid; LCMS (m/z): 539 (M+H).

### Example 44

### N¹-(4-{[(2-chloro-4-fluorophenyl)sulfonyl]amino}butyl)-N²-(tetrahydro-2H-thiopyran-4-ylacetyl)-L-leucinamide

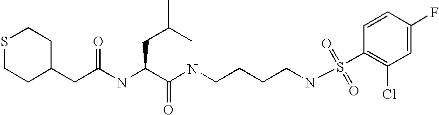

The title compound was prepared following the general procedure of Example 26 except substituting 3-cyclopentylpropanoic acid with tetrahydro-2H-thiopyran-4-ylacetic acid; LCMS (m/z): 536 (M+H)

### Example 45

### N-((1S)-1-{[(4-{[(2-chloro-4-fluorophenyl)sulfonyl]amino}butyl)amino]carbonyl}-3-methylbutyl)-2,3-dihydro-1H-indole-2-carboxamide

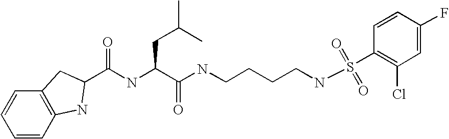

The title compound was prepared following the general procedure of Example 26 except substituting 3-cyclopentylpropanoic acid with 1-{[(1,1-dimethylethyl)oxy]carbonyl}-2,3-dihydro-1H-indole-2-carboxylic acid, followed by removal of BOC using 4 N. HCl in 1,4-dioxane; LCMS (m/z): 539 (M+H).

### Example 46

### 1-Acetyl-N-((1S)-1-{[(4-{[(2-chloro-4-fluorophenyl)sulfonyl]amino}butyl)amino]carbonyl}-3-methylbutyl)-2,3-dihydro-1H-indole-2-carboxamide

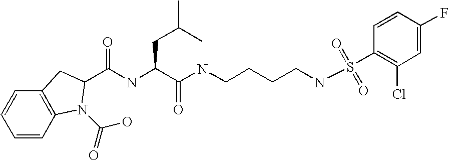

The title compound was prepared from Example 45 by the addition of acetyl chloride; LCMS (m/z): 581 (M+H).

### Example 47

### N¹-(4-{[(2-chloro-4-fluorophenyl)sulfonyl]amino}butyl)-N²-(2,2-dimethyl-3-phenylpropanoyl)-L-leucinamide

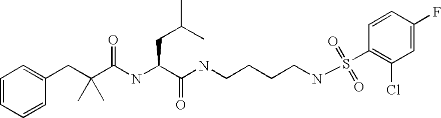

The title compound was prepared following the general procedure of Example 18 except substituting 3-cyclopentylpropanoic acid with 2,2-dimethyl-3-phenylpropanoic acid; LCMS (m/z): 554 (M+H).

### Example 48

### N²-[(4-Acetylphenyl)carbonyl]-N¹-(4-{[(2-chloro-4-fluorophenyl)sulfonyl]amino}butyl)-L-leucinamide

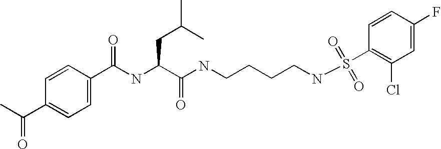

The title compound was prepared following the general procedure of Example 26 except substituting 3-cyclopentylpropanoic acid with 4-acetylbenzoic acid; LCMS (m/z): 540 (M+H).

### Example 49

### N¹-(4-{[(2-Chloro-4-fluorophenyl)sulfonyl]amino}butyl)-N²-[3-(3-nitrophenyl)propanoyl]-L-leucinamide

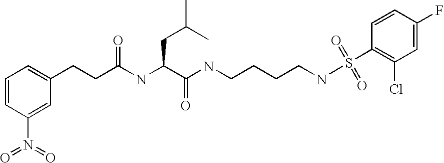

The title compound was prepared following the general procedure of Example 26 except substituting 3-cyclopentylpropanoic acid with 3-(3-nitrophenyl)propanoic acid; LCMS (m/z): 571 (M+H).

### Example 50

### N-((1S)-1-{[(4-{[(2-Chloro-4-fluorophenyl)sulfonyl]amino}butyl)amino]carbonyl}-3-methylbutyl)-1,3-benzothiazole-2-carboxamide

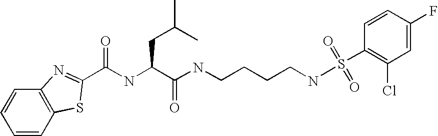

The title compound was prepared following the general procedure of Example 26 except substituting 3-cyclopentylpropanoic acid with 1,3-benzothiazole-2-carboxylic acid and substituting 4-fluoro-2-(trifluoromethyl)benzenesulfonyl chloride with 2-chloro-4-florobenzenesulfonyl chloride; LCMS (m/z): 555 (M+H).

### Example 51

### N¹-(4-{[(2-Chloro-4-fluorophenyl)sulfonyl]amino}butyl)-N²-(3-cyclohexyl-2,2-dimethylpropanoyl)-L-leucinamide

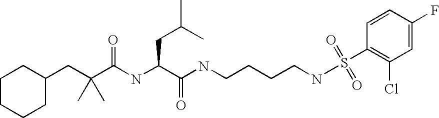

The title compound was prepared following the general procedure of Example 26 except substituting 3-cyclopentylpropanoic acid with 3-cyclohexyl-2,2-dimethylpropanoic acid; LCMS (m/z): 560 (M+H).

### Example 52

### N¹-(4-{[(2-Chloro-4-fluorophenyl)sulfonyl]amino}butyl)-N²-(cyclopentylcarbonyl)-L-leucinamide

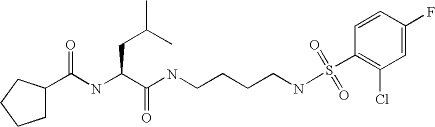

The title compound was prepared following the general procedure of Example 26 except substituting 3-cyclopentylpropanoic acid with cyclopentanecarboxylic acid; LCMS (m/z): 490 (M+H).

### Example 53

### N-((1S)-1-{[(4-{[(2-chloro-4-fluorophenyl)sulfonyl]amino}butyl)amino]carbonyl}-3-methylbutyl)-2-(phenylmethyl)-1,2,3,4-tetrahydro-5-isoquinolinecarboxamide

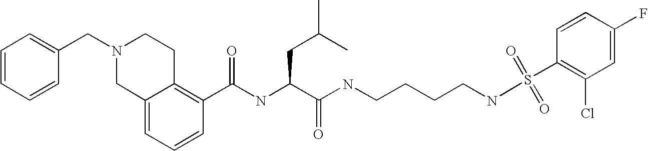

### a. N-((1S)-1-{[(4-{[(2-chloro-4-fluorophenyl)sulfonyl]amino}butyl)amino]carbonyl}-3-methylbutyl)-1,2,3,4-tetrahydro-5-isoquinolinecarboxamide

To a solution of 1,1-dimethylethyl 5-{[((1S)-1-{[(4-{[(2-chloro-4-fluorophenyl)sulfonyl]amino}butyl)amino]carbonyl}-3-methylbutyl)amino]carbonyl}-3,4-dihydro-2(1H)-isoquinolinecarboxylate (Example 22, 300 mg, 0.470 mmol) in methanol (5 mL) was added 4N HCl in 1,4-dioxane (1 mL). The reaction mixture was stirred for 1 hour at room temperature. Evaporating solvent gave the crude product as a white solid in a quantitative yield; LCMS (m/z): 553 (M+H).

### b. N-((1S)-1-{[(4-{[(2-chloro-4-fluorophenyl)sulfonyl]amino}butyl)amino]carbonyl}-3-methylbutyl)-2-(phenylmethyl)-1,2,3,4-tetrahydro-5-isoquinolinecarboxamide

To a solution of N-((1S)-1-{[(4-{[(2-chloro-4-fluorophenyl)sulfonyl]amino}butyl)amino]carbonyl}-3-methylbutyl)-1,2,3,4-tetrahydro-5-isoquinolinecarboxamide (146 mg, 0.255 mmol) in DCM (2.5 mL) were added (chloromethyl)benzene (39 mg, 0.306 mmol) and triethylamine (0.18 mL, 1.275 mmol). The reaction mixture was stirred at room temperature for 4 hours followed by concentration under reduced pressure. The residue was purified by silica gel column chromatography (1%-5% MeOH/CH₂Cl₂) to give the product as a white solid in 98% yield (158 mg); LCMS (m/z): 643 (M+H)

### Example 54

### N-((1S)-1-{[(4-{[(2-Chloro-4-fluorophenyl)sulfonyl]amino}butyl)amino]carbonyl}-3-methylbutyl)-2-(phenylcarbonyl)-1,2,3,4-tetrahydro-5-isoquinolinecarboxamide

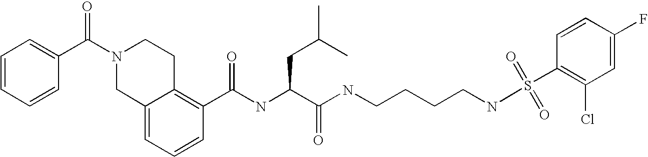

The title compound was prepared following the general procedure of Example 53 (step b) except substituting (chloromethyl)benzene with benzoyl chloride; LCMS (m/z): 657 (M+H).

### Example 55

### N-((1S)-1-{[(4-{[(2-chloro-4-fluorophenyl)sulfonyl]amino}butyl)amino]carbonyl}-3-methylbutyl)-1-benzofuran-2-carboxamide

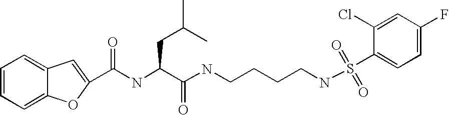

The title compound was prepared following the general procedure of Example 26 except substituting 3-cyclopentylpropanoic acid with benzofuran-2-carboxylic acid; LCMS (m/z): 538.2 (M+H).

### Example 56

### N¹-(4-{[(2,4-dichlorophenyl)sulfonyl]amino}butyl)-N²-[(phenyloxy)acetyl]-L-leucinamide

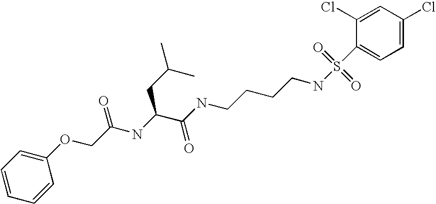

The title compound was prepared following the procedure of Example 9 except for the use of (phenyloxy)acetic acid in place of 3-methylfuro[3,2-b]pyridine-2-carboxylic acid; LCMS: [MH]⁺=544.2.

### Example 57

### N¹-(4-{[(2,4-dichlorophenyl)sulfonyl]amino}butyl)-N²-({[2-(methyloxy)phenyl]oxy}acetyl)-L-leucinamide

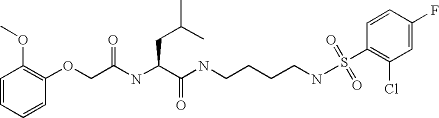

The title compound was prepared following the procedure of Example 9 except for the use of {[2-(methyloxy)phenyl]oxy}acetic acid in place of 3-methylfuro[3,2-b]pyridine-2-carboxylic acid; LCMS: [MH]⁺=574.2.

### Example 58

### N²-{[(4-chloro-2-methylphenyl)oxy]acetyl}-N¹-(4-{[(2,4-dichlorophenyl)sulfonyl]amino}butyl)-L-leucinamide

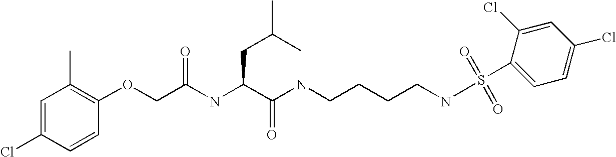

The title compound was prepared following the procedure of Example 9 except for the use of [(4-chloro-2-methylphenyl)oxy]acetic acid in place of 3-methylfuro[3,2-b]pyridine-2-carboxylic acid; LCMS: [MH]⁺=592.2, 594.2.

### Example 59

### N¹-(4-{[(2,4-dichlorophenyl)sulfonyl]amino}butyl)-N²-{[(2-methylphenyl)oxy]acetyl}-L-leucinamide

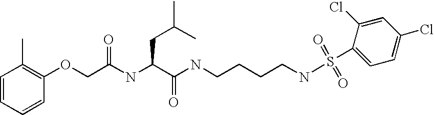

The title compound was prepared following the procedure of Example 9 except for the use of [(2-methylphenyl)oxy]acetic acid in place of 3-methylfuro[3,2-b]pyridine-2-carboxylic acid; LCMS: [MH]⁺=558.2.

### Example 60

### N¹-(4-{[(2,4-dichlorophenyl)sulfonyl]amino}butyl)-N²-{[(2-methylphenyl)oxy]acetyl}-L-leucinamide

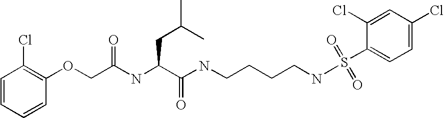

The title compound was prepared following the procedure of Example 9 except for the use of [(2-chlorophenyl)oxy]acetic acid in place of 3-methylfuro[3,2-b]pyridine-2-carboxylic acid; LCMS: [MH]⁺=578.2, 580.2.

### Example 61

### N¹-(4-{[(2,4-dichlorophenyl)sulfonyl]amino}butyl)-N²-[(1-methyl-1H-imidazol-4-yl)sulfonyl]-L-leucinamide

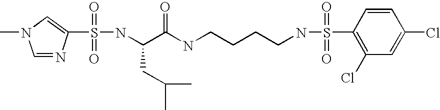

The title compound was prepared following the procedure of Example 9 except for the use of 1-methyl-1H-imidazole-4-sulfonyl chloride in place of 3-methylfuro[3,2-b]pyridine-2-carboxylic acid; LCMS: [MH]⁺=596.

### Example 62

### N²-[(cyclohexylamino)carbonyl]-N¹-(4-{[(2,4-dichlorophenyl)sulfonyl]amino}butyl)-L-leucinamide

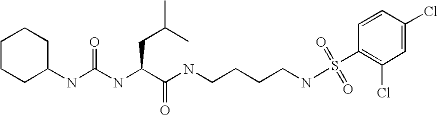

The title compound was prepared following the general procedure of Example 9 except substituting 3-methylfuro[3,2-b]pyridine-2-carboxylic acid with isocyanatocyclohexane; LCMS (m/z): 535 (M+H).

### Example 63

### N¹-(4-{[(2-chloro-4-fluorophenyl)sulfonyl]amino}butyl)-N²-[(cyclohexylamino)carbonyl]-L-leucinamide

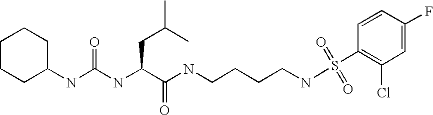

The title compound was prepared following the general procedure of Example 9 except substituting 3-methylfuro[3,2-b]pyridine-2-carboxylic acid with isocyanatocyclohexane and 2,4-dichlorobenzenesulfonyl chloride with 2-chloro-4-fluorobenzenesulfonyl chloride; LCMS (m/z): 519 (M+H).

### Example 64

### N-((1S)-1-{[(4-{[(2-chloro-4-fluorophenyl)sulfonyl]amino}butyl)amino]carbonyl}-3-methylbutyl)octahydro-2(1H)-isoquinolinecarboxamide

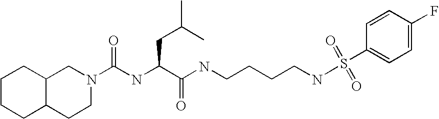

The title compound was prepared following the general procedure of Example 63 except substituting isocyanatocyclohexane with the reaction intermediate generated from decahydroisoquinoline and N,N′-carbonyldimidazole; LCMS (m/z): 559 (M+H).

### Example 65

### N-((1S)-1-{[(4-{[(2-chloro-4-fluorophenyl)sulfonyl]amino}butyl)amino]carbonyl}-3-methylbutyl)-3,4-dihydro-2(1H)-isoquinolinecarboxamide

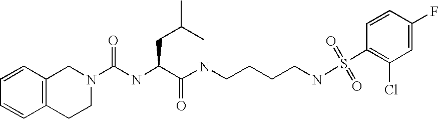

The title compound was prepared following the general procedure of Example 63 except substituting isocyanatocyclohexane with the reaction intermediate generated from 1,2,3,4-tetrahydroisoquinoline and N,N′-carbonyldimidazole; LCMS (m/z): 553 (M+H).

### Example 66

### N²-[(cyclohexylamino)carbonyl]-N¹-[4-({[4-fluoro-2-(trifluoromethyl)phenyl]sulfonyl}amino)butyl]-L-leucinamide

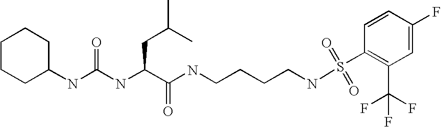

The title compound was prepared following the general procedure of Example 64 except substituting 2,4-dichlorobenzenesulfonyl chloride with 4-fluoro-2-(trifluoromethyl)benzenesulfonyl chloride; LCMS (m/z): 553 (M+H).

### Example 67

### N¹-[4-({[4-fluoro-2-(trifluoromethyl)phenyl]sulfonyl}amino)butyl]-N²-[(phenylamino)carbonyl]-L-leucinamide

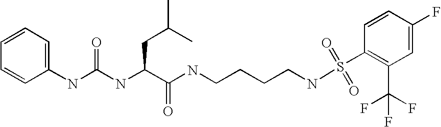

The title compound was prepared following the general procedure of Example 66 except substituting isocyanatocyclohexane with isocyanatobenzene; LCMS: [MH]⁺=547.

### Example 68

### N²-[(cyclopentylamino)carbonyl]-N¹-[4-({[4-fluoro-2-(trifluoromethyl)phenyl]sulfonyl}amino)butyl]-L-leucinamide

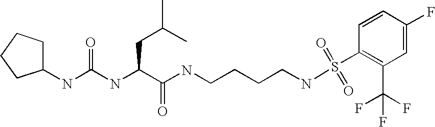

The title compound was prepared following the general procedure of Example 66 except substituting isocyanatocyclohexane with isocyanatocyclopentane; LCMS (m/z): 539 (M+H).

### Example 69

### N²-{[(cyclohexylmethyl)amino]carbonyl}-N¹-[4-({[4-fluoro-2-(trifluoromethyl)phenyl]sulfonyl}amino)butyl]-L-leucinamide

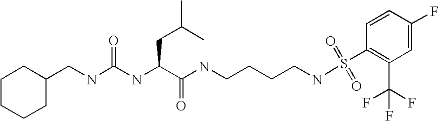

The title compound was prepared following the general procedure of Example 66 except substituting isocyanatocyclohexane with the reaction intermediate generated from cyclohexylmethylamine and N,N′-carbonyldimidazole as the following procedure; To a solution of N,N′-carbonyldimidazole (58 mg, 0.356 mmol) in THF (2 mL) was added N¹-[4-({[4-fluoro-2-(trifluoromethyl)phenyl]sulfonyl}amino)butyl]-L-leucinamide (150 mg, 0.324 mmol) and triethylamine (0.05 mL, 0.356 mmol). The reaction mixture was stirred at RT for 2 hours and then added 1-cyclohexylmethylamine (36 mg, 0.324 mmol). The reaction mixture was heated to reflux for 3 hours. After concentration under reduced pressure, the residue was purified by silica gel column chromatography (20%-85% EtOAc/Hexane) to give the title product as a white solid in 76% yield (140 mg); LCMS (m/z): 567 (M+H).

### Example 70

### N²-{[cyclohexyl(methyl)amino]carbonyl}-N¹-[4-({[4-fluoro-2-(trifluoromethyl)phenyl]sulfonyl}amino)butyl]-L-leucinamide

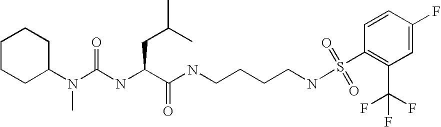

The title compound was prepared following the general procedure of Example 69 except substituting cyclohexylmethylamine with N-methylcyclohexylamine; LCMS (m/z): 567 (M+H).

### Example 71

### N²-{[cyclopentyl(methyl)amino]carbonyl}-N¹-[4-({[4-fluoro-2-(trifluoromethyl)phenyl]sulfonyl}amino)butyl]-L-leucinamide

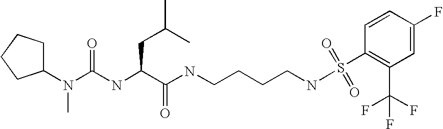

The title compound was prepared following the general procedure of Example 69 except substituting cyclohexylmethylamine with N-methylcyclopentylamine; LCMS (m/z): 553 (M+H).

### Example 72

### N¹-[4-({[4-fluoro-2-(trifluoromethyl)phenyl]sulfonyl}amino)butyl]-N²-{[(phenylmethyl)amino]carbonyl}-L-leucinamide

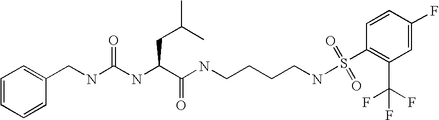

The title compound was prepared following the general procedure of Example 66 except substituting isocyanatocyclohexane with (isocyanatomethyl)benzene; LCMS (m/z): 561 (M+H).

### Example 73

### N²-{[(cyclopentylmethyl)oxy]carbonyl}-N¹-[4-({[4-fluoro-2-(trifluoromethyl)phenyl]sulfonyl}amino)butyl]-L-leucinamide

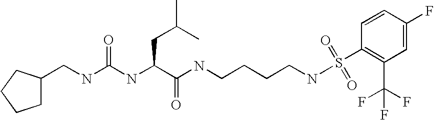

The title compound was prepared following the general procedure of Example 69 except substituting cyclohexylmethanamine with cyclopentanemethanol; LCMS (m/z): 554 (M+H).

### Example 74

### N²-{[(cyclohexylmethyl)oxy]carbonyl}-N¹-[4-({[4-fluoro-2-(trifluoromethyl)phenyl]sulfonyl}amino)butyl]-L-leucinamide

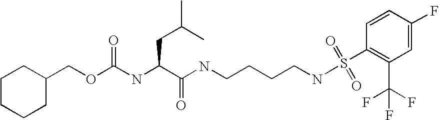

The title compound was prepared following the general procedure of Example 69 except substituting cyclohexylmethanamine with cyclohexylmethanol; LCMS (m/z): 570 (M+H).

### Example 75

### N¹-(4-{[(2-chloro-4-fluorophenyl)sulfonyl]amino}butyl)-N²-{[(cyclopentylmethyl)oxy]carbonyl}-L-leucinamide

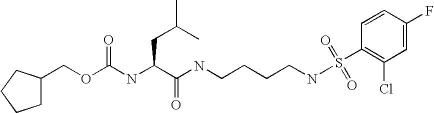

The title compound was prepared following the general procedure of Example 71 except substituting 4-fluoro-2-trifluoromethyl)benzenesulfonyl chloride with 2-chloro-4-fluorobenzenesulfonyl chloride; MS (m/z): 520 (M+H).

### Example 76

### N¹-(4-{[(2-chloro-4-fluorophenyl)sulfonyl]amino}butyl)-N²-{[(cyclohexylmethyl)oxy]carbonyl}-L-leucinamide

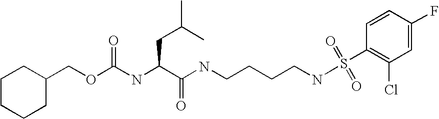

The title compound was prepared following the general procedure of Example 75 except substituting cyclopentylmethanol with cyclohexylmethanol; LCMS (m/z): 534 (M+H).

### Example 77

### N-((1S)-1-{[(4-{[(2,4-dichlorophenyl)sulfonyl]amino}butyl)amino]carbonyl}-3-methyl-3-buten-1-yl)-1-benzothiophene-2-carboxamide

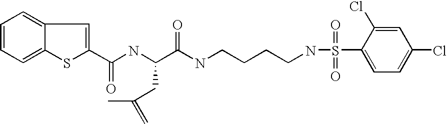

The title compound was prepared following the procedure of Example 9 except for the use of Boc-(L)-dehydro-leucine in place of Boc-(L)-leucine and replacing 3-methylfuro[3,2-b]pyridine-2-carboxylic acid with 1-benzothiophene-2-carboxylic acid; LCMS: [MH]⁺=568.

### Example 78

### N-[(1S)-2-[(4-{[(2,4-dichlorophenyl)sulfonyl]amino}butyl)amino]-2-oxo-1-(1,3-thiazol-4-ylmethyl)ethyl]-1-benzothiophene-2-carboxamide

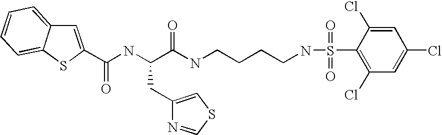

The title compound was prepared following the procedure of Example 9 except for the use of N-Boc-3-(1,3-thiazol-4-yl)-L-alanine in place of N-Boc-(L)-leucine and replacing 3-methylfuro[3,2-b]pyridine-2-carboxylic acid with 1-benzothiophene-2-carboxylic acid; LCMS: [MH]⁺=611.

### Example 79

### N-((1S)-2-[(4-{[(2-chloro-4-fluorophenyl)sulfonyl]amino}butyl)amino]-1-{[(1,1-dimethylethyl)oxy]methyl}-2-oxoethyl)-1-benzothiophene-2-carboxamide

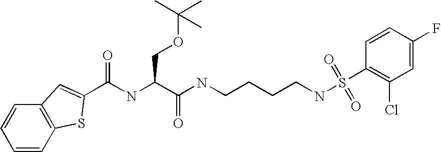

The title compound was prepared following the general procedure of Example 69 except substituting Boc-(L)-dehydro-leucine with O-(1,1-dimethylethyl)-N-{[(9H-fluoren-9-ylmethyl)oxy]carbonyl}-L-serine (using piperidine in DMF to remove FMOC) and substituting 2,4-dichlorobenzenesulfonyl chloride with 2-chloro-4-florobenzenesulfonyl chloride; LCMS (m/z): 584 (M+H).

### Example 80

### N-{(1R)-2-[(4-{[(2-chloro-4-fluorophenyl)sulfonyl]amino}butyl)amino]-1-[(methylthio)methyl]-2-oxoethyl}-1-benzothiophene-2-carboxamide

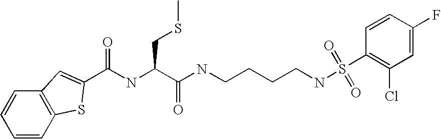

The title compound was prepared following the general procedure of Example 78 except substituting Boc-(L)-dehydro-leucine with N-{[(1,1-dimethylethyl)oxy]carbonyl}-S-methyl-L-cysteine and substituting 2,4-dichlorobenzenesulfonyl chloride with 2-chloro-4-florobenzenesulfonyl chloride; LCMS (m/z): 558 (M+H).

### Example 81

### N-((1S,2R)-1-{[(4-{[(2-chloro-4-fluorophenyl)sulfonyl]amino}butyl)amino]carbonyl}-2-hydroxy-3-methylbutyl)-1-benzothiophene-2-carboxamide

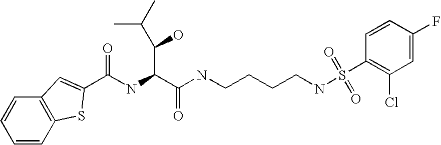

The title compound was prepared following the general procedure of Example 78 except substituting Boc-(L)-dehydro-leucine with (2S,3R)-2-({[(1,1-dimethylethyl)oxy]carbonyl}amino)-3-hydroxy-4-methylpentanoic acid and substituting 2,4-dichlorobenzenesulfonyl chloride with 2-chloro-4-florobenzenesulfonyl chloride; LCMS (m/z): 570 (M+H).

### Example 82

### N-((1S,2S)-1-{[(4-{[(2-chloro-4-fluorophenyl)sulfonyl]amino}butyl)amino]carbonyl}-2-hydroxy-3,3-dimethylbutyl)-1-benzothiophene-2-carboxamide

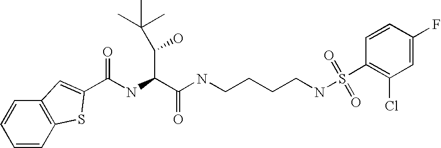

The title compound was prepared following the general procedure of Example 78 except substituting Boc-(L)-dehydro-leucine with (2S,3S)-2-({[(1,1-dimethylethyl)oxy]carbonyl}amino)-3-hydroxy-4,4-dimethylpentanoic acid and substituting 2,4-dichlorobenzenesulfonyl chloride with 2-chloro-4-florobenzenesulfonyl chloride; LCMS (m/z): 584 (M+H).

### Example 83

### N-((1S)-3,3-dichloro-1-{[(4-{[(2,4-dichlorophenyl)sulfonyl]amino}butyl)amino]-carbonyl}propyl)-1-benzothiophene-2-carboxamide

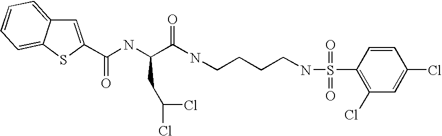

### a. 1-[(1-Benzothien-2-ylcarbonyl)oxy]-2,5-pyrrolidinedione

To a solution of 1-benzothiophene-2-carboxylic acid (10 g, 56.18 mmol) in CH₂Cl₂ (281 mL) in a dried 1 L round bottom flask, N-hydroxysuccinimide (7.11 g, 61.8 mmol) and 1-[3-(dimethylamino)propyl]-3-ethylcarbodiimide hydrochloride (12.92 g, 67.40 mmol) were added. The reaction mixture was stirred under nitrogen at RT for 3 hr. The mixture was washed with brine (2×150 mL), and the organic layer was dried over MgSO₄. After filtration and concentration under reduced pressure, the white solid (15.4 g) was carried to the next step without further purification.

### b. (2S)-2-[(1-benzothien-2-ylcarbonyl)amino]-4,4-dichlorobutanoic acid

Triethylamine (0.29 mL, 2.1 mmol) was slowly added at 0° C., to a solution of 1-[(1-benzothien-2-ylcarbonyl)oxy]-2,5-pyrrolidinedione (0.414 g, 1.51 mmol) and (2S)-2-amino-4,4-dichloro-butanoic acid [D. Winkler, K. Burger, Synthesis, 1419 (1996)] (0.286 g, 1.67 mmol) in EtOH (5 mL), CH₂Cl₂ (3.0 mL), and deionized water (2.0 mL). The mixture was warmed up to rt and stirred for 18 hr, then concentrated in vacuo. After dilution with 20 mL of water, the mixture was basified to pH˜12 using 1N aq. NaOH, and extracted with dichloromethane (50 mL×3). Next, the aqueous layer was acidified to pH ˜2 and extracted with dichloromethane (50 mL×3). The organic layer was dried over MgSO₄, filtered, and concentrated under reduced pressure. The title compound (0.269 g) was carried to the next step without further purification; LCMS: [MH]⁺=332.

### c. N-(4-aminobutyl)-2,4-dichlorobenzenesulfonamide

To a solution of 1,4-diaminobutane (3.60 g, 40.84 mmol) in CH₂Cl₂ (100 mL) were added triethylamine (5.70 mL, 40.84 mmol) and 2,4-dichlorobenzene sulfonyl chloride (5.01 g, 20.4 mmol) at 0° C. After stirring for 2 h at 0° C., deionized water was added and the reaction mixture was acidified to pH ˜2 with 1N aq. HCl. After extraction with CH₂Cl₂, the aqueous solution was basified to pH ˜10-11 with 1N. aq. NaOH, then extracted twice with dichloromethane and once with ethyl acetate. The combined organic layer was dried over MgSO₄, filtered, and concentrated under reduced pressure to yield the title compound (5.18 g, 85%, oil/solid) which was used without further purification; LCMS: [MH]⁺=297.

### d. N-((1S)-3,3-dichloro-1-{[(4-{[(2,4-dichlorophenyl)sulfonyl]amino}butyl)amino]-carbonyl}propyl)-1-benzothiophene-2-carboxamide

To a solution of N-(4-aminobutyl)-2,4-dichlorobenzenesulfonamide (0.264 g, 0.894 mmol) in CH₂Cl₂ (10 mL) were added (2S)-2-[(1-benzothien-2-ylcarbonyl)amino]-4,4-dichlorobutanoic acid (0.269 g, 0.813 mmol), and HOOBt (3.30 mg, 0.02 mmol). After the mixture was cooled to 0° C., NMM (0.268 mL, 2.44 mmol) and EDC-HCl (172 mg, 0.894 mmol) were added. After stirring for 18 h at RT, the reaction mixture was quenched with 10% aq. citric acid (˜15 mL) and extracted with dichloromethane (50 mL×2). The organic layer was washed with saturated aq. NaHCO₃ solution and brine, then dried over MgSO₄, filtered, concentrated and purified by flash column chromatography (THF/CH₂Cl₂, 0% to 4.5%) to provide the title compound (302 mg, 61%); LCMS: [MH]⁺=610.

### Example 84

### N-((1S)-3,3-dichloro-1-{[(4-{[(2-chloro-4-fluorophenyl)sulfonyl]amino}butyl)amino]-carbonyl}propyl)-1-benzothiophene-2-carboxamide

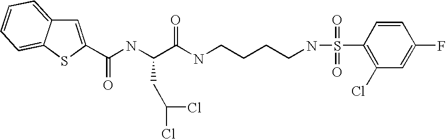

The title compound was prepared following the procedure of example 83 except for the substitution of 2-chloro-4-fluorobenzenesulfonyl chloride for 2,4-dichlorobenzenesulfonyl chloride; LCMS: [MH]⁺=594.

### Example 85

### (2S)-4,4-dichloro-N-(4-{[(2-chloro-4-fluorophenyl)sulfonyl]amino}butyl)-2-{[(cyclohexylamino)carbonyl]amino}butanamide

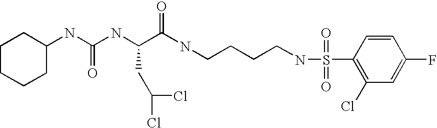

The title compound was prepared following the procedure of Example 9 with the following exceptions. In the first step 2-chloro-4-fluoro-benzene sulfonyl chloride was used in place of 2,4-dichlorobenzenesulfonyl chloride. In the next step (2S)-4,4-dichloro-2-({[(1,1-dimethylethyl)oxy]carbonyl}amino)butanoic acid is used in place of Boc-(L)-leucine. (2S)-4,4-dichloro-2-({[(1,1-dimethylethyl)oxy]carbonyl}amino)butanoic acid is prepared by BOC protection of (2S)-2-amino-4,4-dichloro-butanoic acid using ([D. Winkler, K. Burger, Synthesis, 1419 (1996)]) (BOC)₂O and K₂CO₃ in THF/H₂O. Finally, after deprotection, cyclohexylisocyanate was coupled using triethylamine in dichloromethane, in place of coupling 3-methylfuro[3,2-b]pyridine-2-carboxylic acid; LCMS: [MH]⁺=561.

### Example 86

### N-(1-{[(4-{[(2,4-dichlorophenyl)sulfonyl]amino}butyl)amino]carbonyl}-3,3,3-trifluoropropyl)-1-benzothiophene-2-carboxamide

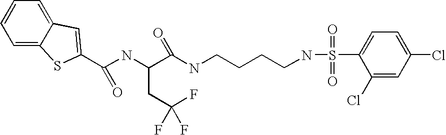

The title compound was prepared following the procedure of Example 83 except for the use of 2-amino-4,4,4-trifluorobutanoic acid in place of (2S)-2-amino-4,4-dichloro-butanoic acid. LCMS; [MH]⁺=596.

### Example 87

### N-((1S)-1-{[(4-{[(2,4-dichlorophenyl)sulfonyl]amino}butyl)amino]carbonyl}-3,3-difluoropropyl)-1-benzothiophene-2-carboxamide

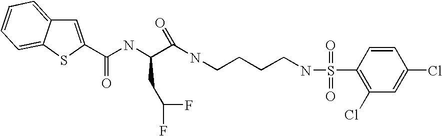

The title compound was prepared following the procedure of Example 83 except for the use of (2S)-2-amino-4,4-difluorobutanoic acid [D. Winkler, K. Burger, Synthesis, 1419 (1996)] in place of (2S)-2-amino-4,4-dichloro-butanoic acid; LCMS: [MH]⁺=578.

### Example 88

### N-((1R)-{[(4-{[(2,4-dichlorophenyl)sulfonyl]amino}butyl)amino]carbonyl}-3-methylbutyl)-1-benzothiophene-2-carboxamide

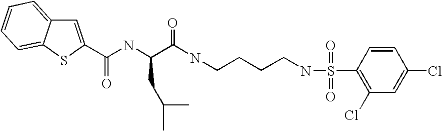

The title compound was prepared following the procedure of Example 83 except for the use of D-leucine in place of (2S)-2-amino-4,4-dichloro-butanoic acid; LCMS: [MH]⁺=570.

### Example 89

### N-{(1S)-2-[(4-{[(2-Chloro-4-fluorophenyl)sulfonyl]amino}butyl)amino]-2-oxo-1-phenylethyl}-1-benzothiophene-2-carboxamide

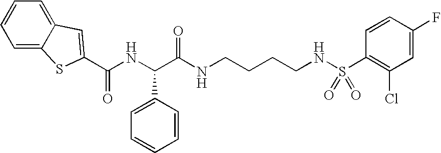

### a) N-(4-Aminobutyl)-2-chloro-4-fluorobenzenesulfonamide

To a stirred CH₂Cl₂ solution (0.05 M, 750 mL) of 1,4-diaminobutane (13.1 g, 148.4 mmol) and triethylamine (6.7 mL, 48.24 mmol) was added 2-chloro-4-fluorobenzenesulfonyl chloride (8.5 g, 37.1 mmol) in 100 mL of CH₂Cl₂ at a slow dropwise rate via an addition funnel resulting in the formation of a white precipitate. After 18 h, another portion of triethyl amine (6.7 mL, 48.24 mmol) was added, followed by dropwise addition of 2-chloro-4-fluorobenzenesulfonyl chloride (5 g, 21.83 mmol) in 100 mL of CH₂Cl₂ via an addition funnel. The reaction was quenched by the addition of water (500 mL, pH 11). The phases were separated and the organic portion was washed successively with 2 portions of water (500 mL) and brine (500 mL). The organic portion was then dried over Na₂SO₄, filtered, and concentrated under vacuum to provide the title compound as a yellow solid (16.18 g, 98%); LCMS (m/z): 281 (M+H).

### b) 1,1-Dimethylethyl{(1S)-2-[(4-{[(2-chloro-4-fluorophenyl)sulfonyl]amino}butyl)amino]-2-oxo-1-phenylethyl}carbamate

To a stirred CH₂Cl₂ solution (0.1 M, 7.5 mL) of N-(4-aminobutyl)-2-chloro-4-fluorobenzenesulfonamide (0.25 g, 0.89 mmol) were added EDC hydrogen chloride (0.19 g, 0.98 mmol), HOBt (0.13 g, 0.98 mmol), N-Boc-phenylglycine (0.22 g, 0.89 mmol) and triethylamine (0.3 mL, 2.23 mmol). The resulting solution was stirred at RT for 3 days, after which time 1 N HCl solution was added to the mixture. The layers were separated and the organic portion was washed with 5% aq. NaHCO₃ and brine. The organic phase was dried over Na₂SO₄, filtered and concentrated to a residue, which was purified by flash column chromatography (SiO₂, 50% ethyl acetate/hexanes). The title compound was isolated as a white solid in 58% yield (0.27 g); LCMS (m/z): 514 (M+H).

### c) (2S)-2-Amino-N-(4-{[(2-chloro-4-fluorophenyl)sulfonyl]amino}butyl)-2-phenylethanamide

To a solution of 1,1-dimethylethyl{(1S)-2-[(4-{[(2-chloro-4-fluorophenyl)sulfonyl]amino}butyl)amino]-2-oxo-1-phenylethyl}carbamate (0.27 g, 0.52 mmol) in CH₂Cl₂ (3.5 mL) was added 2N HCl in diethyl ether (0.8 mL, 1.56 mmol). The mixture was stirred at room temperature overnight. Evaporation of the solvent under reduced pressure gave the title compound as a white solid in 85% yield (0.2 g); LCMS (m/z): 414 (M+H)

### d) N-{(1S)-2-[(4-{[(2-Chloro-4-fluorophenyl)sulfonyl]amino}butyl)amino]-2-oxo-1-phenylethyl}-1-benzothiophene-2-carboxamide

To a solution of (2S)-2-amino-N-(4-{[(2-chloro-4-fluorophenyl)sulfonyl]amino}butyl)-2-phenylethanamide (0.050 g, 0.11 mmol) in dichloromethane (0.5 mL) were added 1-[(1-benzothien-2-ylcarbonyl)oxy]-2,5-pyrrolidinedione (0.032 g, 0.117 mmol) and triethylamine (0.034 mL, 0.244 mmol). After stirring for 2 h at room temperature, the reaction mixture was washed with 1N HCl, 5% aq. NaHCO₃ and brine. The organic solution was dried over Na₂SO₄, concentrated under reduced pressure, and purified by flash column chromatography (SiO₂, 50% ethyl acetate/hexanes) to provide the title compound as a white solid (0.05 g, 78%); LCMS (m/z): 574 (M+H).

### Example 90

### N-(1-Benzothien-2-ylcarbonyl)-N-(4-{[(2-chloro-4-fluorophenyl)sulfonyl]amino}butyl)-L-phenylalaninamide

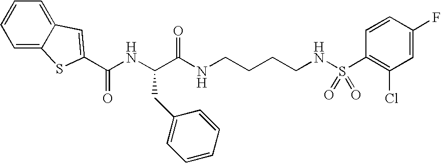

The title compound was prepared following the general procedure of Example 81 except substituting N-Boc-phenylalanine for N-Boc-phenylglycine; LCMS (m/z): 588 (M+H).

### Example 91

### N-((1S)-1-{[(4-{[(2,4-difluorophenyl)sulfonyl]amino}butyl)amino]carbonyl}-3-methylbutyl)-1-benzothiophene-2-carboxamide

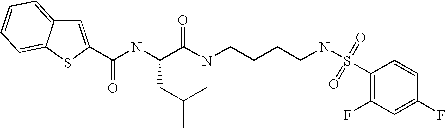

### a. 1-[(1-Benzothien-2-ylcarbonyl)oxy]-2,5-pyrrolidinedione

To a solution of 1-benzothiophene-2-carboxylic acid (10 g, 56.18 mmol) in CH₂Cl₂ (281 mL) in a dried 1 L round bottom flask, N-hydroxysuccinimide (7.11 g, 61.8 mmol) and 1-[3-(dimethylamino)propyl]-3-ethylcarbodiimide hydrochloride (12.92 g, 67.40 mmol) were added. The reaction mixture was stirred under nitrogen at RT for 4 hr. After evaporating CH₂Cl₂ (up to ½) under reduced pressure, the residue was washed with brine twice. The organic solution was dried over MgSO₄. After filtration, concentration, and drying under reduced pressure, the white solid (15.4 g) was carried to the next step without further purification.

### b. N-(1-Benzothien-2-ylcarbonyl)-L-leucine

To a solution of 1-[(1-benzothien-2-ylcarbonyl)oxy]-2,5-pyrrolidinedione (15.4 g, 56.18 mmol) and L-leucine (7.66 g, 58.43 mmol) in EtOH (140 mL), CH₂Cl₂ (85 mL) and deionized water (55 mL), triethyl amine (9.4 mL, 67.42 mmol) was slowly added at between 5° C. and 10° C. After 10 min, the mixture was warmed up to rt and stirred for 18 hr. The mixture was diluted with 50 mL of water, and then pH was adjusted to 1 with 6N HCl, followed by extraction with methylene chloride (2×100 mL). The organic solution was dried over MgSO₄, filtered, and concentrated. After drying under reduced pressure, the white solid product (16.4 g) was carried to the next step without further purification.

### c. 1,1-Dimethylethyl (4-{[N-(1-benzothien-2-ylcarbonyl)-L-leucyl]amino}butyl)carbamate

To a solution of N-(1-benzothien-2-ylcarbonyl)-L-leucine (crude, 407 mg, 1.40 mmol) and N-(4-aminobutyl)carbamic acid tert-butyl ester (2.24 mL, 1.27 mmol) in CH₂Cl₂ (10 mL) were added HOOBt (10 mg, 0.06 mmol), EDC.HCl (280 mg, 1.46 mmol), and NMM (0.18 mL, 1.65 mmol) at rt. After stirring for 5 hr at rt, the reaction was quenched with cold 1N HCl. After extraction with CH₂Cl₂ (20 mL×2), the organic solution was washed with sat'd aq. NaHCO₃ and brine, followed by drying over MgSO₄, filtration, and concentration. The resultant residue was purified by flash column chromatography (Biotage, 20% to 60% EtOAc/Hex) to provide 387 mg (66%) of the desired product.

### d. N-((1S)-1-{[(4-Aminobutyl)amino]carbonyl}-3-methylbutyl)-1-benzothiophene-2-carboxamide hydrochloride

To a solution of 1,1-dimethylethyl (4-{[N-(1-benzothien-2-ylcarbonyl)-L-leucyl]amino}butyl)carbamate (110 mg, 0.239 mmol) in CH₂Cl₂ (1.5 mL) was added 4N HCl in dioxane (0.7 mL) at rt. After stirring for 30 min at rt, the reaction mixture was concentrated and dried under reduced pressure. The resultant amorphous solid was carried to the next reaction without further purification.

### e. N-((1S)-1-{[(4-{[(2,4-Difluorophenyl)sulfonyl]amino}butyl)amino]carbonyl}-3-methylbutyl)-1-benzothiophene-2-carboxamide

To a solution of N-((1S)-1-{[(4-aminobutyl)amino]carbonyl}-3-methylbutyl)-1-benzothiophene-2-carboxamide hydrochloride (0.15 mmol) in CH₂Cl₂ (2 mL) were added 2,4-difluorobenzenesulfonyl chloride (36 mg, 0.169 mmol) and Et₃N (0.10 mL, 0.74 mmol) at rt. After stirring for 30 min at rt, the reaction mixture was concentrated and purified by flash column chromatography (Biotage, 20% EtOAC/Hex to 60% EtOAC/Hex) without aqueous work-up to provide 76 mg of a desired product (95% for two steps); LCMS: [MH]⁺=537.2.

### Example 92

### N-((1S)-1-{[(4-{[(4-fluoro-2-methylphenyl)sulfonyl]amino}butyl)amino]carbonyl}-3-methylbutyl)-1-benzothiophene-2-carboxamide

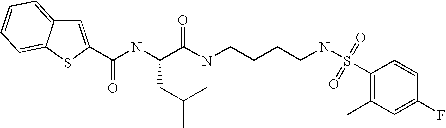

The title compound was prepared following the procedure of Example 91 except for the use of 2-methyl-4-difluorobenzenesulfonyl chloride in place of 2,4-difluorobenzenesulfonyl chloride; LCMS: [MH]⁺=534.2.

### Example 93

### N-{(1S)-1-[({4-[{[4-fluoro-2-(trifluoromethyl)phenyl]sulfonyl}(methyl)amino]butyl}amino)carbonyl]-b 3-methylbutyl}-1-benzothiophene-2-carboxamide

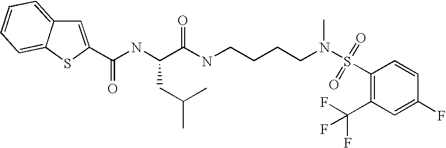

The title compound was prepared following the procedure of Example 91 except for the use of 2-trifluoromethyl-4-fluorobenzenesulfonyl chloride in place of 2,4-difluorobenzenesulfonyl chloride and 1,1-dimethylethyl (4-aminobutyl)methylcarbamate in place of N-(4-aminobutyl)carbamic acid tert-butyl ester; LCMS: [MH]⁺=602.2

### Example 94

### N-{(1S)-1-[({4-[[(2,4-dichlorophenyl)sulfonyl(methyl)amino]butyl}amino)carbonyl]-3-methylbutyl}-1-benzothiophene-2-carboxamide

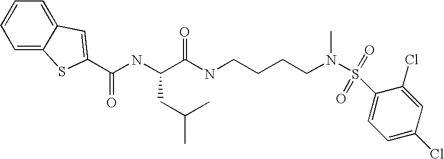

The title compound was prepared following the procedure of Example 91 except for the use of 2,4-dichlorobenzenesulfonyl chloride in place of 2,4-difluorobenzenesulfonyl chloride and 1,1-dimethylethyl (4-aminobutyl)methylcarbamate in place of N-(4-aminobutyl)carbamic acid tert-butyl ester; LCMS: [MH]⁺=583.2.

### Example 95

### N-{(1S)-1-[({4-[[(2-chloro-4-fluorophenyl)sulfonyl](methyl)amino]butyl}amino)carbonyl]-3-methylbutyl}-1-benzothiophene-2-carboxamide

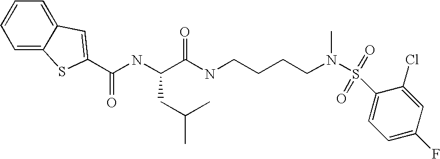

The title compound was prepared following the procedure of example 83 except for the use of 2-chlorol-4-fluorobenzenesulfonyl chloride in place of 2,4-difluorobenzenesulfonyl chloride and 1,1-dimethylethyl (4-aminobutyl)methylcarbamate in place of N-(4-aminobutyl)carbamic acid tert-butyl ester; LCMS: [MH]⁺=568.2.

### Example 96

### N-{(1S)-1-[({4-[[(2-cyanophenyl)sulfonyl](methyl)amino]butyl}amino)carbonyl]-3-methylbutyl}-1-benzothiophene-2-carboxamide

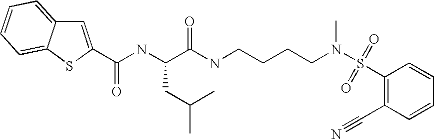

The title compound was prepared following the procedure of Example 91 except for the use of 2-cyanobenzenesulfonyl chloride in place of 2,4-difluorobenzenesulfonyl chloride and 1,1-dimethylethyl (4-aminobutyl)methylcarbamate in place of N-(4-aminobutyl)carbamic acid tert-butyl ester; LCMS: [MH]⁺=541.2.

### Example 97

### 2-(3-Biphenylyl)-N-(4-{[(2,4-dichlorophenyl)sulfonyl]amino}butyl)-4-methylpentanamide

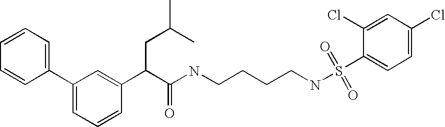

### a. 2-(3-Biphenylyl)-4-methyl-4-pentenoic acid

To a solution of diisopropylamine (1 g, 10.6 mmol) in THF (35 mL) at 0° C. was added n-BuLi (5.6 mL, 10.36 mmol, 1.84 M in THF) dropwise. After 15 minutes, the reaction mixture was cooled down to −78° C., followed by the addition of 3-biphenylylacetic acid (1 g, 4.71 mmol) in THF (6 mL) through a double ended needle. After warming up the reaction mixture to −20° C., 3-bromo-2-methyl-1-propene (0.79 mL, 8 mmol) was added. Stirred at −20° C. for 2 hr, and then this mixture was allowed to warm to room temperature and stirred overnight. The mixture was washed by saturated aq. NH₄Cl, saturated aq. NaHCO₃, and brine. The organic solution was dried over MgSO₄, filtered, and concentrated. Purification of the residue by Biotage silica gel column chromatography (1% to 5% MeOH/DCM) provided 153 mg of the title compound (13%); LCMS (m/z): 266.3 (M+H).

### b. 2-(3-Biphenylyl)-4-methylpentanoic acid

2-(3-Biphenylyl)-4-methyl-4-pentenoic acid (153 mg, 0.58 mmol) was dissolved in EtOAc (2 mL) and EtOH (3 mL). After Pd/carbon (10%, 61 mg, 0.058 mmol) was added, the reaction mixture was vigorously stirred under hydrogen (balloon) for 4 hr. The mixture was filtered through celite, which was rinsed with MeOH. The combined filtrate was concentrated and the crude material was used for the next step without further purification; LCMS (m/z): 268.5 (M+H).

### c. 2-(3-biphenylyl)-N-(4-{[(2,4-dichlorophenyl)sulfonyl]amino}butyl)-4-methylpentanamide

To a solution 2-(3-biphenylyl)-4-methylpentanoic acid (42 mg, 0.157 mmol) in CH₂Cl₂ (2 mL) were added N-(4-aminobutyl)-2,4-dichlorobenzenesulfonamide (51 mg, 0.172 mmol), HOOBt (0.5 mg, 0.003 mmol) and N-methylmorpholine (0.05 mL, 0.47 mmol) at 0° C. The mixture was stirred for several minutes whereupon EDC.HCl (36 mg, 0.188 mmol) was added. Allowed the mixture to warm up to room temperature and kept stirring overnight. The reaction mixture was washed with 10% (w/w) aqueous citric acid solution, saturated aq. NaHCO₃ solution, and brine. The organic layer was dried over MgSO₄, filtered, and concentrated. Purification of the residue by Biotage silica gel column chromatography (10%-60% EtOAc/Hexane) provided 70 mg of the title compound (82%); LCMS (m/z): 547.5 (M+H).

### Example 98

### N-((1S)-1-{[((2S)-4-{[(2-chloro-4-fluorophenyl)sulfonyl]amino}-2-hydroxybutyl)amino]carbonyl}-3-methylbutyl)-1-benzothiophene-2-carboxamide

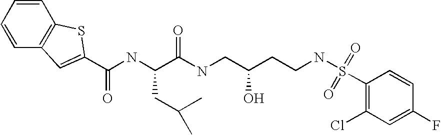

### a. 2-[(2S)-2-Oxiranylmethyl]-1H-isoindole-1,3(2H)-dione

To a solution of triphenylphosphine (17.7 g, 67.5 mmol), (R)-(+)-glycidol (4.0 g, 54.0 mmol), and phthalamide in THF (220 mL) was added DEAD (11.7 g, 67.5 mmol). The reaction mixture was stirred under nitrogen at RT for 4 hr. After evaporating solvent, the crude product was purified by flash column chromatography to provide 5.8 g (53%) of the desired product; LCMS: [MH]⁺=204.

### b. (3S)-4-(1,3-Dioxo-1,3-dihydro-2H-isoindol-2-yl)-3-hydroxybutanenitrile

A solution of 2-[(2S)-2-oxiranylmethyl]-1H-isoindole-1,3(2H)-dione (0.10 g, 0.50 mmol)), 2-hydroxy-2-methylpropane nitrile (0.051 g, 0.60 mmol), and TEA (0.083 ml, 0.60 mmol) in 1 mL THF was heated in a sealed tube at 75° C. overnight. The reaction mixture was cooled to room temperature, poured into 10 mL of H₂O, and extracted with ethyl acetate. The organic layer was washed with water and brine, dried over MgSO₄, followed by purification by flash column chromatography to provide 0.098 g (87%) of the desired product; LCMS: [MH]⁺=231.

### c. 2-[(2S)-4-Amino-2-hydroxybutyl]-1H-isoindole-1,3(2H)-dione

To a solution of (3S)-4-(1,3-dioxo-1,3-dihydro-2H-isoindol-2-yl)-3-hydroxybutanenitrile (90 mg, 0.39 mmol) in 2 mL of EtOH were added PtO₂ (6 mg, 0.05 mmol) and conc. HCl (0.1 ml). The reaction mixture was stirred under H₂ (a balloon pressure) at room temperature for 6 hours. After filtration and concentration, the mixture was carried out to the next step without further purification.

### d. 2-Chloro-N-[(3S)-4-(1,3-dioxo-1,3-dihydro-2H-isoindol-2-yl)-3-hydroxybutyl]-4-fluorobenzenesulfonamide

To a solution of 2-[(2S)-4-amino-2-hydroxybutyl]-1H-isoindole-1,3(2H)-dione (70 mg, 0.30 mmol) in CH₂Cl₂ (2 mL) were added 2-chloro-4-fluorobenzenesulfonyl chloride (66 mg, 0.29 mmol) and Et₃N (0.13 mL, 0.90 mmol) at rt. After stirring for 30 min at rt, the reaction mixture was purified by flash column chromatography (Biotage, 20% to 60% EtOAc/Hex) without aqueous work-up to provide 65 mg (55%) of a desired product LCMS: [MH]⁺=427.

### e. N-[(3S)-4-Amino-3-hydroxybutyl]-2-chloro-4-fluorobenzenesulfonamide

To a solution of 2-chloro-N-[(3S)-4-(1,3-dioxo-1,3-dihydro-2H-isoindol-2-yl)-3-hydroxybutyl]-4-fluorobenzenesulfonamide (0.40 g, 1.0 mmol) in 10 mL of EtOH was added NH₂NH₂(0.13 g, 4.0 mmol). The reaction mixture was stirred at room temperature overnight. The reaction mixture was filtered and the solvent was evaporated. The crude material was carried to next step without further purification.

### f. N-((1S)-1-{[((2S)-4-{[(2-Chloro-4-fluorophenyl)sulfonyl]amino}-2-hydroxybutyl)amino]carbonyl}-3-methylbutyl)-1-benzothiophene-2-carboxamide

To a solution of N-(1-benzothien-2-ylcarbonyl)-L-leucine (crude, 52 mg, 0.18 mmol) and N-[(3S)-4-amino-3-hydroxybutyl]-2-chloro-4-fluorobenzenesulfonamide (44 mg, 0.15 mmol) in CH₂Cl₂ (2 mL) were added HOOBt (2 mg, 0.012 mmol), EDC.HCl (34 mg, 0.18 mmol), and NMM (0.050 mL, 0.45 mmol) at rt. After stirring for 5 hr at rt, the reaction mixture was quenched with cold 1N HCl. After extraction with CH₂Cl₂ (20 mL×2), the organic solution was washed with sat'd aq. NaHCO₃ and brine, followed by drying over MgSO₄, filtration, and concentration. The resultant residue was purified by flash column chromatography (Biotage, 20% to 80% EtOAc/Hex) to provide 50 mg (59%) of the desired product; LCMS: [MH]⁺=570.2.

### Example 99

### N-((1S)-1-{[((2R)-4-{[(2-chloro-4-fluorophenyl)sulfonyl]amino}-2-hydroxybutyl)amino]carbonyl}-3-methylbutyl)-1-benzothiophene-2-carboxamide

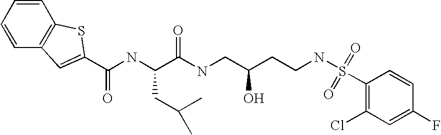

The title compound was prepared following the procedure of Example 98 except for the use of S-(−)-glycidol in place of R-(+)-glycidol; LCMS: [MH]⁺=570.2.

### Example 100

### N-((1S)-3,3-dichloro-1-{[((2S)-4-{[(2,4-dichlorophenyl)sulfonyl]amino}-2-hydroxybutyl)amino]carbonyl}propyl)-1-benzothiophene-2-carboxamide

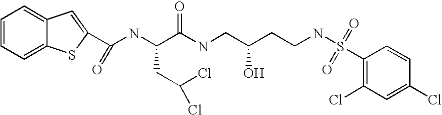

The title compound was prepared following the procedure of Example 98 except for the use of (2S)-2-[(1-benzothien-2-ylcarbonyl)amino]-4,4-dichlorobutanoic acid in place of N-(1-benzothien-2-ylcarbonyl)-L-leucine and 2,4-dichlorobenzenesulfonyl chloride in place of 2-chloro-4-fluorobenzenesulfonyl chloride; LCMS: [MH]⁺=626.2.

### Example 101

### N²-[(cyclohexylamino)carbonyl]-N¹-((2S)-4-{[(2,4-dichlorophenyl)sulfonyl]amino}-2-hydroxybutyl)-L-leucinamide

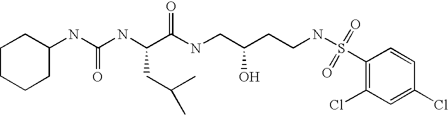

### a. N¹-((2S)-4-{[(2,4-dichlorophenyl)sulfonyl]amino}-2-hydroxybutyl)-N²-{[(1,1-dimethylethyl)oxy]carbonyl}-L-leucinamide

The title compound was prepared following the procedure of Example 1 except for the use of BocLeu in place of N-(1-benzothien-2-ylcarbonyl)-L-leucine.

### b. N¹-((2S)-4-{[(2,4-dichlorophenyl)sulfonyl]amino}-2-hydroxybutyl)-L-leucinamide

To a solution of N²-[(cyclohexylamino)carbonyl]-N-((2S)-4-{[(2,4-dichlorophenyl)sulfonyl]amino}-2-hydroxybutyl)-L-leucinamide (89 mg, 0.17 mmol) in CH₂Cl₂ (1.0 mL) was added 4N. HCl in dioxane (0.5 mL) at rt. After stirring for 30 min at rt, the reaction mixture was concentrated and dried under the reduced pressure. The resultant amorphous solid was carried out to the next reaction without further purification.

### c. N²-[(cyclohexylamino)carbonyl]-N¹-((2S)-4-{[(2,4-dichlorophenyl)sulfonyl]amino}-2-hydroxybutyl)-L-leucinamide

To a solution of N¹-((2S)-4-{[(2,4-dichlorophenyl)sulfonyl]amino}-2-hydroxybutyl)-L-leucinamide (37 mg, 0.08 mmol) in 1 ml CH₂Cl₂ was added cyclohexane isocyanate (10 mg, 0.08 mmol). After stirring for 12 hr at rt, the reaction mixture was purified by flash column chromatography (Biotage, 20% to 90% EtOAc/Hex) without aqueous work-up to provide 35 mg (80%) of a desired product; LCMS: [MH]⁺=551.2.

### Example 102

### N-(1S)-1-{[((2S)-4-{[(2-chloro-4-fluorophenyl)sulfonyl]amino}-2-hydroxybutyl)amino]carbonyl}-3-methylbutyl)-1-benzofuran-2-carboxamide

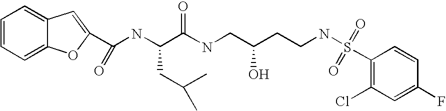

The title compound was prepared following the procedure of Example 98 except for the use of N-(1-benzofuran-2-ylcarbonyl)-L-leucine in place of N-(1-benzothien-2-ylcarbonyl)-L-leucine; LCMS: [MH]⁺=554.2.

### Example 103

### N-((1S)-1-{[((3S)-4-{[(2-chloro-4-fluorophenyl)sulfonyl]amino}-3-hydroxybutyl)amino]carbonyl}-3-methylbutyl)-1-benzothiophene-2-carboxamide

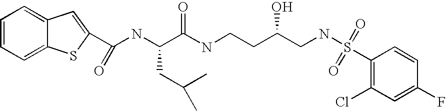

### a. 1,1-Dimethylethyl[(1S)-1-({[(3S)-3-hydroxy-4-(1-oxo-1,3-dihydro-2H-isoindol-2-yl)butyl]amino}carbonyl)-3-methylbutyl]carbamate

To a solution of 2-[(2S)-4-amino-2-hydroxybutyl]-1H-isoindole-1,3(2″-dione (0.10 g, 0.37 mmol) in dichloromethane (5 mL) was added N-Boc-L-leucine (0.10 g, 0.44 mmol) and HOOBt (5.0 mg, 0.22 mmol) at rt. After cooling the reaction mixture in an ice-bath, NMM (0.16 g, 1.48 mmol) and EDC.HCl (0.084 g, 0.44 mmol) were added. After stirring overnight at rt, the reaction mixture was washed with 10% (w/w) aqueous citric acid solution (2 mL) and brine. The organic solution was dried over MgSO₄, concentrated under reduced pressure, and then purified by silica gel column chromatography (30% to 70% EtOAc/Hex) to give the title compound (0.081 g, white solid, 51%); LCMS: [MH]⁺=434.2.

### b. N¹-[(3S)-4-Amino-3-hydroxybutyl]-N²-{[(1,1-dimethylethyl)oxy]carbonyl}-L-leucinamide

To a solution of 1,1-dimethylethyl[(1S)-1-({[(3S)-3-hydroxy-4-(1-oxo-1,3-dihydro-2H-isoindol-2-yl)butyl]amino}carbonyl)-3-methylbutyl]carbamate (0.15 g, 0.36 mmol) in 5 mL of EtOH was added NH₂NH₂ (0.13 g, 4.0 mmol). The reaction mixture was stirred at room temperature overnight. The white solid was filtered off and the filtrate was evaporated. The crude material was carried to next step without purification.

### c. N/1-((3S)-4-{[(2-Chloro-4-fluorophenyl)sulfonyl]amino}-3-hydroxybutyl)-N²-{[(1,1-dimethylethyl)oxy]carbonyl}-L-leucinamide

To a solution of N¹-[(3S)-4-amino-3-hydroxybutyl]-N²-{[(1,1-dimethylethyl)oxy]carbonyl}-L-leucinamide (57 mg, 0.18 mmol) in 2 ml of CH₂Cl₂ were added 2-chloro-4-fluorobenzenesulfonyl chloride (41 mg, 0.18 mmol) and Et₃N (54 mg, 0.54 mmol) at rt. After stirring for 30 min at rt, the reaction mixture was purified by flash column chromatography (Biotage, 20% to 60% EtOAc/Hex) without aqueous work-up to provide 84 mg of a desired product (91%); [MH]⁺=510.2.

### d. N¹-((3S)-4-{[(2-Chloro-4-fluorophenyl)sulfonyl]amino}-3-hydroxybutyl)-L-leucinamide

To a solution of N¹-((3S)-4-{[(2-chloro-4-fluorophenyl)sulfonyl]amino}-3-hydroxybutyl)-N²-{[(1,1-dimethylethyl)oxy]carbonyl}-L-leucinamide (92 mg, 0.18 mmol) in CH₂Cl₂ (1.0 mL) was added 4N. HCl in dioxane (0.5 mL) at rt. After stirring for 30 min at rt, the reaction mixture was concentrated and dried under the reduced pressure. The resultant amorphous solid was carried to the next reaction without further purification.

### e. N-((1S)-1-{[((3S)-4-{[(2-chloro-4-fluorophenyl)sulfonyl]amino}-3-hydroxybutyl)amino]carbonyl}-3-methylbutyl)-1-benzothiophene-2-carboxamide

To a solution of N¹-((3S)-4-{[(2-chloro-4-fluorophenyl)sulfonyl]amino}-3-hydroxybutyl)-L-leucinamide (67 mg, 0.15 mmol) in dichloromethane (2 mL) was added benzothiophene carboxylic acid (29 mg, 0.18 mmol) and HOOBt (2.0 mg, 0.01 mmol) at rt. After cooling the reaction mixture in an ice-bath, NMM (0.45 mmol) and EDC.HCl (34 mg, 0.18 mmol) were added. After stirring overnight at rt, the reaction mixture was washed with 10% (w/w) aqueous citric acid solution (1 mL) and brine. The organic solution was dried over MgSO₄, concentrated under reduced pressure, and then purified by silica gel column chromatography (30%-90% EtOAc/Hex) to give the title compound (0.060 g, 71%, white solid); LCMS: [MH]⁺=570.2.

### Example 104

### N-((1S)-1-{[((3R)-4-{[(2-chloro-4-fluorophenyl)sulfonyl]amino}-3-hydroxybutyl)amino]carbonyl}-3-methylbutyl)-1-benzothiophene-2-carboxamide

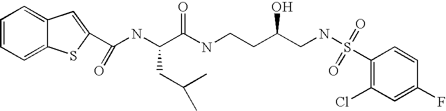

The title compound was prepared following the procedure of Example 103 except for the use of (S)-(−)-glycidol in place of (R)-(+)-glycidol; LCMS: [MH]⁺=570.2.

### Example 105

### N-((1S)-1-{[((4R)-4-{[(2-chloro-4-fluorophenyl)sulfonyl]amino}-5-hydroxypentyl)amino]carbonyl}-3-methylbutyl)-1-benzothiophene-2-carboxamide

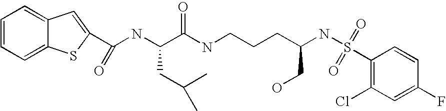

### a. N²-[(2-Chloro-4-fluorophenyl)sulfonyl]-N⁵-{[(1,1-dimethylethyl)oxy]carbonyl}-D-ornithine

To a solution of N⁵-{[(1,1-dimethylethyl)oxy]carbonyl}-D-ornithine (1.6 g, 6.7 mmol) in 2N NaOH (8 mL) at 0° C. were added 2-chloro-4-fluorobenzenesulfonyl chloride (1.8 g, 8.1 mmol) and i-Pr₂NEt (1.48 mL, 8.1 mmol), followed by the addition of acetone (8 mL). The homogenous solution was stirred at room temperature for overnight. The reaction mixture was extracted with ether. The organic layer was extracted with 2N NaOH. The combined aqueous layer was cooled to −10° C. Concentrated HCl was added to adjust pH to 1. The aqueous solution was extracted with CH₂Cl₂. The combined organic layer was washed with brine and dried over MgSO₄. The solution was concentrated under reduced pressure and the crude material was carried to the next reaction without further purification.

### b. 1,1-Dimethylethyl ((4R)-4-{[(2-chloro-4-fluorophenyl)sulfonyl]amino}-5-hydroxypentyl)carbamate

To a solution of N²-[(2-chloro-4-fluorophenyl)sulfonyl]-N⁵-{[(1,1-dimethylethyl)oxy]carbonyl}-D-ornithine (0.21 g, 0.5 mmol) in 1 mL of THF at −10° C. was added 3.0 mL of BH₃ (1M in THF). The reaction mixture was stirred at this temperature for 5 h. The solution was diluted with EtOAc, followed by washing with H₂O and brine, drying over MgSO₄, filtration, and concentration. The resultant residue was purified by flash column chromatography to provide 58 mg of the desired product (65%); LCMS: [MH]⁺=424.2.

### c. N-[(1R)-4-Amino-1-(hydroxymethyl)butyl]-2-chloro-4-fluorobenzenesulfonamide

To a solution of 1,1-dimethylethyl ((4R)-4-{[(2-chloro-4-fluorophenyl)sulfonyl]amino}-5-hydroxypentyl)carbamate (58 mg, 0.15 mmol) in CH₂Cl₂ (0.5 mL) was added 4N. HCl in dioxane (0.7 mL) at rt. After stirring for 30 min at rt, the reaction mixture was concentrated. After drying under reduced pressure, the resultant amorphous solid was carried to the next reaction without further purification.

### d. N-((1S)-1-{[((4R)-4-{[(2-Chloro-4-fluorophenyl)sulfonyl]amino}-5-hydroxypentyl)amino]carbonyl}-3-methylbutyl)-1-benzothiophene-2-carboxamide

To a solution of N-(1-benzothien-2-ylcarbonyl)-L-leucine (crude, 45 mg, 0.15 mmol) and N-[(1R)-4-amino-1-(hydroxymethyl)butyl]-2-chloro-4-fluorobenzenesulfonamide (60 mg, 0.12 mmol) in CH₂Cl₂ (2 mL) were added HOOBt (2 mg, 0.012 mmol), EDC.HCl (35 mg, 0.18 mmol), and NMM (0.10 mL, 1.65) at rt. After stirring for 5 hr at rt, the reaction mixture was quenched with cold 1N HCl. After extraction with CH₂Cl₂ (20 mL×2), the organic solution was washed with sat'd aq. NaHCO₃ and brine, followed by drying over MgSO₄, filtration, and concentration. The resultant residue was purified by flash column chromatography to provide 53 mg (66%) of the desired product; LCMS: [MH]⁺=584.2.

### Example 106

### N-((1S)-1-{[((4S)-4-{[(2-chloro-4-fluorophenyl)sulfonyl]amino}-5-hydroxypentyl)amino]carbonyl}-3-methylbutyl)-1-benzothiophene-2-carboxamide

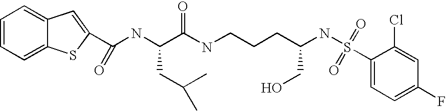

The title compound was prepared following the procedure of Example 105 except for the use of N⁵-{[(1,1-dimethylethyl)oxy]carbonyl}-L-ornithine in place of N⁵-{[(1,1-dimethylethyl)oxy]carbonyl}-D-ornithine; LCMS: [MH]⁺=584.2.

### Example 107

### Methyl N⁵-[N-(1-benzothien-2-ylcarbonyl)-L-leucyl]-N²-[(2-chloro-4-fluorophenyl)sulfonyl]-D-ornithinate

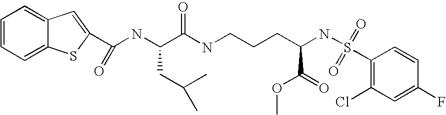

### a. Methyl N²-[(2-chloro-4-fluorophenyl)sulfonyl]-N⁵-{[(1,1-dimethylethyl)oxy]carbonyl}-D-ornithinate

To a solution of N²-[(2-chloro-4-fluorophenyl)sulfonyl]-N⁵-{[(1,1-dimethylethyl)oxy]carbonyl}-D-ornithine (1.86 g, 4.4 mmol) in toluene/MeOH (30 mL/15 mL) was added 2M Me₃SiCHN₂ in heptane (6.6 mL, 13.2 ml) at 0° C. The reaction mixture was stirred at 0° C. for 30 min. The solvent was removed and the residue was purified by flash column chromatography to provide 1.3 g of the desired product (70%); LCMS: [MH]⁺=339.3.

### b. Methyl N²-[(2-chloro-4-fluorophenyl)sulfonyl]-D-ornithinate

To a solution of methyl N²-[(2-chloro-4-fluorophenyl)sulfonyl]-N-{[(1,1-dimethylethyl)oxy]carbonyl}-D-ornithinate (0.50 g, 1.5 mmol) in CH₂Cl₂ (5 mL) was added 4N. HCl in dioxane (2 mL) at rt. After stirring for 30 min at rt, the reaction mixture was concentrated and dried under reduced pressure. The resultant amorphous solid was carried to the next reaction without further purification.

### c. Methyl N⁵—[N-(1-benzothien-2-ylcarbonyl)-L-leucyl]-N²-[(2-chloro-4-fluorophenyl)sulfonyl]-D-ornithinate

To a solution of N-(1-benzothien-2-ylcarbonyl)-L-leucine (crude, 0.35 g, 1.20 mmol) and Methyl N²-[(2-chloro-4-fluorophenyl)sulfonyl]-D-ornithinate (0.45 g, 1.20 mmol) in CH₂Cl₂ (10 mL) were added HOOBt (10 mg, 0.060 mmol), EDC.HCl (0.27 g, 1.40 mmol), and NMM (0.61 mL, 5.0 mmol) at rt. After stirring for 5 hr at rt, the reaction mixture was quenched with cold 1N HCl. After extraction with CH₂Cl₂ (20 mL×2), the organic solution was washed with sat'd aq. NaHCO₃ and brine, followed by drying over MgSO₄, filtration, and concentration. The resultant residue was purified by flash column chromatography to provide 0.61 g of the desired product (83%); LCMS: [MH]⁺=612.2.

### Example 108

### N⁵-[N-(1-benzothien-2-ylcarbonyl)-L-leucyl]-N²-[(2-chloro-4-fluorophenyl))sulfonyl]-N¹-methyl-D-ornithinamide

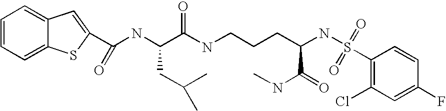

### a. N⁵-[N-(1-Benzothien-2-ylcarbonyl)-L-leucyl]-N²-[(2-chloro-4-fluorophenyl)sulfonyl]-D-ornithine

To a solution of methyl N⁵—[N-(1-benzothien-2-ylcarbonyl)-L-leucyl]-N²-[(2-chloro-4-fluorophenyl)sulfonyl]-D-ornithinate (0.50 g, 0.82 mmol) in MeOH/H₂O (8 mL/4 mL) was added K₂CO₃ (0.35 g, 2.5 mmol). The solution stirred at room temperature for 6 hours. The solvent was evaporated and the residue was acidified by 1 N HCl to pH 2. The resultant white solid was collected and was dried under reduced pressure to provide 0.48 g of the title product (99%); LCMS: [MH]⁺=598.2.

### b. N⁵—[N-(1-Benzothien-2-ylcarbonyl)-L-leucyl]-N²-[(2-chloro-4-fluorophenyl)sulfonyl]-N¹-methyl-D-ornithinamide

To a solution of N N⁵—[N-(1-benzothien-2-ylcarbonyl)-L-leucyl]-N²-[(2-chloro-4-fluorophenyl)sulfonyl]-D-ornithine (0.10 g, 0.17 mmol) and methyl amine (6.2 mg, 0.20 mmol) in CH₂Cl₂ (2 mL) were added HOOBt (1 mg, 0.005 mmol), EDC.HCl (0.038 g, 0.20 mmol), and NMM (0.10 mL, 0.80 mmol) at rt. After stirring for 5 hr at rt, the reaction mixture was quenched with cold 1N HCl. After extraction with CH₂Cl₂ (10 mL×2), the organic solution was washed with sat'd aq. NaHCO₃ and brine, followed by drying over MgSO₄, filtration, and concentration. The resultant residue was purified by flash column chromatography to provide 0.075 g of the desired product (72%); LCMS: [MH]⁺=611.2.

### Example 109

### N-{(1S)-1-[({[(4R,5R)-5-({[(2,4-dichlorophenyl)sulfonyl]amino}methyl)-2,2-dimethyl-1,3-dioxolan-4-yl]methyl}amino)carbonyl]-3-methylbutyl}-1-benzothiophene-2-carboxamide

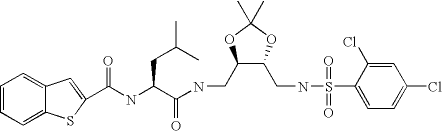

### a. [(4R,5R)-5-(Azidomethyl)-2,2-dimethyl-1,3-dioxolan-4-yl]methyl 4-methylbenzenesulfonate

To a solution of [(4R,5R)-2,2-dimethyl-1,3-dioxolane-4,5-diyl]dimethanediyl bis(4-methylbenzenesulfonate) (4 g, 8.5 mmol) in DMF (60 mL) was added sodium azide (368 mg, 5.667 mmol). The reaction mixture was stirred at 65° C. overnight. Most of the solvent was removed under reduced pressure and the residue was diluted in dichloromethane. The organic solution was washed by water, saturated aq. NaHCO₃, and brine. The organic layer was dried over MgSO₄, filtered and concentrated. Purification of the residue by Biotage silica gel column chromatography (10% to 60% EA/Hexane) provided 1.08 g of the title compound (63%).

### b. 2-{[(4R,5R)-5-(Azidomethyl)-2,2-dimethyl-1,3-dioxolan-4-yl]methyl}-1H-isoindole-1,3(2H)-dione

To a solution of [(4R,5R)-5-(azidomethyl)-2,2-dimethyl-1,3-dioxolan-4-yl]methyl 4-methylbenzenesulfonate (1.08 g, 3.17 mmol) in DMF (25 mL) was added potassium phthalimide (880 mg, 4.75 mmol). The reaction mixture was stirred at 100° C. overnight. After most of the solvent was removed under reduced pressure, the residue was diluted in dichloromethane. The reaction mixture was washed with water, saturated aq. NaHCO₃, brine. The organic layer was dried over MgSO₄, filtered, and concentrated. Purification of the residue by Biotage silica gel column chromatography (5% to 40% EA/Hexane) provided 674 mg of the title compound (67%).

### c. 1,1-Dimethylethyl ({(4R,5R)-5-[(1,3-dioxo-1,3-dihydro-2H-isoindol-2-yl)methyl]-2,2-dimethyl-1,3-dioxolan-4-yl}methyl)carbamate

Pd/carbon (10%, 225 mg, 0.213 mmol) in ethyl acetate (10 mL) was vigorously stirred under hydrogen for 10 minutes. A mixture of 2-{[(4R,5R)-5-(azidomethyl)-2,2-dimethyl-1,3-dioxolan-4-yl]methyl}-1H-isoindole-1,3(2H)-dione (674 mg, 2.13 mmol) and bis(1,1-dimethylethyl) dicarbonate (931 mg, 4.26 mmol) in ethyl acetate were added to the above suspension. The reaction mixture was stirred under hydrogen (balloon) at room temperature for 4 hours. The mixture was filtered through celite, which was rinsed with MeOH. The combined filtrates were concentrated and the residue was purified by Biotage silica gel column chromatography (5% to 30% EA/Hexane) to provide 787 mg of the title compound (95%); LCMS (m/z): 391.5 (M+H).

### d. 1,1-Dimethylethyl {[(4R,5R)-5-(aminomethyl)-2,2-dimethyl-1,3-dioxolan-4-yl]methyl}carbamate

To a solution of 1,1-dimethylethyl ({(4R,5R)-5-[(1,3-dioxo-1,3-dihydro-2H-isoindol-2-yl)methyl]-2,2-dimethyl-1,3-dioxolan-4-yl}methyl)carbamate (600 mg, 1.54 mmol) in ethanol (15 mL) was added hydrazine (0.38 mL, 12.32 mmol). The reaction mixture was stirred at room temperature under nitrogen overnight. The white precipitate was filtered off and was rinsed once with ethanol. The filtrate was concentrated down and the crude material was used for the next step without further purification; LCMS (m/z): 261.2 (M+H).

### e. 1,1-dimethylethyl {[(4R,5R)-5-({[(2,4-dichlorophenyl)sulfonyl]amino}methyl)-2,2-dimethyl-1,3-dioxolan-4-yl]methyl}carbamate

To a solution of 1,1-dimethylethyl {[(4R,5R)-5-(aminomethyl)-2,2-dimethyl-1,3-dioxolan-4-yl]methyl}carbamate (410 mg, 1.54 mmol) in dichloromethane (10 ml) were added 2,4-dichlorobenzenesulfonyl chloride (1.13 g, 4.6 mmol) and triethylamine (0.86 mL, 6.16 mmol). The mixture was stirred at room temperature overnight. The reaction mixture was washed by saturated aq. NaHCO₃ and brine. The organic layer was dried over MgSO₄, filtered, and concentrated. Purification of the residue by Biotage silica gel column chromatography (10% to 60% EA/Hexane) provided 611 mg of the title compound (85%); LCMS (m/z): 468.3 (M+H).

### f. N-{[(4R,5R)-5-(Aminomethyl)-2,2-dimethyl-1,3-dioxolan-4-yl]methyl}-2,4-dichlorobenzenesulfonamide (TFA salt)

To a solution of 1,1-dimethylethyl {[(4R,5R)-5-({[(2,4-dichlorophenyl)sulfonyl]amino}methyl)-2,2-dimethyl-1,3-dioxolan-4-yl]methyl}carbamate (303 mg, 0.646 mmol) in dichloromethane (10 mL) solution was added TFA (0.5 mL, 6.46 mmol). The reaction mixture was stirred at rt for overnight. After evaporating the solvent, the crude material was used for the next step without further purification; LCMS (m/z): 368.4 (M+H).

### g. N-{(1S)-1-[({[(4R,5R)-5-({[(2,4-Dichlorophenyl)sulfonyl]amino}methyl)-2,2-dimethyl-1,3-dioxolan-4-yl]methyl}amino)carbonyl]-3-methylbutyl}-1-benzothiophene-2-carboxamide

To a solution N-(1-benzothien-2-ylcarbonyl)-L-leucine (0.197 g, 0.68 mmol) in CH₂Cl₂ (5 mL) was added N-{[(4R,5R)-5-(aminomethyl)-2,2-dimethyl-1,3-dioxolan-4-yl]methyl}-2,4-dichlorobenzenesulfonamide (TFA salt, 311 mg, 0.646 mmol) and HOOBt (2 mg, 0.013 mmol). After cooling the mixture to 0° C., N-methylmorpholine (0.36 mL, 3.23 mmol) was added. The mixture was stirred ten minutes whereupon EDC.HCl (0.149 g, 0.78 mmol) was added. Allowed the mixture to warm up to room temperature and kept stirring overnight. The reaction mixture was washed with 10% citric acid aqueous solution, saturated aq. NaHCO₃ solution, and brine. The organic layer was dried over MgSO₄, filtered, and concentrated down. Purification of the residue by Biotage silica gel column chromatography (10% to 60% EtOAc/Hexane) provided 247 mg of the title compound (60%); LCMS (m/z): 642.6 (M+H).

### Example 110

### N-((1S)-1-{[((2R,3R)-4-{[(2,4-dichlorophenyl)sulfonyl]amino}-2,3-dihydroxybutyl)amino]carbonyl}-3-methylbutyl)-1-benzothiophene-2-carboxamide

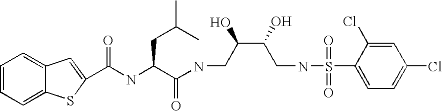

To a solution of N-{(1S)-1-[({[(4R,5R)-5-({[(2,4-dichlorophenyl)sulfonyl]amino}methyl)-2,2-dimethyl-1,3-dioxolan-4-yl]methyl}amino)carbonyl]-3-methylbutyl}-1-benzothiophene-2-carboxamide (Example 109, 113 mg, 0.176 mmol) in MeOH (3 mL) were added 4-methylbenzenesulfonic acid (25 mg, 0.131 mmol) and water (0.16 mg, 0.88 mmol). The reaction mixture was stirred at RT for overnight. After removing solvent, the crude material was purified by Biotage silica gel column chromatography (10% to 80% EA/Hexane) to provide 65 mg of the title compound (60%); LCMS (m/z): 602.4 (M+H).

### Example 111

### N-{(1S)-1-[({[(4R,5R)-5-({[(2-chloro-4-fluorophenyl)sulfonyl]amino}methyl)-2,2-dimethyl-1,3-dioxolan-4-yl]methyl}amino)carbonyl-3-methylbutyl}-1-benzothiophene-2-carboxamide

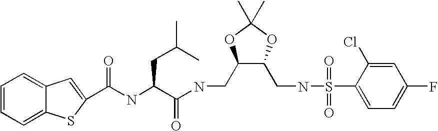

The title compound was prepared following the general procedure of Example 109 except substituting 2-chloro-4-fluorobenzenesulfonyl chloride for 2,4-dichlorobenzenesulfonyl chloride; LCMS (m/z): 626.1 (M+H).

### Example 112

### N-((1S)-1-{[((2R,3R)-4-{[(2-chloro-4-fluorophenyl)sulfonyl]amino}-2,3-dihydroxybutyl)amino]carbonyl}-3-methylbutyl)-1-benzothiophene-2-carboxamide

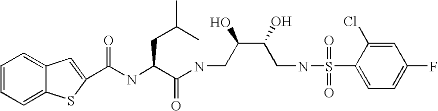

The title compound was prepared following the general procedure of Example 111 except substituting 2-chloro-4-fluorobenzenesulfonyl chloride for 2,4-dichlorobenzenesulfonyl chloride; LCMS (m/z): 586.2 (M+H).

### Example 113

### N-{[(1S)-1-[({[(4R,5R)-5-({[(2,4-dichlorophenyl)sulfonyl]amino}methyl)-1,3-dioxolan-4-yl]methyl}amino)carbonyl]-3-methylbutyl}-1-benzothiophene-2-carboxamide

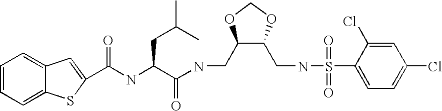

### a. (2R,3R)-2,3-Dihydroxy-1,4-butanediyl bis(4-methylbenzenesulfonate)

To a solution of [(4R,5R)-2,2-dimethyl-1,3-dioxolane-4,5-diyl]dimethanediyl bis(4-methylbenzenesulfonate) (2.0 g, 4.25 mmol) in THF (22 mL) was added 4-methylbenzenesulfonic acid (1.62 g, 8.5 mmol) and water (1 mL). The reaction mixture was stirred at 50° C. overnight. After cooling down to rt, the reaction mixture was washed by saturated aq. NaHCO₃ and brine. The organic layer was dried over MgSO₄, filtered, and concentrated. Purification of the residue by Biotage silica gel chromatography (10% to 80% EA/Hexane) provided 1.83 g of the title compound (100%); LCMS (m/z): 431.5 (M+H).

### b. (4R,5R)-1,3-Dioxolane-4,5-diyldimethanediyl bis(4-methylbenzenesulfonate)

To a solution of (2R,3R)-2,3-dihydroxy-1,4-butanediyl bis(4-methylbenzenesulfonate) (800 mg, 1.86 mmol) in isopropyl acetate (8 mL) were added boron trifluoride-diethyl ether (0.7 mL, 5.58 mmol) and bis(methyloxy)methane (0.65 mL, 7.44 mmol). The reaction mixture was refluxed overnight. After cooling down to rt, the reaction was quenched by slow addition of saturated aq. NaHCO₃. After extraction with EtOAc twice, the combined organic layer was washed by brine and dried over MgSO₄, filtered, and concentrated. Purification of the residue by Biotage silica gel column chromatography (10% to 60% EtOAc/Hexane) provided 752 mg of the title compound (91%); LCMS (m/z): 442.7 (M+H).

### c. N-{(1S)-1-[({[(4R,5R)-5-({[(2,4-dichlorophenyl)sulfonyl]amino}methyl)-1,3-dioxolan-4-yl]methyl}amino)carbonyl]-3-methylbutyl}-1-benzothiophene-2-carboxamide

The title compound was prepared following the general procedure of Example 109 (step a to step g) except substituting (4R,5R)-1,3-dioxolane-4,5-diyldimethanediyl bis(4-methylbenzenesulfonate) for [(4R,5R)-2,2-dimethyl-1,3-dioxolane-4,5-diyl]dimethanediyl bis(4-methylbenzenesulfonate): LCMS (m/z): 614.5 (M+H).

### Example 114

### N-{(1S)-1-[({[(4R,5R)-5-({[(2-chloro-4-fluorophenyl sulfonyl]amino}methyl)-1,3-dioxolan-4-yl]methyl}amino)carbonyl]-3-methylbutyl}-1-benzothiophene-2-carboxamide

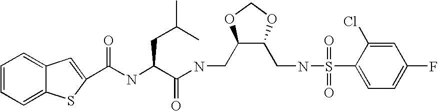

The title compound was prepared following the general procedure of Example 113 except substituting 2-chloro-4-fluorobenzenesulfonyl chloride for 2,4-dichlorobenzenesulfonyl chloride; LCMS (m/z): 598.1 (M+H).

### Example 115

### N-{(1S)-1-[({[(2S,3R)-2-({[(2-chloro-4-fluorophenyl)sulfonyl]amino}methyl)tetrahydro-3-furanyl]methyl}amino)carbonyl]-3-methylbutyl}-1-benzothiophene-2-carboxamide

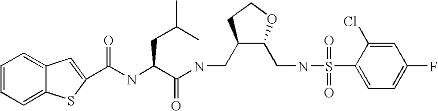

### a. ((2S,3R)-3-{[(Phenylmethyl)oxy]methyl}-2-oxiranyl)methanol

To a cold (−20° C.) stirred suspension of powdered 4A molecular sieves (4.34 g) in dry CH₂Cl₂ (280 mL) under N₂ were added titanium isopropoxide (1.60 g, 5.6 mmol), L-(−)-di-1-propyl tartrate (1.8 g, 7.7 mmol), and 10.5 ml of tert-butyl hydroperoxide (5.5 M in decane). The slurry was stirred at −20° C. for 20 min. After allylic alcohol (5.0 g, 28.0 mmol) was added, the reaction mixture was stirred at −20° C. for 30 min and then stored in −20° C. freezer overnight. After addition of 30 ml of water, the solution was stirred at room temperature for 40 min and then 30% aq. NaOH in brine (10 mL) was added and stirred for another 30 min. The aqueous layer was extracted with CH₂Cl₂, dried over MgSO₄, filtered, concentrated, and purified by flash column chromatography to provide 3.0 g of the title product (70%).

### b. (2R,3S)-4-[(Phenylmethyl)oxy]-2-(2-propen-1-yl)-1,3-butanediol

To a cold solution (−50° C.) of allylmagnesium bromide (11 mL, 1M in ethyl ether) in ether (50 mL) under N₂ was added ((2S,3R)-3-{[(phenylmethyl)oxy]methyl}-2-oxiranyl)methanol (0.53 g, 2.73 mmol) in ether (75 mL) dropwise over 20 min. The mixture was stirred for 30 min and then quenched with 1N aq. HCl (50 mL). The mixture was warmed to room temperature and the phase was separated. The aqueous layer was extracted with ether. The combined organic layer was washed with saturated aq. NaHCO₃, dried over MgSO₄, filtered, and concentrated. The crude material was purified by flash column chromatography to provide 0.30 g of the title product (46%); LCMS: [MH]⁺=237.2.

### c. (2R)-2-{(1S)-1-Hydroxy-2-[(phenylmethyl)oxy]ethyl}-4-penten-1-yl benzoate

To a solution of (2R,3S)-4-[(phenylmethyl)oxy]-2-(2-propen-1-yl)-1,3-butanediol (0.79 g, 3.35 mmol) in pyridine (20 mL) at 0° C. was added benzoyl chloride (0.40 mL, 3.42 mmol). The reaction mixture was stirred at this temperature for 20 min. 0.60 mL of water was added to quench the reaction. After the solvent was removed, the resultant solid was dissolved with CH₂Cl₂, washed with 1N HCl, saturated aq. NaHCO₃, and brine followed by drying over MgSO₄, filtration, and concentration. The resultant residue was purified by flash column chromatography to provide 0.75 g of the desired product (66%); LCMS: [MH]⁺=341.2.

### d. Methyl 2,3-dideoxy-3-{[(phenylcarbonyl)oxy]methyl}-5-O-(phenylmethyl)-D-erythro pentofuranoside

To an ice cold solution of (2R)-2-{(1S)-1-hydroxy-2-[(phenylmethyl)oxy]ethyl}-4-penten-1-yl benzoate (0.70 g, 1.93 mmol) and NMMO (0.52 g, 3.86 mmol) in THF/H₂O (9 mL/3 mL) was added 1.0 mL of OSO4 solution (0.08 M in water). After a few min, the ice bath was removed and the reaction mixture was stirred overnight at room temperature under N₂. NaHSO₃ (0.20 g, 2.0 mmol) was then added. The solution was stirred at room temperature for 30 min. The mixture was partitioned between 1N HCl and EtOAc. The organic layer was washed with saturated aq. NaHCO₃, dried over MgSO₄, filtered, and concentrated. The crude compound was dissolved in THF/H₂O (18 mL/6 mL) and treated with NalO₄ (0.80 g) at room temperature for 1 hour. The reaction mixture was partitioned between brine and ether. The organic solution was dried over MgSO₄, filtered, and concentrated. The residue was treated with 0.54 mL of HCl in MeOH (0.57%, w/w) for 20 min, neutralized using Dowex 2×8, filtered, and concentrated. The resultant residue was purified by flash column chromatography to provide 0.48 g of the desired product (67%); LCMS: [MH]⁺=371.0.

### e. 1,4-Anhydro-2,3-dideoxy-3-{[(phenylcarbonyl)oxy]methyl}-5-O-(phenylmethyl)-D-erythropentitol

To a solution of methyl 2,3-dideoxy-3-{[(phenylcarbonyl)oxy]methyl}-5-O-(phenylmethyl)-D-erythro-pentofuranoside (0.48 g, 1.36 mmol) in CH₃CN/CH₂Cl₂ (14 mL/7 mL) at 0° C. was added Et₃SiH (0.21 mL, 1.36 mmol) followed by the addition of BF₃.OEt (0.21 mL, 1.36 mmol). The reaction mixture was stirred at 0° C. for 1 hour. K₂CO₃ (0.33 g, 2.40 mmol) was added. The reaction mixture was filtered, concentrated, and purified by flash column chromatography to provide 0.40 g of the desired product (86%); LCMS: [MH]⁺=341.2.

### f. 1,4-Anhydro-2,3-dideoxy-3-(hydroxymethyl)-5-(phenylmethyl)-D-erythropentitol

To a solution of solution of 1,4-anhydro-2,3-dideoxy-3-{[(phenylcarbonyl)oxy]methyl}-5-0(phenylmethyl)-D-erythropentitol (0.30 g, 0.92 mmol) in 35 mL of MeOH was added NaOH (0.25 g, 6.2 mmol). The reaction mixture was stirred at room temperature for 1 h. The reaction mixture was diluted with EtOAc, washed with water and brine followed by drying over MgSO₄, filtration, and concentration. 0.18 g of the crude product was obtained (83%) and was carried to the next reaction without further purification; LCMS: [MH]⁺=223.0.

### g. 1,4-Anhydro-3-(azidomethyl)-2,3-dideoxy-5-O-(phenylmethyl)-D-erythropentitol

To a solution of 1,4-anhydro-2,3-dideoxy-3-(hydroxymethyl)-5-O-(phenylmethyl)-D-erythropentitol (0.17 g, 0.77 mmol) in 5 mL of CH₂Cl₂ at 0° C. were added Et₃N (0.22 mL, 1.54 mmol) and MsCl (0.096 g, 0.85 mmol). The reaction mixture was stirred at 0° C. for 2 hours. The solution was washed with water and brine, dried over MgSO₄, filtered, and concentrated. The residue was then dissolved in 10 mL of DMF. After NaN₃ (0.15 g, 2.31 mmol) was added, the solution was stirred at 70° C. for 2 hours. The reaction mixture was diluted with EtOAc, washed with water and brine, and dried over MgSO₄. After filtration and concentration, the resultant residue was purified by flash column chromatography to provide 0.15 g of the desired product (74%).

### h. 3-(Aminomethyl)-1,4-anhydro-2,3-dideoxy-5-0(phenylmethyl)-D-erythropentitol

To a solution of 1,4-anhydro-3-(azidomethyl)-2,3-dideoxy-5-O-(phenylmethyl)-D-erythropentitol (0.15 g, 0.61 mmol) in THF (5 mL) was added 0.025 mL of H₂O and PPh₃ (0.17 g, 0.65 mmol). The reaction mixture was stirred at room temperature overnight. The solution was concentrated under reduced pressure. The crude material was carried to the next reaction without further purification.

### i. N-[(1S)-3-Methyl-1-({[((2S,3R)-2-{[(phenylmethyl)oxy]methyl}tetrahydro-3-furanyl)methyl]amino}carbonyl)butyl]-1-benzothiophene-2-carboxamide

To a solution of N-(1-benzothien-2-ylcarbonyl)-L-leucine (crude, 170 mg, 0.58 mmol) and 3-(aminomethyl)-1,4-anhydro-2,3-dideoxy-5-O-(phenylmethyl)-D-erythro pentitol (110 g, 0.46 mmol) in CH₂Cl₂ (5 mL) were added HOOBt (10 mg, 0.060 mmol), EDC.HCl (110 mg, 0.58 mmol) and NMM (0.20 mL, 1.65 mmol) at rt. After stirring for 5 hr at rt, the reaction mixture was quenched with cold 1N HCl. After extraction with CH₂Cl₂ (20 mL×2), the organic solution was washed with sat'd aq. NaHCO₃ and brine, followed by drying over MgSO₄, filtration, and concentration. The resultant residue was purified by flash column chromatography to provide 150 mg of the desired product (66%); LCMS: [MH]⁺=495.2.

### j. N-{(1S)-1-[({[(2S,3R)-2-(Hydroxymethyl)tetrahydro-3-furanyl]methyl}amino)carbonyl]-3-methylbutyl}-1-benzothiophene-2-carboxamide

To a solution of N-[(1S)-3-methyl-1-({[((2S,3R)-2-{[(phenylmethyl)oxy]methyl}tetrahydro-3-furanyl)methyl]amino}carbonyl)butyl]-1-benzothiophene-2-carboxamide (0.15 g, 0.31 mmol) in 5 mL of EtOH was added 30 mg (0.028 mmol) of 10% Pd on carbon followed by the addition of a few drops of formic acid. The mixture was stirred under H₂ (balloon) for 5 hours. The suspension was filtered and concentrated under reduced pressure. The crude material was carried to the next reaction without further purification.

### k. N-{(1S)-1-[({[(2S,3R)-2-(aminomethyl)tetrahydro-3-furanyl]methyl}amino)carbonyl]-3-methylbutyl}-1-benzothiophene-2-carboxamide

The title compound was prepared following the procedure of Example 18g-18h except for the use of N-[(1S)-3-methyl-1-({[((2S,3R)-2-{[(phenylmethyl)oxy]methyl}tetrahydro-3-uranyl)methyl]amino}carbonyl)butyl]-1-benzothiophene-2-carboxamide in place of 1,4-anhydro-2,3-dideoxy-3-(hydroxymethyl)-5-O-(phenylmethyl)-D-etythropentitol.

### l. N-{(1S)-1-[({[(2S,3R)-2-({[(2-chloro-4-fluorophenyl)sulfonyl]amino}methyl)tetrahydro-3-furanyl]methyl}amino)carbonyl]-3-methylbutyl}-1-benzothiophene-2-carboxamide

To a solution of N-{(1S)-1-[({[(2S,3R)-2-(aminomethyl)tetrahydro-3-furanyl]methyl}amino)carbonyl]-3-methylbutyl}-1-benzothiophene-2-carboxamide (30 mg, 0.074 mmol) in CH₂Cl₂ (2 mL) were added 2-chloro-4-fluorobenzenesulfonyl chloride (30 mg, 0.13 mmol) and Et₃N (0.1 mL, 0.60 mmol) at rt. After stirring for 30 min at rt, the reaction mixture was purified by flash column chromatography without aqueous work-up to provide 30 mg of a desired product (71%); LCMS: [MH]⁺=596.2.

### Example 116

### N-{(1S)-1-[({[(2S,3R)-3-({[(2-chloro-4-fluorophenyl)sulfonyl]amino}methyl)tetrahydro-2-furanyl]methyl}amino)carbonyl]-3-methylbutyl}-1-benzothiophene-2-carboxamide

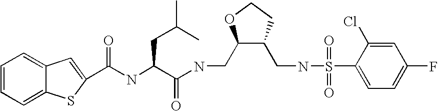

### a. 1,4-Anhydro-2,3-dideoxy-3-[({[(1,1-dimethylethyl)oxy]carbonyl}amino)methyl]-5-O-(phenylmethyl)-D-etythro-pentitol

To a solution of 3-(aminomethyl)-1,4-anhydro-2,3-dideoxy-5-O-(phenylmethyl)-D-etythropentitol (170 mg, 0.77 mmol) in 3 mL of CH₃CN was added (Boc)₂O (250 mg, 1.15 mmol). The reaction mixture stirred at room temperature overnight. After the solution was concentrated under vacuum, the crude material was dissolved in EtOH (5 mL) followed by the addition of Pd on carbon (10%, 20 mg, 0.019 mmol). After stirring for 4 hr under hydrogen (balloon) at rt, the suspension was filtered. After the filtrate was concentrated and dried under reduced pressure, the resultant residue was used for the next reaction without further purification.

### b. 1,4-Anhydro-5-{[N-(1-benzothien-2-ylcarbonyl)-L-leucyl]amino}-2,3,5-trideoxy-3-[({[(1,1-dimethylethyl)oxy]carbonyl}amino)methyl]-D-erythro-pentitol

The title compound was prepared following the procedure of Example 18g-18i except for the use of 1,4-anhydro-5-azido-2,3,5-trideoxy-3-[({[(1,1-dimethylethyl)oxy]carbonyl}amino)methyl]-D-erythropentitol in place of 1,4-anhydro-2,3-dideoxy-3-(hydroxymethyl)-5-O-(phenylmethyl)-D-erythropentitol.

### c. N-{(1S)-1-[({[(2S,3R)-3-(Aminomethyl)tetrahydro-2-furanyl]methyl}amino)carbonyl]-3-methylbutyl}-1-benzothiophene-2-carboxamide

To a solution of 1,1-dimethylethyl ((4R)-4-{[(2-chloro-4-fluorophenyl)sulfonyl]amino}-5-hydroxypentyl)carbamate (75 mg, 0.15 mmol) in CH₂Cl₂ (0.5 mL) was added 4N. HCl in dioxane (0.5 mL) at rt. After stirring for 30 min at rt, the reaction mixture was concentrated and dried under reduced pressure. The resultant amorphous solid was carried to the next reaction without further purification.

### d. N-{(1S)-1-[({[(2S,3R)-3-({[(2-Chloro-4-fluorophenyl)sulfonyl]amino}methyl)tetrahydro-2-furanyl]methyl}amino)carbonyl]-3-methylbutyl}-1-benzothiophene-2-carboxamide

To a solution of N-{(1S)-1-[({[(2S,3R)-3-(aminomethyl)tetrahydro-2-furanyl]methyl}amino)carbonyl]-3-methylbutyl}-1-benzothiophene-2-carboxamide (62 mg, 0.14 mmol) in 2 mL of CH₂Cl₂ were added 2-chloro-4-floruobenzenesulfonyl chloride (35 mg, 0.15 mmol) and Et₃N (0.10 mL, 0.53 mmol) at rt. After stirring for 30 min at rt, the reaction mixture was purified by flash column chromatography without aqueous work-up to provide 33 mg of a desired product (75%); LCMS: [MH]⁺=596.2.

### Example 117

### N-{(1S)-1-[({[2-({[(2,4-dichlorophenyl)sulfonyl]amino}methyl)cyclopentyl]methyl}amino)carbonyl]-3-methylbutyl}-1-benzothiophene-2-carboxamide

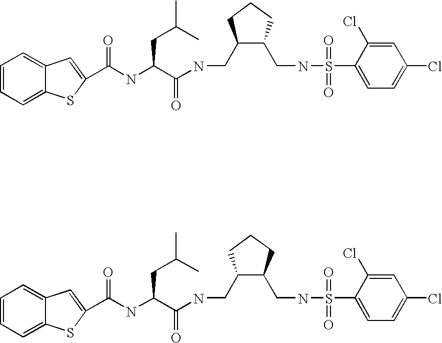

### a. (1R,2R)-1,2-Cyclopentanediyldimethanol

To a solution of trans-(+/−)-1,2-cyclopentanedicarboxylic acid (1 g, 6.32 mmol) in THF (10 mL) at −10° C. was slowly added BH₃ (63 mL, 1M in THF). The mixture was allowed to warm to room temperature and kept stirring for 5 hours. After concentration, the crude material was used for the next step without further purification.

### b. (1R,2R)-1,2-Cyclopentanediyidimethanediyi dimethanesulfonate

To a solution of (1R,2R)-1,2-cyclopentanediyidimethanol (from step a) in dichloromethane (30 mL) at −20° C. were added Et₃N (1.94 mL, 13.9 mmol) and DMAP (7.72 mg, 0.63 mmol). After 10 minutes, MsCl (1.08 mL, 13.9 mmol) was added. The reaction mixture was allowed to warm to room temperature and stirred overnight. The mixture was then washed by sat'd aq. NH₄Cl, saturated aq. NaHCO₃, and brine. The organic layer was dried over MgSO₄. After filtration and concentration, the crude material was used for the next step without further purification; LCMS (m/z): 287.7 (M+H).

### c. [(1R,2R)-2-(Azidomethyl)cyclopentyl]methyl methanesulfonate

To a solution of (1R,2R)-1,2-cyclopentanediyldimethanediyl dimethanesulfonate in DMF (40 mL) was added sodium azide (274 mg, 4.21 mmol). The reaction mixture was stirred at 65° C. overnight. After removing most of the solvent under reduced pressure, the residue was diluted in dichloromethane. The reaction mixture was washed by water, saturated aq. NaHCO₃, and brine. The organic layer was dried over MgSO₄, filtered, and concentrated. Purification of the residue by Biotage silica gel column chromatography (20% to 60% EA/Hexane) provided 244 mg of the title compound (25% for 3 steps); LCMS (m/z): 233.2 (M+H).

### d. 2-{[(1S,2S)-2-(Azidomethyl)cyclopentyl]methyl}-1H-isoindole-1,3(2H)-dione

To a solution of [(1R,2R)-2-(azidomethyl)cyclopentyl]methyl methanesulfonate (244 mg, 1.57 mmol) in DMF (10 ml) was added potassium phthalimide (437 mg, 2.36 mmol). The reaction mixture was stirred at 100° C. overnight. After removing most of the solvent under reduced pressure, the residue was diluted in dichloromethane. The reaction mixture was washed by water, saturated aq. NaHCO₃, and brine. The organic layer was dried over MgSO₄, filtered, and concentrated. Purification of the residue by Biotage silica gel column chromatography (10% to 50% EtOAc/Hexane) provided 241 mg of the title compound (81%); LCMS (m/z): 285.3 (M+H).

### e. N-{(1S)-1-[({[2-({[(2,4-Dichlorophenyl)sulfonyl]amino}methyl)cyclopentyl]methyl}amino)carbonyl]-3-methylbutyl}-1-benzothiophene-2-carboxamide

The title compound was prepared following the general procedure of Example 109c-12g except substituting 2-{[(4R,5R)-5-(azidomethyl)-2,2-dimethyl-1,3-dioxolan-4-yl]methyl}-1H-isoindole-1,3(2H)-dione for 2-{[(4R,5R)-5-(azidomethyl)-2,2-dimethyl-1,3-dioxolan-4-yl]methyl}-1H-isoindole-1,3(2h)-dione; LCMS (m/z): 610.6 (M+H).

### Example 118

### N-{(1S)-1-[({[2-({[(2-chloro-4-fluorophenyl)sulfonyl]amino}methyl)cyclopentyl]methyl}amino)carbonyl]-3-methylbutyl}-1-benzothiophene-2-carboxamide

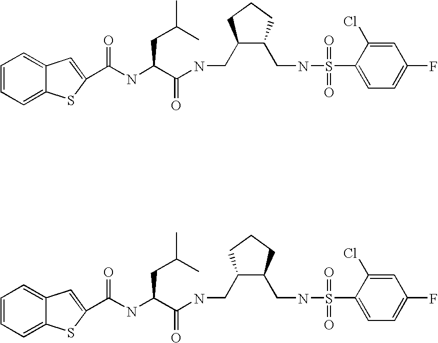

The title compound was prepared following the general procedure of Example 118 except substituting 2-chloro-4-fluorobenzenesulfonyl chloride for 2,4-dichlorobenzenesulfonyl chloride; LCMS (m/z): 594.2 (M+H).

### Example 119

### 2,4-dichloro-N-{4-[[(cyclohexylamino)carbonyl]((2S)-4-methyl-2-{[(phenylamino)-carbonyl]-amino}pentyl)amino]butyl}benzenesulfonamide

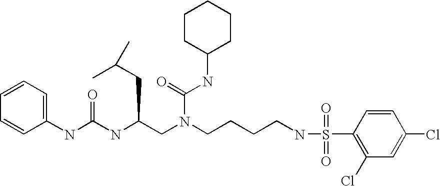

### a. 1,1-Dimethylethyl[(1S)-1-formyl-3-methylbutyl]carbamate

To a solution of BOC-(L)-Leu-OMe (3.0 g, 12.22 mmol) in toluene (20 mL) at −78° C. was slowly added 30.57 mL (30.57 mmol) of DIBAH solution (1M in toluene) over 30 minutes. After the mixture was stirred for additional 30 min at −78° C., the reaction was slowly quenched with MeOH (2.48 mL), stirred for 5 min, and then allowed to reach ambient temperature. A solution of Rochelle's salt (18 g) in water (25 mL) was added at RT and stirred for 5 min. The resulting mixture was washed with brine (20 mL×2), dried over MgSO₄, filtered, concentrated, and azeotroped using toluene under reduced pressure to yield the title compound as an oil that was used with no further purification (2.68 g, quantitative).

### b. 1,1-Dimethylethyl ((1S)-1-{[(4-{[(2,4-dichlorophenyl)sulfonyl]amino}butyl)amino]methyl}-3-methylbutyl)carbamate

To a solution of 1,1-dimethylethyl[(1S)-1-formyl-3-methylbutyl]carbamate (1.02 g, 4.76 mmol) in MeOH (20 mL) at RT were added N-(4-aminobutyl)-2,4-dichlorobenzenesulfonamide (1.69 g, 5.71 mmol), NaCNBH₃ (0.329 g, 5.23 mmol), and acetic acid (0.653 mL, 11.42 mmol). After stirring the reaction mixture for 2 h at rt, it was quenched using cold 1 N NaOH (˜11 mL), then extracted with ethyl acetate (60 mL×2), dried over MgSO₄, filtered, and concentrated in vacuo, then purified by flash column chromatography (MeOH/CH₂Cl₂, 0% to 3.5%) to yield the title compound (0.92 g, 39%); LCMS (m/z): 496 (M+H).

### c. 9H-Fluoren-9-ylmethyl (4-{[(2,4-dichlorophenyl)sulfonyl]amino}butyl)[(2S)-2-({[(1,1-dimethylethyl)oxy]carbonyl}amino)-4-methylpentyl]carbamate

To a cooled solution of 1,1-dimethylethyl ((1S)-1-{[(4-{[(2,4-dichlorophenyl)sulfonyl]-amino}-butyl)amino]methyl}-3-methylbutyl)carbamate (0.709 g, 1.43 mmol) in dichloromethane (15 mL) at 0° C. were added triethylamine (0.260 mL, 1.86 mmol) and FMOC-Cl (0.445 g, 1.72 mmol). The reaction mixture was stirred for 30 min followed by concentration in vacuo. The residue was purified by flash column chromatography (ethyl acetate/hexane, 0% to 50%) to yield the title compound as white foam (0.940 g, 92%); LCMS (m/z): 718 (M+H).

### d. 9H-Fluoren-9-ylmethyl (4-{[(2,4-dichlorophenyl)sulfonyl]amino}butyl)((2S)-4-methyl-2-{[(phenylamino)carbonyl]amino}pentyl)carbamate

To a solution of 9H-fluoren-9-ylmethyl (4-{[(2,4-dichlorophenyl)sulfonyl]amino}-butyl)[(2S)-2-({[(1,1-dimethylethyl)oxy]carbonyl}amino)-4-methylpentyl]carbamate (0.709 g, 0.989 mmol) in dichloromethane (8 mL) was added TFA (4 mL). After stirring for 30 min, the solvent was evaporated and the residue azeotroped with toluene. To the resulting solid were added dichloromethane (10 mL), triethylamine (0.551 mL, 3.96 mmol), and phenylisocyanate (0.129 mL, 1.19 mmol). After stirring for 30 min at rt, the solvent was evaporated and the residue was purified by flash column chromatography (ethyl acetate/hexane, 10% to 60%) to yield the title compound (0.548 g, 75%); LCMS (m/z): 737 (M+H).

### e. 2,4-Dichloro-N-{4-[((2S)-4-methyl-2-{[(phenylamino)carbonyl]amino}pentyl)-amino]butyl}benzenesulfonamide

To a solution of 9H-fluoren-9-ylmethyl (4-{[(2,4-dichlorophenyl)sulfonyl]amino}butyl)((2S)-4-methyl-2-{[(phenylamino)carbonyl]amino}pentyl)carbamate (0.548 g, 0.746 mmol) in DMF (6.0 mL) was added piperidine (0.20 mL). The mixture was stirred for 1.0 h, and then concentrated in vacuo. The residue was purified by flash column chromatography (MeOH/CH₂Cl₂, 0% to 8.0%) to yield the title compound as a white solid (0.325 g, 85%); LCMS (m/z): 515 (M+H).

### f. 2,4-Dichloro-N-{4-[[(cyclohexylamino)carbonyl]((2S)-4-methyl-2-{[(phenylamino)carbonyl]amino}pentyl)amino]butyl}benzenesulfonamide

To solution of 2,4-dichloro-N-{4-[((2S)-4-methyl-2-{[(phenylamino)carbonyl]-amino}pentyl)-amino]butyl}benzenesulfonamide (30 mg, 0.0584 mmol) in dichloromethane (2.0 mL) was added cyclohexylisocyanate (11 mg, 0.088 mmol) at rt. After 1 h, the mixture was concentrated in vacuo, and then purified by flash chromatography (ethyl acetate/hexane, 10% to 75%) to yield the title compound (0.034 g, 91%); LCMS: [MH]⁺=640.

### Example 120

### 2,4-Dichloro-N-{4-[[(4-fluorophenyl)sulfonyl]((2S)-4-methyl-2-{[(phenylamino)-carbonyl]amino}pentyl)amino]butyl}benzenesulfonamide

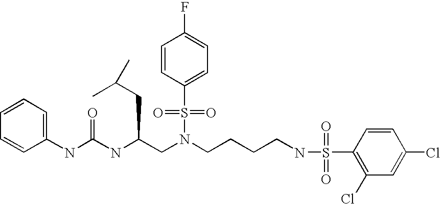

The title compound was prepared following the procedure of Example 119 except for the substitution of 4-fluorobenzenesulfonyl chloride for cyclohexylisocyanate in the last step; LCMS: [MH]⁺=673.

### Example 121

### N-(4-{[(2,4-Dichlorophenyl)sulfonyl]amino}butyl)-N-((2S)-4-methyl-2-{[(phenylamino)carbonyl]amino}pentyl)acetamide

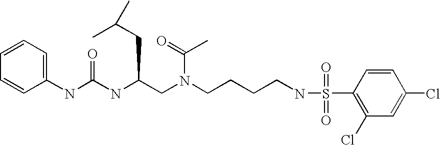

The title compound was prepared following the procedure of Example 119 except for the substitution of acetic anhydride for cyclohexylisocyanate in the last step; LCMS: [MH]⁺=557.

### Example 122

### Methyl (4-{[(2,4-dichlorophenyl)sulfonyl]amino}butyl)((2S)-4-methyl-2-{[(phenylamino)carbonyl]amino}pentyl)carbamate

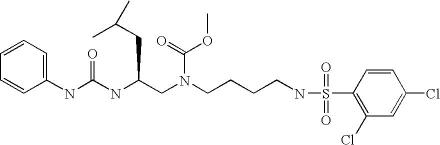

The title compound was prepared following the procedure of Example 119 except for the substitution of methyl chloroformate for cyclohexylisocyanate in the last step; LCMS: [MH]⁺=573.

### Example 123

### 2,4-dichloro-N-{4-[[(ethylamino)carbonyl]((2S)-4-methyl-2-{[(phenylamino)carbonyl]-amino}pentyl)amino]butyl}benzenesulfonamide

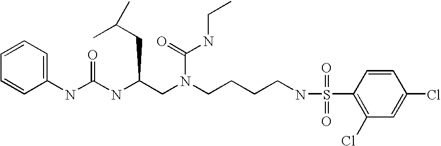

The title compound was prepared following the procedure of Example 119 except for the substitution of ethylisocyanate for cyclohexylisocyanate in the last step; LCMS: [MH]⁺=586.

### Example 124

### 2,4-dichloro-N-{4-[{[(1-methylethyl)amino]carbonyl}((2S)-4-methyl-2-{[(phenylamino)-carbonyl]amino}pentyl)amino]butyl}benzenesulfonamide

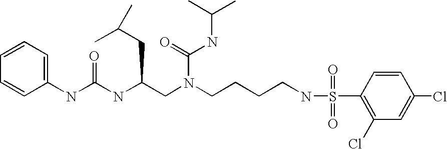

The title compound was prepared following the procedure of Example 119 except for the substitution of isopropylisocyanate for cyclohexylisocyanate in the last step; LCMS: [MH]⁺=600.

### Example 125

### 2,4-dichloro-N-(4-{((2S)-4-methyl-2-{[(phenylamino)carbonyl]amino}pentyl)-[(phenylamino)-carbonyl]amino}butyl)benzenesulfonamide

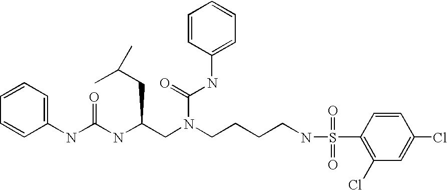

The title compound was prepared following the procedure of Example 119 except for the substitution of phenylisocyanate for cyclohexylisocyanate in the last step; LCMS: [MH]⁺=634.

### Example 126

### N-(4-{[(2,4-dichlorophenyl)sulfonyl]amino}butyl)-N-((2S)-4-methyl-2-{[phenylamino)-carbonyl]amino}pentyl)cyclohexanecarboxamide

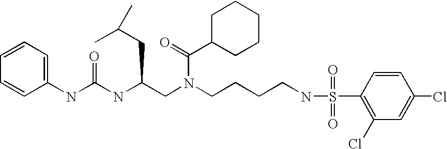

The title compound was prepared following the procedure of Example 119 except for the substitution of cyclohexanecarbonyl chloride for cyclohexylisocyanate in the last step; LCMS: [MH]⁺=625.

### Example 127

### 2,4-dichloro-N-(4-{{[(1-methylethyl)amino]carbonyl}[(2S)-4-methyl-2-({[(1-methylethyl)amino]-carbonyl}amino)pentyl]amino}butyl)benzenesulfonamide

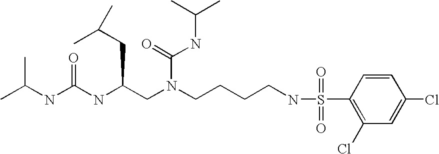

The title compound was prepared following the procedure of Example 119 except for the substitution of isopropylisocyanate for Fmoc-Cl in step c and substitution of isopropylisocyanate for phenylisocyanate; LCMS: [MH]⁺=566.

### Example 128

### 2,4-dichloro-N-[4-(((2S)-2-{[(cyclohexylamino)carbonyl]amino}-4-methylpentyl){[(1-methylethyl)amino]carbonyl}amino)butyl]benzenesulfonamide

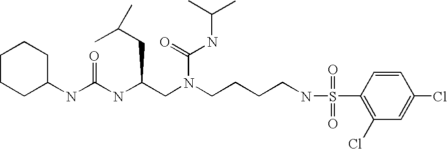

The title compound was prepared following the procedure of Example 127 except for the substitution of cyclohexylisocyanate for isopropylisocyanate; LCMS: [MH]⁺=606.

### Example 129

### 2,4-dichloro-N-[4-(((2S)-2-{[(ethylamino)carbonyl]amino}-4-methylpentyl){[(1-methylethyl)amino]carbonyl}amino)butyl]benzenesulfonamide

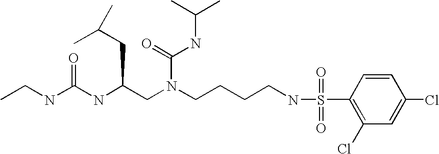

The title compound was prepared following the procedure of Example 129 except for the substitution of ethylisocyanate for isopropylisocyanate; LCMS: [MH]⁺=552.

### Example 130

### 2,4-dichloro-N-[4-([(2S)-2-({[(4-fluorophenyl)amino]carbonyl}amino)-4-methylpentyl]{[1-methylethyl)amino]carbonyl}amino)butyl]benzenesulfonamide

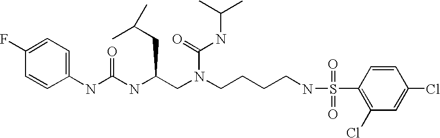

The title compound was prepared following the procedure of Example 127 except for the substitution of p-fluorophenylisocyanate for isopropylisocyanate; LCMS: [MH]⁺=618.

### Example 131

### 2,4-dichloro-N-[4-({[(1-methylethyl)amino]carbonyl}{(2S)-4-methyl-2-[({[4-(1H-pyrrol-1-1-yl)phenyl]amino}carbonyl)amino]pentyl}amino)butyl]benzenesulfonamide

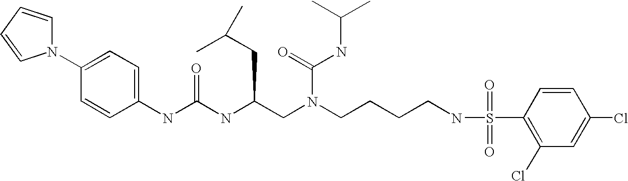

The title compound was prepared following the procedure of Example 128 except for the substitution of 1-(4-isocyanatophenyl)-1H-pyrrole for isopropylisocyanate; LCMS: [MH]⁺=665.

### Example 132

### 2,4-dichloro-N-(4-{{[(1-methylethyl)amino]carbonyl}[(2S)-4-methyl-2-({[(1,3,5-trimethyl-1H-pyrazol-4-yl)amino]carbonyl}amino)pentyl]amino}butyl)benzenesulfonamide

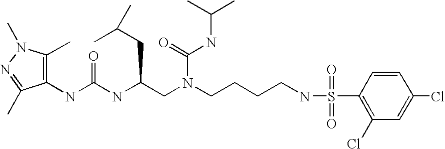

The title compound was prepared following the procedure of Example 127 except for the substitution of 4-isocyanato-1,3,5-trimethyl-1H-pyrazole for isopropylisocyanate; LCMS: [MH]⁺=632.

### Example 133

### 2,4-dichloro-N-{4-[[(cyclopropylamino)carbonothioyl]((2S)-4-methyl-2-{[(phenylamino)carbonyl]-amino}pentyl)amino]butyl}benzenesulfonamide

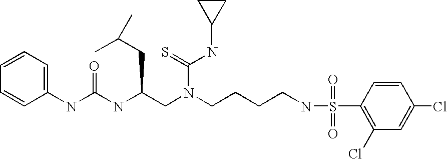

The title compound was prepared following the procedure of Example 119 except for the substitution of cyclopropylisothiocyanate for cyclohexylisocyanate in the last step; LCMS: [MH]⁺=614.

### Example 134

### 2,4-dichloro-N-{4-[{[(1,1-dimethylethyl)amino]carbonyl}((2S)-4-methyl-2-{[(phenylamino) carbonyl]amino}pentyl)amino]butyl}benzenesulfonamide

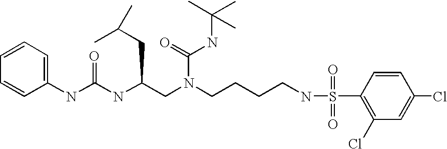

The title compound was prepared following the procedure of Example 119 except for the substitution of 2-isocyanato-2-methylpropane for cyclohexylisocyanate in the last step; LCMS: [MH]⁺=614.

### Example 135

### 2,4-dichloro-N-{4-[{[(1,1-dimethylethyl)amino]carbonyl}((2S)-4-methyl-2-{[(phenylamino carbonyl]amino}pentyl)amino]butyl}benzenesulfonamide

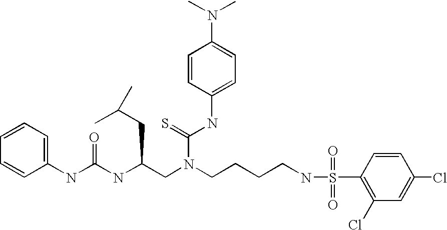

The title compound was prepared following the procedure of Example 119 except for the substitution of (4-isocyanatophenyl)dimethylamine for cyclohexylisocyanate in the last step; LCMS: [MH]⁺=677.

### Example 136

### 2,4-dichloro-N-{4-[{[(2-furanylmethyl)amino]carbonyl}((2S)-4-methyl-2-{[(phenylamino) carbonyl]amino}pentyl)amino]butyl}benzenesulfonamide

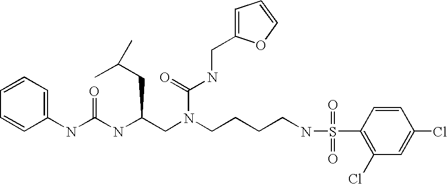

The title compound was prepared following the procedure of Example 120 except for the substitution of 2-(isocyanatomethyl)furan for cyclohexylisocyanate in the last step; LCMS: [MH]⁺=638

### Example 137

### 2,4-dichloro-N-(4-{((2S)-4-methyl-2-{[(phenylamino)carbonyl]amino}pentyl)[(3-thienylamino)carbonyl]amino}butyl)benzenesulfonamide

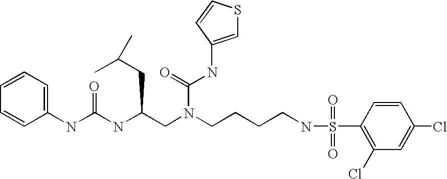

The title compound was prepared following the procedure of Example 119 except for the substitution of 3-thienyl isocyanate for cyclohexylisocyanate in the last step; LCMS: [MH]⁺=640.

### Example 138

### 2,4-dichloro-N-{4-[((2S)-4-methyl-2-{[(phenylamino)carbonyl]amino}pentyl)({[1-(trifluoroacetyl)-4-piperidinyl]amino}carbonyl)amino]butyl}benzenesulfonamide

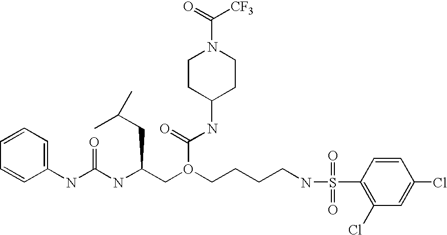

The title compound was prepared following the procedure of Example 119 except for the substitution of 4-isocyanato-1-(trifluoroacetyl)piperidine for cyclohexylisocyanate in the last step; LCMS: [MH]⁺=737.

### Example 139

### 2,4-dichloro-N-{4-[{[(1,1-dioxidotetrahydro-3-thienyl)amino]carbonyl}((2S)-4-methyl-2-{[(phenylamino)carbonyl]amino}pentyl)amino}butyl]benzenesulfonamide

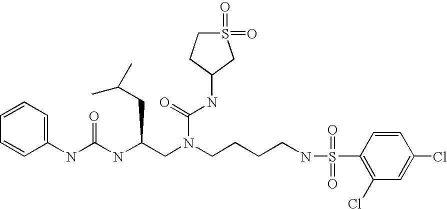

The title compound was prepared following the procedure of Example 119 except for the substitution of sulfolane-3-isocyanate for cyclohexylisocyanate in the last step; LCMS: [MH]⁺=676.

### Example 140

### 2,4-dichloro-N-{4-[[(methylamino)carbonothioyl]((2S)-4-methyl-2-{[(phenylamino) carbonyl]amino}pentyl)amino]butyl}benzenesulfonamide

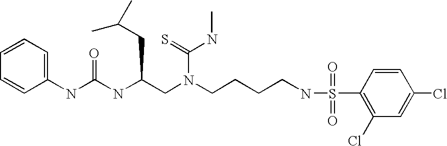

The title compound was prepared following the procedure of Example 119 except for the substitution of methylisothiocyanate for cyclohexylisocyanate in the last step; LCMS: [MH]⁺=588.

### Example 141

### 2,4-dichloro-N-{4-[{[(1-methylethyl)amino]carbonyl}((2S)-4-methyl-2-{[(2-pyridinylamino) carbonothioyl]amino}pentyl)amino]butyl}benzenesulfonamide

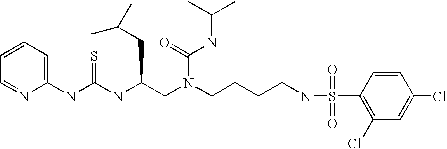

The title compound was prepared following the procedure of Example 127 except for the substitution of 3-pyridyl-isothiocyanate for isopropylisocyanate; LCMS: [MH]⁺=617.

### Example 142

### 2,4-dichloro-N-{4-[{[(1-methylethyl)amino]carbonyl}((2S)-4-methyl-2-{[(phenylamino) carbonothioyl]amino}pentyl)amino]butyl}benzenesulfonamide

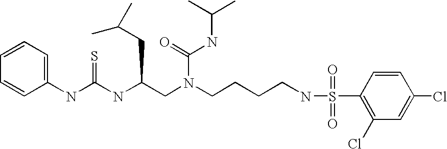

The title compound was prepared following the procedure of Example 127 except for the substitution of phenylisothiocyanate for isopropylisocyanate; LCMS: [MH]⁺=616.

### Example 143

### 2,4-dichloro-N-{4-[{[(1-methylethyl)amino]carbonyl}((2S)-4-methyl-2-{[(3-thienylamino) carbonyl]amino}pentyl)amino]butyl}benzenesulfonamide

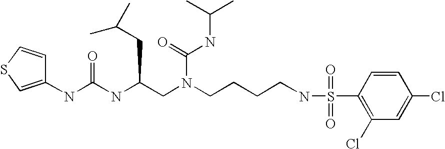

The title compound was prepared following the procedure of Example 127 except for the substitution of 3-thienyl-isocyanate for isopropylisocyanate; LCMS: [MH]⁺=606.

### Example 144

### 2,4-dichloro-N-[4-({[(1-methylethyl)amino]carbonyl}{(2S)-4-methyl-2-[({[1-(trifluoroacetyl)-4-piperidinyl]amino}carbonyl]amino]pentyl}amino)butyl}benzenesulfonamide

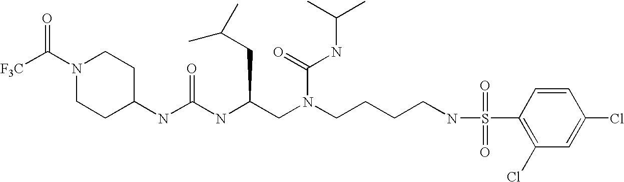

The title compound was prepared following the procedure of Example 119 except for the substitution of 4-isocyanato-1-(trifluoroacetyl)piperidine for isopropylisocyanate; LCMS: [MH]⁺=703.

### Example 145

### 2,4-dichloro-N-{4-[{[(1-methylethyl)amino]carbonyl}((2S)-4-methyl-2-{[(2-pyridinyl amino)carbonyl]amino}pentyl)amino]butyl}benzenesulfonamide

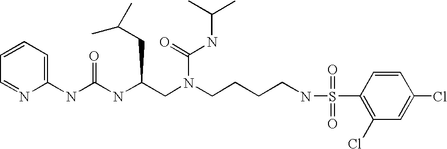

The title compound was prepared following the procedure of Example 127 except for the substitution of pyridine-3-isocyanate for isopropylisocyanate; LCMS: [MH]⁺=601.

### Example 146

### 2,4-dichloro-N-[4-(((2S)-2-{[(cyclopentylamino)carbonyl]amino}-4-methylpentyl){[(1-methylethyl)amino]carbonyl}amino)butyl]benzenesulfonamide

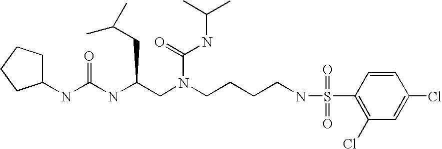

The title compound was prepared following the procedure of Example 127 except for the substitution of isocyanatocyclopentane for isopropylisocyanate; LCMS: [MH]⁺=592.

### Example 147

### N-(4-{[(2,4-dichlorophenyl)sulfonyl]amino}butyl)-N-((2S)-4-methyl-2-{[(phenylamino)carbonyl]amino}pentyl)-2-phenylacetamide

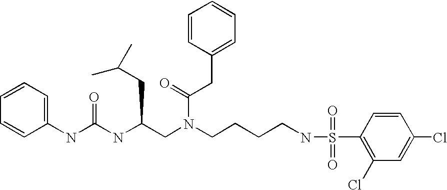

The title compound was prepared following the procedure of Example 119 except for the substitution of phenacetyl chloride for cyclohexylisocyanate in the last step; LCMS: [MH]⁺=633.

### Example 148

### 2,4-Dichloro-N-[4-([(2R)-2-hydroxy-3-(methyloxy)propyl]{[(1-methylethyl)amino]carbonyl}amino)butyl]benzenesulfonamide

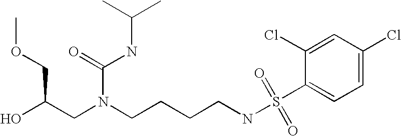

### a. 2,4-Dichloro-N-(4-{[(2R)-2-hydroxy-3-(methyloxy)propyl]amino}butyl)benzenesulfonamide

To a solution of (R)-(−)-glycidyl methyl ether (0.1 mL, 1.135 mmol) in i-PrOH (6 mL) was added N-(4-aminobutyl)-2,4-dichlorobenzenesulfonamide (500 mg, 1.7 mmol). The reaction mixture was heated at reflux for 2.5 hr. After evaporation under reduced pressure, the resultant residue was purified by column chromatography (Biotage) on silica gel (0% to 8.5% MeOH/CH₂Cl₂) to provide 200 mg (52%) of the title compound.

### b. 2,4-Dichloro-N-[4-([(2R)-2-hydroxy-3-(methyloxy)propyl]{[(1-methylethyl)amino]carbonyl}amino)butyl]benzenesulfonamide

To a solution of 2,4-dichloro-N-(4-{[(2R)-2-hydroxy-3-(methyloxy)propyl]amino}butyl)benzenesulfonamide (200 mg, 0.52 mmol) in CHCl₃ (3 mL) was added 2-isocyanatopropane (0.05 mL, 0.05 mmol) at RT. After stirring for 1.5 hr at RT, the reaction mixture was concentrated under reduced pressure followed by purification on silica gel column chromatography (Biotage, 0% to 2.5% MeOH/CH₂Cl₂) to provide 201 mg (82%) of the title compound; LCMS: [MH]⁺=470.2.

### Example 149

### (1R)-2-((4-{[(2,4-Dichlorophenyl)sulfonyl]amino}butyl){[(1-methylethyl)amino]carbonyl}amino)-1-[(methyloxy)methyl]ethyl phenylcarbamate

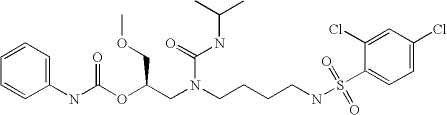

The title compound was prepared by the following procedure from Example 148 (Scheme 26); to a solution of Example 148 (40 mg, 0.085 mmol) in THF (1.5 mL) was added NaH (60% in mineral oil, 12 mg, 0.298 mmol) at 0° C. After stirring for 10 min at 0° C., isocyanatobenzene (0.014 mL, 0.128 mmol) was added. The reaction mixture was stirred for 10 min at 0° C. followed by quenching with cold 1N HCl. After extraction with EtOAc (×2), the combined organic solution was washed by saturated aq. NaHCO₃ solution and brine. The organic solution was dried over MgSO4, filtered, and concentrated under the reduced pressure. Purification of the residue by Biotage silica gel chromatography (0%-11.5% MeOH/DCM) provided 49 mg (98%) of the title compound; LCMS (m/z): 589.2 (M+H).

### Example 150

### (1R)-2-{[(4-{[(2,4-dichlorophenyl)sulfonyl]amino}butyl)[(ethylamino)carbonyl]amino}-1-[(methyloxy)methyl]ethyl phenylcarbamate

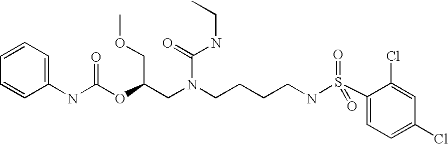

The title compound was prepared following the general procedure of Example 149 except substituting isocyanatoethane for 2-isocyanatopropane; LCMS (m/z): 575 (M+H).

### Example 151

### (1R)-2-{[(4-{[(2,4-Dichlorophenyl)sulfonyl]amino}butyl)[(phenylamino)carbonyl]amino}-1-[(methyloxy)methyl]ethyl phenylcarbamate

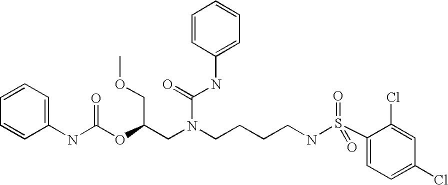

The title compound was prepared following the general procedure of Example 150 except substituting phenylisocyanate for 2-isocyanatopropane; LCMS (m/z): 623 (M+H).

### Example 152

### 2,4-Dichloro-N-[4-({(2R)-2-hydroxy-3-[(phenylmethyl)oxy]propyl}{[(1-methylethyl)amino]carbonyl}amino)butyl]benzenesulfonamide

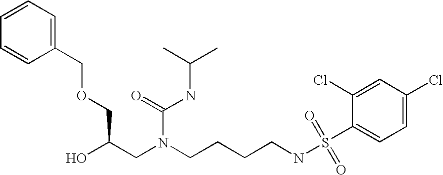

The title compound was prepared following the general procedure of Example 149 except substituting (R)-(−)-glycidyl benzyl ether for (R)-(−)-glycidyl methyl ether; LCMS (m/z): 461 (M+H).

### Example 153

### (1R)-2-((4-{[(2,4-Dichlorophenyl)sulfonyl]amino}butyl){[(1-methylethyl)amino]carbonyl}amino)-1-{[(phenylmethyl)oxy]methyl}ethyl phenylcarbamate

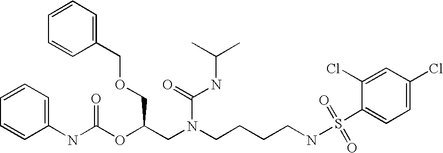

The title compound was prepared following the general procedure of Example 149 except substituting (R)-(−)-glycidyl benzyl ether for (R)-(−)-glycidyl methyl ether; LCMS (m/z): 589 (M+H).

### Example 154

### 2,4-Dichloro-N-[4-([(2S)-2-hydroxy-3-(4-morpholinyl)propyl]{[(1-methylethyl)amino]carbonyl}amino)butyl]benzenesulfonamide

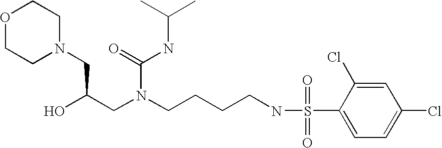

The title compound was prepared following the general procedure of Example 149 except substituting 4-[(2S)-2-oxiranylmethyl]morpholine ether (which prepared from (2R)-2-oxiranylmethyl 3-nitrobenzenesulfonate and morpholine in the presence of NaH in THF at rt) for (R)-(−)-glycidyl methyl ether; LCMS (m/z): 525 (M+H).

### Example 155

### (1S)-2-((4-{[(2,4-Dichlorophenyl)sulfonyl]amino}butyl){[(1-methylethyl)amino]carbonyl}amino)-1-(4-morpholinylmethyl)ethyl phenylcarbamate

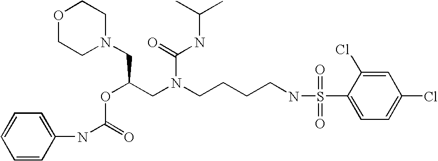

The title compound was prepared following the general procedure of Example 150 except substituting 4-[(2S)-2-oxiranylmethyl]morpholine ether for (R)-(−)-glycidyl methyl ether; LCMS (m/z): 644 (M+H).

### Example 156

### (1R)-2-((4-{[(2-chloro-4-fluorophenyl)sulfonyl]amino}butyl){[(1-methylethyl)amino]carbonyl}amino)-1-[(methyloxy)methyl]ethyl phenylcarbamate

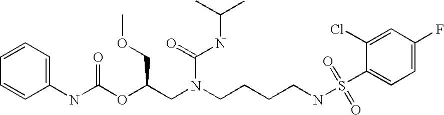

### a. 1,1-Dimethylethyl (4-{[(2R)-2-hydroxy-3-(methyloxy)propyl]amino}butyl)carbamate

To a solution of (2R)-2-[(methyloxy)methyl]oxirane (3 g, 34 mmol) in 2-methyl-2-propanol (150 mL) was added 1,1-dimethylethyl (4-aminobutyl)carbamate (9.6 g, 51 mmol). The reaction mixture was heated at 100° C. for 5 hours. The reaction mixture was concentrated. Purification of the residue by Biotage silica gel chromatography (1% to 10% MeOH/DCM) provided 6 g of the title compound (64%); LCMS (m/z): 277.3 (M+H).

### b. 1,1-Dimethylethyl[4-([(2R)-2-hydroxy-3-(methyloxy)propyl]{[(1-methylethyl)amino]carbonyl}amino)butyl]carbamate

To a solution of 1,1-dimethylethyl (4-aminobutyl)carbamate (1 g, 3.62 mmol) in DCM (15 mL) was added 2-isocyanatopropane (0.43 mL, 4.35 mmol). The reaction mixture was stirred at room temperature for 4 hours. The reaction mixture was concentrated. Purification of the residue by Biotage silica gel chromatography (1% to 7% MeOH/DCM) provided 1.3 g of the title compound (100%); LCMS (m/z): 361.7 (M+H).

### c. 1,1-Dimethylethyl {4-[{[(1-methylethyl)amino]carbonyl}((2R)-3-(methyloxy)-2-{[(phenylamino)carbonyl]oxy}propyl)amino]butyl}carbamate

To a solution of 1,1-dimethylethyl[4-([(2R)-2-hydroxy-3-(methyloxy)propyl]{[(1-methylethyl)amino]carbonyl}amino)butyl]carbamate (0.7 g, 1.93 mmol) in THF (10 mL) was added NaH (60% in mineral oil, 271 mg, 6.77 mmol) at 0° C. Kept stirring for S minutes, then added isocyanatobenzene (345 mg, 2.9 mmol). The reaction mixture was allowed to warm to room temperature and stirred for 4 hours. The reaction mixture was quenched with cold 1N HCl, extracted with EtOAc twice. The combined organic layer was washed by saturated aq. NaHCO₃ solution and brine. The organic solution was dried over MgSO₄, filtered, and concentrated under reduced pressure. Purification of the residue by Biotage silica gel chromatography (1% to 10% MeOH/DCM) provided 64 mg of the title compound (60%); LCMS (m/z): 481.2 (M+H).

### d. (1R)-2-((4-Aminobutyl){[(1-methylethyl)amino]carbonyl}amino)-1-[(methyloxy)methyl]ethyl phenylcarbamate (TFA salt)

To a solution of 1,1-dimethylethyl {4-[{[(1-methylethyl)amino]carbonyl}((2R)-3-(methyloxy)-2-{[(phenylamino)carbonyl]oxy}propyl)amino]butyl}carbamate (178 mg, 0.415 mmol) in dichloromethane (4 mL) was added TFA (2 mL) at RT. The reaction mixture was stirred at RT for 4 hours. After evaporation and drying under a vacuum pump, the crude material was used for the next step without further purification.

### e. (1R)-2-((4-{[(2-Chloro-4-fluorophenyl)sulfonyl]amino}butyl){[(1-methylethyl)amino]carbonyl}amino)-1-[(methyloxy)methyl]ethyl phenylcarbamate

To a solution of (1R)-2-((4-aminobutyl){[(1-methylethyl)amino]carbonyl}amino)-1-[(methyloxy)methyl]ethyl phenylcarbamate (TFA salt, 0.21 mmol) in DCM (2 mL) were added 2-chloro-4-fluorobenzenesulfonyl chloride (58 mg, 0.252 mmol) and triethylamine (0.09 mL, 0.6 mmol). The reaction mixture was stirred at room temperature for 5 hours. The reaction mixture was concentrated. Purification of the residue by Biotage silica gel chromatography (1% to 10% MeOH/DCM) provided 78 mg of the title compound (65%); LCMS (m/z): 573.5 (M+H).

### Example 157

### (1R)-2-([4-({[4-fluoro-2-(trifluoromethyl)phenyl]sulfonyl}amino)butyl]{[(1-methylethyl)amino]carbonyl}amino)-1-[(methyloxy)methyl]ethyl phenylcarbamate

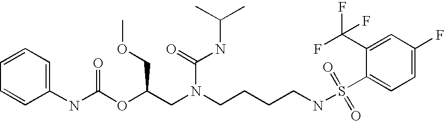

The title compound was prepared following the general procedure of Example 156 except substituting 4-fluoro-2-(trifluoromethyl)benzenesulfonyl chloride for 2-chloro-4-fluorobenzenesulfonyl chloride; MS (m/z): 606.6 (M+H).

### Example 158

### (1R)-2-[{[(1-methylethyl)amino]carbonyl}(4-{[(2-nitrophenyl)sulfonyl]amino}butyl)amino]-1-[(methyloxy)methyl]ethyl phenylcarbamate

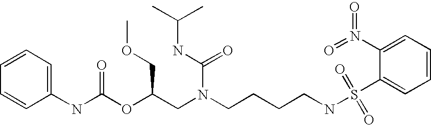

The title compound was prepared following the general procedure of Example 156 except substituting 2-nitrobenzenesulfonyl chloride for 2-chloro-4-fluorobenzenesulfonyl chloride; LCMS (m/z): 566.1 (M+H).

### Example 159

### (1R)-2-((4-{[(2,4-dichlorophenyl)sulfonyl]amino}butyl){[(1-methylethyl)amino]carbonyl}amino)-1-[(methyloxy)methyl]ethyl(4-fluorophenyl)carbamate

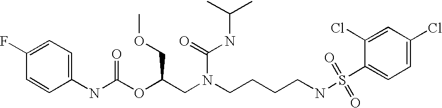

The title compound was prepared following the general procedure of Example 156 except substituting 1-fluoro-4-isocyanatobenzene for isocyanatobenzene and 2,4-dichlorobenzenesulfonyl chloride for 2-chloro-4-fluorobenzenesulfonyl chloride; LCMS (m/z): 607.5 (M+H).

### Example 160

### (1S)-2-((4-{[(2,4-dichlorophenyl)sulfonyl]amino}butyl)){[(1-methylethyl)amino]carbonyl}amino)-1-[(methyloxy)methyl]ethyl phenylcarbamate

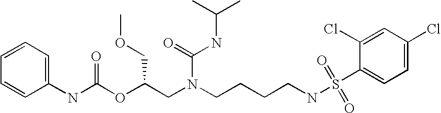

The title compound was prepared following the general procedure of Example 156 except substituting (2S)-2-[(methyloxy)methyl]oxirane for (2R)-2-[(methyloxy)methyl]oxirane and 2,4-dichlorobenzenesulfonyl chloride for 2-chloro-4-fluorobenzenesulfonyl chloride; LCMS (m/z): 589.5 (M+H).

### Example 161

### (1R)-2-{[(4-{[(2-chloro-4-fluorophenyl)sulfonyl]amino}butyl)[(ethylamino)carbonyl]amino}-1-[(methyloxy)methyl]ethyl phenylcarbamate

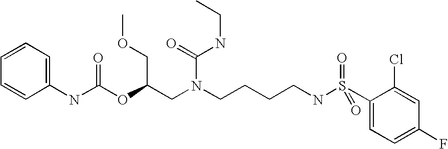

The title compound was prepared following the general procedure of Example 156 except substituting isocyanatoethane for 2-isocyanatopropane; LCMS (m/z): 559.2 (M+H).

### Example 162

### (1R)-2-{[(Ethylamino)carbonyl][4-({[4-fluoro-2-(trifluoromethyl)phenyl]sulfonyl}amino)butyl]amino}-1-[(methyloxy)methyl]ethyl phenylcarbamate

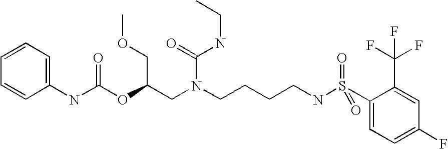

The title compound was prepared following the general procedure of Example 156 except substituting isocyanatoethane for 2-isocyanatopropane and 4-fluoro-2-(trifluoromethyl)benzenesulfonyl chloride for 2-chloro-4-fluorobenzenesulfonyl chloride; LCMS (m/z): 593.1 (M+H).

### Example 163

### O-{(1R)-2-{(4-{[(2,4-Dichlorophenyl)sulfonyl]amino}butyl)[(ethylamino)carbonyl]amino}-1-[(methyloxy)methyl]ethyl}phenylthiocarbamate

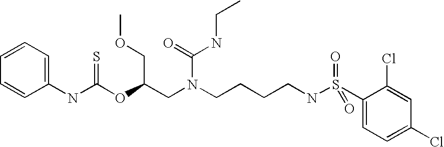

The title compound was prepared following the general procedure of Example 156 except substituting isothiocyanatobenzene for isocyanatobenzene; LCMS (m/z): 591.6 (M+H).

### Example 164

### (1R)-2-[{[(1-Methylethyl)amino]carbonyl}(4-{methyl[(2-nitrophenyl)sulfonyl]amino}butyl)amino]-1-[(methyloxy)methyl]ethyl phenylcarbamate

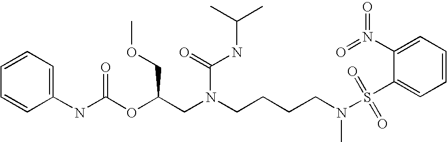

The title compound was prepared by the following procedure from (1R)-2-[{[(1-methylethyl)amino]carbonyl}(4-{[(2-nitrophenyl)sulfonyl]amino}butyl)amino]-1-[(methyloxy)methyl]ethyl phenylcarbamate (Example 40); To a solution of Example 40 (151.3 mg, 0.27 mmol) in acetone (5 mL) were added potassium carbonate (186 mg, 1.35 mmol) and MeI (0.05 mL, 0.81 mmol). The reaction mixture was stirred at room temperature for 5 hours. The reaction mixture was diluted with DCM, and then filtered the solid off. After concentration, the residue was purified by Biotage silica gel chromatography (1% to 5% MeOH/DCM) provided 119 mg of the title compound (76%); LCMS (m/z): 579.7 (M+H).

### Example 165

### (1R)-2-({4-[[(2-Cyanophenyl)sulfonyl(methyl)amino]butyl}{[(1-methylethyl)amino]carbonyl}amino)-1-[(methyloxy)methyl]ethyl phenylcarbamate

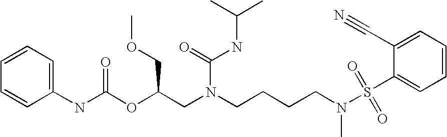

### a. (1R)-2-([4-(methylamino)butyl]{[(1-methylethyl)amino]carbonyl}amino)-1-[(methyloxy)methyl]ethyl phenylcarbamate

To a solution of (1R)-2-[{[(1-methylethyl)amino]carbonyl}(4-{methyl[(2-nitrophenyl)sulfonyl]amino}butyl)amino]-1-[(methyloxy)methyl]ethyl phenylcarbamate (Example 41, 105 mg, 0.18 mmol) in DMF (2 mL) were added benzenethiol (0.03 mL, 0.27 mmol) and potassium carbonate (75 mg, 0.543 mmol). The reaction mixture was stirred at room temperature for 4 hr. To the above solution was added ice water, and then adjusted pH to 1 with 1N HCl (3 mL). Extracted twice with DCM, the combined organic layer was washed with 1N HCl. After the aqueous solution was adjusted to pH 12-12.5 by 1N NaOH, the mixture was extracted with dichloromethane three times. The combined organic layer was washed with brine, dried over MgSO₄, filtered, and concentrated. The residue was carried to the next step without further purification.

### b. (1R)-2-({4-[[(2-cyanophenyl)sulfonyl](methyl)amino]butyl}{[(1-methylethyl)amino]carbonyl}amino)-1-[(methyloxy)methyl]ethyl phenylcarbamate

To a solution of (1R)-2-([4-(methylamino)butyl]{[(1-methylethyl)amino]carbonyl}amino)-1-[(methyloxy)methyl]ethyl phenylcarbamate (33.5 mg, 0.085 mmol) in DCM (1 mL) were added 2-cyanobenzenesulfonyl chloride (21 mg, 0.102 mmol) and triethylamine (0.035 mL, 0.255 mmol). The reaction mixture was stirred at room temperature for 5 hr. The reaction mixture was concentrated. Purification of the residue by Biotage silica gel chromatography (1% to 10% MeOH/DCM) provided 32 mg of the title compound (68%); LCMS (m/z): 560.2 (M+H).

### Example 166

### 2-[[(Cyclohexylamino)carbonyl](4-{[(2,4-dichlorophenyl)sulfonyl]amino}butyl)amino]ethyl phenylcarbamate

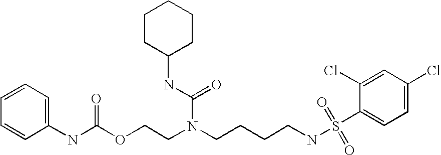

### a. 2,4-Dichloro-N-[4-({2-[(phenylmethyl)oxy]ethyl}amino)butyl]benzenesulfonamide

To a solution of 1-[(phenylmethyl)oxy]-2-propanone (0.15 g, 0.10 mmol) and N-(4-aminobutyl)-2,4-dichlorobenzenesulfonamide (0.33 g, 0.11 mmol) in 10 mL of MeOH were added NaCNBH₃ (0.070 g, 0.12 mmol) and AcOH (0.11 mL, 0.2 mmol). The reaction mixture was stirred at room temperature for 3 hours. 3N NaOH (3 mL) was added to the solution. The reaction mixture was extracted with EtOAc, dried over Na₂SO₄, filtered, and concentrated. The resultant residue was purified by flash column chromatography to provide 0.32 g (74%) of the desired product; LCMS (m/z): 431.2 (M+H).

### b. 2,4-Dichloro-N-[4-([(cyclohexylamino)carbonyl]{2-[(phenylmethyl)oxy]ethyl}amino)butyl]benzenesulfonamide

To a solution of 2,4-dichloro-N-[4-({2-[(phenylmethyl)oxy]ethyl}amino)butyl]benzenesulfonamide (0.60 g, 1.1 mmol) in 10 mL of CH₂Cl₂ was added isocyanatocyclohexane (0.62 g, 5 mmol). The reaction mixture was stirred at room temperature overnight. The solvent was removed and the crude material was used for the next step without purification.

### c. 2,4-Dichloro-N-{4-[[(cyclohexylamino)carbonyl]2-hydroxyethyl)amino]butyl}benzenesulfonamide

To solution of 2,4-dichloro-N-[4-([(cyclohexylamino)carbonyl]{b 2-[(phenylmethyl)oxy]ethyl}amino)butyl]benzenesulfonamide (0.70 g, 1.26 mmol) in 10 mL of CH₂Cl₂ at −60° C. was added 7.6 mL of BBr₃ (7.6 mmol, 1M in CH₂Cl₂). The reaction mixture was stirred at this temperature for 2 hr. The reaction was quenched with 2 mL of sat'd aq. NaHCO₃, followed by extraction with methylene chloride (2×20 mL). The organic solution was dried over MgSO₄, filtered, and concentrated. The resultant residue was purified by flash column chromatography to provide 0.15 g (46%) of the desired product; LCMS (m/z): 466.2 (M+H).

### d. 2-[[(Cyclohexylamino)carbonyl](4-{[(2,4-dichlorophenyl)sulfonyl]amino}butyl)amino]ethyl phenylcarbamate

To a solution of 2,4-dichloro-N-{4-[[(cyclohexylamino)carbonyl](2-hydroxyethyl)amino]butyl}benzenesulfonamide (46 mg, 0.1 mmol) in 1 mL of CH₂Cl₂ was added phenyl isocyanate (24 mg, 0.2 mmol). The reaction mixture was stirred at room temperature overnight. After evaporation, the resultant residue was purified by flash column chromatography without aqueous work-up to provide 34 mg of a desired product (60%); LCMS: [MH]⁺=585.2.

### Example 167

### 2-[[(cyclohexylamino)carbonyl](4-{[(2,4-dichlorophenyl)sulfonyl]amino}butyl)amino]ethyl cyclohexylcarbamate

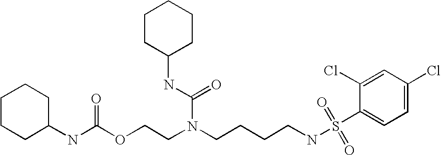

The title compound was prepared following the procedure of Example 164 except for the use of cyclohexyl isocyanate in place of phenyl isocyanate; LCMS: [MH]⁺=591.2.

### Example 168

### 2-[[(cyclohexylamino)carbonyl](4-{[(2,4-dichlorophenyl)sulfonyl]amino}butyl)amino]ethyl methylcarbamate

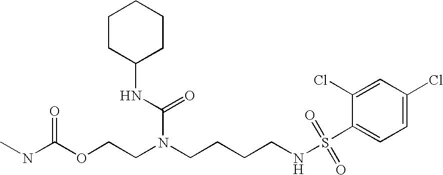

The title compound was prepared following the procedure of Example 164 except for the use of methyl isocyanate in place of phenyl isocyanate; LCMS: [MH]⁺=523.2.

### Example 169

### 2-Chlorophenyl[2-((4-{[(2,4-dichlorophenyl)sulfonyl]amino}butyl){[(1-methylethyl)amino]carbonyl}amino)ethyl]carbamate

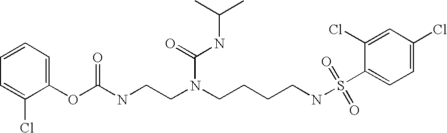

### a. 1,1-Dimethylethyl {2-[(4-{[(2,4-dichlorophenyl)sulfonyl]amino}butyl)amino]ethyl}carbamate

To a solution of 1,1-dimethylethyl (2-oxoethyl)carbamate (1 g, 6.29 mmol) in MeOH (20 mL) were added N-(4-aminobutyl)-2,4-dichlorobenzenesulfonamide (2.23 g, 7.55 mmol), NaCNBH₃ (592 mg, 9.44 mmol) and AcOH (0.72 mL, 12.58 mmol). The reaction mixture was stirred at room temperature for 4 hrs. After adjusting pH to 12 with 1N NaOH, the mixture was extracted with EtOAC three times. The organic layer was washed with brine, dried over MgSO₄, filtered, and concentrated. Purification of the residue by Biotage silica gel column chromatography (1% to 10% MeOH/DCM) provided 1.30 g of the title compound (47%); LCMS (m/z): 440.4 (M+H).

### b. 1,1-Dimethylethyl[2-((4-{[(2,4-dichlorophenyl)sulfonyl]amino}butyl){[(1-methylethyl)amino]carbonyl}amino)ethyl]carbamate

To the solution of 1,1-dimethylethyl {2-[(4-{[(2,4-dichlorophenyl)sulfonyl]amino}butyl)amino]ethyl}carbamate (450 mg, 1.02 mmol) in DCM (10 mL) was added 2-isocyanatopropane (0.12 mL, 1.23 mmol). The reaction mixture was stirred at room temperature for 4 hours. The reaction mixture was concentrated. Purification of the residue by Biotage silica gel column chromatography (1% to 7% MeOH/DCM) provided 452 mg of the title compound (84%); LCMS (m/z): 525.5 (M+H).

### c. 2-Chlorophenyl[2-((4-{[(2,4-dichlorophenyl)sulfonyl]amino}butyl){[(1-methylethyl)amino]carbonyl}amino)ethyl]carbamate

Followed the general de-Boc procedure using TFA from 1,1-dimethylethyl[2-((4-{[(2,4-dichlorophenyl)sulfonyl]amino}butyl){[(1-methylethyl)amino]carbonyl}amino)ethyl]carbamate. To a solution of the crude material (TFA salt, 116 mg, 0.22 mmol) in DCM (3 mL) were added 2-chlorophenyl chloroformate (0.03 mL, 0.24 mmol) and TEA (0.06 mL, 0.43 mmol). The reaction mixture was stirred at room temperature for 5 hr. The reaction mixture was concentrated. Purification of the residue by Biotage silica gel column chromatography (1% to 10% MeOH/DCM) provided 33 mg of the title compound (27%); LCMS (m/z): 579.5 (M+H).

### Example 170

### 2,4-Dichloro-N-{4-[{[(1-methylethyl)amino]carbonyl}((2R)-3-(methyloxy)-2-{[(phenylamino)carbonyl]amino}propyl)amino]butyl}benzenesulfonamide

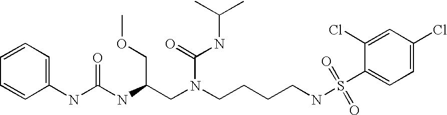

### a. 1,1-Dimethylethyl (4-{[(2S)-2-hydroxy-3-(methyloxy)propyl]amino}butyl)carbamate

To a solution of (2S)-2-[(methyloxy)methyl]oxirane (1 g, 11.3 mmol) in 2-methyl-2-propanol (60 mL) was added 1,1-dimethylethyl (4-aminobutyl)carbamate (3.2 g, 17 mmol). The reaction mixture was stirred at 100° C. for 5 hr. The reaction mixture was concentrated. Purification of the residue by Biotage silica gel column chromatography (1% to 10% MeOH/DCM) provided 1.9 g of the title compound (61%); LCMS (m/z): 277.3 (M+H).

### b. 1,1-Dimethylethyl[4-([(2S)-2-hydroxy-3-(methyloxy)propyl]{[(1-methylethyl)amino]carbonyl}amino)butyl]carbamate

To a solution of 1,1-dimethylethyl (4-{[(2S)-2-hydroxy-3-(methyloxy)propyl]amino}butyl)carbamate (310 mg, 1.12 mmol) in DCM (8 mL) was added 2-isocyanatopropane (0.13 mL, 1.2 mmol). The reaction mixture was stirred at room temperature for 4 hr. The reaction mixture was concentrated. Purification of the residue by Biotage silica gel column chromatography (1% to 7% MeOH/DCM) provided 406 mg of the title compound (100%); LCMS (m/z): 361.7 (M+H).

### c. (1S)-2-([4-({[(1,1-Dimethylethyl)oxy]carbonyl}amino)butyl]{[(1-methylethyl)amino]carbonyl}amino)-1-[(methyloxy)methyl]ethyl methanesulfonate

To a solution of 1,1-dimethylethyl[4-([(2S)-2-hydroxy-3-(methyloxy)propyl]{[(1-methylethyl)amino]carbonyl}amino)butyl]carbamate (172 mg, 0.475 mmol) in THF (6 mL) at −20° C. were added Et₃N (0.15 mL, 1.0 mmol) and DMAP (0.32 mg, 0.03 mmol). After 10 min, MsCl (0.044 ml, 0.57 mmol) was slowly added. The reaction mixture was allowed to warm to room temperature and stirred overnight. The reaction mixture was washed by saturated aq. NH₄Cl, saturated aq. NaHCO₃, and brine. The organic layer was dried over MgSO₄. After filtration and concentration, the crude material was used for the next step without further purification; LCMS (m/z): 440.1 (M+H).

### d. 1,1-Dimethylethyl[4-([(2R)-2-azido-3-(methyloxy)propyl]{[(1-methylethyl)amino]carbonyl}amino)butyl]carbamate

To the solution of (1S)-2-([4-({[(1,1-dimethylethyl)oxy]carbonyl}amino)butyl]{[(1-methylethyl)amino]carbonyl}amino)-1-[(methyloxy)methyl]ethyl methanesulfonate (209 mg, 0.475 mmol) in DMF (5 mL) was added sodium azide (37 mg, 0.57 mmol). The reaction mixture was stirred at 100° C. overnight. Removing most of the solvent under reduced pressure, the residue was diluted in dichloromethane. The reaction mixture was washed with water, saturated aq. NaHCO₃, and brine. The organic layer was dried over MgSO₄, filtered, and concentrated. Purification of the residue by Biotage silica gel column chromatography (1% to 10% MeOH/DCM) provided 139 mg of the title compound (76%); LCMS (m/z): 387.1 (M+H).

### e. 1,1-Dimethylethyl[4-([(2R)-2-amino-3-(methyloxy)propyl]{[(1-methylethyl)amino]carbonyl}amino)butyl]carbamate

1,1-Dimethylethyl[4-([(2R)-2-azido-3-(methyloxy)propyl]{[(1-methylethyl)amino]carbonyl}amino)butyl]carbamate (139 mg, 0.36 mmol) was dissolved MeOH (4 mL). After adding Pd/carbon (10%, 38 mg, 0.036 mmol), the reaction mixture was vigorously stirred under hydrogen (balloon) for 4 hr. The mixture was filtered through celite, which was rinsed with MeOH. The combined filtrate was concentrated and the crude material was used for the next step without further purification; LCMS (m/z): 361.5 (M+H).

### f. 1,1-Dimethylethyl {4-[{[(1-methylethyl)amino]carbonyl}((2R)-3-(methyloxy)-2-{[(phenylamino)carbonyl]amino}propyl)amino]butyl}carbamate

To a solution of 1,1-dimethylethyl[4-([(2R)-2-amino-3-(methyloxy)propyl]{[(1-methylethyl)amino]carbonyl}amino)butyl]carbamate (130 mg, 0.36 mmol) in DCM (4 mL) was added isocyanatobenzene (86 mg, 0.72 mmol). The reaction mixture was stirred at room temperature for 4 hr. The reaction mixture was concentrated. Purification of the residue by Biotage silica gel column chromatography (10% to 95% MeOH/DCM) provided 109 mg of the title compound (63%); LCMS (m/z): 480.3 (M+H).

### g. 2,4-Dichloro-N-{4-[{[(1-methylethyl)amino]carbonyl}((2R)-3-(methyloxy)-2-{[(phenylamino)carbonyl]amino}propyl)amino]butyl}benzenesulfonamide

The title compound was prepared following the procedure such as Example 38d-38e except substituting 2,4-dichlorobenzenesulfonyl chloride for 2-chloro-4-fluorobenzenesulfonyl chloride; LCMS (m/z): 588.3 (M+H).

### Example 171

### 2-[((4-{[(2,4-dichlorophenyl)sulfonyl]amino}butyl){[(1-methylethyl)amino]carbonyl}amino)methyl]-N-phenyl-1-piperidinecarboxamide

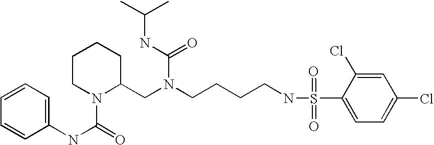

The title compound was prepared following the procedure of example 51 except for the use of 1,1-dimethylethyl-2-formyl-1-piperidinecarboxylate (described the preparation below) in place of 1,1-dimethylethyl (2-oxoethyl)carbamate and the use of phenyl isocyanate in place of 2-chlorophenyl chloridocarbonate; LCMS: [MH]⁺=598.

### a. 1,1-Dimethylethyl 2-formyl-1-piperidinecarboxylate

To a solution of 2-piperidinylmethanol (730 mg, 6.33 mmol) in THF (30 mL) at rt were added (BOC)₂O (1.66 g, 7.6 mmol), Et₃N (1.32 mL, 9.50 mmol), and DMAP (20 mg). After 1 hr at rt, cold 1N HCl (20 mL) was added. After extraction with EtOAc (30 mL×2), the organic solution was washed with 1N HCl, sat'd aq. NaHCO₃, and brine followed by drying over MgSO₄, filtration, and evaporation under reduced pressure. After drying under a vacuum pump, the resultant residue (1,1-dimethylethyl 2-(hydroxymethyl)-1-piperidinecarboxylate, crude, 153 mg, 0.71 mmol) was dissolved in CH₂Cl₂ (4 mL), followed by the addition of Dess-Martin Periodinane (392 mg, 0.93 mmol) at rt. After 1 hr at rt, saturated aq. NaHCO₃ was added and extracted with CH₂Cl₂ (twice). The organic solution was dried over MgSO₄. After filtration and evaporation, the residue was purified by Biotage silica gel column chromatography (CH₂Cl₂ only) to provide 103 mg of a desired product (68% for two steps).

### Example 172

### (3R)-3-((4-{[(2,4-Dichlorophenyl)sulfonyl]amino}butyl){[(1-methylethyl)amino]carbonyl}amino)-N-phenyl-1-piperidinecarboxamide

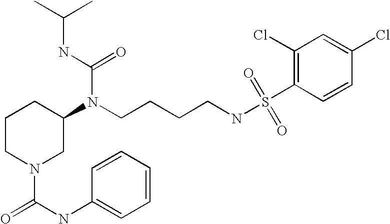

### a. 4-(1,3-Dioxo-1,3-dihydro-2H-isoindol-2-yl)butanal

To a solution of [4,4-bis(ethyloxy)butyl]amine (5.0 g, 30.0 mmol) and Et₃N (4.61 ml, 33.0 mmol) in 40 mL of THF was added ethyl 1,3-dioxo-1,3-dihydro-2H-isoindole-2-carboxylate (6.9 g, 32.0 mmol). The mixture was stirred overnight at room temperature. The reaction mixture was concentrated in vacuo and the residue was extracted with hexane. The hexane solution was concentrated in vacuo and heated at 100° C. under reduced pressure to remove ethylcarbamate by sublimation. The mixed solution of diethyl acetal (2.0 g, 6.95 mmol) thus obtained and 1N aq. HCl (14 mL) in acetone (20 mL) was heated under reflux for 15 min. Acetone was evaporated and aqueous layer was extracted with ether, dried over MgSO₄, and filtered. The solvent was evaporated and the residue was moved to next step.

### b. (3R)-3-Amino-N-phenyl-1-piperidinecarboxamide (TFA salt)

To a solution of 1,1-dimethylethyl (3R)-3-piperidinylcarbamate (0.60 g, 2.9 mmol) in 10 mL of CH₂Cl₂ was added phenyl isocyanate (0.68 g, 5.8 mmol). The reaction mixture was stirred at room temperature overnight. The solvent was removed and the crude material was dissolved in 2 mL of CH₂Cl₂. TFA (2 mL) was added to the solution. The mixture was allowed to stir at room temperature for 1 hr. The solvent was evaporated and the residue was moved to next step without purification.

### c. (3R)-3-{[4-(1,3-Dioxo-1,3-dihydro-2H-isoindol-2-yl)butyl]amino}-N-phenyl-1-piperidinecarboxamide

To a solution of (3R)-3-amino-N-phenyl-1-piperidinecarboxamide (TFA salt, 0.70 g, 2.2 mmol) and 4-(1,3-dioxo-1,3-dihydro-2H-isoindol-2-yl)butanal (0.30 g, 1.4 mmol) in 10 mL of CH₂Cl₂ was added NaBH(OAc)₃ (0.89 g, 4.2 mmol). The reaction mixture was stirred at room temperature overnight. NaOH solution (1.0 N) was added to pH 12. The solution was extracted with CH₂Cl₂. The organic layer was washed with water and brine. The solvent was evaporated and the resultant residue was purified by flash column chromatography to provide 0.30 g of the desired product (51%); LCMS: [MH]⁺=421.2.

### d. (3R)-3-([4-(1,3-Dioxo-1,3-dihydro-2H-isoindol-2-yl)butyl]{[(1-methylethyl)amino]carbonyl}amino)-N-phenyl-1-piperidinecarboxamide

To a solution of (3R)-3-{[4-(1,3-dioxo-1,3-dihydro-2H-isoindol-2-yl)butyl]amino}-N-phenyl-1-piperidinecarboxamide (0.16 g, 0.40 mmol) in 3 mL of CH₂Cl₂ was added i-propyl isocyanate (0.078 g, 1.0 mmol). The reaction mixture was stirred at room temperature overnight. The solvent was evaporated and the crude material was moved to next step without purification; LCMS: [MH]⁺=421.

### e. (3R)-3-((4-{[(2,4-Dichlorophenyl)sulfonyl]amino}butyl){[(1-methylethyl)amino]carbonyl}amino)-N-phenyl-1-piperidinecarboxamide

To a solution of (3R)-3-([4-(1,3-dioxo-1,3-dihydro-2H-isoindol-2-yl)butyl]{[(1-methylethyl)amino]carbonyl}amino)-N-phenyl-1-piperidinecarboxamide (0.050 g, 0.10 mmol) in 10 mL of EtOH was added NH₂NH₂ (0.013 g, 0.40 mmol). The reaction mixture was stirred at room temperature overnight. The solid was filtered off and the filtrate was concentrated. To a solution of the above crude material in 2 mL of CH₂Cl₂ were added 2,4-dichlorobenzenesulfonyl chloride (0.029 g, 0.12 mmol) and Et₃N (0.24 mL, 0.20 mmol) at rt. After stirring for 30 min at rt, the reaction mixture was concentrated and purified by flash column chromatography without aqueous work-up to provide 25 mg of a desired product (43% for 2 steps); LCMS: [MH]⁺=584.2.

### Example 173

### (3S)-3-((4-{[(2,4-dichlorophenyl)sulfonyl]amino}butyl){[(1-methylethyl)amino]carbonyl}amino)-N-phenyl-1-piperidinecarboxamide

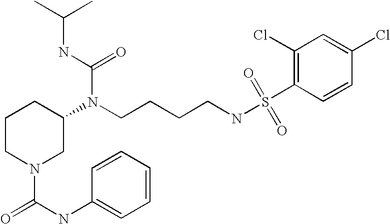

The title compound was prepared following the procedure of Example 172 except for the use of 1,1-dimethylethyl (3S)-3-piperidinylcarbamate in place of 1,1-dimethylethyl (3R)-3-piperidinylcarbamate; LCMS: [MH]⁺=584.2.

### Example 174

### 2-[((4-{[(2,4-dichlorophenyl)sulfonyl]amino}butyl){[(1-methylethyl)amino]carbonyl}amino)methyl]-N-phenyl-1-pyrrolidinecarboxamide

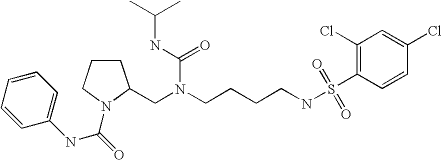

The title compound was prepared following the general procedure of Example 172 except for the use of 1,1-dimethylethyl 2-(aminomethyl)-1-pyrrolidinecarboxylate in place of 1,1-dimethylethyl (3R)-3-piperidinylcarbamate; LCMS (m/z): 584.6 (M+H).

### Example 175

### 2-({(4-{[(2,4-dichlorophenyl)sulfonyl]amino}butyl)[(ethylamino)carbonyl]amino}methyl)-N-phenyl-1-pyrrolidinecarboxamide

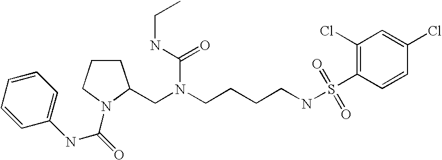

The title compound was prepared following the general procedure of Example 172 except substituting isocyanatoethane for 2-isocyanatopropane; LCMS (m/z): 570.3 (M+H).

### Example 176

### N-(4-{[(2,4-dichlorophenyl)sulfonyl]amino}butyl)-2,2-dimethyl-N-((2S)-4-methyl-2-{[(phenylamino)carbonyl]amino}pentyl)hydrazinecarboxamide

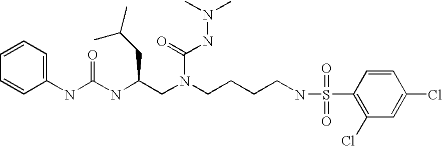

The title compound was prepared following the procedure of Example 119 except for the substitution of N,N-dimethyl-1H-imidazole-1-carbohydrazide for cyclohexylisocyanate in the last step. N,N-dimethyl-1H-imidazole-1-carbohydrazide was prepared by reaction 1,1-dimethylhydrazine with CDl in dichloromethane; LCMS: [MH]⁺=601.

### Example 177

### 2,4-dichloro-N-{4-[{[(2-fluoroethyl)amino]carbonyl}((2S)-4-methyl-2-{[(phenylamino)carbonyl]amino}pentyl)amino]butyl}benzenesulfonamide

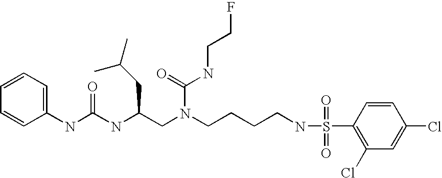

The title compound was prepared following the procedure of Example 119 except for the substitution of N-(2-fluoroethyl)-1H-imidazole-1-carboxamide for FMOC-Cl in step c. N-(2-fluoroethyl)-1H-imidazole-1-carboxamide was prepared by reaction of (2-fluoroethyl) amine with CDl in dichloromethane; LCMS: [MH]⁺=604.

### Example 178

### 2-Chloro-4-fluoro-N-{4-[{[(1-methylethyl)amino]carbonyl}((2S)-4-methyl-2-{[(phenylamino) carbonyl]amino}pentyl)amino]butyl}benzenesulfonamide

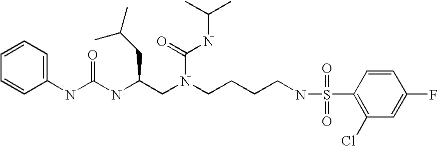

The title compound was prepared following the procedure of Example 119 except for the following changes: In step b, phenylmethyl (4-aminobutyl)carbamate hydrochloride was used in place of N-(4-aminobutyl)-2,4-dichlorobenzenesulfonamide. In step c, isopropylisocyanate was coupled in place of FMOC-Cl. Finally, the Cbz group was removed by hydrogenation over Pd/C in MeOH, and 2-chloro-4-fluorobenzenesulfonyl chloride was coupled using Et₃N in dichloromethane; LCMS: [MH]⁺=584.

### Example 179

### 4-fluoro-N-{4-[{[(1-methylethyl)amino]carbonyl}((2S)-4-methyl-2-{[(phenylamino)carbonyl]amino}pentyl)amino]butyl}-2-(trifluoromethyl)benzenesulfonamide

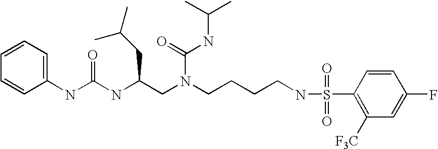

The title compound was prepared following the procedure of Example 119 except for the following changes: In step 2, phenylmethyl (4-aminobutyl)carbamate hydrochloride was used in place of N-(4-aminobutyl)-2,4-dichlorobenzenesulfonamide. In step c, isopropylisocyanate was coupled in place of FMOC-Cl. Finally, the Cbz group was removed by hydrogenation over Pd/C in MeOH, and 4-fluoro-2-(trifluoromethyl)benzenesulfonyl chloride was coupled using Et₃N in dichloromethane; LCMS: [MH]⁺=618.

All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

1. A compound of formula I 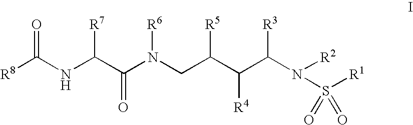
or pharmaceutically acceptable salts, hydrates, or solvates thereof, wherein:
R¹ is aryl optionally substituted with CN, NO₂, halogen, CH₃, CF₃ or H;
R² is H, C₁-C₆ alkyl, C₃-C₇ cycloalkyl, or C₃-C₇ heterocycloalkyl;
R³ is H, OH, C₁-C₆OH, O—C₁-C₆ alkyl, CO₂CH₃, CONHCH₃, SH, S—C₁-C₆ alkyl, or F;
S R⁴ is H, OH, C₁-C₆ OH, O—C₁-C₆ alkyl, SH, S—C₁-C₆ alkyl, or F;
R⁵ is H, OH, C₁-C₆ OH, O—C₁-C₆ alkyl, SH, S—C₁-C₆ alkyl, or F;
R⁶ is H or C₁-C₆ alkyl;
R⁷ is optionally substituted C₁-C₆ alkyl, O—C₁-C₈ alkyl, C—S—C₁-C₆ alkyl cyclohexylmethyl, amide, urea, or cyclopentylmethyl; and
R⁸ is optionally substituted C₃₋₇cycloalkyl, optionally substituted C₃₋₇cycloalkenyl, optionally substituted Het-C₃₋₇alkyl, optionally substituted Het-C₃₋₇alkenyl, optionally substituted aryl, optionally substituted aryloxy; optionally substituted arylamino; optionally substituted heterocycloalkyl, optionally substituted heteroaryl, optionally substituted cylcoalkyl, or optionally substituted indenyl.

2. A compound of formula I 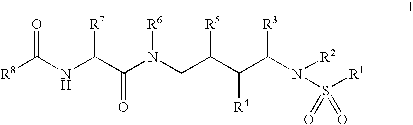
or pharmaceutically acceptable salts, hydrates, or solvates thereof, wherein:
wherein R¹ is aryl substituted with one or more substituents selected from the group consisting of halo, cyano, methyl and CF₃;
R² is H;
R³ is H, C₂OH, CO₂CH₃ or CONHCH₃;
R⁴ is H or OH;
R⁵ is H or OH,
R⁶ is H;
R⁷ is isobutyl, butene, thiazol, C—O—C₁-C₆, alkyl, hydroxydimethylbutyl, dichloropropyl, trifluoropropyl, phenylethyl, or phenylpropyl; and
R⁸ is phenyl, optionally substituted phenyl, benzothienyl, C₁₋₁₂alkyl substituted benzothienyl, benzothiazolyl; alkyl substituted benzothiazolyl; furanyl, halogen substituted furanyl, aryl substituted furanyl; tetrahydrofuran-2-yl; benzofuranyl, alkoxy substituted benzofuranyl, halogen substituted benzofuranyl, alkyl substituted benzofuranyl; benzo[b]thiophenyl, alkoxy substituted benzo[b]thiophenyl; optionally substituted isoquinolinyl, quinolinyl; indolyl, alkyl substituted indolyl; alkyl substituted indolyl further substituted with dimethylethyl carboxylate; indolyl further substituted with one to three carboxy groups, methylphenyl propenoyl, pyridinyl, alkyl substituted pyridinyl, thiopyranyl, pyridazinyl; thienopyridinyl, quinolizinyl, optionally substituted imidazolyl, imidazothiazolyl, pyrrolyl, cylcopenta[b]thiophenyl, cyclopentyl substituted with one to three alkoxy groups, cyclohexyl substituted with one to three alkoxy groups, cylcopentylpropanoyl, cyclohexylpropanoyl, cylcopentylmethyloxy, cyclohexylmethyloxy, cyclohexyldimethylpropanoyl, cyclopentylamino, cyclohexylamino, cyclopentylmethylamino, cyclohexylmethylamino, indenyl, cyclohexene, piperidinyl, propylpiperidinyl further substituted with methylbutylcarbozylate, thiophenyl, thiophenyl further substituted with phenyl, alkyl substituted thiophenyl, halogen substituted thiophenyl, halogen substituted benzothiophenyl, thieno[3,2-b]thiophenyl, isoxazolyl, alkyl substituted isoxazolyl, and oxazolyl; and
pharmaceutically acceptable salts, hydrates, or solvates thereof.

3. The compound of claim 2, wherein R⁸ is phenyl substituted with one to three substituents selected from the group consisting of: C₂O, NO₂, dimethylpropanoyl, methylpiperazinyl, phenyl, piperazine further substituted with dimethylethylcarbonyl, amino, halogen, CH₃, C₁-C₁₂ alkyl, C₁-C₁₂ alkoxy, amino sulfonyl, and alkylsulfonyl groups and pharmaceutically acceptable salts, hydrates, or solvates thereof.

4. The compound of claim 2 wherein R⁸ is isoquinoline further substituted with one to three substituents selected from the group consisting of dimethylethylcarbonyl and phenylcarbonyl and pharmaceutically acceptable salts, hydrates, or solvates thereof.

5. A pharmaceutical composition comprising a compound according to claim 1 and a pharmaceutically acceptable carrier, diluents or excipient.

6. A compound of Formula II 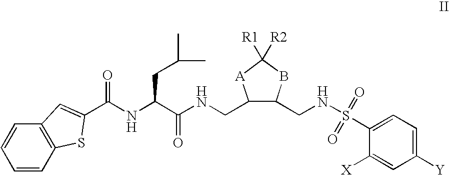
and pharmaceutically acceptable salts, hydrates, or solvates thereof, wherein:
R¹ is H or CH₃;
R² is H or CH₃;
A is C or O;
B is C or O;
X is H, Cl or F; and
Y is H, Cl or F.

7. A pharmaceutical composition comprising a compound according to claim 6 and a pharmaceutically acceptable carrier, diluents or excipient.

8. A compound of Formula III 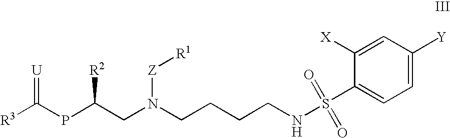
and pharmaceutically acceptable salts, hydrates, or solvates thereof, wherein:
X is H, C₁, CF₃, NO₂, or CN;
Y is H, Cl, or F;
Z is C═S, C═O or O═S═O;
U is O or S;
R¹ is optionally substituted cyloalkyl, C₁-C₁₂ alkyl, C₁-C₁₂ alkoxy, C₁-C₁₂ alkylamino, optionally substituted aryl, optionally substituted arylamino, optionally substituted heteroaryl; or optionally substituted heterocycloalkyl;
R² is H, C₁-C₁₂ alkyl, C₁-C₁₂ alkoxy, optionally substituted heterocycloalkylamino, optionally substituted heteroaryl, or optionally substituted aryl;
P is NH or O; and
R³ is C₁-C₁₂ alkylamino, cycloalkylamino, optionally substituted aryl amino, optionally substituted heteroarylamino, heterocyclicalkyl, optionally substituted aryloxy;
wherein when P is NH, R² may form a five or six member heterocyclic ring with P forming a piperidinyl or pyrrolidinyl group.

9. The compound of claim 8, and pharmaceutically acceptable salts, hydrates, or solvates thereof, wherein:
X is H, C₁, CF₃, NO₂, or CN;
Y is H, Cl, or F;
Z is C═S, C═O or O═S═O;
U is O or S;
R¹ is cyclohexylamino, methyl, carbonyl, ethylamino, methylethylamino, phenylamino, cylcopropylamino, dimethylethylamino, substituted phenyl, furanylmethyl amino, thienyl amino, substituted piperidinyl, azinyl, or fluoroethyl amino;
R² is H, isobutyl, methyloxypropyl, phenylmethyloxypropyl, phenylmethyloxymethyl, morpholinylpropyl, or morpholinylmethyl;
P is NH or O; and
R³ is phenylamino, methylethylamino, cyclohexylamino, ethylamino, substituted phenylamino, pyridinylamino, thienylamino, trifluoroacetylpiperidinyl, cyclopentylamino, methyloxypropyl, phenylmethyloxy, morpholinyl, or methylamino;
wherein when P is NH, R² may form a five or six member heterocyclic ring with P forming a piperidinyl or pyrrolidinyl group.

10. The compound of claim 9, wherein R³ is phenylamino further substituted with a one to three substituents selected from the group of halogen, pyrrolyl, and pyrazolyl.

11. A pharmaceutical composition comprising a compound according to claim 8 and a pharmaceutically acceptable carrier, diluents or excipient.

12. A compound selected from the group consisting of:
N-((1S)-1-{[(4-{[(2,4-Dichlorophenyl)sulfonyl]amino}butyl)amino]carbonyl}-3-methylbutyl)-1-benzothiophene-2-carboxamide;
N-((1S)-1-{[(4-{[(2-Bromo-4-fluorophenyl)sulfonyl]amino}butyl)amino]carbonyl}-3-methylbutyl)-1-benzothiophene-2-carboxamide;
N-((1S)-1-{[(4-{[(4-Bromo-2-chlorophenyl)sulfonyl]amino}butyl)amino]carbonyl}-3-methylbutyl)-1-benzothiophene-2-carboxamide;
N-[(1S)-1-({[4-({[4-Fluoro-2-(trifluoromethyl)phenyl]sulfonyl}amino)butyl]amino}; carbonyl)-3-methylbutyl]-1-benzothiophene-2-carboxamide;
N-((1S)-1-{[(4-{[(2-Chloro-4-fluorophenyl)sulfonyl]amino}butyl)amino]carbonyl}-3-methylbutyl)-1-benzothiophene-2-carboxamide;
N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide;
N-((1S)-1-{[(4-{[(2,4-dichlorophenyl)sulfonyl]amino}butyl)amino]carbonyl}-3-methylbutyl)-1-methyl-1H-indole-2-carboxamide;
N-((1S)-1-{[(4-{[(2,4-dichlorophenyl)sulfonyl]amino}butyl)amino]carbonyl}-3-methylbutyl)-1-benzofuran-2-carboxamide;
N-((1S)-1-{([(4-{[(2,4-Dichlorophenyl)sulfonyl]amino}butyl)amino]carbonyl}-3-methylbutyl)-1-benzothiophene-2-carboxamide;
N-((1S)-1-{[(4-{[(2-Bromo-4-fluorophenyl)sulfonyl]amino}butyl)amino]carbonyl}-3-methylbutyl)-1-benzothiophene-2-carboxamide;
N-((1S)-1-{[(4-{[(4-Bromo-2-chlorophenyl)sulfonyl]amino}butyl)amino]carbonyl}-3-methylbutyl)-1-benzothiophene-2-carboxamide;
N-[(1S)-1-({[4-({[4-Fluoro-2-(trifluoromethyl)phenyl]sulfonyl}amino)butyl]amino}carbonyl)-3-methylbutyl]-1-benzothiophene-2-carboxamide;
N-((1S)-1-{[(4-{[(2-Chloro-4-fluorophenyl)sulfonyl]amino}butyl)amino]carbonyl}-3-methylbutyl)-1-benzothiophene-2-carboxamide;
N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide;
N-((1S)-1-{[(4-{[(2,4-dichlorophenyl)sulfonyl]amino}butyl)amino]carbonyl}-3-methylbutyl)-1-methyl-1H-indole-2-carboxamide;
N-((1S)-1-{[(4-{[(2,4-dichlorophenyl)sulfonyl]amino}butyl)amino]carbonyl}-3-methylbutyl)-1-benzofuran-2-carboxamide;
N-((1S)-1-{[(4-{[(2,4-Dichlorophenyl)sulfonyl]amino}butyl)amino]carbonyl}-3-methylbutyl)-3-methylfuro[3,2-b]pyridine-2-carboxamide;
N-((1S)-1-{[(4-{[(2,4-Dichlorophenyl)sulfonyl]amino}butyl)amino]carbonyl}-3-methylbutyl)imidazo[1,2-b]pyridazine-2-carboxamide;
N-((1S)-1-{[(4-{[(2,4-Dichlorophenyl)sulfonyl]amino}butyl)amino]carbonyl}-3-methylbutyl)thieno[3,2-b]pyridine-2-carboxamide;
(2S)-1-N-((1S)-1-{[(4-{[(2,4-Dichlorophenyl)sulfonyl]amino}butyl)amino]carbonyl}-3-methylbutyl)octahydro-2H-quinolizine-2-carboxamide;
N-((1S)-1-{[(4-{[(2,4-Dichlorophenyl)sulfonyl]amino}butyl)amino]carbonyl}-3-methylbutyl)imidazo[1,2-a]pyridine-2-carboxamide;
N-((1S)-1-{[(4-{[(2,4-Dichlorophenyl)sulfonyl]amino}butyl)amino]carbonyl}-3-methylbutyl)imidazo[2,1-b][1,3]thiazole-6-carboxamide;
N-((1S)-1-{[(4-{[(2,4-Dichlorophenyl)sulfonyl]amino}butyl)amino]carbonyl}-3-methylbutyl)-4H-thieno[3,2-b]pyrrole-5-carboxamide;
N-[(1S)-1-({[4-({[4-Fluoro-2-(trifluoromethyl)phenyl]sulfonyl}amino)butyl]amino}carbonyl)-3-methylbutyl]-4H-thieno[3,2-b]pyrrole-5-carboxamide;
N-((1S)-1-{[(4-{[(2-Chloro-4-fluorophenyl)sulfonyl]amino}butyl)amino]carbonyl}-3-methylbutyl)-4H-thieno[3,2-b]pyrrole-5-carboxamide;
N¹-(4-{[(2,4-Dichlorophenyl)sulfonyl]amino}butyl)-N²-({4-[(4-methyl-1-piperazinyl)methyl]phenyl}carbonyl)-L-leucinamide;
1,1-Dimethylethyl 4-(4-{[((1S)-1-{[(4-{[(2,4-dichlorophenyl)sulfonyl]amino}butyl)amino]carbonyl}-3-methylbutyl)amino]carbonyl}phenyl)-1-piperazinecarboxylate;
N²-[(1S)-1-({[4-({[4-Fluoro-2-(trifluoromethyl)phenyl]sulfonyl}amino)butyl]amino}carbonyl)-3-methylbutyl]-5,6-dihydro-4H-cyclopenta[b]thiophene-2-carboxamide;
N²-(3-Cyclopentylpropanoyl)-N¹-[4-({[4-fluoro-2-(trifluoromethyl)phenyl]sulfonyl}amino)butyl]-L-leucinamide;
N²-(3-Cyclohexylpropanoyl)-N-[4-({[4-fluoro-2-(trifluoromethyl)phenyl]sulfonyl}amino)butyl]-L-leucinamide;
N²-(3-Cyclohexylpropanoyl)-N¹-(4-{[(2,4-dichlorophenyl)sulfonyl]amino}butyl)-L-leucinamide;
N²-(3-Cyclopentylpropanoyl)-N¹-(4-{[(2,4-dichlorophenyl)sulfonyl]amino}butyl)-L-leucinamide;
N¹-(4-{[(2-Chloro-4-fluorophenyl)sulfonyl]amino}butyl)-N²-(3-cyclohexylpropanoyl)-L-leucinamide;
N¹-(4-{[(2-Chloro-4-fluorophenyl)sulfonyl]amino}butyl)-N²-(3-cyclopentylpropanoyl)-L-leucinamide;
N¹-(4-{[(2-Chloro-4-fluorophenyl)sulfonyl]amino}butyl)-N²-(cyclohexylacetyl)-L-leucinamide;
N¹-(4-{[(2-Chloro-4-fluorophenyl)sulfonyl]amino}butyl)-N²-[(4-phenyl-2-thienyl)carbonyl]-L-leucinamide;
N¹-(4-{[(2-Chloro-4-fluorophenyl)sulfonyl]amino}butyl)-N²-[(2E)-3-(4-methylphenyl)-2-propenoyl]-L-leucinamide;
1,1-Dimethylethyl 5-{[((1S)-1-{[(4-{[(2-chloro-4-fluorophenyl)sulfonyl]amino}butyl)amino]carbonyl}-3-methylbutyl)amino]carbonyl}-3,4-dihydro-2(1H)-isoquinolinecarboxylate;
N¹-(4-{[(2-Chloro-4-fluorophenyl)sulfonyl]amino}butyl)-N²-[(5-phenyl-2-thienyl)carbonyl]-L-leucinamide;
N¹-(4-{[(2-Chloro-4-fluorophenyl)sulfonyl]amino}butyl)-N²-(2,3-dihydro-1H-inden-2-ylacetyl)-L-leucinamide;
N¹-(4-{[(2-Chloro-4-fluorophenyl)sulfonyl]amino}butyl)-N²-(1-cyclohexen-1-ylcarbonyl)-L-leucinamide;
1,1-Dimethylethyl 3-{3-[((1S)-1-{[(4-{[(2-chloro-4-fluorophenyl)sulfonyl]amino}butyl)amino]carbonyl}-3-methylbutyl)amino]-3-oxopropyl}-1-piperidinecarboxylate;
N-((1S)-1-{[(4-{[(2-chloro-4-fluorophenyl)sulfonyl]amino}butyl)amino]carbonyl}-3-methylbutyl)-2,3-dihydro-1H-indene-2-carboxamide;
N¹-(4-{[(2-chloro-4-fluorophenyl)sulfonyl]amino}butyl)-N²-[3-(2-chlorophenyl)propanoyl]-L-leucinamide;
N¹-(4-{[(2-chloro-4-fluorophenyl)sulfonyl]amino}butyl)-N²-[3-(3-chlorophenyl)propanoyl]-L-leucinamide;
1,1-Dimethylethyl 2-{3-[((1S)-1-{[(4-{[(2-chloro-4-fluorophenyl)sulfonyl]amino}butyl)amino]carbonyl}-3-methylbutyl)amino]-3-oxopropyl}-1-piperidinecarboxylate;
N¹-(4-{[(2-Chloro-4-fluorophenyl)sulfonyl]amino}butyl)-N²-[3-(4-chlorophenyl)propanoyl]-L-leucinamide;
N¹-(4-{[(2-Chloro-4-fluorophenyl)sulfonyl]amino}butyl)-N²-(cyclopentylacetyl)-L-leucinamide;
1,1-Dimethylethyl 2-{[((1S)-1-{[(4-{[(2-chloro-4-fluorophenyl)sulfonyl]amino}butyl)amino]carbonyl}-3-methylbutyl)amino]carbonyl}octahydro-1H-indole-1-carboxylate;
N¹-(4-{[(2-chloro-4-fluorophenyl)sulfonyl]amino}butyl)-N²-[3-(4-methylphenyl)propanoyl]-L-leucinamide;
N-((1S)-1-{[(4-{[(2-chloro-4-fluorophenyl)sulfonyl]amino}butyl)amino]carbonyl}-3-methylbutyl)furo[3,2-b]pyridine-2-carboxamide;
N¹-(4-{([(2-chloro-4-fluorophenyl)sulfonyl]amino}butyl)-N²-(tetrahydro-2H-thiopyran-4-ylacetyl)-L-leucinamide;
N-((1S)-1-{[(4-{[(2-chloro-4-fluorophenyl)sulfonyl]amino}butyl)amino]carbonyl}-3-methylbutyl)-2,3-dihydro-1H-indole-2-carboxamide;
1-Acetyl-N-((1S)-1-{[(4-{[(2-chloro-4-fluorophenyl)sulfonyl]amino}butyl)amino]carbonyl}-3-methylbutyl)-2,3-dihydro-1H-indole-2-carboxamide;
N¹-(4-{[(2-Chloro-4-fluorophenyl)sulfonyl]amino}butyl)-N²-(2,2-dimethyl-3-phenylpropanoyl)-L-leucinamide;
N²-[(4-Acetylphenyl)carbonyl]-N¹-(4-{[(2-chloro-4-fluorophenyl)sulfonyl]amino}butyl)-L-leucinamide;
N¹-(4-{[(2-chloro-4-fluorophenyl)sulfonyl]amino}butyl)-N²-[3-(3-nitrophenyl)propanoyl]-L-leucinamide;
N¹-((1S)-1-{[(4-([(2-chloro-4-fluorophenyl)sulfonyl]amino}butyl)amino]carbonyl)-3-methylbutyl)-1,3-benzothiazole-2-carboxamide;
N¹-(4-{[(2-chloro-4-fluorophenyl)sulfonyl]amino}butyl)-N²-(3-cyclohexyl-2,2-dimethylpropanoyl)-L-leucinamide;
N¹-(4-{[(2-chloro-4-fluorophenyl)sulfonyl]amino}butyl)-N²-(cyclopentylcarbonyl)-L-leucinamide;
N-((1S)-1-{[(4-{[(2-chloro-4-fluorophenyl)sulfonyl]amino}butyl)amino]carbonyl)-3-methylbutyl)-2-(phenylmethyl)-1,2,3,4-tetrahydro-5-isoquinolinecarboxamide;
N-((1S)-1-{[(4-{[(2-chloro-4-fluorophenyl)sulfonyl]amino}butyl)amino]carbonyl}-3-methylbutyl)-2-(phenylcarbonyl)-1,2,3,4-tetrahydro-5-isoquinolinecarboxamide;
N-((1S)-1-{[(4-{[(2-chloro-4-fluorophenyl)sulfonyl]amino}butyl)amino]carbonyl}-3-methylbutyl)-1-benzofuran-2-carboxamide;
N¹-(4-{[(2,4-dichlorophenyl)sulfonyl]amino}butyl)-N²-[(phenyloxy)acetyl]-L-leucinamide;
N¹-(4-{[(2,4-dichlorophenyl)sulfonyl]amino}butyl)-N²-({[2-(methyloxy)phenyl]oxy}acetyl)-L-leucinamide;
N²-{[(4-chloro-2-methylphenyl)oxy]acetyl}-N¹-(4-{[(2,4-dichlorophenyl)sulfonyl]amino}butyl)-L-leucinamide;
N¹-(4-{[(2,4-dichlorophenyl)sulfonyl]amino}butyl)-N²-([(2-methylphenyl)oxy]acetyl)-L-leucinamide;
N²-{[(2-chlorophenyl)oxy]acetyl}-N¹-(4-{[(2,4-dichlorophenyl)sulfonyl]amino}butyl)-L-leucinamide;
N¹-(4-{[(2,4-dichlorophenyl)sulfonyl]amino}butyl)-N²-[(1-methyl-1H-imidazol-4-yl)sulfonyl]-L-leucinamide;
N²-[(cyclohexylamino)carbonyl]-N¹-(4-{[(2,4-dichlorophenyl)sulfonyl]amino}butyl)-L-leucinamide;
N¹-(4-{[(2-chloro-4-fluorophenyl)sulfonyl]amino}butyl)-N²-[(cyclohexylamino)carbonyl]-L-leucinamide;
N-((1S)-1-{[(4-{[(2-chloro-4-fluorophenyl)sulfonyl]amino}butyl)amino]carbonyl}-3-methylbutyl)octahydro-2(1H)-isoquinolinecarboxamide;
N-((1S)-1-{[(4-{[(2-chloro-4-fluorophenyl)sulfonyl]amino}butyl)amino]carbonyl}-3-methylbutyl)-3,4-dihydro-2(1-isoquinolinecarboxamide;
N²-[(cyclohexylamino)carbonyl]-N¹-[4-({[4-fluoro-2-(trifluoromethyl)phenyl]sulfonyl}amino)butyl]-L-leucinamide;
N¹-[4-({[4-fluoro-2-(trifluoromethyl)phenyl]sulfonyl}amino)butyl]-N²-[(phenylamino)carbonyl]-L-leucinamide;
N²{-[(cyclopentylamino)carbonyl}-N¹-[4-({[4-fluoro-2-(trifluoromethyl)phenyl]sulfonyl}amino)butyl]-L-leucinamide;
N²-{[(cyclohexylmethyl)amino]carbonyl}-N¹-[4-({[4-fluoro-2-(trifluoromethyl)phenyl]sulfonyl}amino)butyl]-L-leucinamide;
N²-{[cyclohexyl(methyl)amino]carbonyl}-N¹-[4-({[4-fluoro-2-(trifluoromethyl)phenyl]sulfonyl}amino)butyl]-L-leucinamide;
N²-{[cyclopentyl(methyl)amino]carbonyl}-N¹-[4-({[4-fluoro-2-(trifluoromethyl)phenyl]sulfonyl}amino)butyl]-L-leucinamide;
N¹-{4-({[4-fluoro-2-(trifluoromethyl)phenyl]sulfonyl}amino)butyl)-N²-{[(phenylmethyl)amino]carbonyl}-L-leucinamide;
N²-([(cyclopentylmethyl)oxy]carbonyl}-N¹-[4-({[4-fluoro-2-(trifluoromethyl)phenyl]sulfonyl}amino)butyl]-L-leucinamide;
N²-{[(cyclohexylmethyl)oxy]carbonyl}-N¹-[4-({[4-fluoro-2-(trifluoromethyl)phenyl]sulfonyl}amino)butyl]-L-leucinamide;
N¹-(4-{[(2-chloro-4-fluorophenyl)sulfonyl]amino}butyl)-N²-{[(cyclopentylmethyl)oxy]carbonyl}-L-leucinamide;
N¹-(4-{[(2-chloro-4-fluorophenyl)sulfonyl]amino}butyl)-N²-{[(cyclohexylmethyl)oxy]carbonyl}-L-leucinamide;
N-((1S)-1-{[(4-{[(2,4-dichlorophenyl)sulfonyl]amino}butyl)amino]carbonyl}-3-methyl-3-buten-1-yl)-1-benzothiophene-2-carboxamide;
N-[(1S)-2-[(4-{[(2,4-dichlorophenyl)sulfonyl]amino}butyl)amino]-2-oxo-1-(1,3-thiazol-4-ylmethyl)ethyl]-1-benzothiophene-2-carboxamide;
N-((1S)-2-[(4-{[(2-chloro-4-fluorophenyl)sulfonyl]amino}butyl)amino]-1-{[(1,1-dimethylethyl)oxy]methyl}-2-oxoethyl)-1-benzothiophene-2-carboxamide;
N-{(1R)-2-[(4-{[(2-chloro-4-fluorophenyl)sulfonyl]amino}butyl)amino]-1-[(methylthio)methyl]-2-oxoethyl}-1-benzothiophene-2-carboxamide;
N-((1S,2R)-1-{[(4-{[(2-chloro-4-fluorophenyl)sulfonyl]amino}butyl)amino]carbonyl}-2-hydroxy-3-methylbutyl)-1-benzothiophene-2-carboxamide;
N-((1S,2S)-1-{[(4-{[(2-chloro-4-fluorophenyl)sulfonyl]amino}butyl)amino]carbonyl}-2-hydroxy-3,3-dimethylbutyl)-1-benzothiophene-2-carboxamide;
N-((1S)-3,3-dichloro-1-{[(4-{[(2,4-dichlorophenyl)sulfonyl]amino}butyl)amino]carbonyl}propyl)-1-benzothiophene-2-carboxamide;
N-((1S)-3,3-dichloro-1-{[(4-{[(2-chloro-4-fluorophenyl)sulfonyl]amino}butyl)amino]carbonyl}propyl)-1-benzothiophene-2-carboxamide;
(2S)-4,4-dichloro-N-(4-{[(2-chloro-4-fluorophenyl)sulfonyl]amino}butyl)-2-{[(cyclohexylamino)carbonyl]amino}butanamide;
N-(1-{[(4-{[(2,4-dichlorophenyl)sulfonyl]amino}butyl)amino]carbonyl}-3,3,3-trifluoropropyl)-1-benzothiophene-2-carboxamide;
N-((1S)-1-{[(4-{[(2,4-dichlorophenyl)sulfonyl]amino}butyl)amino]carbonyl}-3,3-difluoropropyl)-1-benzothiophene-2-carboxamide;
N-((1R)-1-{[(4-{[(2,4-dichlorophenyl)sulfonyl]amino}butyl)amino]carbonyl}-3-methylbutyl)-1-benzothiophene-2-carboxamide;
N-{(1S)-2-[(4-{[(2-chloro-4-fluorophenyl)sulfonyl]amino}butyl)amino]-2-oxo-1-phenylethyl}-1-benzothiophene-2-carboxamide;
N-(1-benzothien-2-ylcarbonyl)-N-(4-{[(2-chloro-4-fluorophenyl)sulfonyl]amino}butyl)-L-phenylalaninamide;
N-((1S)-1-{[(4-{[(2,4-difluorophenyl)sulfonyl]amino}butyl)amino]carbonyl}-3-methylbutyl)-1-benzothiophene-2-carboxamide;
N-((1S)-1-{[(4-{[(4-fluoro-2-methylphenyl)sulfonyl]amino}butyl)amino]carbonyl}-3-methylbutyl)-1-benzothiophene-2-carboxamide;
N-{(1S)-1-[({4-[{[4-fluoro-2-(trifluoromethyl)phenyl]sulfonyl}(methyl)amino]butyl}amino)carbonyl]-3-methylbutyl}-1-benzothiophene-2-carboxamide;
N-{(1S)-1-[({4-[[(2,4-dichlorophenyl)sulfonyl](methyl)amino]butyl}amino)carbonyl]-3-methylbutyl}-1-benzothiophene-2-carboxamide;
N-{(1S)-1-[({4-[[(2-chloro-4-fluorophenyl)sulfonyl](methyl)amino]butyl]amino)carbonyl}-3-methylbutyl}-1-benzothiophene-2-carboxamide;
N-{(1S)-1-[({4-[[(2-cyanophenyl)sulfonyl](methyl)amino]butyl}amino)carbonyl]-3-methylbutyl}-1-benzothiophene-2-carboxamide;
2-(3-biphenylyl)-N-(4-{[(2,4-dichlorophenyl)sulfonyl]amino}butyl)-4-methylpentanamide;
N-((1S)-1-{[((2S)-4-{[(2-chloro-4-fluorophenyl)sulfonyl]amino}-2-hydroxybutyl)amino]carbonyl}-3-methylbutyl)-1-benzothiophene-2-carboxamide;
N-((1S)-1-{[((2R)-4-{[(2-chloro-4-fluorophenyl)sulfonyl]amino}-2-hydroxybutyl)amino]carbonyl}-3-methylbutyl)-1-benzothiophene-2-carboxamide;
N-((1S)-3,3-dichloro-1-{[((2S)-4-{[(2,4-dichlorophenyl)sulfonyl]amino}-2-hydroxybutyl)amino]carbonyl}propyl)-1-benzothiophene-2-carboxamide;
W-[(cyclohexylamino)carbonyl]-N¹-((2S)-4-{[(2,4-dichlorophenyl)sulfonyl]amino}-2-hydroxybutyl)-L-leucinamide;
N-((1S)-1-{[((2S)-4-{[(2-chloro-4-fluorophenyl)sulfonyl]amino}-2-hydroxybutyl)amino]carbonyl}-3-methylbutyl)-1-benzofuran-2-carboxamide;
N-((1S)-1-{[((3S)-4-{[(2-chloro-4-fluorophenyl)sulfonyl]amino}-3-hydroxybutyl)amino]carbonyl}-3-methylbutyl)-1-benzothiophene-2-carboxamide;
N-((1S)-1-{[((3R)-4-{[(2-chloro-4-fluorophenyl)sulfonyl]amino}-3-hydroxybutyl)amino]carbonyl}-3-methylbutyl)-1-benzothiophene-2-carboxamide;
N-((1S)-1-{[((4R)-4-{[(2-chloro-4-fluorophenyl)sulfonyl]amino}-5-hydroxypentyl)amino]carbonyl}-3-methylbutyl)-1-benzothiophene-2-carboxamide;
N-((1S)-1-{[((4S)-4-{[(2-chloro-4-fluorophenyl)sulfonyl]amino}-5-hydroxypentyl)amino]carbonyl}-3-methylbutyl)-1-benzothiophene-2-carboxamide;
Methyl N⁵-[N-(1-benzothien-2-ylcarbonyl)-L-leucyl]-N²-[(2-chloro-4-fluorophenyl)sulfonyl]-D-ornithinate;
N⁵-[N-(1-benzothien-2-ylcarbonyl)-L-leucyl]-N²-[(2-chloro-4-fluorophenyl)sulfonyl]-N¹-methyl-D-ornithinamide;
N-((1S)-1-{[((2R,3R)-4-{[(2,4-dichlorophenyl)sulfonyl]amino}-2,3-dihydroxybutyl)amino]carbonyl}-3-methylbutyl)-1-benzothiophene-2-carboxamide;
N-((1S)-1-{[((2R,3R)-4-{[(2-chloro-4-fluorophenyl)sulfonyl]amino}-2,3-dihydroxybutyl)amino]carbonyl}-3-methylbutyl)-1-benzothiophene-2-carboxamide;
N-{(1S)-1-[({[(4R,5R)-5-({[(2,4-dichlorophenyl)sulfonyl]amino}methyl)-2,2-dimethyl-1,3-dioxolan-4-yl]methyl}amino)carbonyl]-3-methylbutyl}-1-benzothiophene-2-carboxamide;
N-{(1S)-1-[({[(4R,5R)-5-({[(2-chloro-4-fluorophenyl)sulfonyl]amino}methyl)-2,2-dimethyl-1,3-dioxolan-4-yl]methyl}amino)carbonyl]-3-methylbutyl}-1-benzothiophene-2-carboxamide;
N-{(1S)-1-[({[(4R,5R)-5-({[(2,4-dichlorophenyl)sulfonyl]amino}methyl)-1,3-dioxolan-4-yl]methyl}amino)carbonyl]-3-methylbutyl}-1-benzothiophene-2-carboxamide;
N-{(1S)-1-[({[(4R,5R)-5-({[(2-chloro-4-fluorophenyl)sulfonyl]amino}methyl)-1,3-dioxolan-4-yl]methyl}amino)carbonyl]-3-methylbutyl}-1-benzothiophene-2-carboxamide;
N-{(1S)-1-[({[(2S,3R)-2-([[(2-chloro-4-fluorophenyl)sulfonyl]amino}methyl)tetrahydro-3-furanyl]methyl}amino)carbonyl]-3-methylbutyl]-1-benzothiophene-2-carboxamide; and
N-{(1S)-1-[({[(2,3R)-3-({[(2-chloro-4-fluorophenyl)sulfonyl]amino}methyl)tetrahydro-2-furanyl]methyl}amino)carbonyl]-3-methylbutyl}-1-benzothiophene-2-carboxamide;
N-{(1S)-1-[({[2-({[(2,4-dichlorophenyl)sulfonyl]amino}methyl)cyclopentyl]methyl}amino)carbonyl]-3-methylbutyl}-1-benzothiophene-2-carboxamide;
N-{(1S)-1-[({[2-({[(2-chloro-4-fluorophenyl)sulfonyl]amino}methyl)cyclopentyl]methyl}amino)carbonyl]-3-methylbutyl}-1-benzothiophene-2-carboxamide;
2,4-dichloro-N-{4-[[(cyclohexylamino)carbonyl]((2S)-4-methyl-2-{[(phenylamino) carbonyl]amino}pentyl)amino]butyl}benzenesulfonamide;
2,4-dichloro-N-{4-[[(4-fluorophenyl)sulfonyl]((2S)-4-methyl-2-{[(phenylamino) carbonyl]amino}pentyl)amino]butyl}benzenesulfonamide;
N-(4-{[(2,4-dichlorophenyl)sulfonyl]amino}butyl)-N-((2S)-4-methyl-2-{[(phenylamino) carbonyl]amino}pentyl)acetamide;
methyl (4-{[(2,4-dichlorophenyl)sulfonyl]amino}butyl)-N-((2S)-4-methyl-2-{[(phenylamino) carbonyl]amino}pentyl)carbamate;
2,4-dichloro-N-{4-[[(ethylamino)carbonyl]((2S)-4-methyl-2-{[(phenylamino) carbonyl]amino}pentyl)amino]butyl}benzenesulfonamide;
2,4-dichloro-N-{4-[{[(1-methylethyl)amino]carbonyl}((2S)-4-methyl-2-{[(phenylamino)carbonyl]amino}pentyl)amino]butyl)benzenesulfonamide;
2,4-dichloro-N-(4-{((2S)-4-methyl-2-{[(phenylamino)carbonyl]amino}pentyl)[(phenylamino) carbonyl]amino}butyl)benzenesulfonamide;
N-(4-{[(2,4-dichlorophenyl)sulfonyl]amino}butyl)-N-((2S)-4-methyl-2-{[(phenylamino)carbonyl]amino}pentyl)cyclohexanecarboxamide; 2,4-dichloro-N-(4-({[(1-methylethyl)amino]carbonyl}[(2S)-4-methyl-2-({[(1-methylethyl)amino]carbonyl}amino)pentyl]amino}butyl)benzenesulfonamide;
2,4-dichloro-N-[4-(((2S)-2-{[(cyclohexylamino)carbonyl]amino}-4-methylpentyl){[(1-methylethyl)amino]carbonyl}amino)butyl]benzenesulfonamide;
2,4-dichloro-N-[4-(((2S)-2-{[(ethylamino)carbonyl]amino}-4-methylpentyl){[(1-methylethyl)amino]carbonyl}amino)butyl]benzenesulfonamide;
2,4-dichloro-N-[4-([(2S)-2-({[(4-fluorophenyl)amino]carbonyl}amino)-4-methylpentyl]{[(1-methylethyl)amino]carbonyl}amino)butyl]benzenesulfonamide;
2,4-dichloro-N-[4-({[(1-methylethyl)amino]carbonyl}{(2S)-4-methyl-2-[({[4-(1H-pyrrol-1-yl)phenyl]amino}carbonyl)amino]pentyl}amino)butyl]benzenesulfonamide;
2,4-dichloro-N-(4-{{[(1-methylethyl)amino]carbonyl}[(2S)-4-methyl-2-({[{1,3,5-trimethyl-1H-pyrazol-4-yl)amino]carbonyl}amino)pentyl]amino}butyl)benzenesulfonamide;
2,4-dichloro-N-{4-[[(cyclopropylamino)carbonothioyl]((2S)-4-methyl-2-{[(phenylamino) carbonyl]amino}pentyl)amino]butyl}benzenesulfonamide;
2,4-dichloro-N-{4-[{[(1,1-dimethylethyl)amino]carbonyl}((2S)-4-methyl-2-{[(phenylamino) carbonyl]amino}pentyl)amino]butyl}benzenesulfonamide;
2,4-dichloro-N-{4-[({[4-(dimethylamino)phenyl]amino}carbonyl}((2S)-4-methyl-2-{[(phenylamino)carbonyl]amino}pentyl)amino]butyl}benzenesulfonamide;
2,4-dichloro-N-{4-[{[(2-furanylmethyl)amino]carbonyl}((2S)-4-methyl-2-{[(phenylamino)carbonyl]amino}pentyl)amino]butyl}benzenesulfonamide;
2,4-dichloro-N-(4-{((2S)-4-methyl-2-{[(phenylamino)carbonyl]amino}pentyl)[(3-thienylamino) carbonyl]amino}butyl)benzenesulfonamide;
2,4-dichloro-N-{4-[((2S)-4-methyl-2-{[(phenylamino)carbonyl]amino}pentyl)({[1-(trifluoroacetyl)-4-piperidinyl]amino}carbonyl)amino]butyl}benzenesulfonamide;
2,4-dichloro-N-{4-[{[(1,1-dioxidotetrahydro-3-thienyl)amino]carbonyl}((2S)-4-methyl-2-{[(phenylamino)carbonyl]amino}pentyl)amino]butyl}benzenesulfonamide;
2,4-dichloro-N-{4-[[(methylamino)carbonothioyl]((2S)-4-methyl-2-{[(phenylamino) carbonyl]amino}pentyl)amino]butyl}benzenesulfonamide;
2,4-dichloro-N-{4-[{[(1-methylethyl)amino]carbonyl}((2S)-4-methyl-2-{[(2-pyridinylamino)carbonothioyl]amino}pentyl)amino]butyl}benzenesulfonamide;
2,4-dichloro-N-{4-[{[(1-methylethyl)amino]carbonyl}((2S)-4-methyl-2-{[(phenylamino) carbonothioyl]amino}pentyl)amino]butyl}benzenesulfonamide;
2,4-dichloro-N-{4-[{[(1-methylethyl)amino]carbonyl}{(2S)-4-methyl-2-{[(3-thienylamino) carbonyl]amino}pentyl)amino]butyl}benzenesulfonamide;
2,4-dichloro-N-[4-({[(1-methylethyl)amino]carbonyl)}{(2S)-4-methyl-2-[({[1-(trifluoroacetyl)-4-piperidinyl]amino}carbonyl)amino]pentyl}amino)butyl]benzenesulfonamide;
2,4-dichloro-N-{4-[{[(1-methylethyl)amino]carbonyl}((2S)-4-methyl-2-{[(2-pyridinylamino)carbonyl]amino}pentyl)amino]butyl}benzenesulfonamide;
2,4-dichloro-N-[4-(((2S)-2-{[(cyclopentylamino)carbonyl]amino}-4-methylpentyl){[(1-methyl ethyl)amino]carbonyl}amino)butyl]benzenesulfonamide;
N-(4-{[(2,4-dichlorophenyl)sulfonyl]amino}butyl)-N-((2S)-4-methyl-2-{[(phenylamino)carbonyl]amino}pentyl)-2-phenylacetamide;
2,4-dichloro-N-[4-([(2R)-2-hydroxy-3-(methyloxy)propyl]{[(1-methylethyl)amino]carbonyl}amino)butyl]benzenesulfonamide;
(1R)-2-((4-{[(2,4-dichlorophenyl)sulfonyl]amino}butyl){[(1-methylethyl)amino]carbonyl}amino)-1-[(methyloxy)methyl]ethyl phenylcarbamate;
(1R)-2-{(4-{[(2,4-dichlorophenyl)sulfonyl]amino}butyl)[(ethylamino)carbonyl]amino}-1-[(methyloxy)methyl]ethyl phenylcarbamate;
(1R)-2-{(4-{[(2,4-dichlorophenyl)sulfonyl]amino}butyl)[(phenylamino)carbonyl]amino}-1-[(methyloxy)methyl]ethyl phenylcarbamate;
2,4-dichloro-N-[4-({(2R)-2-hydroxy-3-[(phenylmethyl)oxy]propyl}{[(1-methylethyl)amino]carbonyl}amino)butyl]benzenesulfonamide;
(1R)-2-((4-{[(2,4-dichlorophenyl)sulfonyl]amino}butyl){[(1-methylethyl)amino]carbonyl}amino)-1-{[(phenylmethyl)oxy]methyl}ethyl phenylcarbamate;
2,4-dichloro-N-[4-([(2S)-2-hydroxy-3-(4-morpholinyl)propyl]{[(1-methylethyl)amino]carbonyl}amino)butyl]benzenesulfonamide;
(1S)-2-((4-{[(2,4-dichlorophenyl)sulfonyl]amino}butyl){[(1-methylethyl)amino]carbonyl}amino)-1-(4-morpholinylmethyl)ethyl phenylcarbamate;
(1R)-2-((4-{[(2-chloro-4-fluorophenyl)sulfonyl]amino}butyl){[(1-methylethyl)amino]carbonyl}amino)-1-[(methyloxy)methyl]ethyl phenylcarbamate;
(1R)-2-([4-({[4-fluoro-2-(trifluoromethyl)phenyl]sulfonyl}amino)butyl]{[(1-methylethyl)amino]carbonyl}amino)-1-[(methyloxy)methyl]ethyl phenylcarbamate;
(1R)-2-[{[(1-methylethyl)amino]carbonyl}(4-{[(2-nitrophenyl)sulfonyl]amino}butyl)amino]-1-[(methyloxy)methyl]ethyl phenylcarbamate;
(1R)-2-((4-{[(2,4-dichlorophenyl)sulfonyl]amino}butyl){[(1-methylethyl)amino]carbonyl}amino)-1-[(methyloxy)methyl]ethyl (4-fluorophenyl)carbamate;
(1S)-2-((4-{[(2,4-dichlorophenyl)sulfonyl]amino}butyl){[(1-methylethyl)amino]carbonyl}amino)-1-[(methyloxy)methyl]ethyl phenylcarbamate;
(1R)-2-{(4-{[(2-chloro-4-fluorophenyl)sulfonyl]amino}butyl)[(ethylamino)carbonyl]amino}-1-[(methyloxy)methyl]ethyl phenylcarbamate;
(1R)-2-{[(ethylamino)carbonyl][4-({[4-fluoro-2-(trifluoromethyl)phenyl]sulfonyl}amino)butyl]amino}-1-[(methyloxy)methyl]ethyl phenylcarbamate;
O-{(1R)-2-{(4{[(2,4-dichlorophenyl)sulfonyl]amino}butyl)[(ethylamino)carbonyl]amino}-1-[(methyloxy)methyl]ethyl}phenylthiocarbamate;
(1R)-2-[{[(1-methylethyl)amino]carbonyl}(4-{methyl[(2-nitrophenyl)sulfonyl]amino}butyl)amino]-1-[(methyloxy)methyl]ethyl phenylcarbamate;
(1R)-2-({4-[[(2-cyanophenyl)sulfonyl](methyl)amino]butyl}{[(1-methylethyl)amino]carbonyl}amino)-1-[(methyloxy)methyl]ethyl phenylcarbamate;
2-[[(cyclohexylamino)carbonyl](4-{[(2,4-dichlorophenyl)sulfonyl]amino}butyl)amino]ethyl phenylcarbamate;
2-[[(cyclohexylamino)carbonyl](4-{[(2,4-dichlorophenyl)sulfonyl]amino}butyl)amino]ethyl cyclohexylcarbamate;
2-[[(cyclohexylamino)carbonyl](4-{[(2,4-dichlorophenyl)sulfonyl]amino}butyl)amino]ethyl methylcarbamate;
2-chlorophenyl[2-((4-{[(2,4-dichlorophenyl)sulfonyl]amino}butyl){[(1-methylethyl)amino]carbonyl}amino)ethyl]carbamate;
2,4-dichloro-N-{4-[{[(1-methylethyl)amino]carbonyl}((2R)-3-(methyloxy)-2-{[(phenylamino)carbonyl]amino}propyl)amino]butyl}benzenesulfonamide;
2-[((4-{[(2,4-dichlorophenyl)sulfonyl]amino}butyl){[(1-methylethyl)amino]carbonyl}amino)methyl]-N-phenyl-1-piperidinecarboxamide;
(3R)-3-((4-{[(2,4-dichlorophenyl)sulfonyl]amino}butyl){[(1-methylethyl)amino]carbonyl}amino)-N-phenyl-1-piperidinecarboxamide;
(3S)-3-((4-{[(2,4-dichlorophenyl)sulfonyl]amino}butyl){[(1-methylethyl)amino]carbonyl}amino)-N-phenyl-1-piperidinecarboxamide;
2-[((4-{[(2,4-dichlorophenyl)sulfonyl]amino}butyl){[(1-methylethyl)amino]carbonyl}amino)methyl]-N-phenyl-1-pyrrolidinecarboxamide;
2-({(4-{[(2,4-dichlorophenyl)sulfonyl]amino}butyl)[(ethylamino)carbonyl]amino}methyl)-N-phenyl-1-pyrrolidinecarboxamide;
N-(4-[[(2,4-dichlorophenyl)sulfonyl]amino}butyl)-2,2-dimethyl-N-((2S)-4-methyl-2-{[(phenylamino)carbonyl]amino}pentyl)hydrazinecarboxamide;
2,4-dichloro-N-{4-[{[(2-fluoroethyl)amino]carbonyl}((2S)-4-methyl-2-{[(phenylamino)carbonyl]amino}pentyl)amino]butyl}benzenesulfonamide;
2-chloro-4-fluoro-N-{4-[{[(1-methylethyl)amino]carbonyl}((2S)-4-methyl-2-{[(phenylamino)carbonyl]amino}pentyl)amino]butyl}benzenesulfonamide; and
4-fluoro-N-{4-[{[(1-methylethyl)amino]carbonyl}((2S)-4-methyl-2-{[(phenylamino)carbonyl]amino}pentyl)amino]butyl}-2-(trifluoromethyl)benzenesulfonamide
and pharmaceutically acceptable salts, hydrates, or solvates thereof.

13. A method of activating a TRPV4 channel receptor in a patient, comprising administering to the patient an effective amount of a compound according to claim 12.

14. A method for treating a patient suffering from a disease affecting cartilage or matrix degradation comprising the step of contacting at least one cell expressing a TRPV4 channel receptor of the patient with a therapeutically effective amount of a compound of claim 12.

15. The method of claim 14, wherein the patient is suffering from a disease selected from the group consisting of: chronic pain, neuropathic pain, postoperative pain, osteoarthritis, neuralgia, neuropathies, algesia, nerve injury, ischaemia, neurodegeneration, cartilage degeneration, and inflammatory disorders.

16. The method according to claim 15 wherein said disease is osteoarthritis.

17. The method according to claim 15 wherein said disease is rheumatoid arthritis.

18. The compound of claim 1, wherein R⁸ is phenyl substituted with one to three substituents selected from the group consisting of: C₂O, NO₂, dimethylpropanoyl, methylpiperazinyl, phenyl, piperazine further substituted with dimethylethylcarbonyl, amino, halogen, CH₃, C₁-C₁₂ alkyl, C₁-C₁₂ alkoxy, amino sulfonyl, and alkylsulfonyl groups and pharmaceutically acceptable salts, hydrates, or solvates thereof.

19. The compound of claim 1 wherein R⁸ is isoquinoline further substituted with one to three substituents selected from the group consisting of dimethylethylcarbonyl and phenylcarbonyl and pharmaceutically acceptable salts, hydrates, or solvates thereof.

20. A pharmaceutical composition comprising a compound according to claim 2 and a pharmaceutically acceptable carrier, diluents or excipient.
